(19) United States
(12) Patent Application Publication
(10) Pub. No.: US 2024/0130149 A1
(43) Pub. Date: Apr. 18, 2024

(54) AMINE COMPOUND AND ORGANIC LIGHT-EMITTING DEVICE USING THE SAME

(71) Applicant: Samsung Display Co., Ltd., Yongin-si (KR)

(72) Inventors: Dongjun Kim, Yongin-si (KR); Beomjoon Kim, Yongin-si (KR); Chaeyeong Kim, Yongin-si (KR); Eunjae Jeong, Yongin-si (KR); Hanna Cha, Yongin-si (KR); Sanghyun Han, Yongin-si (KR)

(21) Appl. No.: 18/463,072

(22) Filed: Sep. 7, 2023

(30) Foreign Application Priority Data
Sep. 14, 2022 (KR) 10-2022-0115803

(51) Int. Cl. H10K 50/11; C07C 211/61; H10K 50/15; H10K 50/16; H10K 85/60

(52) U.S. Cl. CPC H10K 50/11; C07C 211/61; H10K 50/15; H10K 50/16; H10K 85/633

(57) ABSTRACT

A light-emitting device may include a first electrode, a second electrode facing the first electrode, an interlayer between the first electrode and the second electrode and including an emission layer, and an amine-containing compound represented by Formula 1:

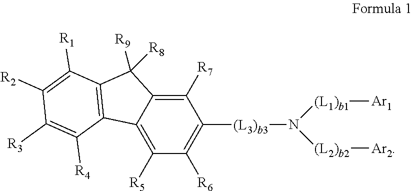

### CROSS-REFERENCE TO RELATED APPLICATION

This application claims priority to and the benefit of Korean Patent Application No. 10-2022-0115803, filed on Sep. 14, 2022, in the Korean Intellectual Property Office, the content of which is incorporated by reference herein in its entirety.

### BACKGROUND

### 1. Field

One or more embodiments of the present disclosure relate to an amine-containing compound, a light-emitting device including the same, an electronic device including the light-emitting device, and an electronic apparatus including the electronic device.

### 2. Description of the Related Art

An organic light-emitting device may have wider viewing angles, higher contrast ratios, and shorter response times, compared to an inorganic light-emitting device. An organic light-emitting device may include a first electrode, a hole transport region, an emission layer, an electron transport region, and a second electrode, which are arranged sequentially in the stated order. Holes injected from the first electrode may move to the emission layer through the hole transport region. Electrons injected from the second electrode may move to the emission layer through the electron transport region. Carriers such as the holes and the electrons may combine in the emission layer. The carriers may combine to generate excitons. These excitons transition and relax from an excited state to a ground state to generate light.

### SUMMARY

One or more aspects of embodiments of the present disclosure are directed toward an amine-containing compound with improved hole transport characteristics and a light-emitting device having a low driving voltage, a high luminance, a high luminescence efficiency, and a long lifespan by employing the amine-containing compound. One or more aspects of embodiments of the present disclosure are directed toward a high-quality electronic apparatus and electronic device including the light-emitting device.

Additional aspects will be set forth in part in the description which follows and, in part, will be apparent from the description, or may be learned by practice of the presented embodiments of the disclosure.

According to one or more embodiments of the present disclosure, provided is an amine-containing compound represented by Formula 1:

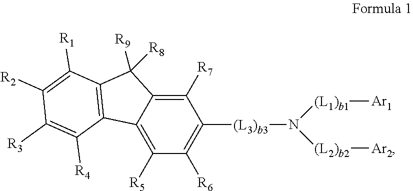

wherein, in Formula 1,
R₁ to R₉ may each independently be a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one selected from among R₁ to R₄ may be the group represented by Formula 2,
R₈ and R₉ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
at least one selected from among Ar₁ and Ar₂ may be a saturated C₃-C₆₀ cyclic group unsubstituted or substituted with at least one R₁₀ₐ,
L₁ to L₃ may each independently be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 to b3 may each independently be an integer from 0 to 5,

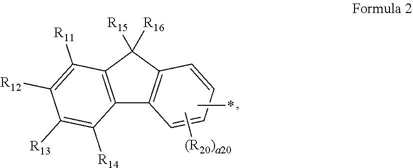

wherein, in Formula 2,
R₁₁ to R₁₆ and R₂₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
* may indicate a binding site to a neighboring atom,
a20 may be an integer from 0 to 3,
R₁₅ and R₁₆ may optionally be bonded to each other to form a C₃-C₆₀ carbocyclic group unsubstituted or substituted with at least one R₁₀ₐ or a C₁-C₁₀ heterocyclic group unsubstituted or substituted with at least one R₁₀ₐ,

R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments of the present disclosure, a light-emitting device may include the amine-containing compound represented by Formula 1.

According to one or more embodiments of the present disclosure, an electronic device may include the light-emitting device.

According to one or more embodiments of the present disclosure, provided is an electronic apparatus utilizing the electronic device.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic diagram of a light-emitting device according to one or more embodiments of the present disclosure;

FIG. 2 is a schematic diagram of an electronic device according to one or more embodiments of the present disclosure;

FIG. 3 is a schematic diagram of an electronic device according to one or more embodiments of the present disclosure;

FIG. 4 is a schematic perspective view of an electronic apparatus including a light-emitting device according to one or more embodiments of the present disclosure;

FIG. 5 is a schematic diagram of an exterior of a vehicle as an electronic apparatus including a light-emitting device according to one or more embodiments of the present disclosure; and

FIGS. 6A-6C are schematic diagrams of an interior of a vehicle according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

According to one or more aspects of embodiments of the present disclosure, a light-emitting device may include a first electrode, a second electrode facing the first electrode, an interlayer between the first electrode and the second electrode and including an emission layer, and an amine-containing compound represented by Formula 1.

According to one or more embodiments, the first electrode may be an anode. The second electrode may be a cathode. The emission layer may include a dopant and a host and may be to emit light. The dopant and the host are to be described later.

The term “interlayer” as utilized herein may refer to a single layer and/or all layers between a first electrode and a second electrode of a light-emitting device.

According to one or more embodiments, the interlayer may further include a hole transport region between the first electrode and the emission layer and an electron transport region between the emission layer and the second electrode. The hole transport region may include a hole injection layer, a hole transport layer, an emission auxiliary layer, an electron-blocking layer, or any combination thereof. The electron transport region may include a buffer layer, a hole-blocking layer, an electron control layer, an electron transport layer, an electron injection layer, or any combination thereof.

For example, in some embodiments, the hole transport region may include a hole injection layer on the first electrode and a hole transport layer between the hole injection layer and the emission layer. The hole injection layer may have a single-layer structure or a multi-layer structure. The hole transport layer may have a single-layer structure or a multi-layer structure. For example, in some embodiments, the hole transport layer may include a first hole transport layer, a second hole transport layer, and a third hole transport layer, which are arranged in the stated order from the hole injection layer.

For example, in some embodiment, the electron transport region may include an electron transport layer on the emission layer, and an electron injection layer between the electron transport layer and the second electrode.

According to one or more embodiments, the amine-containing compound may be included in the interlayer.

According to one or more embodiments, the amine-containing compound may be included in the hole transport region.

According to one or more embodiments, the amine-containing compound may be included in the hole transport layer, and the hole transport layer and the emission layer may be in direct contact with each other. For example, in one embodiment, the amine-containing compound may be included in the third hole transport layer. In another embodiment, the amine-containing compound may be included in the first hole transport layer and the third hole transport layer. In yet another embodiment, the amine-containing compound may be included in the first to third hole transport layers.

According to one or more embodiments, the light-emitting device may further include a capping layer outside of the first electrode, and the amine-containing compound may be included in the capping layer.

According to one or more embodiments, the light-emitting device may further include a first capping layer outside of the first electrode and a second capping layer outside of the second electrode, and the amine-containing compound may be included in the first capping layer and/or the second capping layer. For example, in some embodiments, the amine-containing compound may be included in the first capping layer, among the first capping layer, the first electrode, and the interlayer, which are arranged in the stated order. In some embodiments, the amine-containing compound may be included in the second capping layer, among the interlayer, the second electrode, and the second capping layer, which are arranged in the stated order. In some embodiments, the amine-containing compound may be included in both (e.g., simultaneously) of the first capping layer and the second capping layer.

According to one or more aspects of embodiments of the present disclosure, provided is an electronic device including the light-emitting device.

According to one or more embodiments, the electronic device may further include a thin-film transistor electrically connected with the light-emitting device, and a color filter, a color conversion layer, a touch screen layer, a polarization layer, or any combination thereof. For example, in some embodiments, the electronic device may include the light-emitting device, the thin-film transistor, and the color filter. In some embodiments, the electronic device may include the light-emitting device, the thin-film transistor, the color filter, and the color conversion layer.

According to one or more aspects of embodiments of the present disclosure, provided is an electronic apparatus including the electronic device. The electronic apparatus may be at least one selected from a flat panel display, a curved display, a computer monitor, a medical monitor, a TV, a billboard, indoor or outdoor illuminations and/or signal light, a head-up display, a fully or partially transparent display, a flexible display, a rollable display, a foldable display, a stretchable display, a laser printer, a phone, a cell phone, a tablet, a phablet, a personal digital assistant (PDA), a wearable device, laptop computers, digital cameras, camcorders, viewfinders, micro displays, three-dimensional (3D) displays, virtual or augmented reality displays, vehicles, a video wall including multiple displays tiled together, a theater or stadium screen, a phototherapy device, and a signage.

According to one or more aspects of embodiments of the present disclosure, provided is an amine-containing compound represented by Formula 1:

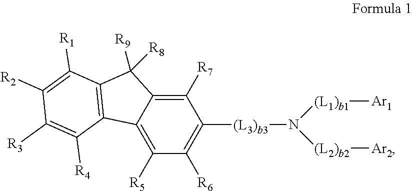

wherein, in Formula 1,
R₁ to R₉ may each independently be a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one selected from among R₁ to R₄ may be the group represented by Formula 2,
R₈ and R₉ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
at least one selected from among Ar₁ and Ar₂ may be a saturated C₃-C₆₀ cyclic group unsubstituted or substituted with at least one R₁₀ₐ,
L₁ to L₃ may each independently be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 to b3 may each independently be an integer from 0 to 5,

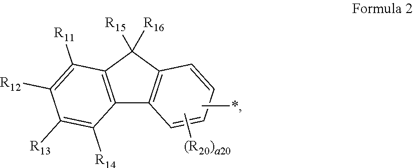

wherein, in Formula 2,
R₁₁ to R₁₆ and R₂₀ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
* may indicate a binding site to a neighboring atom,
a20 may be an integer from 0 to 3,
R₁₅ and R₁₆ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,

R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, at least one selected from among Ar₁ and Ar₂ may be a C₃-C₃₀ cycloalkyl group unsubstituted or substituted with at least one R₁₀ₐ.

According to one or more embodiments, at least one selected from among Ar₁ and Ar₂ may be a cyclohexyl group, a cycloheptyl group, a cyclooctyl group, an adamantanyl group, or a norbornanyl group, each unsubstituted or substituted with at least one R₁₀ₐ.

According to one or more embodiments, at least one selected from among Ar₁ and Ar₂ may be: a cyclohexyl group, a cycloheptyl group, or a cyclooctyl group, each unsubstituted or substituted with at least one R₁₀ₐ; or
a group represented by any one selected from Formulae 3-1 to 3-5:

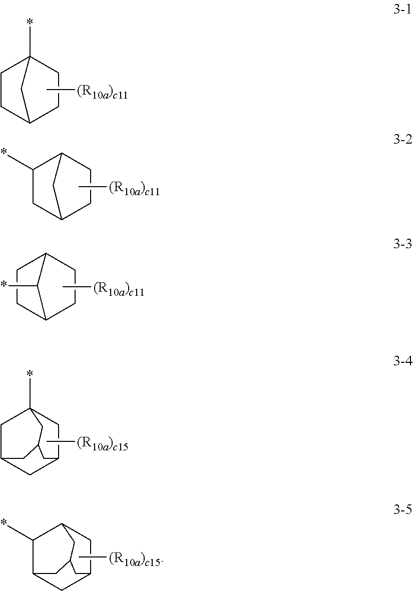

In Formulae 3-1 to 3-5,
R₁₀ₐ may be the same as described herein,
c11 may be an integer from 0 to 11,
c15 may be an integer from 0 to 15, and
* may indicate a binding site to a neighboring atom.

According to one or more embodiments, Ar₁ and Ar₂ may each independently be a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group, each unsubstituted or substituted with at least one R₁₀ₐ.

According to one or more embodiments, Ar₁ and Ar₂ may each independently be: a cyclohexyl group, a cycloheptyl group, a cyclooctyl group, or a phenyl group, each unsubstituted or substituted with at least one R₁₀ₐ; or a group represented by any one of Formulae 4-1 to 4-15:

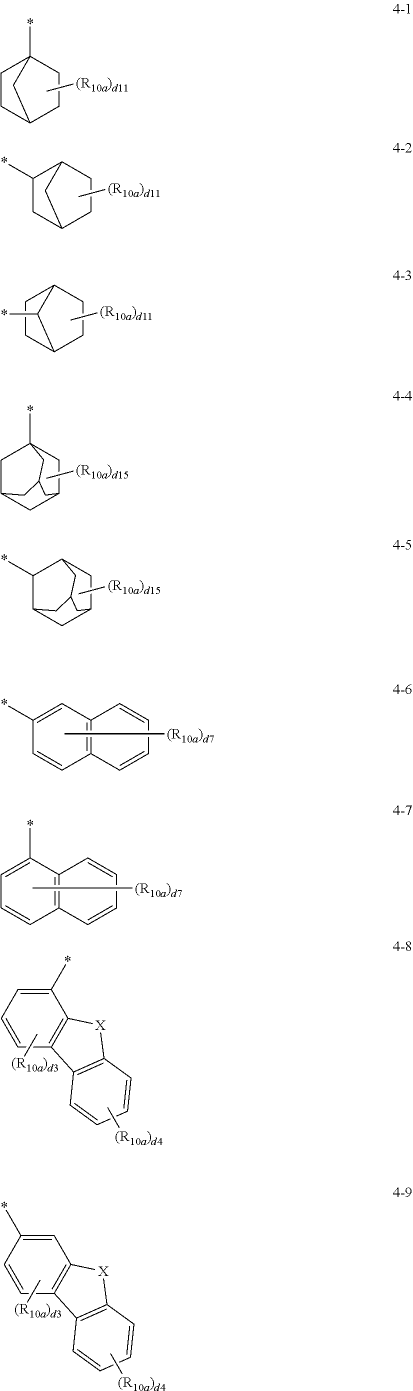

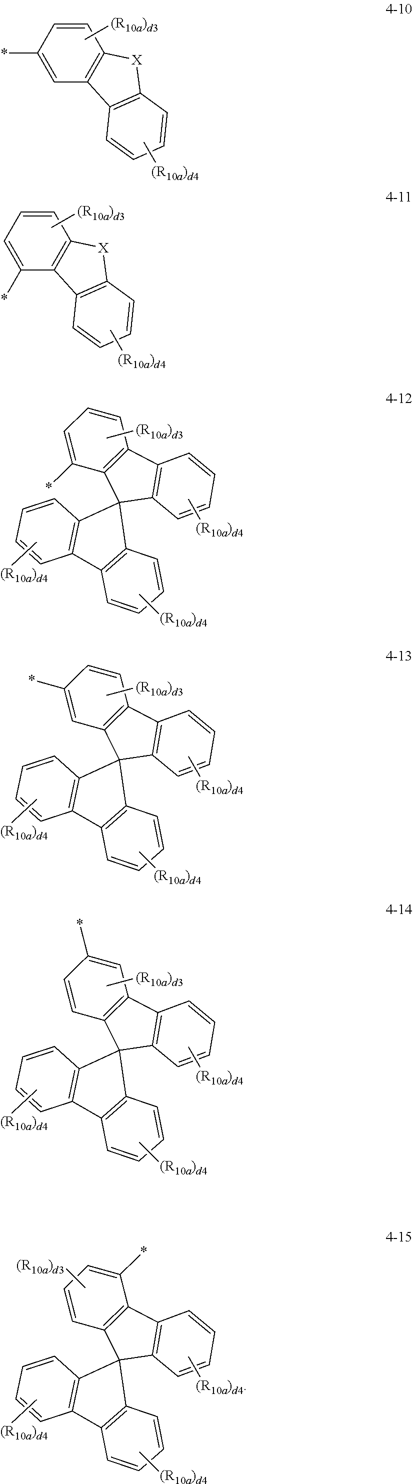

In Formulae 4-1 to 4-15,
R₁₀ₐ may be the same as described herein,
X may be C(R_{10b})(R_{10c}), N(R_{10d}), O, or S,
R_{10b}, R_{10c}, and R_{10d} may each be the same as described herein with respect to R₁₀ₐ,
d3 may be an integer from 0 to 3,
d4 may be an integer from 0 to 4,
d7 may be an integer from 0 to 7,
d11 may be an integer from 0 to 11,
d15 may be an integer from 0 to 15, and
* may indicate a binding site to a neighboring atom.

According to one or more embodiments, L₁ to L₃ may each independently be: a single bond; or a benzene group, a naphthalene group, an anthracene group, a phenanthrene group, a triphenylene group, a pyrene group, a chrysene group, cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

According to one or more embodiments, L₁ to L₃ may each independently be: a single bond; or a benzene group, a naphthalene group, an anthracene group, a phenanthrene group, a triphenylene group, a pyrene group, or a chrysene group, each unsubstituted or substituted with at least one R₁₀ₐ.

According to one or more embodiments, L₁ to L₃ may each independently be: a single bond; or a group represented by Formulae 5-1 to 5-13:

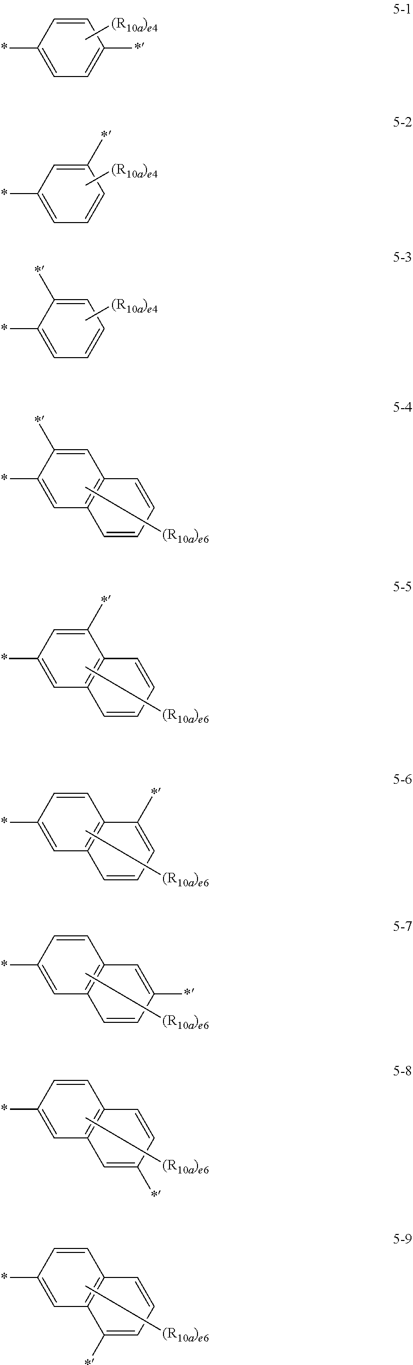

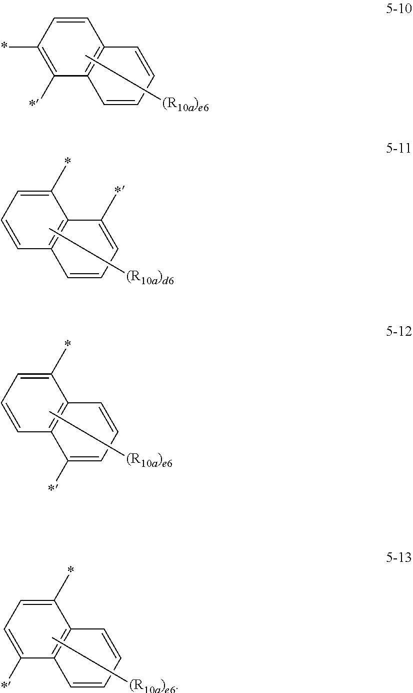

In Formulae 5-1 to 5-13,
R₁₀ₐ may be the same as described herein,
e4 may be an integer from 0 to 4,
e6 may be an integer from 0 to 6, and
* and *′ may each indicate a binding site to a neighboring atom.

According to one or more embodiments, b3 may be 0.

According to one or more embodiments, any one selected from R₁, R₃ and R₄ may be a group represented by Formula 2.

According to one or more embodiments, the group represented by Formula 2 may be a group represented by any one selected from Formulae 2-1 to 2-4:

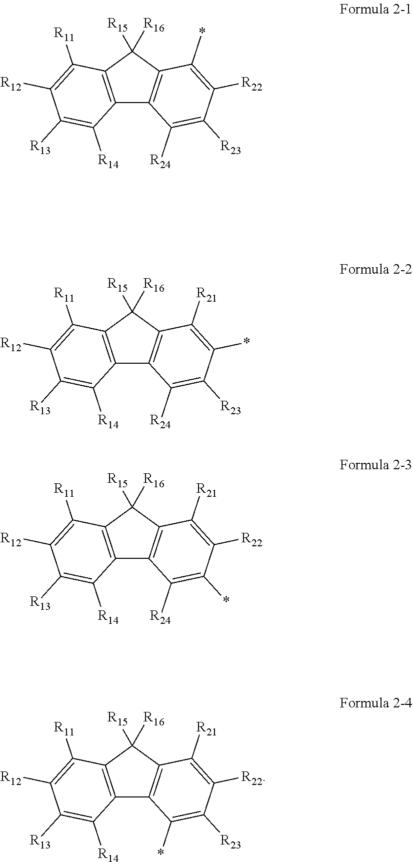

In Formulae 2-1 to 2-4,
R₁₁ to R₁₆ may each be the same as described herein,
R₂₁ to R₂₄ may each independently be the same as described herein with respect to R₂₀, and
* may indicate a binding site to a neighboring atom.

In one or more embodiments, Formula 1 may be represented by any one selected from Formulae 1-1 to 1-16:

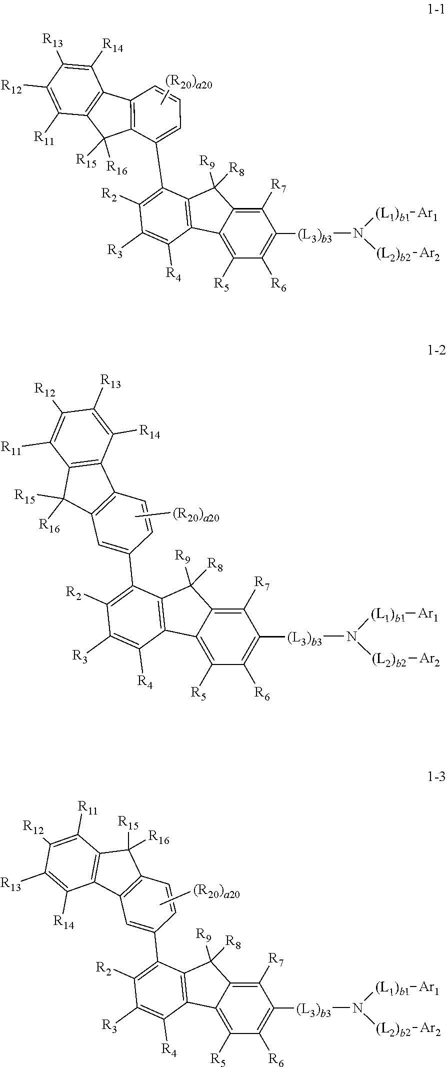

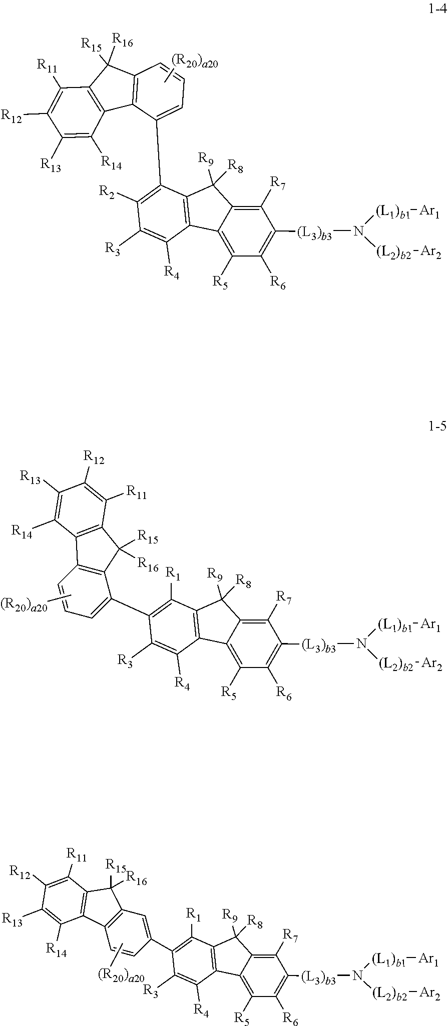

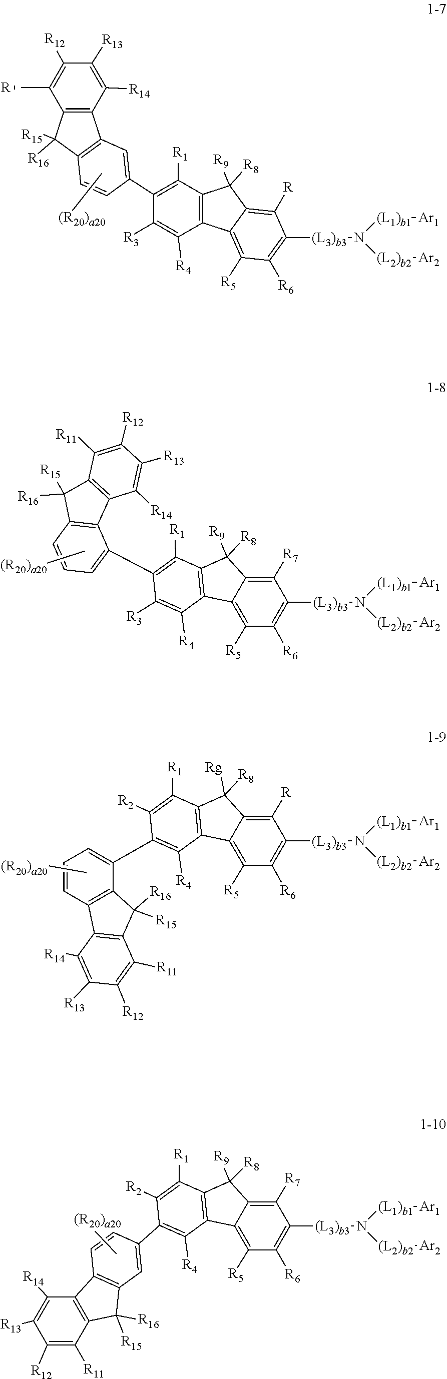

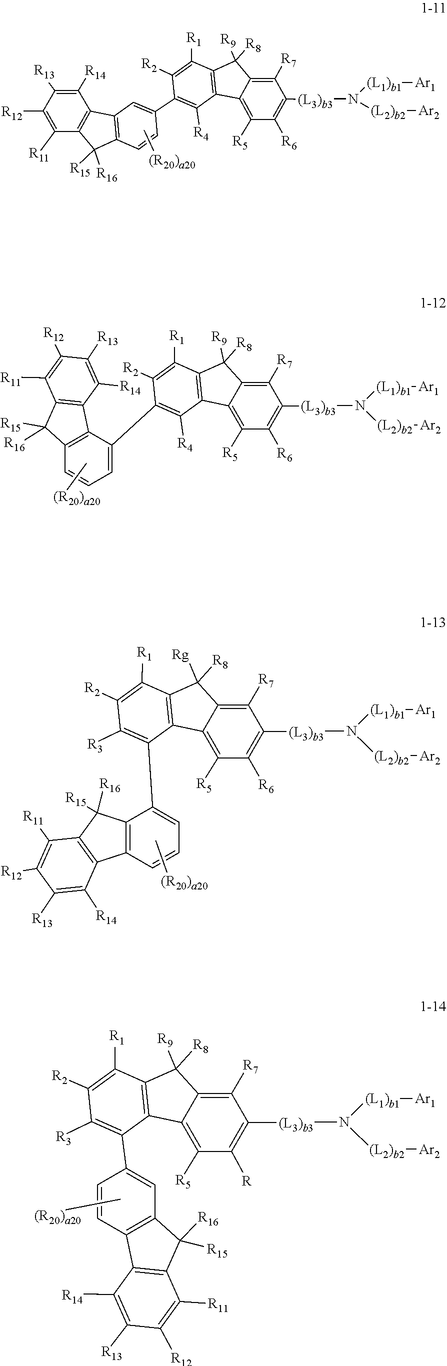

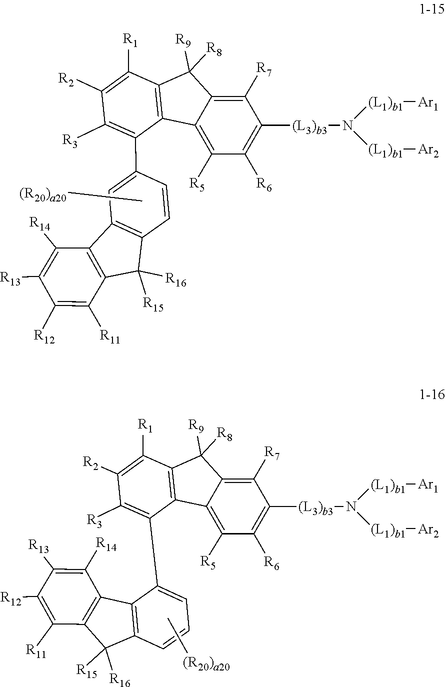

In Formulae 1-1 to 1-16,
R₁ to R₉, Ar₁, Ar₂, L₁ to L₃, b1 to b3, R₁₁ to R₁₆, R₂₀, and a20 may each be the same as described herein.

According to one or more embodiments, the amine-containing compound may be one selected from Compounds 1 to 240:

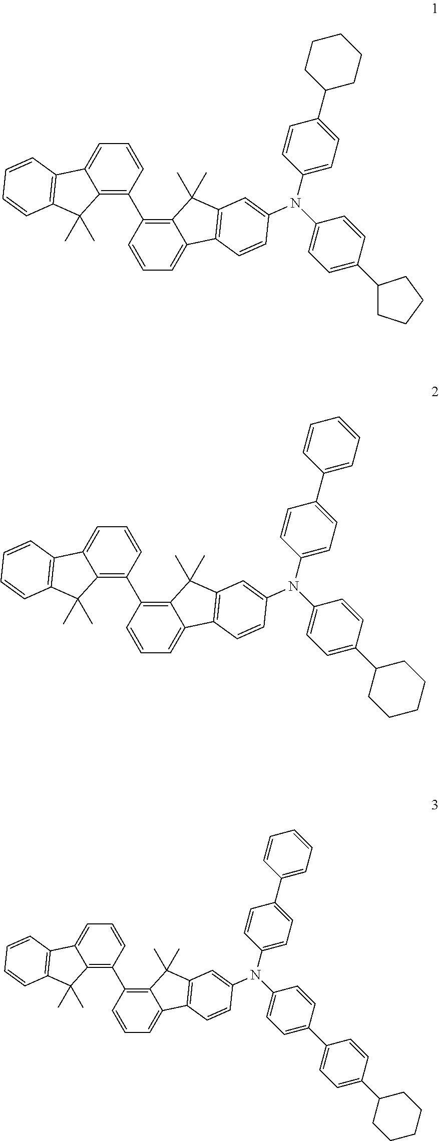 [SMILES: CC1(C)c2ccccc2-c2cccc(-c3cccc4c3C(C)(C)c3cc(N(c5ccc(C6CCCCC6)cc5)c5ccc(C6CCCC6)cc5)ccc3-4)c21 ; CC1(C)c2ccccc2-c2cccc(-c3cccc4c3C(C)(C)c3cc(N(c5ccc(-c6ccccc6)cc5)c5ccc(C6CCCCC6)cc5)ccc3-4)c21 ; CC1(C)c2ccccc2-c2cccc(-c3cccc4c3C(C)(C)c3cc(N(c5ccc(-c6ccccc6)cc5)c5ccc(-c6ccc(C7CCCCC7)cc6)cc5)ccc3-4)c21]

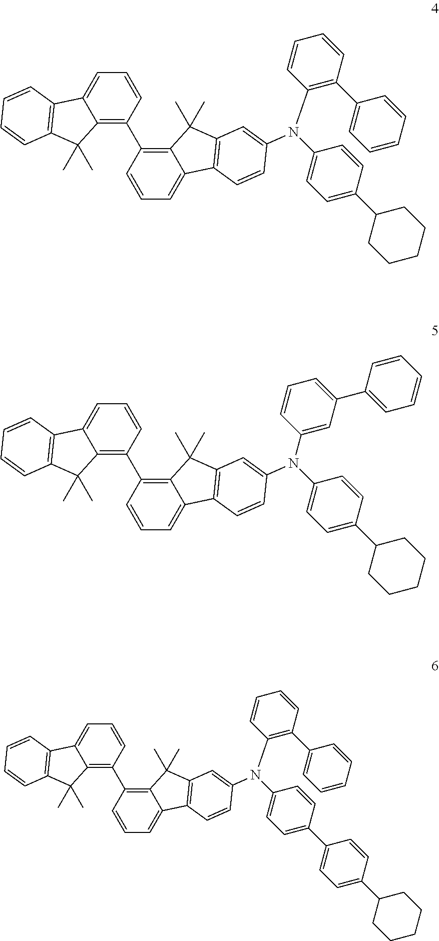 [SMILES: CC1(C)c2ccccc2-c2cccc(-c3cccc4c3C(C)(C)c3cc(N(c5ccc(C6CCCCC6)cc5)c5ccccc5-c5ccccc5)ccc3-4)c21 ; CC1(C)c2ccccc2-c2cccc(-c3cccc4c3C(C)(C)c3cc(N(c5ccc(C6CCCCC6)cc5)c5cccc(-c6ccccc6)c5)ccc3-4)c21 ; CC1(C)c2ccccc2-c2cccc(-c3cccc4c3C(C)(C)c3cc(N(c5ccc(-c6ccc(C7CCCCC7)cc6)cc5)c5ccccc5-c5ccccc5)ccc3-4)c21]

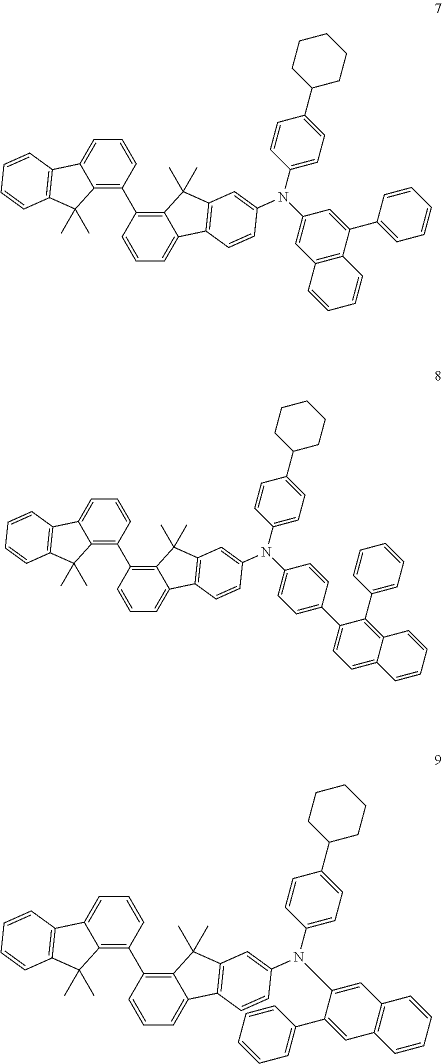 [SMILES: CC1(C)c2ccccc2-c2cccc(-c3cccc4c3C(C)(C)c3cc(N(c5ccc(C6CCCCC6)cc5)c5cc(-c6ccccc6)c6ccccc6c5)ccc3-4)c21 ; CC1(C)c2ccccc2-c2cccc(-c3cccc4c3C(C)(C)c3cc(N(c5ccc(-c6ccc7ccccc7c6-c6ccccc6)cc5)c5ccc(C6CCCCC6)cc5)ccc3-4)c21 ; CC1(C)c2ccccc2-c2cccc(-c3cccc4c3C(C)(C)c3cc(N(c5ccc(C6CCCCC6)cc5)c5cc6ccccc6cc5-c5ccccc5)ccc3-4)c21]

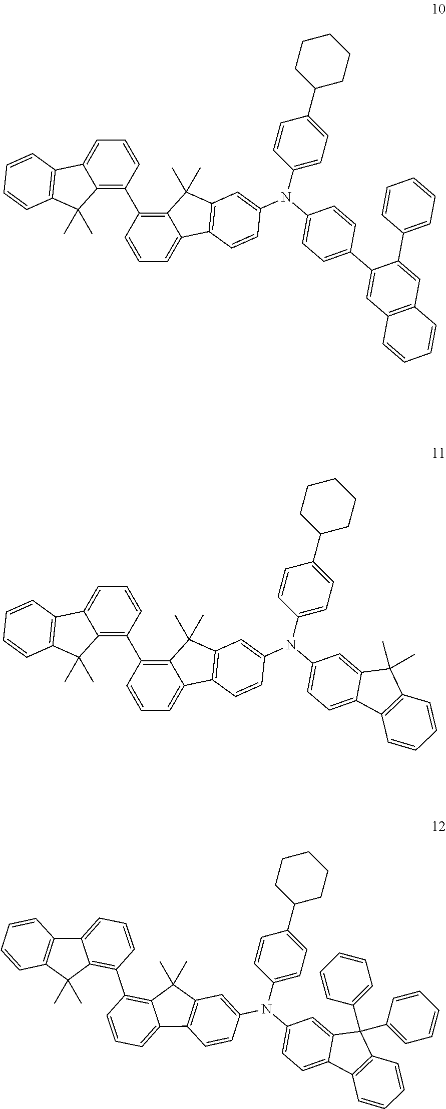 [SMILES: CC1(C)c2ccccc2-c2cccc(-c3cccc4c3C(C)(C)c3cc(N(c5ccc(-c6cc7ccccc7cc6-c6ccccc6)cc5)c5ccc(C6CCCCC6)cc5)ccc3-4)c21 ; CC1(C)c2ccccc2-c2ccc(N(c3ccc(C4CCCCC4)cc3)c3ccc4c(c3)C(C)(C)c3c-4cccc3-c3cccc4c3C(C)(C)c3ccccc3-4)cc21 ; CC1(C)c2ccccc2-c2cccc(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)c21]

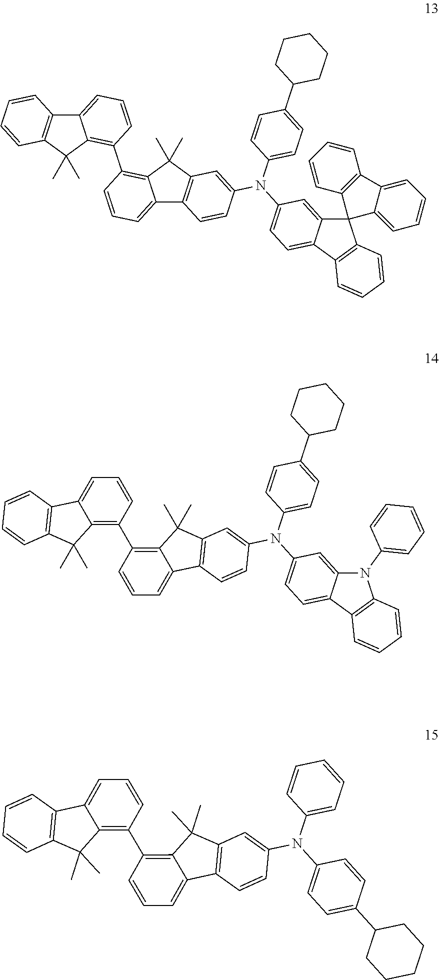 [SMILES: CC1(C)c2ccccc2-c2cccc(-c3cccc4c3C(C)(C)c3cc(N(c5ccc(C6CCCCC6)cc5)c5ccc6c(c5)C5(c7ccccc7-c7ccccc75)c5ccccc5-6)ccc3-4)c21 ; CC1(C)c2ccccc2-c2cccc(-c3cccc4c3C(C)(C)c3cc(N(c5ccc(C6CCCCC6)cc5)c5ccc6c7ccccc7n(-c7ccccc7)c6c5)ccc3-4)c21 ; CC1(C)c2ccccc2-c2cccc(-c3cccc4c3C(C)(C)c3cc(N(c5ccccc5)c5ccc(C6CCCCC6)cc5)ccc3-4)c21]

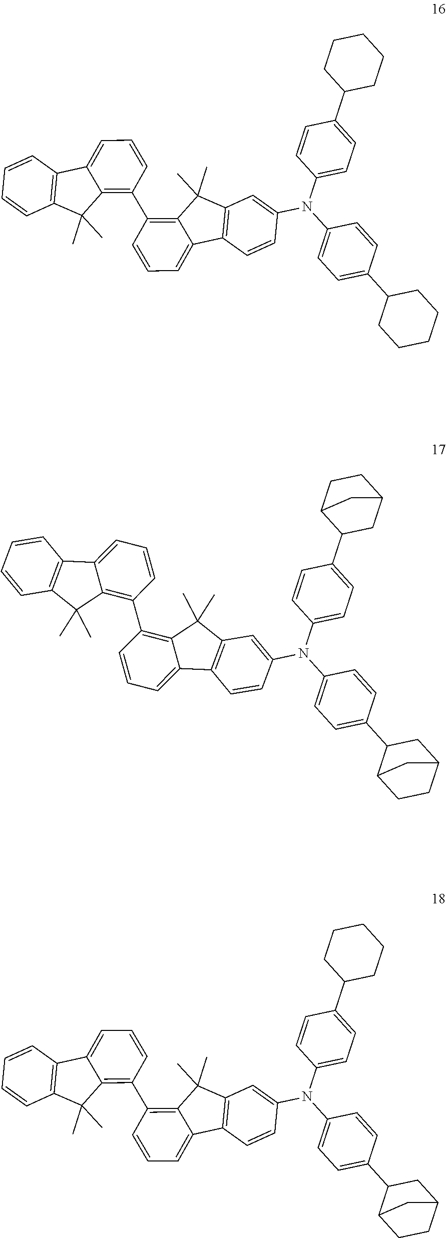 [SMILES: CC1(C)c2ccccc2-c2cccc(-c3cccc4c3C(C)(C)c3cc(N(c5ccc(C6CCCCC6)cc5)c5ccc(C6CCCCC6)cc5)ccc3-4)c21 ; CC1(C)c2ccccc2-c2cccc(-c3cccc4c3C(C)(C)c3cc(N(c5ccc(C6CC7CCC6C7)cc5)c5ccc(C6CC7CCC6C7)cc5)ccc3-4)c21 ; CC1(C)c2ccccc2-c2cccc(-c3cccc4c3C(C)(C)c3cc(N(c5ccc(C6CCCCC6)cc5)c5ccc(C6CC7CCC6C7)cc5)ccc3-4)c21]

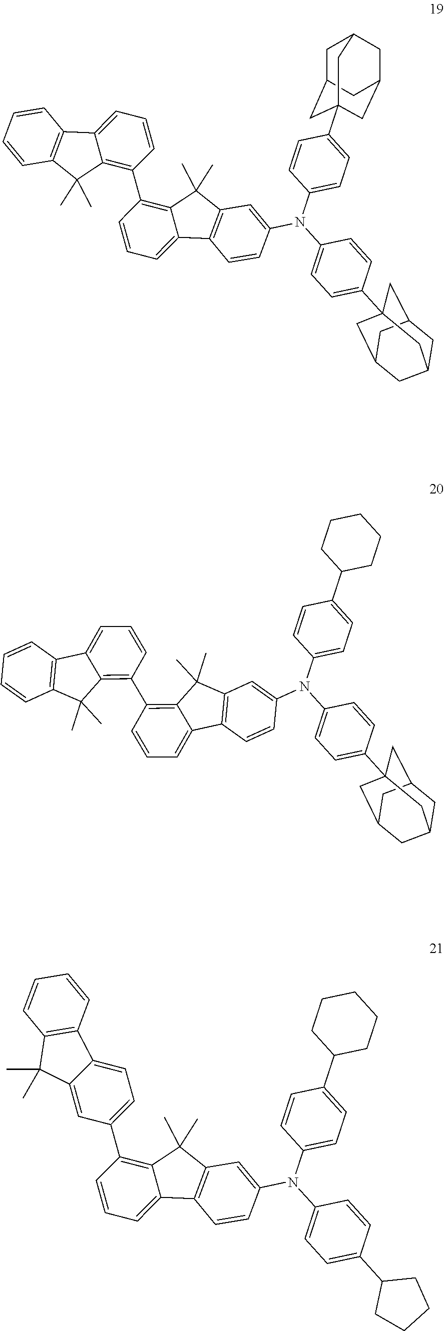 [SMILES: CC1(C)c2ccccc2-c2cccc(-c3cccc4c3C(C)(C)c3cc(N(c5ccc(C67CC8CC(CC(C8)C6)C7)cc5)c5ccc(C67CC8CC(CC(C8)C6)C7)cc5)ccc3-4)c21 ; CC1(C)c2ccccc2-c2cccc(-c3cccc4c3C(C)(C)c3cc(N(c5ccc(C6CCCCC6)cc5)c5ccc(C67CC8CC(CC(C8)C6)C7)cc5)ccc3-4)c21 ; CC1(C)c2ccccc2-c2ccc(-c3cccc4c3C(C)(C)c3cc(N(c5ccc(C6CCCCC6)cc5)c5ccc(C6CCCC6)cc5)ccc3-4)cc21]

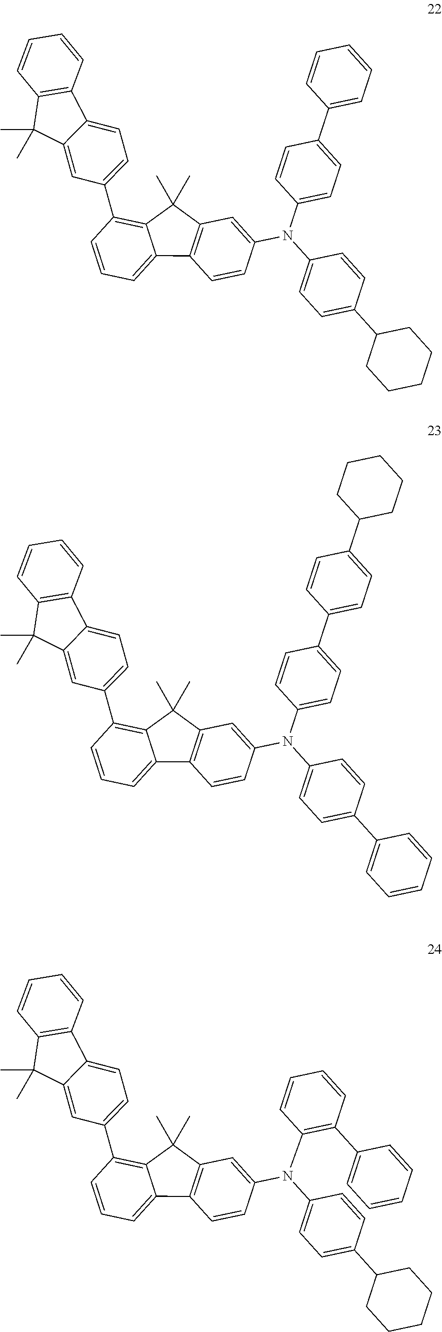 [SMILES: CC1(C)c2ccccc2-c2ccc(-c3cccc4c3C(C)(C)c3cc(N(c5ccc(-c6ccccc6)cc5)c5ccc(C6CCCCC6)cc5)ccc3-4)cc21 ; CC1(C)c2ccccc2-c2ccc(-c3cccc4c3C(C)(C)c3cc(N(c5ccc(-c6ccccc6)cc5)c5ccc(-c6ccc(C7CCCCC7)cc6)cc5)ccc3-4)cc21 ; CC1(C)c2ccccc2-c2ccc(-c3cccc4c3C(C)(C)c3cc(N(c5ccc(C6CCCCC6)cc5)c5ccccc5-c5ccccc5)ccc3-4)cc21]

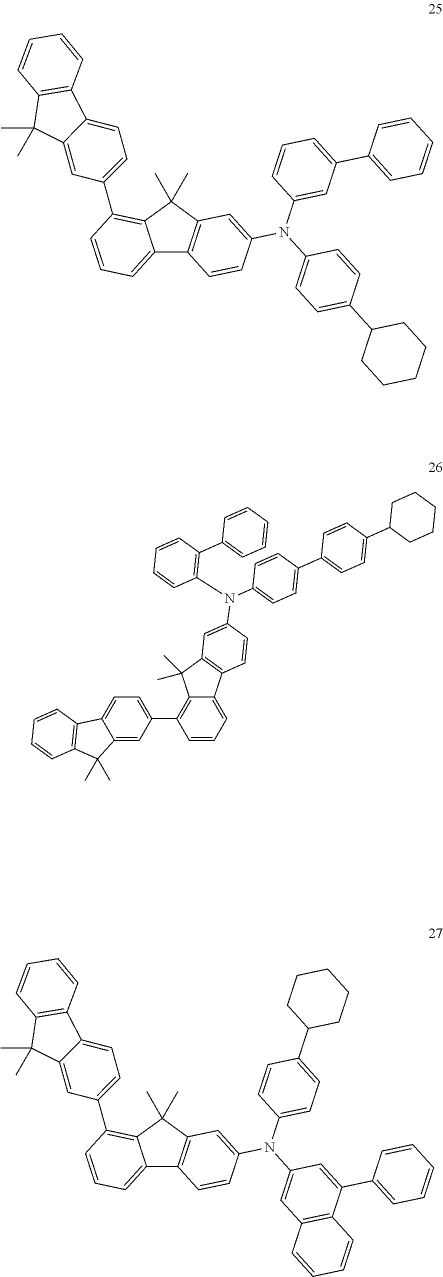 [SMILES: CC1(C)c2ccccc2-c2ccc(-c3cccc4c3C(C)(C)c3cc(N(c5ccc(C6CCCCC6)cc5)c5cccc(-c6ccccc6)c5)ccc3-4)cc21 ; CC1(C)c2ccccc2-c2ccc(-c3cccc4c3C(C)(C)c3cc(N(c5ccc(-c6ccc(C7CCCCC7)cc6)cc5)c5ccccc5-c5ccccc5)ccc3-4)cc21 ; CC1(C)c2ccccc2-c2ccc(-c3cccc4c3C(C)(C)c3cc(N(c5ccc(C6CCCCC6)cc5)c5cc(-c6ccccc6)c6ccccc6c5)ccc3-4)cc21]

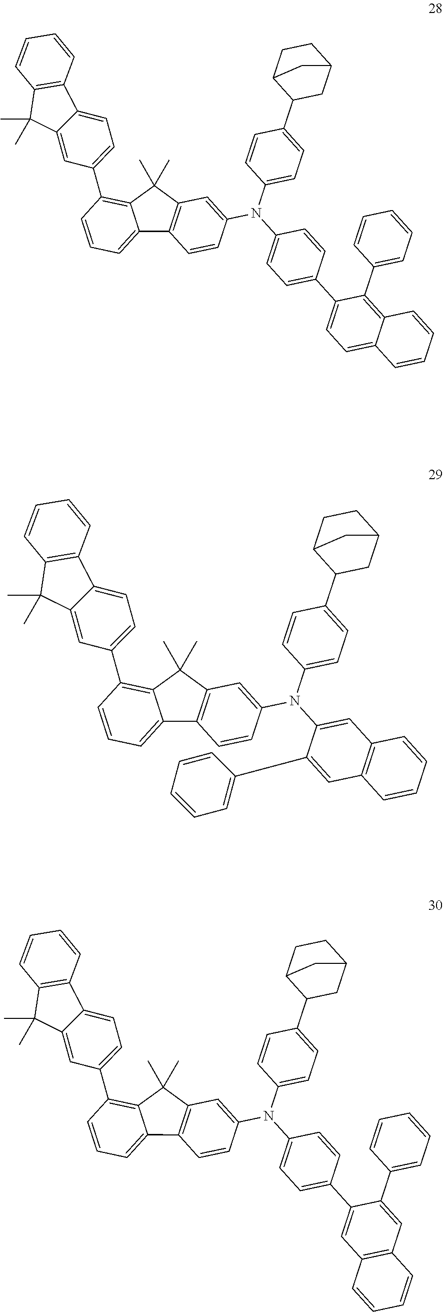 [SMILES: CC1(C)c2ccccc2-c2ccc(-c3cccc4c3C(C)(C)c3cc(N(c5ccc(-c6ccc7ccccc7c6-c6ccccc6)cc5)c5ccc(C6CC7CCC6C7)cc5)ccc3-4)cc21 ; CC1(C)c2ccccc2-c2ccc(-c3cccc4c3C(C)(C)c3cc(N(c5ccc(C6CC7CCC6C7)cc5)c5cc6ccccc6cc5-c5ccccc5)ccc3-4)cc21 ; CC1(C)c2ccccc2-c2ccc(-c3cccc4c3C(C)(C)c3cc(N(c5ccc(-c6cc7ccccc7cc6-c6ccccc6)cc5)c5ccc(C6CC7CCC6C7)cc5)ccc3-4)cc21]

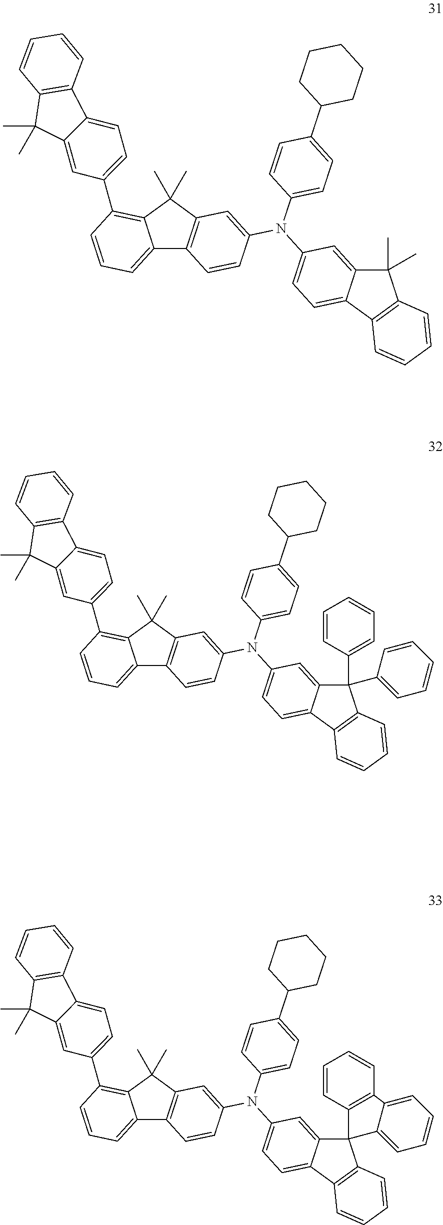 [SMILES: CC1(C)c2ccccc2-c2ccc(-c3cccc4c3C(C)(C)c3cc(N(c5ccc(C6CCCCC6)cc5)c5ccc6c(c5)C(C)(C)c5ccccc5-6)ccc3-4)cc21 ; CC1(C)c2ccccc2-c2ccc(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)cc21 ; CC1(C)c2ccccc2-c2ccc(-c3cccc4c3C(C)(C)c3cc(N(c5ccc(C6CCCCC6)cc5)c5ccc6c(c5)C5(c7ccccc7-c7ccccc75)c5ccccc5-6)ccc3-4)cc21]

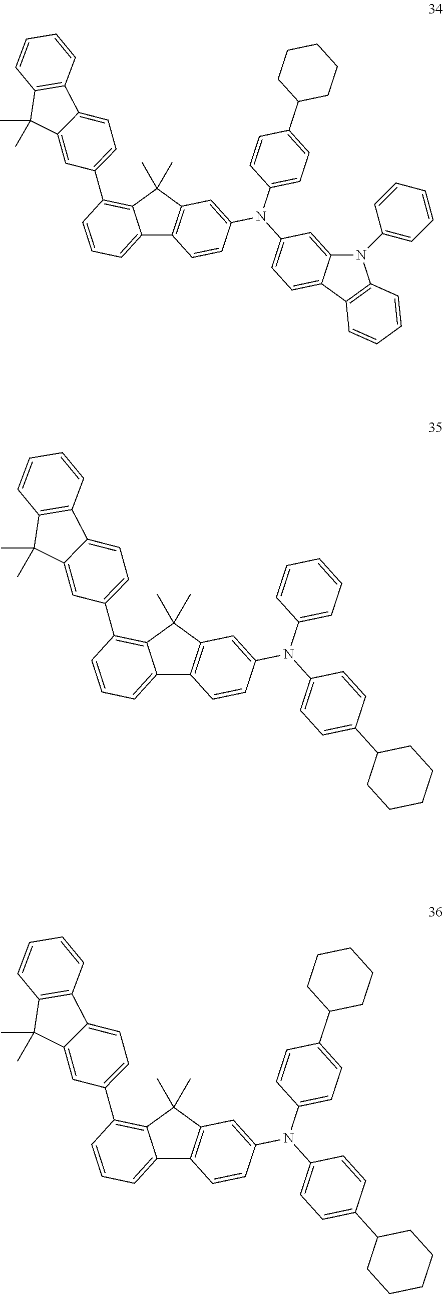 [SMILES: CC1(C)c2ccccc2-c2ccc(-c3cccc4c3C(C)(C)c3cc(N(c5ccc(C6CCCCC6)cc5)c5ccc6c7ccccc7n(-c7ccccc7)c6c5)ccc3-4)cc21 ; CC1(C)c2ccccc2-c2ccc(-c3cccc4c3C(C)(C)c3cc(N(c5ccccc5)c5ccc(C6CCCCC6)cc5)ccc3-4)cc21 ; CC1(C)c2ccccc2-c2ccc(-c3cccc4c3C(C)(C)c3cc(N(c5ccc(C6CCCCC6)cc5)c5ccc(C6CCCCC6)cc5)ccc3-4)cc21]

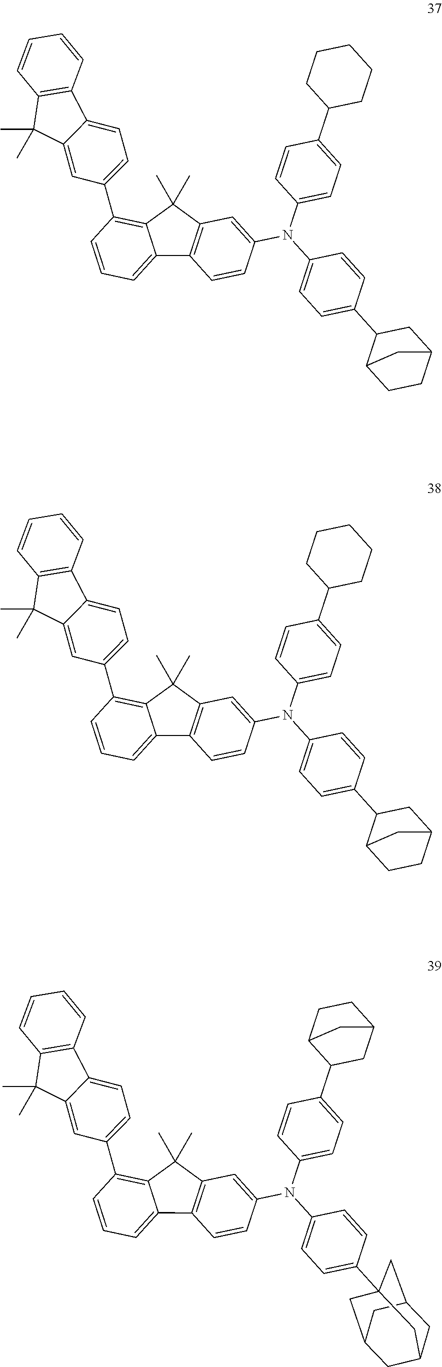 [SMILES: CC1(C)c2ccccc2-c2ccc(-c3cccc4c3C(C)(C)c3cc(N(c5ccc(C6CCCCC6)cc5)c5ccc(C6CC7CCC6C7)cc5)ccc3-4)cc21 ; CC1(C)c2ccccc2-c2ccc(-c3cccc4c3C(C)(C)c3cc(N(c5ccc(C6CCCCC6)cc5)c5ccc(C6CC7CCC6C7)cc5)ccc3-4)cc21 ; CC1(C)c2ccccc2-c2ccc(-c3cccc4c3C(C)(C)c3cc(N(c5ccc(C6CC7CCC6C7)cc5)c5ccc(C67CC8CC(CC(C8)C6)C7)cc5)ccc3-4)cc21]

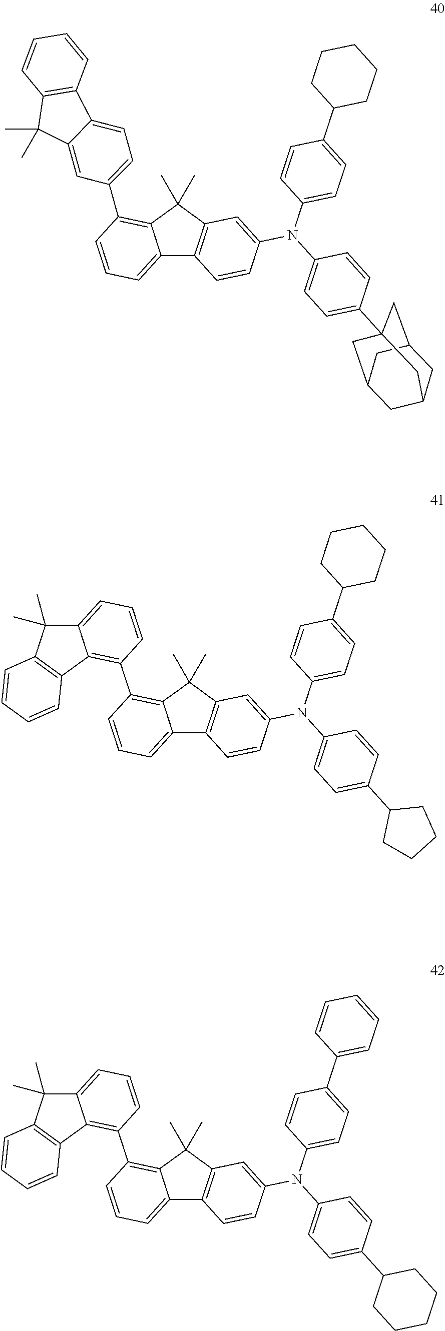 [SMILES: CC1(C)c2ccccc2-c2ccc(-c3cccc4c3C(C)(C)c3cc(N(c5ccc(C6CCCCC6)cc5)c5ccc(C67CC8CC(CC(C8)C6)C7)cc5)ccc3-4)cc21 ; CC1(C)c2ccccc2-c2c(-c3cccc4c3C(C)(C)c3cc(N(c5ccc(C6CCCCC6)cc5)c5ccc(C6CCCC6)cc5)ccc3-4)cccc21 ; CC1(C)c2ccccc2-c2c(-c3cccc4c3C(C)(C)c3cc(N(c5ccc(-c6ccccc6)cc5)c5ccc(C6CCCCC6)cc5)ccc3-4)cccc21]

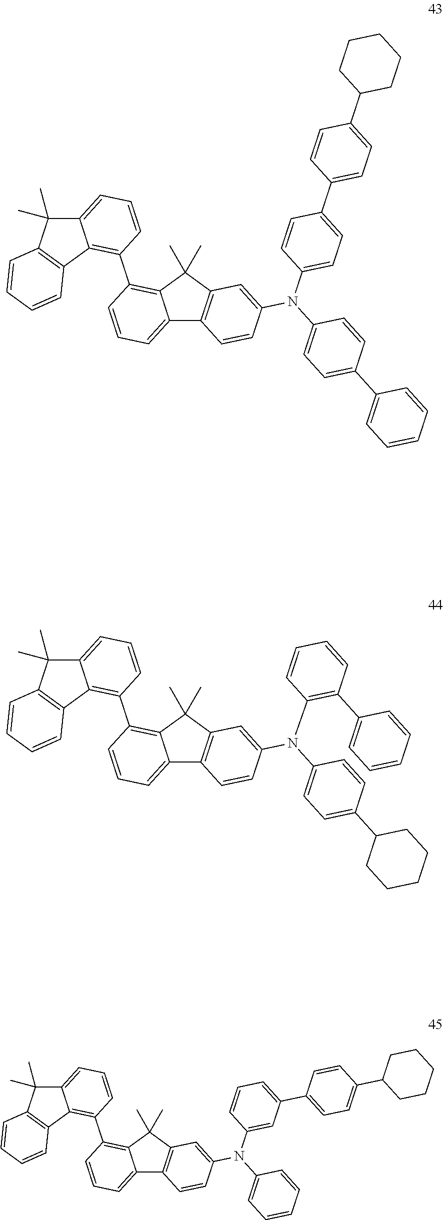 [SMILES: CC1(C)c2ccccc2-c2c(-c3cccc4c3C(C)(C)c3cc(N(c5ccc(-c6ccccc6)cc5)c5ccc(-c6ccc(C7CCCCC7)cc6)cc5)ccc3-4)cccc21 ; CC1(C)c2ccccc2-c2c(-c3cccc4c3C(C)(C)c3cc(N(c5ccc(C6CCCCC6)cc5)c5ccccc5-c5ccccc5)ccc3-4)cccc21 ; CC1(C)c2ccccc2-c2c(-c3cccc4c3C(C)(C)c3cc(N(c5ccccc5)c5cccc(-c6ccc(C7CCCCC7)cc6)c5)ccc3-4)cccc21]

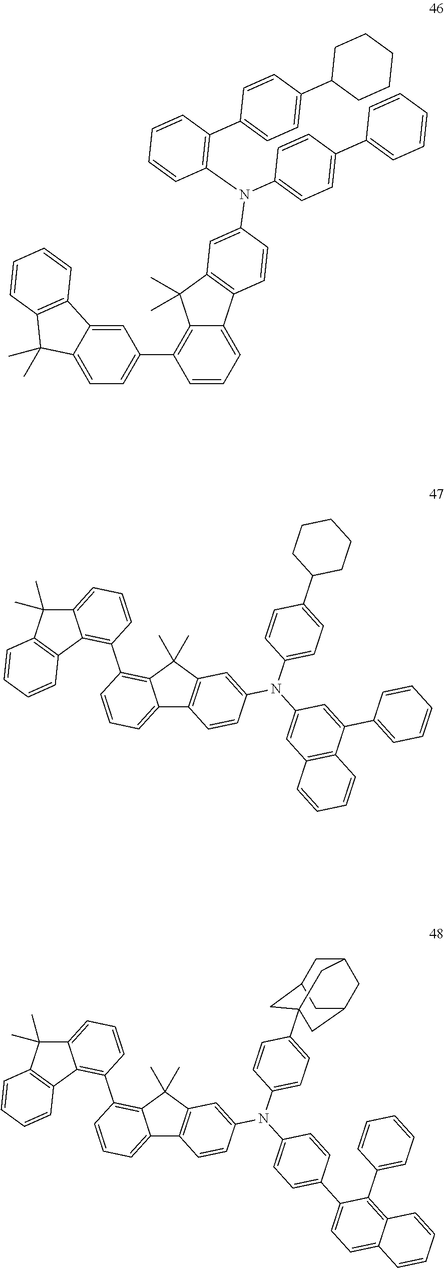 [SMILES: CC1(C)c2ccccc2-c2cc(-c3cccc4c3C(C)(C)c3cc(N(c5ccc(-c6ccccc6)cc5)c5ccccc5-c5ccc(C6CCCCC6)cc5)ccc3-4)ccc21 ; CC1(C)c2ccccc2-c2c(-c3cccc4c3C(C)(C)c3cc(N(c5ccc(C6CCCCC6)cc5)c5cc(-c6ccccc6)c6ccccc6c5)ccc3-4)cccc21 ; CC1(C)c2ccccc2-c2c(-c3cccc4c3C(C)(C)c3cc(N(c5ccc(-c6ccc7ccccc7c6-c6ccccc6)cc5)c5ccc(C67CC8CC(CC(C8)C6)C7)cc5)ccc3-4)cccc21]

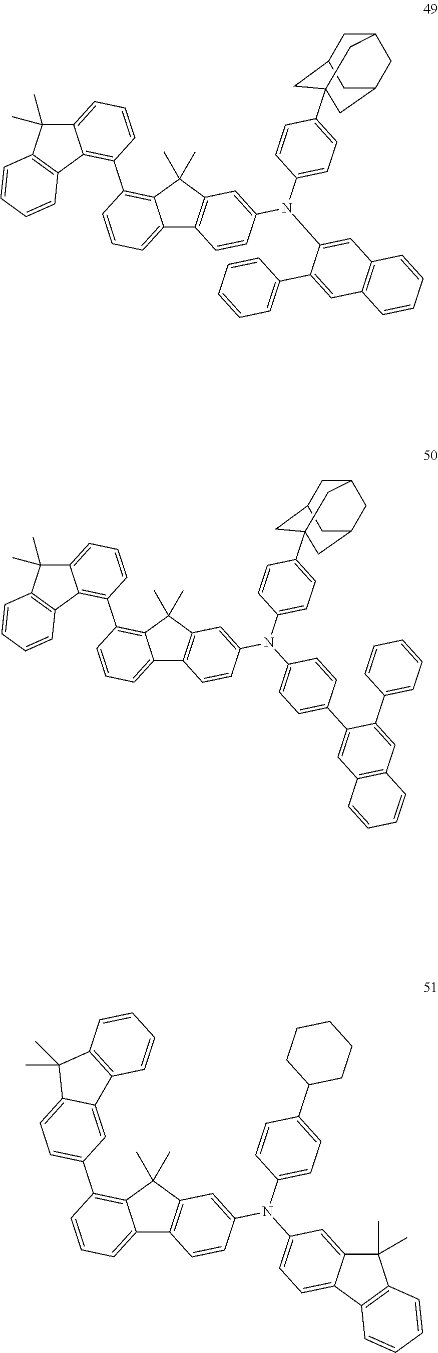 [SMILES: CC1(C)c2ccccc2-c2c(-c3cccc4c3C(C)(C)c3cc(N(c5ccc(C67CC8CC(CC(C8)C6)C7)cc5)c5cc6ccccc6cc5-c5ccccc5)ccc3-4)cccc21 ; CC1(C)c2ccccc2-c2c(-c3cccc4c3C(C)(C)c3cc(N(c5ccc(-c6cc7ccccc7cc6-c6ccccc6)cc5)c5ccc(C67CC8CC(CC(C8)C6)C7)cc5)ccc3-4)cccc21 ; CC1(C)c2ccccc2-c2cc(-c3cccc4c3C(C)(C)c3cc(N(c5ccc(C6CCCCC6)cc5)c5ccc6c(c5)C(C)(C)c5ccccc5-6)ccc3-4)ccc21]

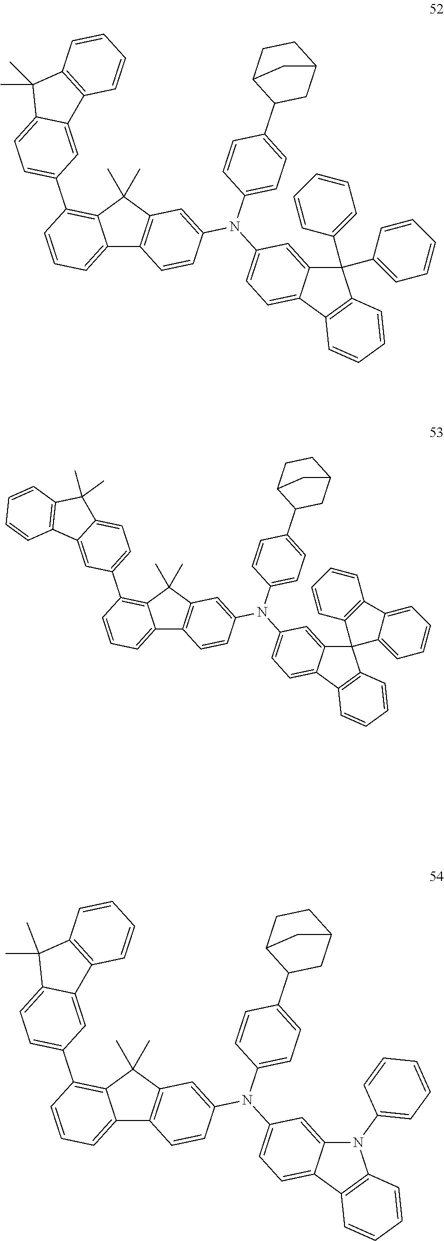 [SMILES: CC1(C)c2ccccc2-c2cc(-c3cccc4c3C(C)(C)c3cc(N(c5ccc(C6CC7CCC6C7)cc5)c5ccc6c(c5)C(c5ccccc5)(c5ccccc5)c5ccccc5-6)ccc3-4)ccc21 ; CC1(C)c2ccccc2-c2cc(-c3cccc4c3C(C)(C)c3cc(N(c5ccc(C6CC7CCC6C7)cc5)c5ccc6c(c5)C5(c7ccccc7-c7ccccc75)c5ccccc5-6)ccc3-4)ccc21 ; CC1(C)c2ccccc2-c2cc(-c3cccc4c3C(C)(C)c3cc(N(c5ccc(C6CC7CCC6C7)cc5)c5ccc6c7ccccc7n(-c7ccccc7)c6c5)ccc3-4)ccc21]

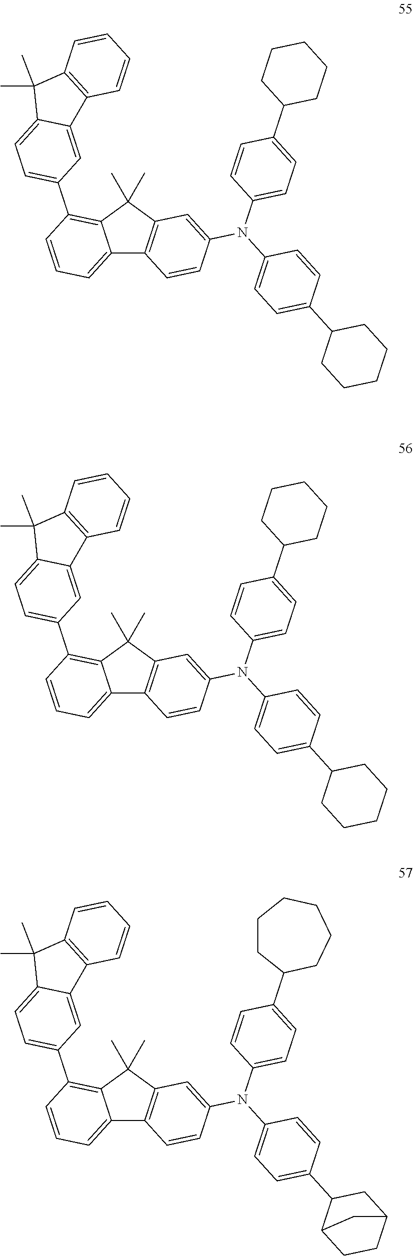 [SMILES: CC1(C)c2ccccc2-c2cc(-c3cccc4c3C(C)(C)c3cc(N(c5ccc(C6CCCCC6)cc5)c5ccc(C6CCCCC6)cc5)ccc3-4)ccc21 ; CC1(C)c2ccccc2-c2cc(-c3cccc4c3C(C)(C)c3cc(N(c5ccc(C6CCCCC6)cc5)c5ccc(C6CCCCC6)cc5)ccc3-4)ccc21 ; CC1(C)c2ccccc2-c2cc(-c3cccc4c3C(C)(C)c3cc(N(c5ccc(C6CCCCCC6)cc5)c5ccc(C6CC7CCC6C7)cc5)ccc3-4)ccc21]

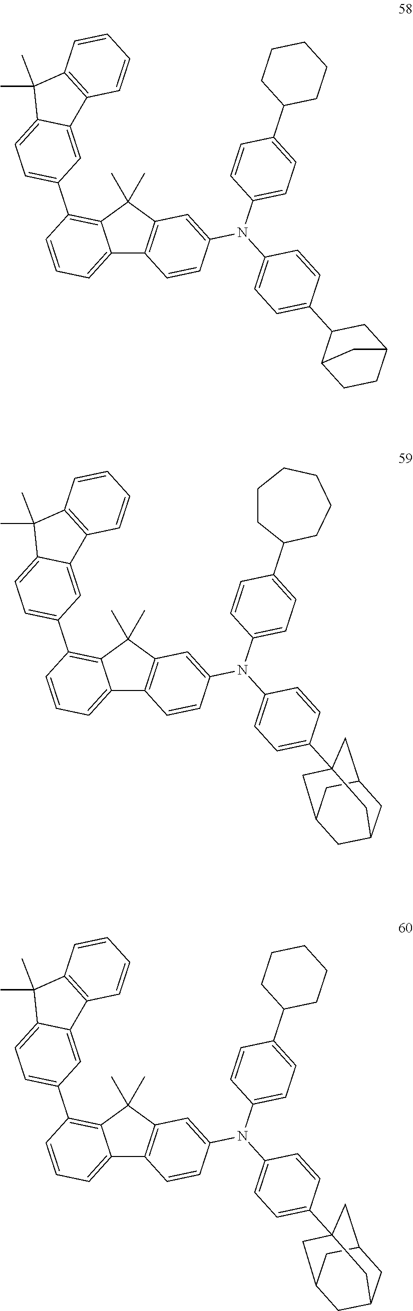 [SMILES: CC1(C)c2ccccc2-c2cc(-c3cccc4c3C(C)(C)c3cc(N(c5ccc(C6CCCCC6)cc5)c5ccc(C6CC7CCC6C7)cc5)ccc3-4)ccc21 ; CC1(C)c2ccccc2-c2cc(-c3cccc4c3C(C)(C)c3cc(N(c5ccc(C6CCCCCC6)cc5)c5ccc(C67CC8CC(CC(C8)C6)C7)cc5)ccc3-4)ccc21 ; CC1(C)c2ccccc2-c2cc(-c3cccc4c3C(C)(C)c3cc(N(c5ccc(C6CCCCC6)cc5)c5ccc(C67CC8CC(CC(C8)C6)C7)cc5)ccc3-4)ccc21]

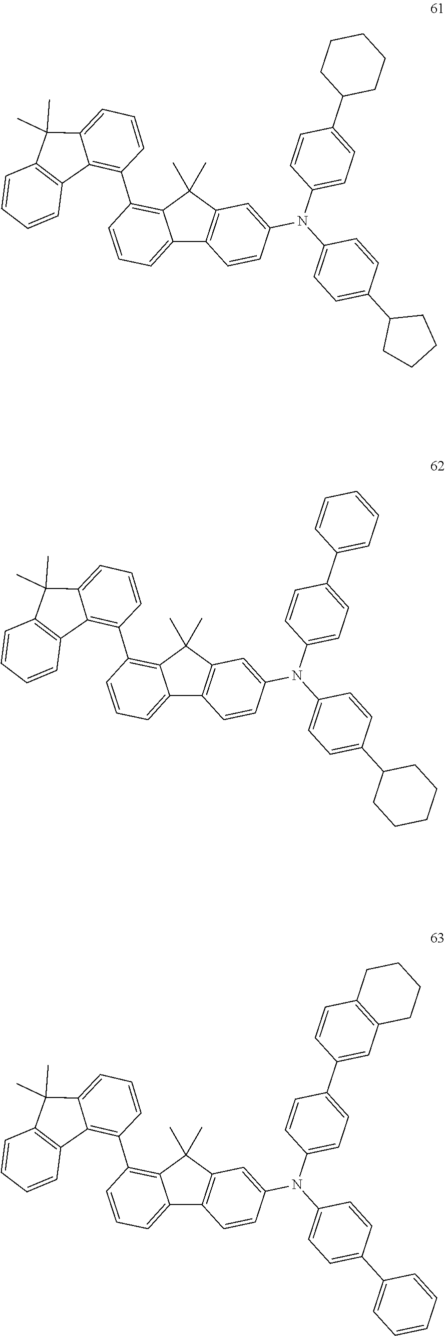 [SMILES: CC1(C)c2ccccc2-c2c(-c3cccc4c3C(C)(C)c3cc(N(c5ccc(C6CCCCC6)cc5)c5ccc(C6CCCC6)cc5)ccc3-4)cccc21 ; CC1(C)c2ccccc2-c2c(-c3cccc4c3C(C)(C)c3cc(N(c5ccc(-c6ccccc6)cc5)c5ccc(C6CCCCC6)cc5)ccc3-4)cccc21 ; CC1(C)c2ccccc2-c2c(-c3cccc4c3C(C)(C)c3cc(N(c5ccc(-c6ccccc6)cc5)c5ccc(-c6ccc7c(c6)CCCC7)cc5)ccc3-4)cccc21]

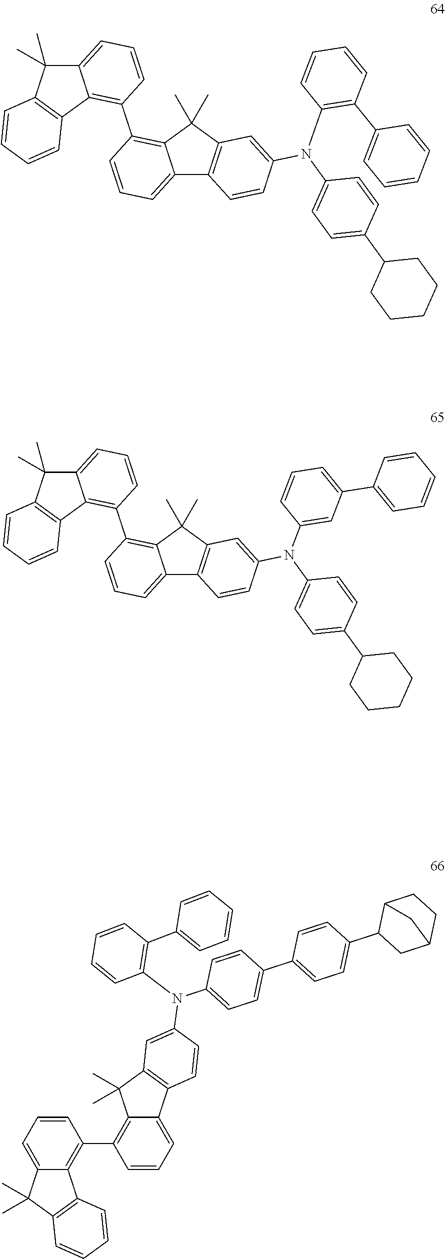 [SMILES: CC1(C)c2ccccc2-c2c(-c3cccc4c3C(C)(C)c3cc(N(c5ccc(C6CCCCC6)cc5)c5ccccc5-c5ccccc5)ccc3-4)cccc21 ; CC1(C)c2ccccc2-c2c(-c3cccc4c3C(C)(C)c3cc(N(c5ccc(C6CCCCC6)cc5)c5cccc(-c6ccccc6)c5)ccc3-4)cccc21 ; CC1(C)c2ccccc2-c2c(-c3cccc4c3C(C)(C)c3cc(N(c5ccc(-c6ccc(C7CC8CCC7C8)cc6)cc5)c5ccccc5-c5ccccc5)ccc3-4)cccc21]

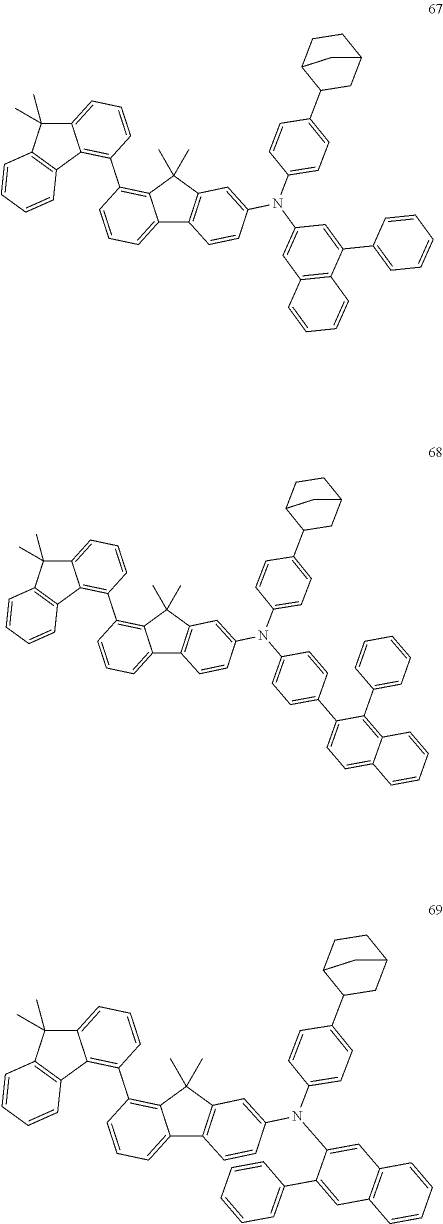 [SMILES: CC1(C)c2ccccc2-c2c(-c3cccc4c3C(C)(C)c3cc(N(c5ccc(C6CC7CCC6C7)cc5)c5cc(-c6ccccc6)c6ccccc6c5)ccc3-4)cccc21 ; CC1(C)c2ccccc2-c2c(-c3cccc4c3C(C)(C)c3cc(N(c5ccc(-c6ccc7ccccc7c6-c6ccccc6)cc5)c5ccc(C6CC7CCC6C7)cc5)ccc3-4)cccc21 ; CC1(C)c2ccccc2-c2c(-c3cccc4c3C(C)(C)c3cc(N(c5ccc(C6CC7CCC6C7)cc5)c5cc6ccccc6cc5-c5ccccc5)ccc3-4)cccc21]

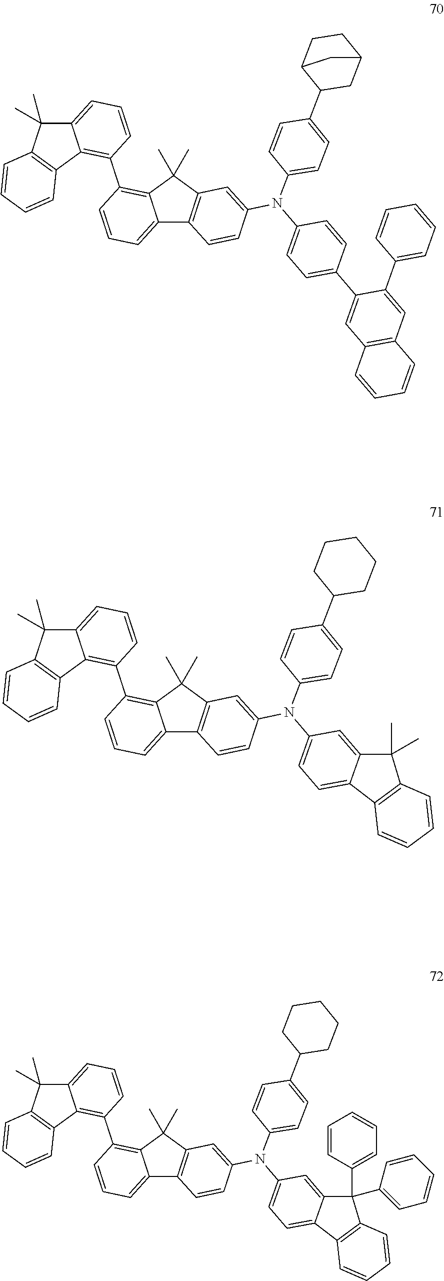 [SMILES: CC1(C)c2ccccc2-c2c(-c3cccc4c3C(C)(C)c3cc(N(c5ccc(-c6cc7ccccc7cc6-c6ccccc6)cc5)c5ccc(C6CC7CCC6C7)cc5)ccc3-4)cccc21 ; CC1(C)c2ccccc2-c2ccc(N(c3ccc(C4CCCCC4)cc3)c3ccc4c(c3)C(C)(C)c3c-4cccc3-c3cccc4c3-c3ccccc3C4(C)C)cc21 ; CC1(C)c2ccccc2-c2c(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)cccc21]

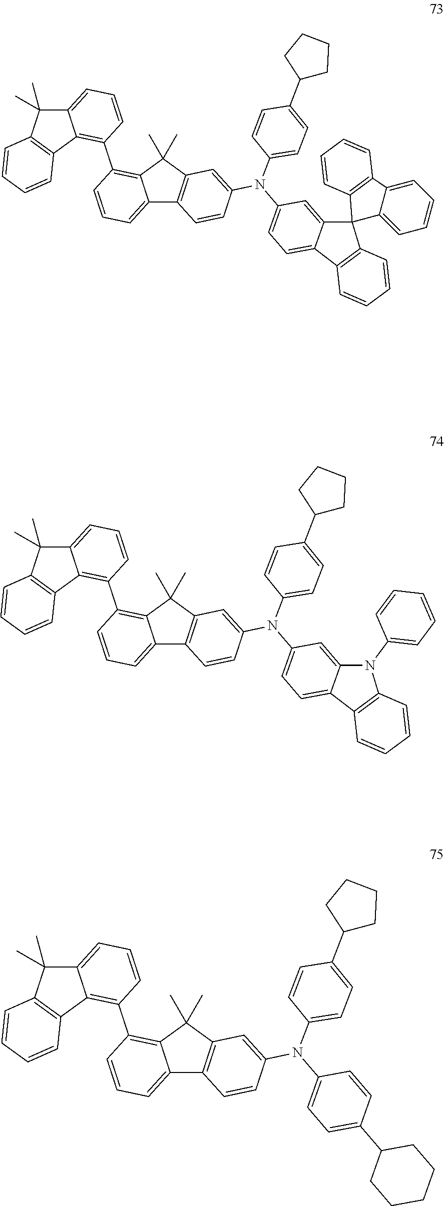 [SMILES: CC1(C)c2ccccc2-c2c(-c3cccc4c3C(C)(C)c3cc(N(c5ccc(C6CCCC6)cc5)c5ccc6c(c5)C5(c7ccccc7-c7ccccc75)c5ccccc5-6)ccc3-4)cccc21 ; CC1(C)c2ccccc2-c2c(-c3cccc4c3C(C)(C)c3cc(N(c5ccc(C6CCCC6)cc5)c5ccc6c7ccccc7n(-c7ccccc7)c6c5)ccc3-4)cccc21 ; CC1(C)c2ccccc2-c2c(-c3cccc4c3C(C)(C)c3cc(N(c5ccc(C6CCCCC6)cc5)c5ccc(C6CCCC6)cc5)ccc3-4)cccc21]

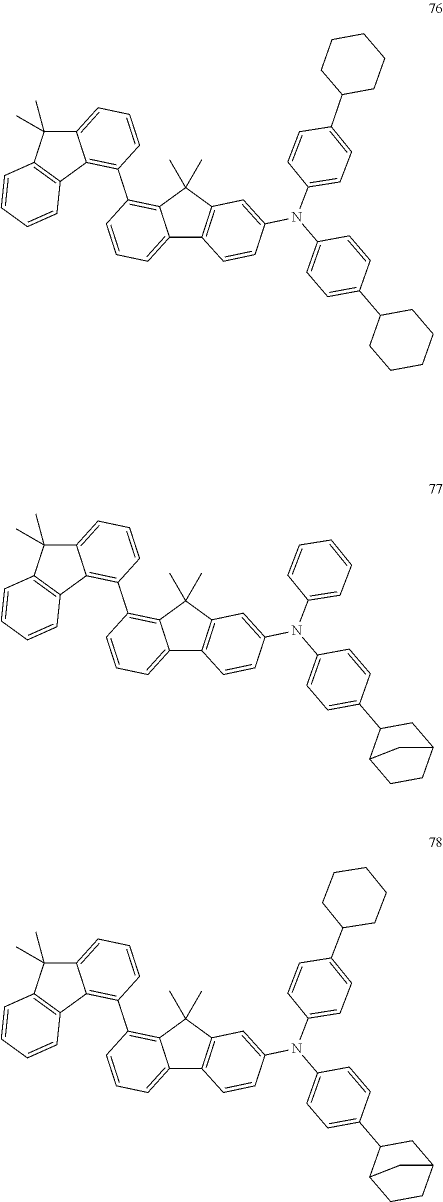 [SMILES: CC1(C)c2ccccc2-c2c(-c3cccc4c3C(C)(C)c3cc(N(c5ccc(C6CCCCC6)cc5)c5ccc(C6CCCCC6)cc5)ccc3-4)cccc21 ; CC1(C)c2ccccc2-c2c(-c3cccc4c3C(C)(C)c3cc(N(c5ccccc5)c5ccc(C6CC7CCC6C7)cc5)ccc3-4)cccc21 ; CC1(C)c2ccccc2-c2c(-c3cccc4c3C(C)(C)c3cc(N(c5ccc(C6CCCCC6)cc5)c5ccc(C6CC7CCC6C7)cc5)ccc3-4)cccc21]

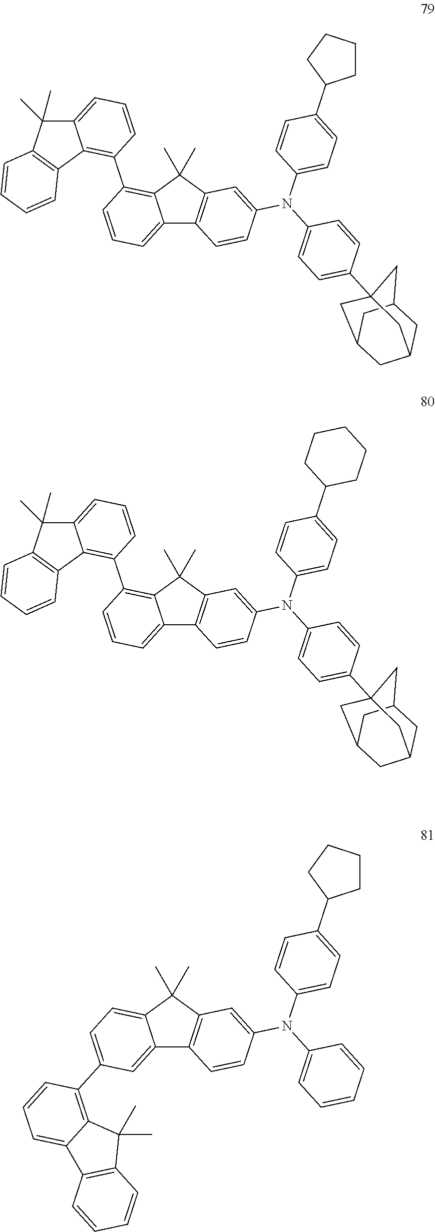 [SMILES: CC1(C)c2ccccc2-c2c(-c3cccc4c3C(C)(C)c3cc(N(c5ccc(C6CCCC6)cc5)c5ccc(C67CC8CC(CC(C8)C6)C7)cc5)ccc3-4)cccc21 ; CC1(C)c2ccccc2-c2c(-c3cccc4c3C(C)(C)c3cc(N(c5ccc(C6CCCCC6)cc5)c5ccc(C67CC8CC(CC(C8)C6)C7)cc5)ccc3-4)cccc21 ; CC1(C)c2ccc(-c3cccc4c3C(C)(C)c3ccccc3-4)cc2-c2ccc(N(c3ccccc3)c3ccc(C4CCCC4)cc3)cc21]

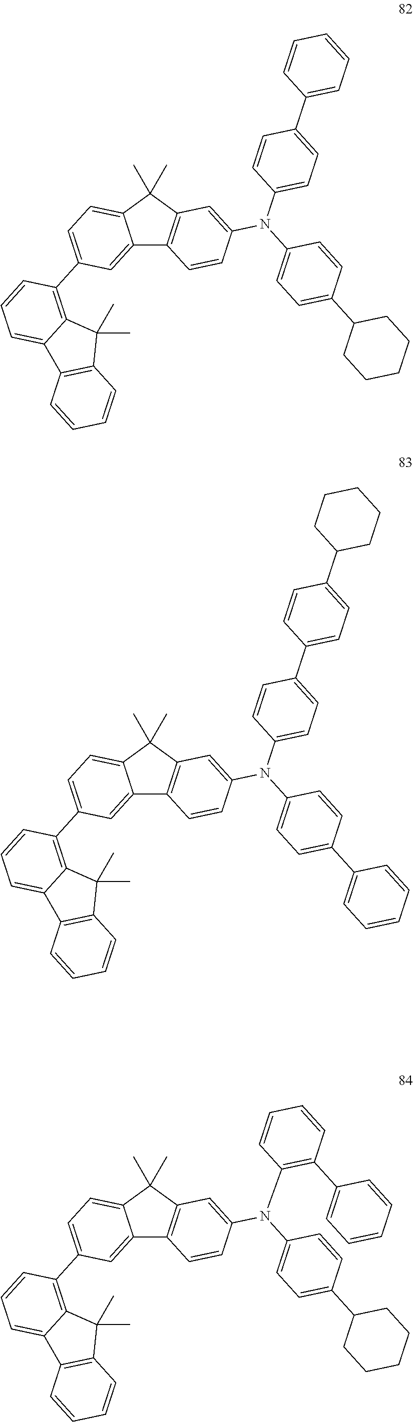 [SMILES: CC1(C)c2ccc(-c3cccc4c3C(C)(C)c3ccccc3-4)cc2-c2ccc(N(c3ccc(-c4ccccc4)cc3)c3ccc(C4CCCCC4)cc3)cc21 ; CC1(C)c2ccc(-c3cccc4c3C(C)(C)c3ccccc3-4)cc2-c2ccc(N(c3ccc(-c4ccccc4)cc3)c3ccc(-c4ccc(C5CCCCC5)cc4)cc3)cc21 ; CC1(C)c2ccc(-c3cccc4c3C(C)(C)c3ccccc3-4)cc2-c2ccc(N(c3ccc(C4CCCCC4)cc3)c3ccccc3-c3ccccc3)cc21]

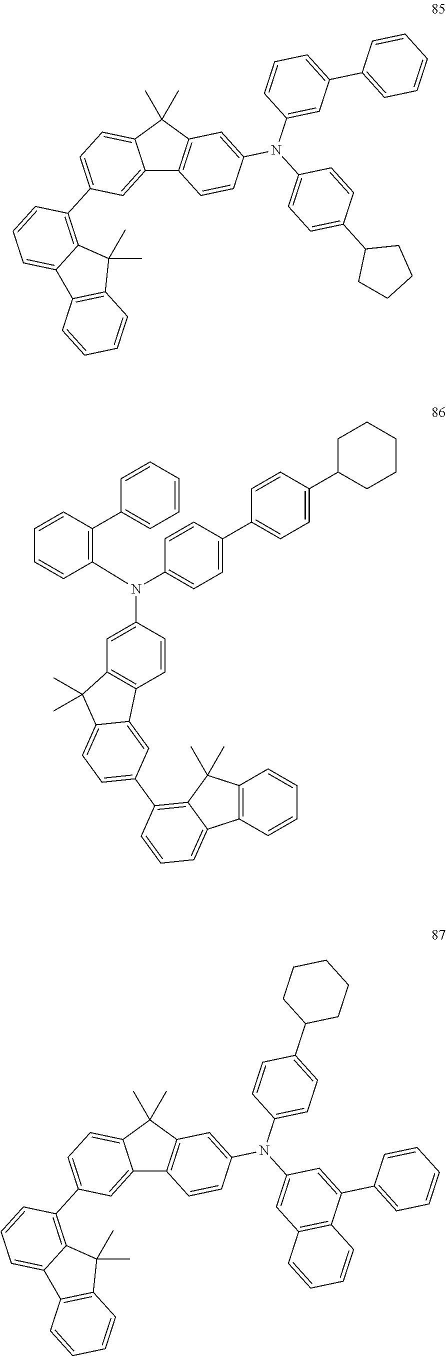 [SMILES: CC1(C)c2ccc(-c3cccc4c3C(C)(C)c3ccccc3-4)cc2-c2ccc(N(c3ccc(C4CCCC4)cc3)c3cccc(-c4ccccc4)c3)cc21 ; CC1(C)c2ccc(-c3cccc4c3C(C)(C)c3ccccc3-4)cc2-c2ccc(N(c3ccc(-c4ccc(C5CCCCC5)cc4)cc3)c3ccccc3-c3ccccc3)cc21 ; CC1(C)c2ccc(-c3cccc4c3C(C)(C)c3ccccc3-4)cc2-c2ccc(N(c3ccc(C4CCCCC4)cc3)c3cc(-c4ccccc4)c4ccccc4c3)cc21]

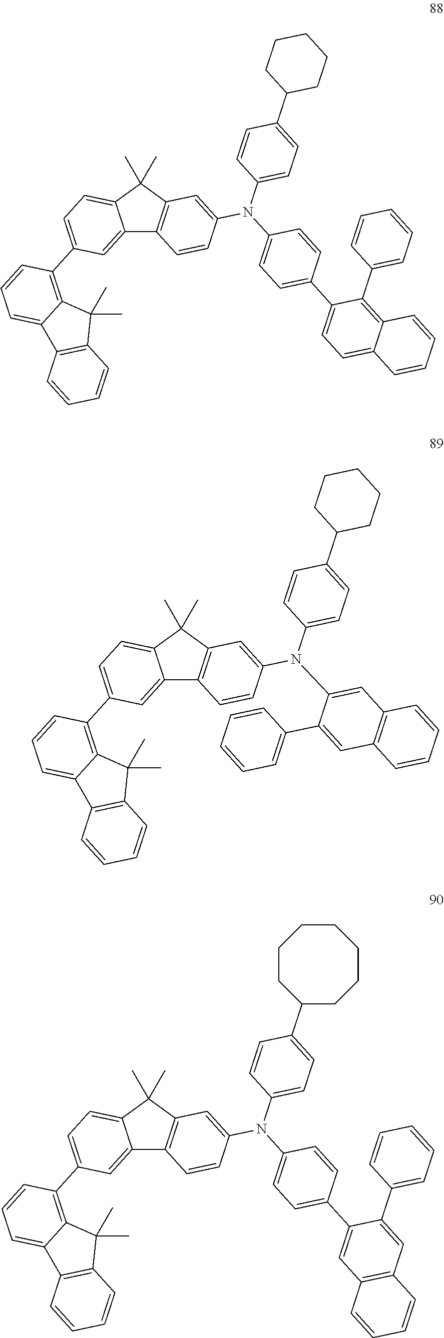 [SMILES: CC1(C)c2ccc(-c3cccc4c3C(C)(C)c3ccccc3-4)cc2-c2ccc(N(c3ccc(-c4ccc5ccccc5c4-c4ccccc4)cc3)c3ccc(C4CCCCC4)cc3)cc21 ; CC1(C)c2ccc(-c3cccc4c3C(C)(C)c3ccccc3-4)cc2-c2ccc(N(c3ccc(C4CCCCC4)cc3)c3cc4ccccc4cc3-c3ccccc3)cc21 ; CC1(C)c2ccc(-c3cccc4c3C(C)(C)c3ccccc3-4)cc2-c2ccc(N(c3ccc(-c4cc5ccccc5cc4-c4ccccc4)cc3)c3ccc(C4CCCCCCC4)cc3)cc21]

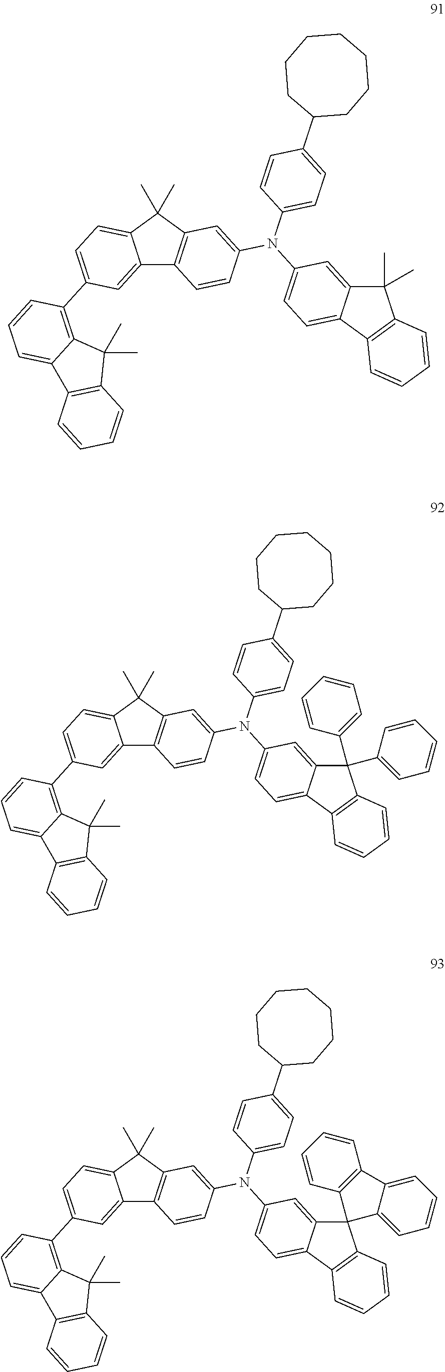 [SMILES: CC1(C)c2ccccc2-c2ccc(N(c3ccc(C4CCCCCCC4)cc3)c3ccc4c(c3)C(C)(C)c3ccc(-c5cccc6c5C(C)(C)c5ccccc5-6)cc3-4)cc21 ; CC1(C)c2ccc(-c3cccc4c3C(C)(C)c3ccccc3-4)cc2-c2ccc(N(c3ccc(C4CCCCCCC4)cc3)c3ccc4c(c3)C(c3ccccc3)(c3ccccc3)c3ccccc3-4)cc21 ; CC1(C)c2ccc(-c3cccc4c3C(C)(C)c3ccccc3-4)cc2-c2ccc(N(c3ccc(C4CCCCCCC4)cc3)c3ccc4c(c3)C3(c5ccccc5-c5ccccc53)c3ccccc3-4)cc21]

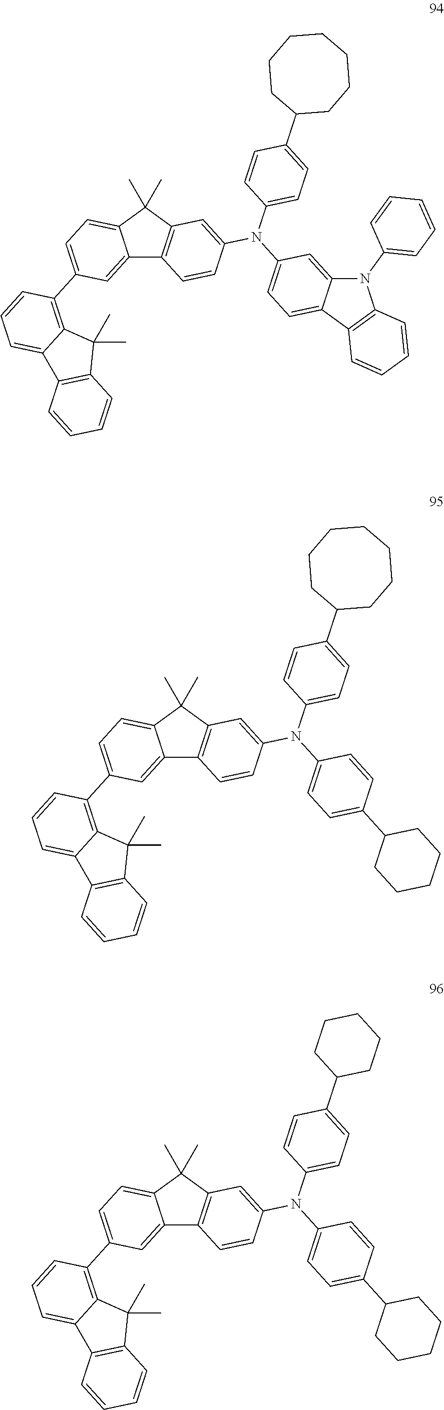 [SMILES: CC1(C)c2ccc(-c3cccc4c3C(C)(C)c3ccccc3-4)cc2-c2ccc(N(c3ccc(C4CCCCCCC4)cc3)c3ccc4c5ccccc5n(-c5ccccc5)c4c3)cc21 ; CC1(C)c2ccc(-c3cccc4c3C(C)(C)c3ccccc3-4)cc2-c2ccc(N(c3ccc(C4CCCCCCC4)cc3)c3ccc(C4CCCCC4)cc3)cc21 ; CC1(C)c2ccc(-c3cccc4c3C(C)(C)c3ccccc3-4)cc2-c2ccc(N(c3ccc(C4CCCCC4)cc3)c3ccc(C4CCCCC4)cc3)cc21]

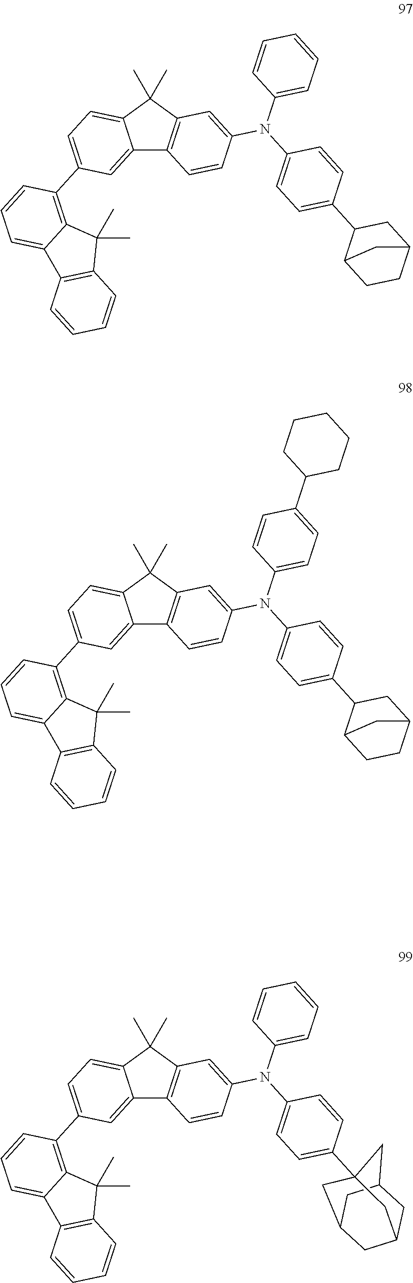 [SMILES: CC1(C)c2ccc(-c3cccc4c3C(C)(C)c3ccccc3-4)cc2-c2ccc(N(c3ccccc3)c3ccc(C4CC5CCC4C5)cc3)cc21 ; CC1(C)c2ccc(-c3cccc4c3C(C)(C)c3ccccc3-4)cc2-c2ccc(N(c3ccc(C4CCCCC4)cc3)c3ccc(C4CC5CCC4C5)cc3)cc21 ; CC1(C)c2ccc(-c3cccc4c3C(C)(C)c3ccccc3-4)cc2-c2ccc(N(c3ccccc3)c3ccc(C45CC6CC(CC(C6)C4)C5)cc3)cc21]

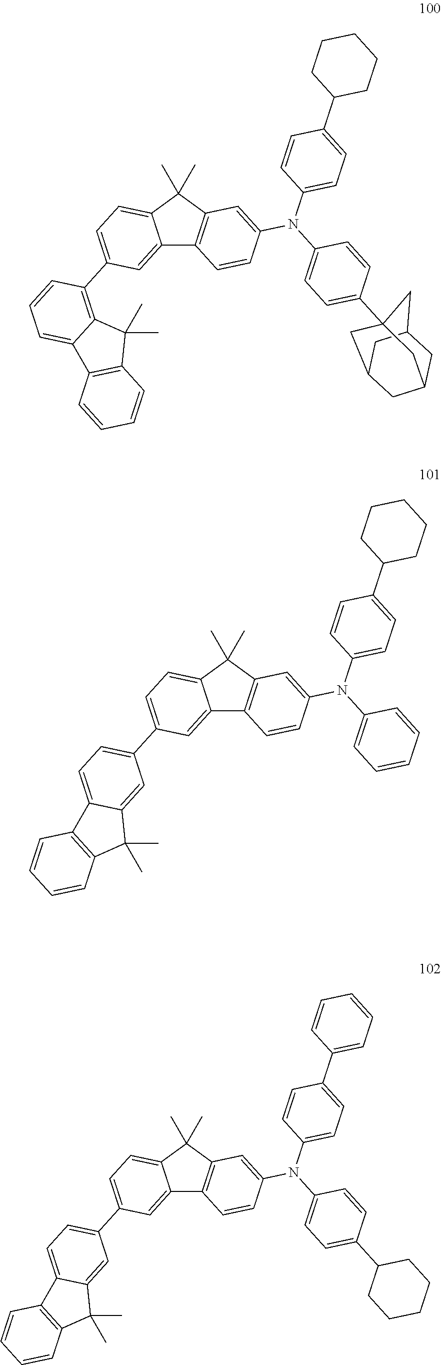 [SMILES: CC1(C)c2ccc(-c3cccc4c3C(C)(C)c3ccccc3-4)cc2-c2ccc(N(c3ccc(C4CCCCC4)cc3)c3ccc(C45CC6CC(CC(C6)C4)C5)cc3)cc21 ; CC1(C)c2ccccc2-c2ccc(-c3ccc4c(c3)-c3ccc(N(c5ccccc5)c5ccc(C6CCCCC6)cc5)cc3C4(C)C)cc21 ; CC1(C)c2ccccc2-c2ccc(-c3ccc4c(c3)-c3ccc(N(c5ccc(-c6ccccc6)cc5)c5ccc(C6CCCCC6)cc5)cc3C4(C)C)cc21]

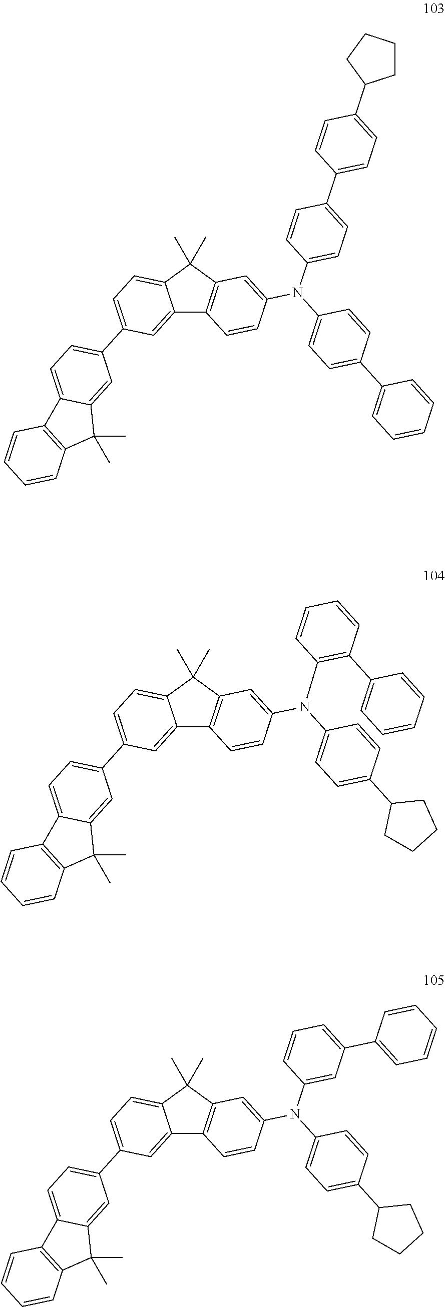 [SMILES: CC1(C)c2ccccc2-c2ccc(-c3ccc4c(c3)-c3ccc(N(c5ccc(-c6ccccc6)cc5)c5ccc(-c6ccc(C7CCCC7)cc6)cc5)cc3C4(C)C)cc21 ; CC1(C)c2ccccc2-c2ccc(-c3ccc4c(c3)-c3ccc(N(c5ccc(C6CCCC6)cc5)c5ccccc5-c5ccccc5)cc3C4(C)C)cc21 ; CC1(C)c2ccccc2-c2ccc(-c3ccc4c(c3)-c3ccc(N(c5ccc(C6CCCC6)cc5)c5cccc(-c6ccccc6)c5)cc3C4(C)C)cc21]

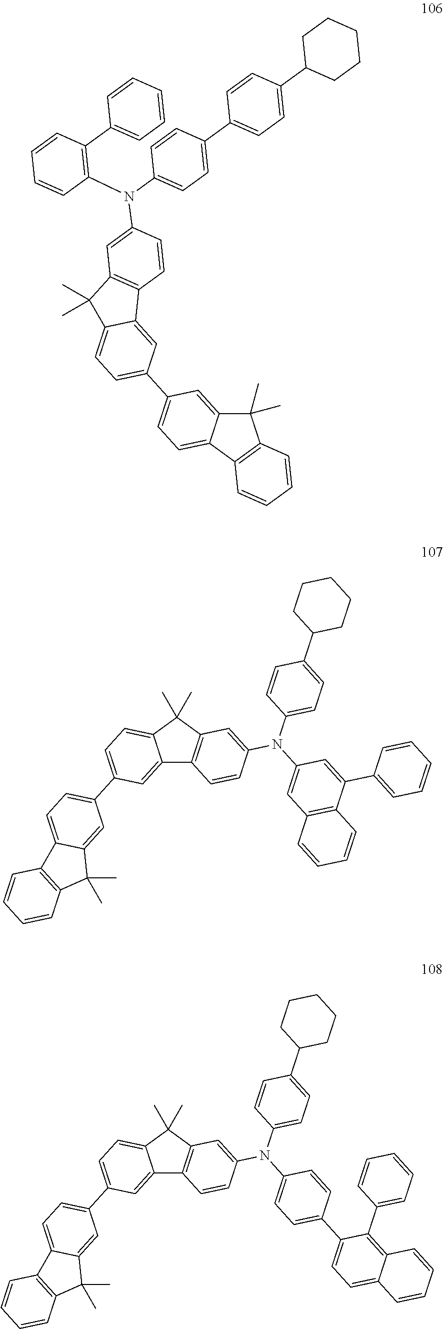 [SMILES: CC1(C)c2ccccc2-c2ccc(-c3ccc4c(c3)-c3ccc(N(c5ccc(-c6ccc(C7CCCCC7)cc6)cc5)c5ccccc5-c5ccccc5)cc3C4(C)C)cc21 ; CC1(C)c2ccccc2-c2ccc(-c3ccc4c(c3)-c3ccc(N(c5ccc(C6CCCCC6)cc5)c5cc(-c6ccccc6)c6ccccc6c5)cc3C4(C)C)cc21 ; CC1(C)c2ccccc2-c2ccc(-c3ccc4c(c3)-c3ccc(N(c5ccc(-c6ccc7ccccc7c6-c6ccccc6)cc5)c5ccc(C6CCCCC6)cc5)cc3C4(C)C)cc21]

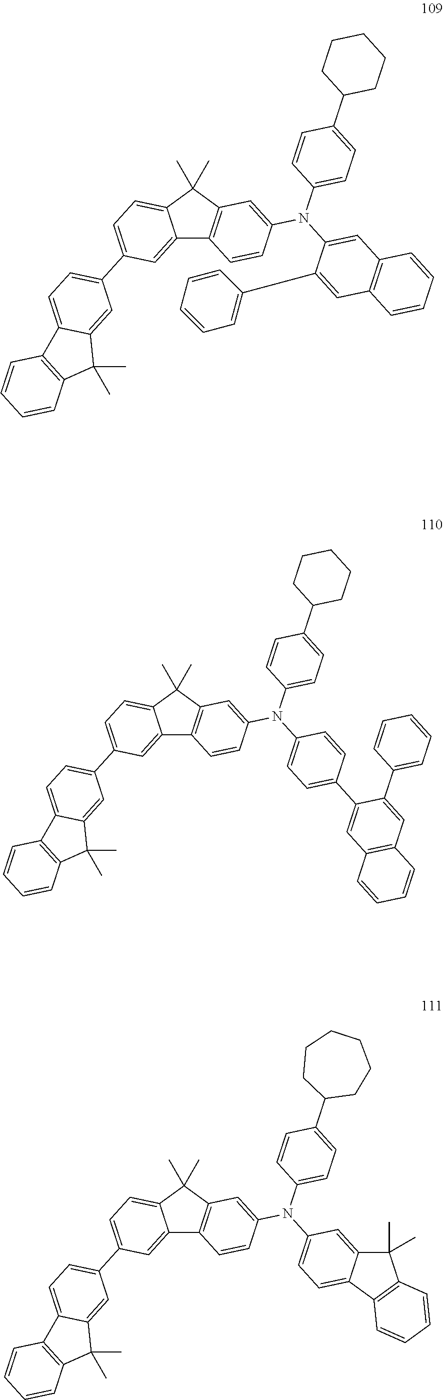 [SMILES: CC1(C)c2ccccc2-c2ccc(-c3ccc4c(c3)-c3ccc(N(c5ccc(C6CCCCC6)cc5)c5cc6ccccc6cc5-c5ccccc5)cc3C4(C)C)cc21 ; CC1(C)c2ccccc2-c2ccc(-c3ccc4c(c3)-c3ccc(N(c5ccc(-c6cc7ccccc7cc6-c6ccccc6)cc5)c5ccc(C6CCCCC6)cc5)cc3C4(C)C)cc21 ; CC1(C)c2ccccc2-c2ccc(-c3ccc4c(c3)-c3ccc(N(c5ccc(C6CCCCCC6)cc5)c5ccc6c(c5)C(C)(C)c5ccccc5-6)cc3C4(C)C)cc21]

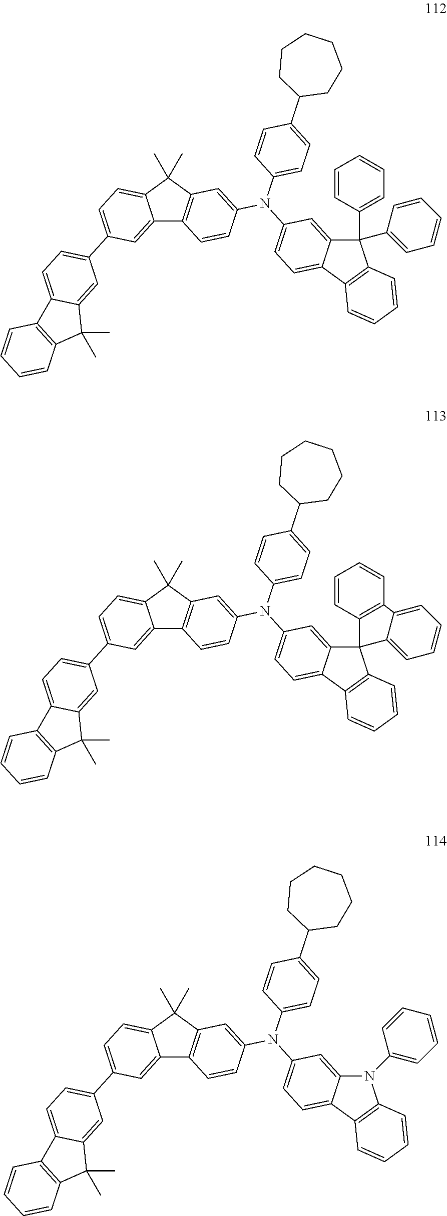 [SMILES: CC1(C)c2ccccc2-c2ccc(-c3ccc4c(c3)-c3ccc(N(c5ccc(C6CCCCCC6)cc5)c5ccc6c(c5)C(c5ccccc5)(c5ccccc5)c5ccccc5-6)cc3C4(C)C)cc21 ; CC1(C)c2ccccc2-c2ccc(-c3ccc4c(c3)-c3ccc(N(c5ccc(C6CCCCCC6)cc5)c5ccc6c(c5)C5(c7ccccc7-c7ccccc75)c5ccccc5-6)cc3C4(C)C)cc21 ; CC1(C)c2ccccc2-c2ccc(-c3ccc4c(c3)-c3ccc(N(c5ccc(C6CCCCCC6)cc5)c5ccc6c7ccccc7n(-c7ccccc7)c6c5)cc3C4(C)C)cc21]

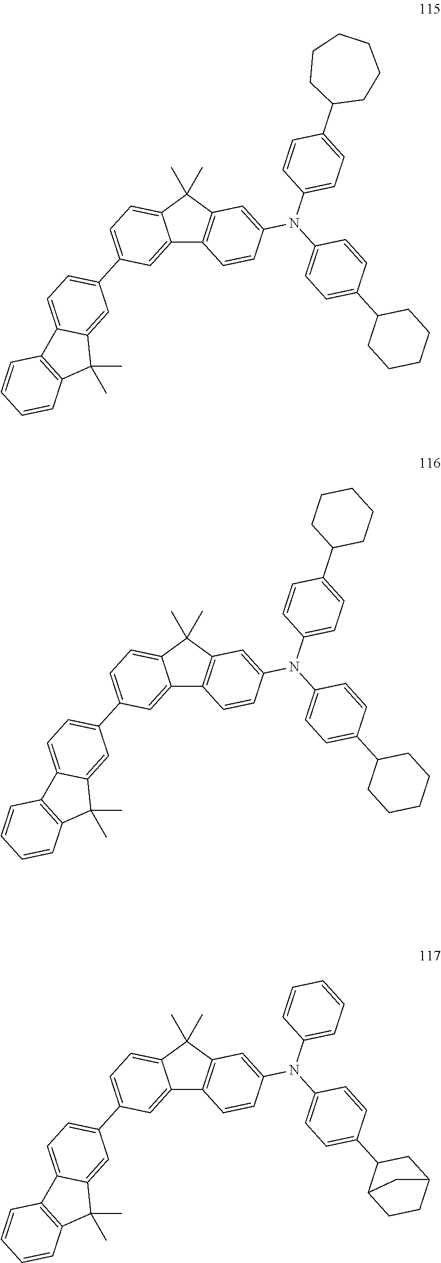 [SMILES: CC1(C)c2ccccc2-c2ccc(-c3ccc4c(c3)-c3ccc(N(c5ccc(C6CCCCCC6)cc5)c5ccc(C6CCCCC6)cc5)cc3C4(C)C)cc21 ; CC1(C)c2ccccc2-c2ccc(-c3ccc4c(c3)-c3ccc(N(c5ccc(C6CCCCC6)cc5)c5ccc(C6CCCCC6)cc5)cc3C4(C)C)cc21 ; CC1(C)c2ccccc2-c2ccc(-c3ccc4c(c3)-c3ccc(N(c5ccccc5)c5ccc(C6CC7CCC6C7)cc5)cc3C4(C)C)cc21]

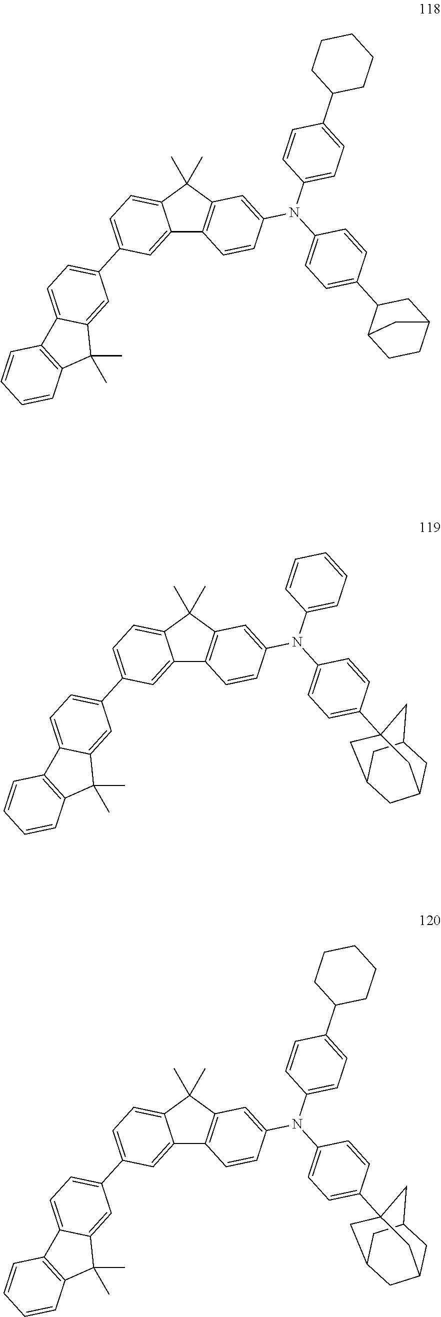 [SMILES: CC1(C)c2ccccc2-c2ccc(-c3ccc4c(c3)-c3ccc(N(c5ccc(C6CCCCC6)cc5)c5ccc(C6CC7CCC6C7)cc5)cc3C4(C)C)cc21 ; CC1(C)c2ccccc2-c2ccc(-c3ccc4c(c3)-c3ccc(N(c5ccccc5)c5ccc(C67CC8CC(CC(C8)C6)C7)cc5)cc3C4(C)C)cc21 ; CC1(C)c2ccccc2-c2ccc(-c3ccc4c(c3)-c3ccc(N(c5ccc(C6CCCCC6)cc5)c5ccc(C67CC8CC(CC(C8)C6)C7)cc5)cc3C4(C)C)cc21]

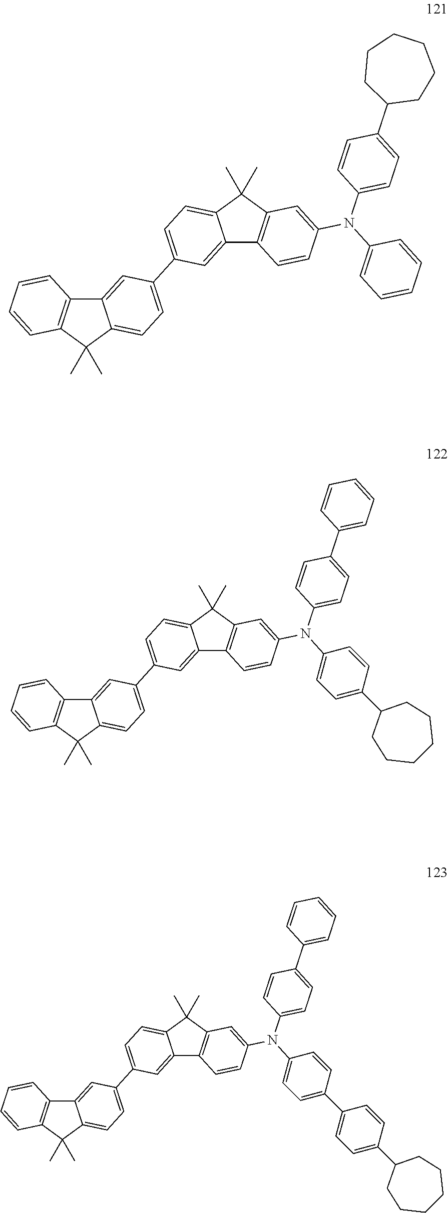 [SMILES: CC1(C)c2ccccc2-c2cc(-c3ccc4c(c3)-c3ccc(N(c5ccccc5)c5ccc(C6CCCCCC6)cc5)cc3C4(C)C)ccc21 ; CC1(C)c2ccccc2-c2cc(-c3ccc4c(c3)-c3ccc(N(c5ccc(-c6ccccc6)cc5)c5ccc(C6CCCCCC6)cc5)cc3C4(C)C)ccc21 ; CC1(C)c2ccccc2-c2cc(-c3ccc4c(c3)-c3ccc(N(c5ccc(-c6ccccc6)cc5)c5ccc(-c6ccc(C7CCCCCC7)cc6)cc5)cc3C4(C)C)ccc21]

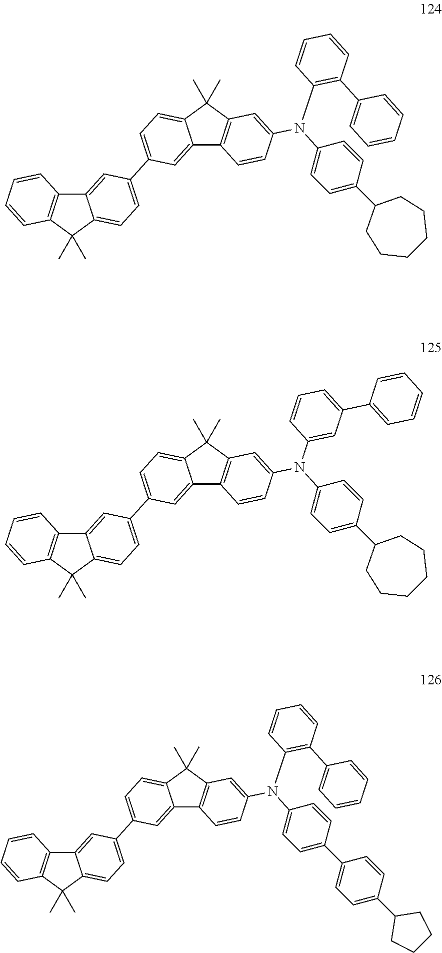 [SMILES: CC1(C)c2ccccc2-c2cc(-c3ccc4c(c3)-c3ccc(N(c5ccc(C6CCCCCC6)cc5)c5ccccc5-c5ccccc5)cc3C4(C)C)ccc21 ; CC1(C)c2ccccc2-c2cc(-c3ccc4c(c3)-c3ccc(N(c5ccc(C6CCCCCC6)cc5)c5cccc(-c6ccccc6)c5)cc3C4(C)C)ccc21 ; CC1(C)c2ccccc2-c2cc(-c3ccc4c(c3)-c3ccc(N(c5ccc(-c6ccc(C7CCCC7)cc6)cc5)c5ccccc5-c5ccccc5)cc3C4(C)C)ccc21]

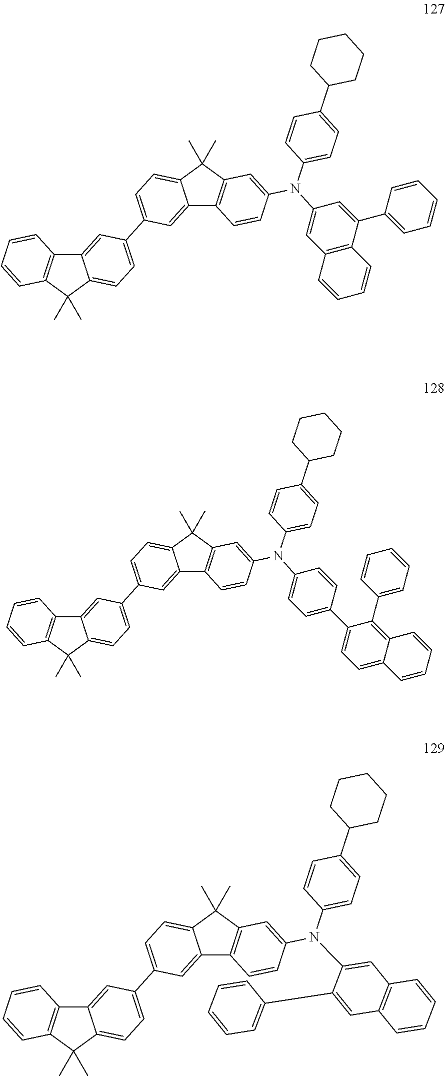 [SMILES: CC1(C)c2ccccc2-c2cc(-c3ccc4c(c3)-c3ccc(N(c5ccc(C6CCCCC6)cc5)c5cc(-c6ccccc6)c6ccccc6c5)cc3C4(C)C)ccc21 ; CC1(C)c2ccccc2-c2cc(-c3ccc4c(c3)-c3ccc(N(c5ccc(-c6ccc7ccccc7c6-c6ccccc6)cc5)c5ccc(C6CCCCC6)cc5)cc3C4(C)C)ccc21 ; CC1(C)c2ccccc2-c2cc(-c3ccc4c(c3)-c3ccc(N(c5ccc(C6CCCCC6)cc5)c5cc6ccccc6cc5-c5ccccc5)cc3C4(C)C)ccc21]

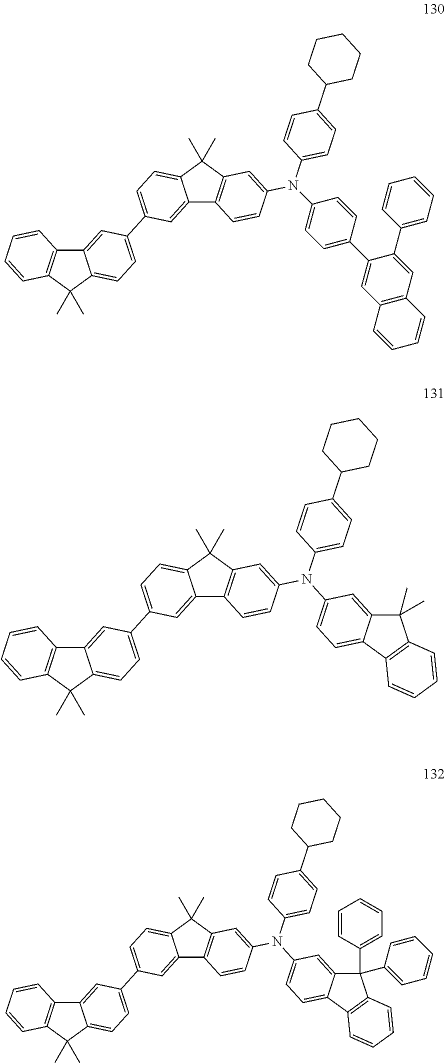 [SMILES: CC1(C)c2ccccc2-c2cc(-c3ccc4c(c3)-c3ccc(N(c5ccc(-c6cc7ccccc7cc6-c6ccccc6)cc5)c5ccc(C6CCCCC6)cc5)cc3C4(C)C)ccc21 ; CC1(C)c2ccccc2-c2cc(-c3ccc4c(c3)-c3ccc(N(c5ccc(C6CCCCC6)cc5)c5ccc6c(c5)C(C)(C)c5ccccc5-6)cc3C4(C)C)ccc21 ; CC1(C)c2ccccc2-c2cc(-c3ccc4c(c3)-c3ccc(N(c5ccc(C6CCCCC6)cc5)c5ccc6c(c5)C(c5ccccc5)(c5ccccc5)c5ccccc5-6)cc3C4(C)C)ccc21]

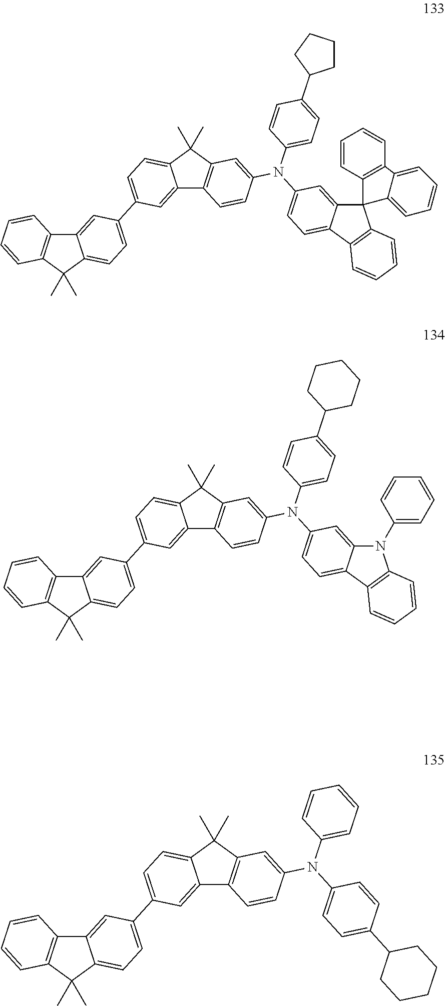 [SMILES: CC1(C)c2ccccc2-c2cc(-c3ccc4c(c3)-c3ccc(N(c5ccc(C6CCCC6)cc5)c5ccc6c(c5)C5(c7ccccc7-c7ccccc75)c5ccccc5-6)cc3C4(C)C)ccc21 ; CC1(C)c2ccccc2-c2cc(-c3ccc4c(c3)-c3ccc(N(c5ccc(C6CCCCC6)cc5)c5ccc6c7ccccc7n(-c7ccccc7)c6c5)cc3C4(C)C)ccc21 ; CC1(C)c2ccccc2-c2cc(-c3ccc4c(c3)-c3ccc(N(c5ccccc5)c5ccc(C6CCCCC6)cc5)cc3C4(C)C)ccc21]

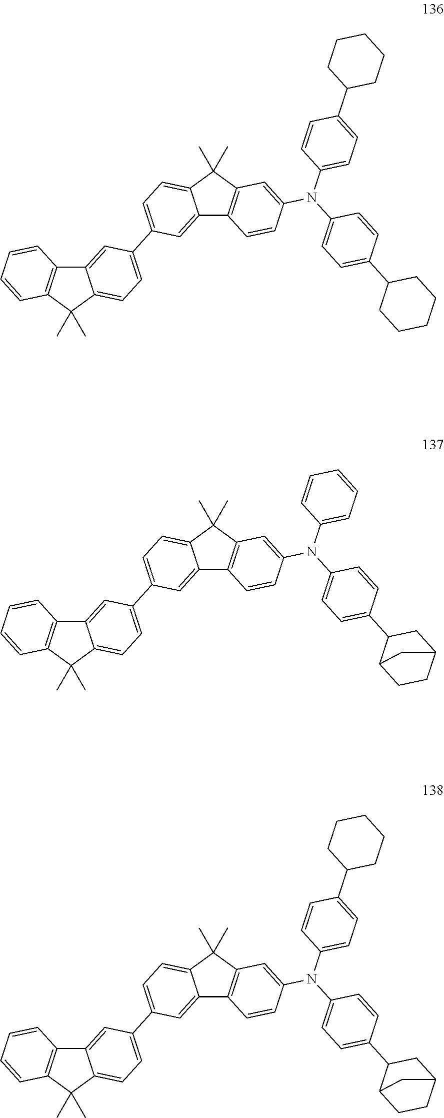 [SMILES: CC1(C)c2ccccc2-c2cc(-c3ccc4c(c3)-c3ccc(N(c5ccc(C6CCCCC6)cc5)c5ccc(C6CCCCC6)cc5)cc3C4(C)C)ccc21 ; CC1(C)c2ccccc2-c2cc(-c3ccc4c(c3)-c3ccc(N(c5ccccc5)c5ccc(C6CC7CCC6C7)cc5)cc3C4(C)C)ccc21 ; CC1(C)c2ccccc2-c2cc(-c3ccc4c(c3)-c3ccc(N(c5ccc(C6CCCCC6)cc5)c5ccc(C6CC7CCC6C7)cc5)cc3C4(C)C)ccc21]

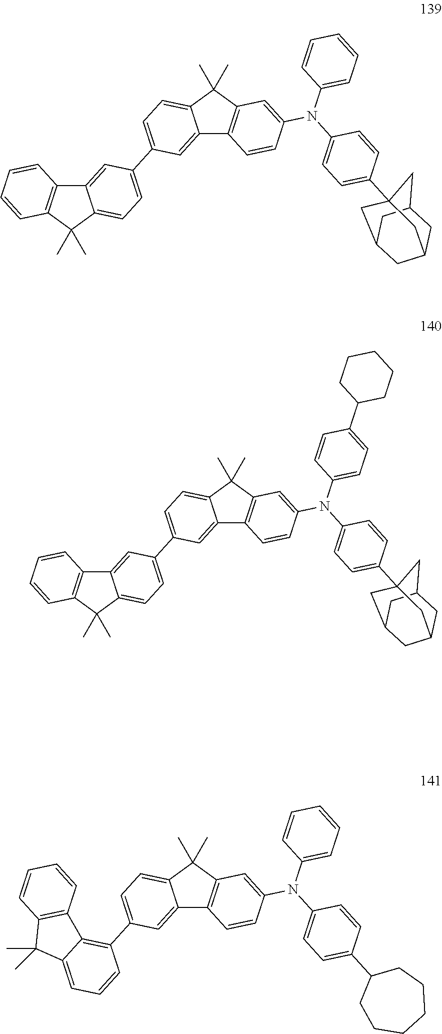 [SMILES: CC1(C)c2ccccc2-c2cc(-c3ccc4c(c3)-c3ccc(N(c5ccccc5)c5ccc(C67CC8CC(CC(C8)C6)C7)cc5)cc3C4(C)C)ccc21 ; CC1(C)c2ccccc2-c2cc(-c3ccc4c(c3)-c3ccc(N(c5ccc(C6CCCCC6)cc5)c5ccc(C67CC8CC(CC(C8)C6)C7)cc5)cc3C4(C)C)ccc21 ; CC1(C)c2ccc(-c3cccc4c3-c3ccccc3C4(C)C)cc2-c2ccc(N(c3ccccc3)c3ccc(C4CCCCCC4)cc3)cc21]

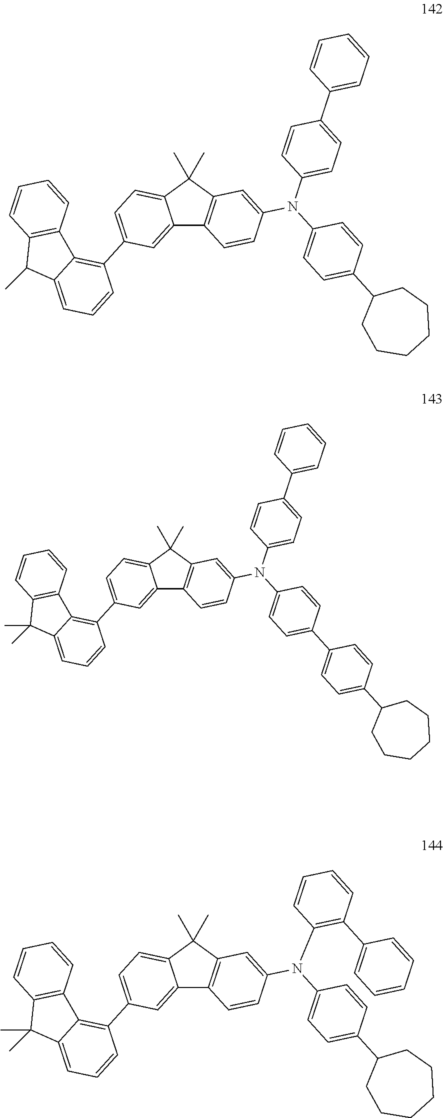 [SMILES: CC1c2ccccc2-c2c(-c3ccc4c(c3)-c3ccc(N(c5ccc(-c6ccccc6)cc5)c5ccc(C6CCCCCC6)cc5)cc3C4(C)C)cccc21 ; CC1(C)c2ccc(-c3cccc4c3-c3ccccc3C4(C)C)cc2-c2ccc(N(c3ccc(-c4ccccc4)cc3)c3ccc(-c4ccc(C5CCCCCC5)cc4)cc3)cc21 ; CC1(C)c2ccc(-c3cccc4c3-c3ccccc3C4(C)C)cc2-c2ccc(N(c3ccc(C4CCCCCC4)cc3)c3ccccc3-c3ccccc3)cc21]

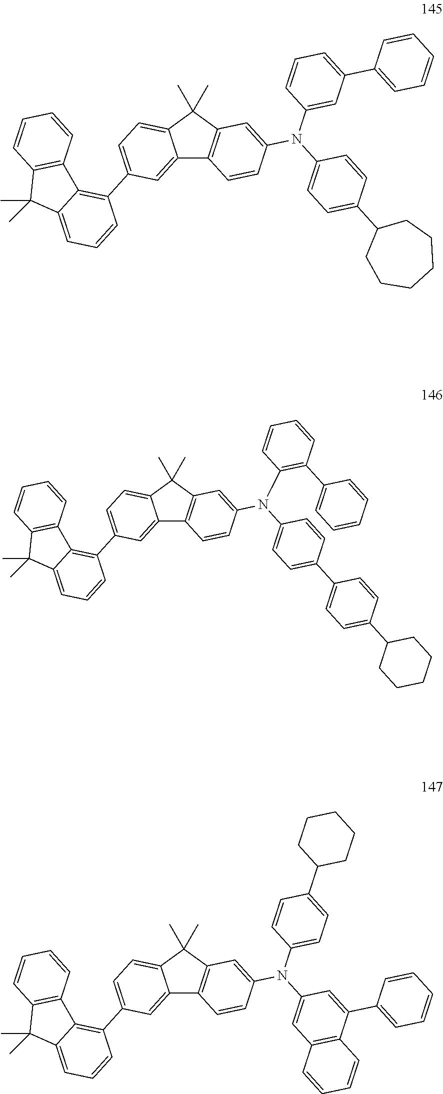 [SMILES: CC1(C)c2ccc(-c3cccc4c3-c3ccccc3C4(C)C)cc2-c2ccc(N(c3ccc(C4CCCCCC4)cc3)c3cccc(-c4ccccc4)c3)cc21 ; CC1(C)c2ccc(-c3cccc4c3-c3ccccc3C4(C)C)cc2-c2ccc(N(c3ccc(-c4ccc(C5CCCCC5)cc4)cc3)c3ccccc3-c3ccccc3)cc21 ; CC1(C)c2ccc(-c3cccc4c3-c3ccccc3C4(C)C)cc2-c2ccc(N(c3ccc(C4CCCCC4)cc3)c3cc(-c4ccccc4)c4ccccc4c3)cc21]

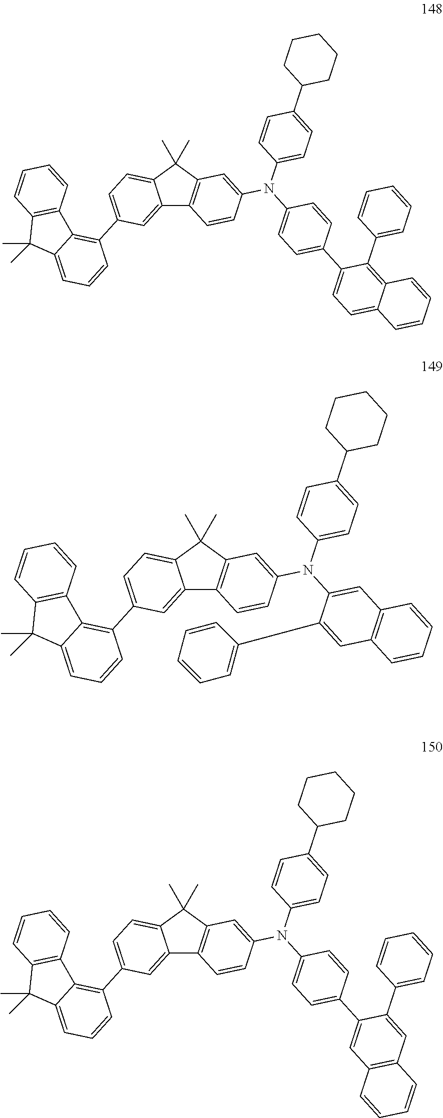 [SMILES: CC1(C)c2ccc(-c3cccc4c3-c3ccccc3C4(C)C)cc2-c2ccc(N(c3ccc(-c4ccc5ccccc5c4-c4ccccc4)cc3)c3ccc(C4CCCCC4)cc3)cc21 ; CC1(C)c2ccc(-c3cccc4c3-c3ccccc3C4(C)C)cc2-c2ccc(N(c3ccc(C4CCCCC4)cc3)c3cc4ccccc4cc3-c3ccccc3)cc21 ; CC1(C)c2ccc(-c3cccc4c3-c3ccccc3C4(C)C)cc2-c2ccc(N(c3ccc(-c4cc5ccccc5cc4-c4ccccc4)cc3)c3ccc(C4CCCCC4)cc3)cc21]

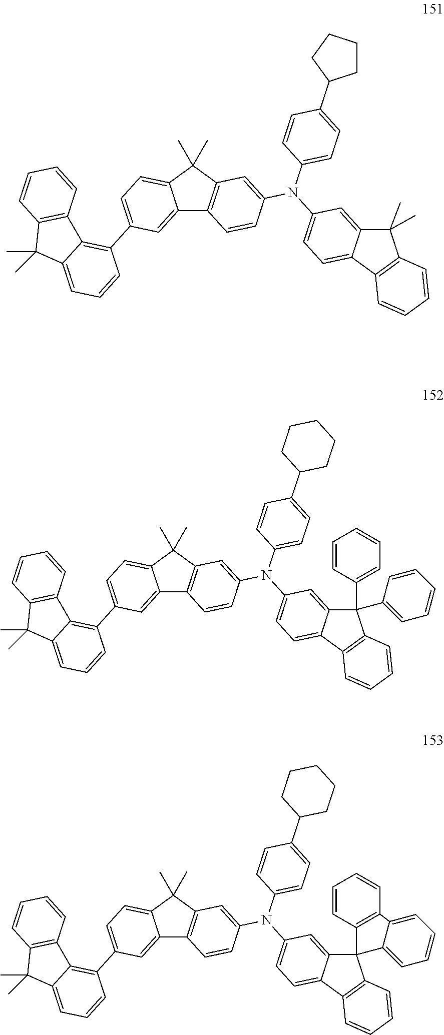 [SMILES: CC1(C)c2ccccc2-c2ccc(N(c3ccc(C4CCCC4)cc3)c3ccc4c(c3)C(C)(C)c3ccc(-c5cccc6c5-c5ccccc5C6(C)C)cc3-4)cc21 ; CC1(C)c2ccc(-c3cccc4c3-c3ccccc3C4(C)C)cc2-c2ccc(N(c3ccc(C4CCCCC4)cc3)c3ccc4c(c3)C(c3ccccc3)(c3ccccc3)c3ccccc3-4)cc21 ; CC1(C)c2ccc(-c3cccc4c3-c3ccccc3C4(C)C)cc2-c2ccc(N(c3ccc(C4CCCCC4)cc3)c3ccc4c(c3)C3(c5ccccc5-c5ccccc53)c3ccccc3-4)cc21]

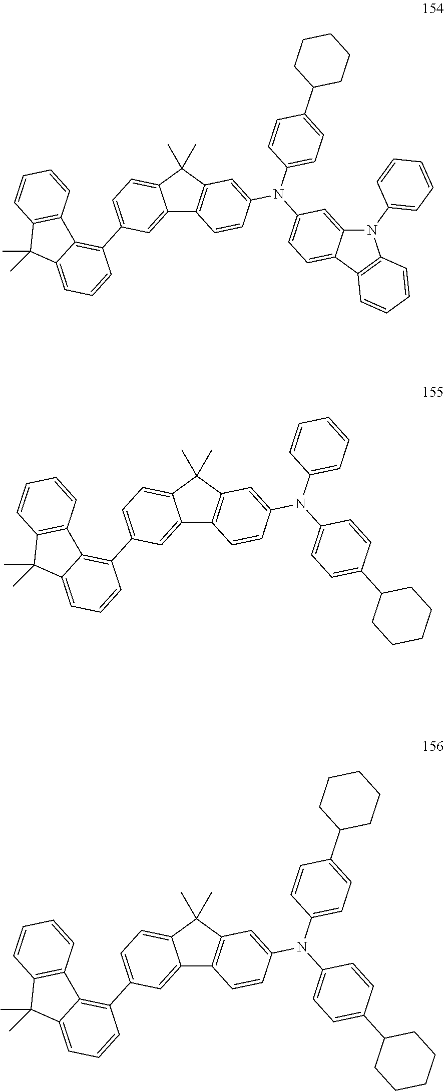 [SMILES: CC1(C)c2ccc(-c3cccc4c3-c3ccccc3C4(C)C)cc2-c2ccc(N(c3ccc(C4CCCCC4)cc3)c3ccc4c5ccccc5n(-c5ccccc5)c4c3)cc21 ; CC1(C)c2ccc(-c3cccc4c3-c3ccccc3C4(C)C)cc2-c2ccc(N(c3ccccc3)c3ccc(C4CCCCC4)cc3)cc21 ; CC1(C)c2ccc(-c3cccc4c3-c3ccccc3C4(C)C)cc2-c2ccc(N(c3ccc(C4CCCCC4)cc3)c3ccc(C4CCCCC4)cc3)cc21]

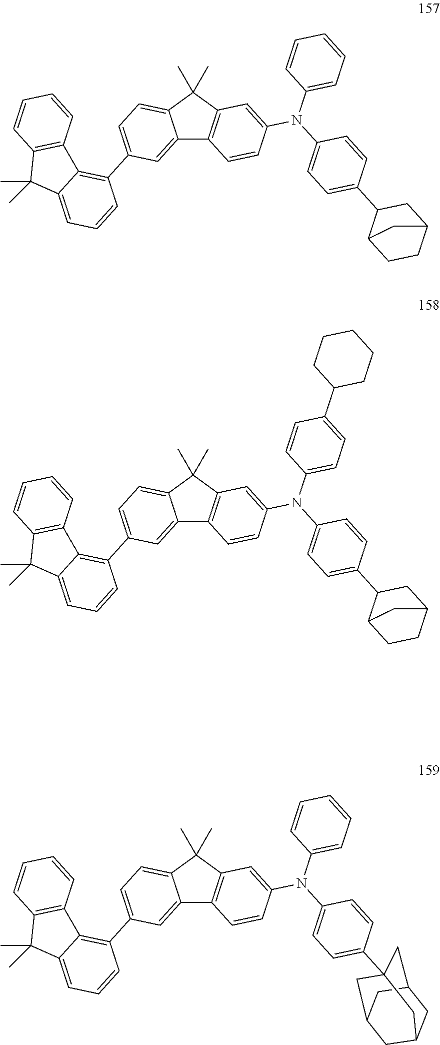 [SMILES: CC1(C)c2ccc(-c3cccc4c3-c3ccccc3C4(C)C)cc2-c2ccc(N(c3ccccc3)c3ccc(C4CC5CCC4C5)cc3)cc21 ; CC1(C)c2ccc(-c3cccc4c3-c3ccccc3C4(C)C)cc2-c2ccc(N(c3ccc(C4CCCCC4)cc3)c3ccc(C4CC5CCC4C5)cc3)cc21 ; CC1(C)c2ccc(-c3cccc4c3-c3ccccc3C4(C)C)cc2-c2ccc(N(c3ccccc3)c3ccc(C45CC6CC(CC(C6)C4)C5)cc3)cc21]

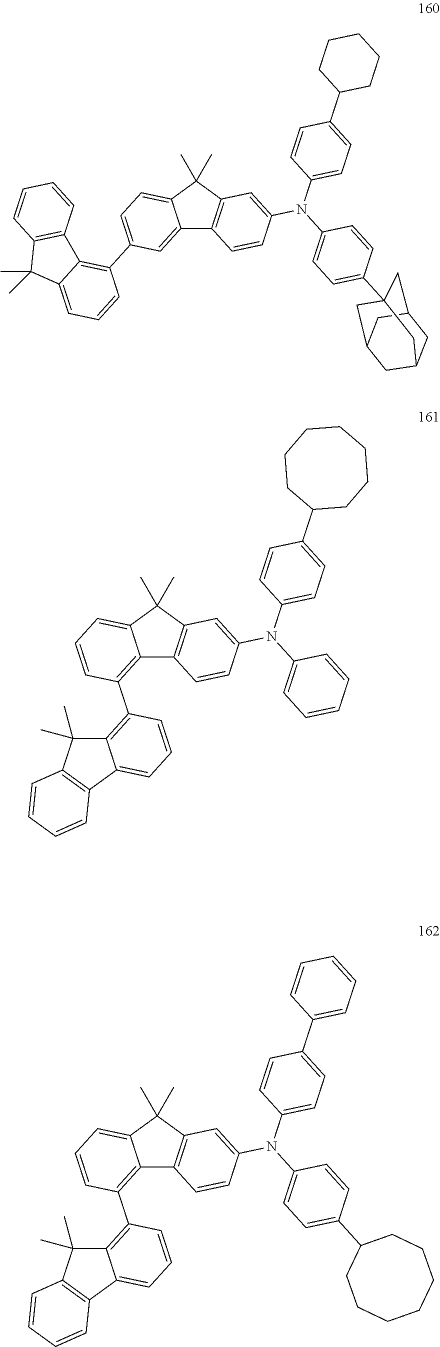 [SMILES: CC1(C)c2ccc(-c3cccc4c3-c3ccccc3C4(C)C)cc2-c2ccc(N(c3ccc(C4CCCCC4)cc3)c3ccc(C45CC6CC(CC(C6)C4)C5)cc3)cc21 ; CC1(C)c2cc(N(c3ccccc3)c3ccc(C4CCCCCCC4)cc3)ccc2-c2c(-c3cccc4c3C(C)(C)c3ccccc3-4)cccc21 ; CC1(C)c2cc(N(c3ccc(-c4ccccc4)cc3)c3ccc(C4CCCCCCC4)cc3)ccc2-c2c(-c3cccc4c3C(C)(C)c3ccccc3-4)cccc21]

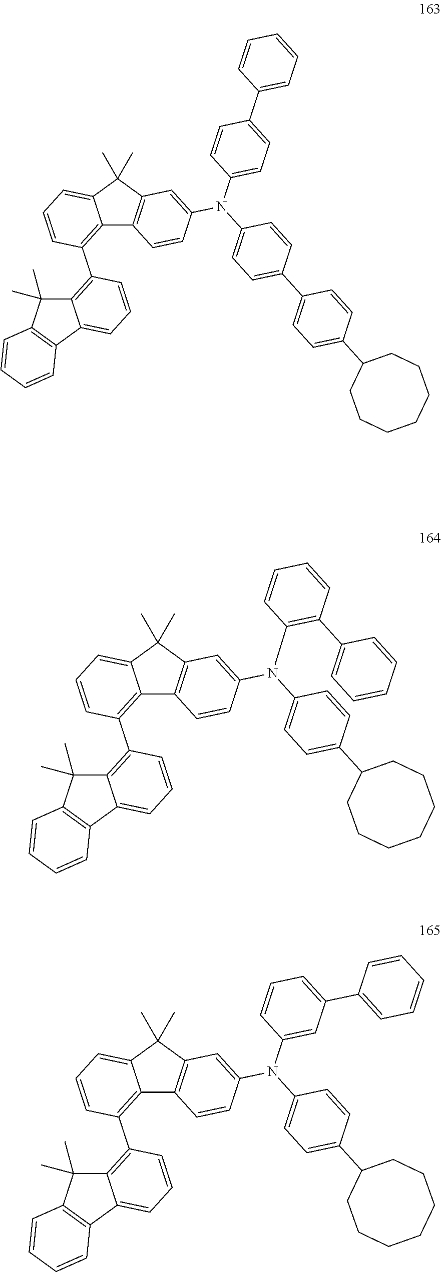 [SMILES: CC1(C)c2cc(N(c3ccc(-c4ccccc4)cc3)c3ccc(-c4ccc(C5CCCCCCC5)cc4)cc3)ccc2-c2c(-c3cccc4c3C(C)(C)c3ccccc3-4)cccc21 ; CC1(C)c2cc(N(c3ccc(C4CCCCCCC4)cc3)c3ccccc3-c3ccccc3)ccc2-c2c(-c3cccc4c3C(C)(C)c3ccccc3-4)cccc21 ; CC1(C)c2cc(N(c3ccc(C4CCCCCCC4)cc3)c3cccc(-c4ccccc4)c3)ccc2-c2c(-c3cccc4c3C(C)(C)c3ccccc3-4)cccc21]

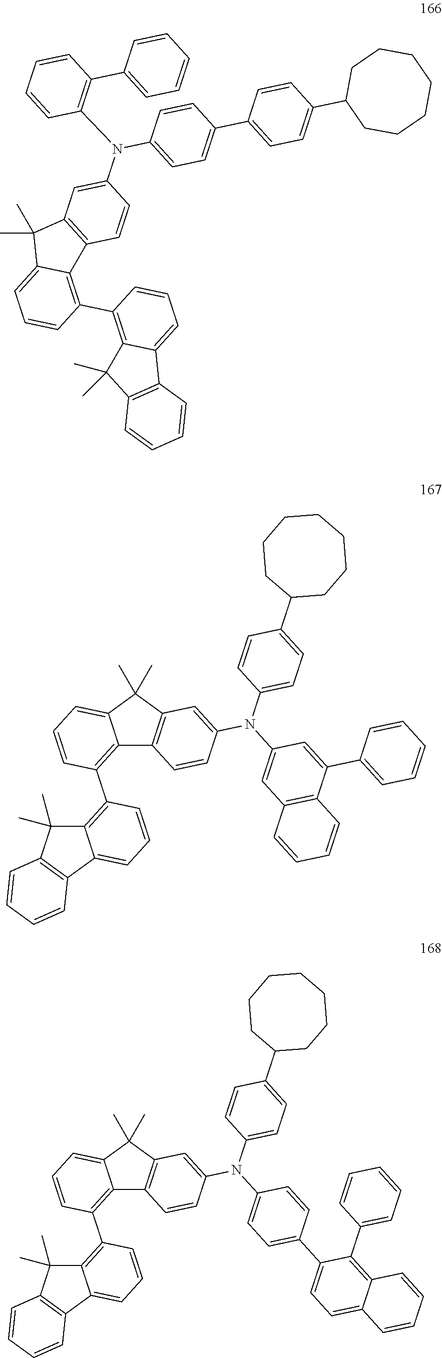 [SMILES: CC1(C)c2cc(N(c3ccc(-c4ccc(C5CCCCCCC5)cc4)cc3)c3ccccc3-c3ccccc3)ccc2-c2c(-c3cccc4c3C(C)(C)c3ccccc3-4)cccc21 ; CC1(C)c2cc(N(c3ccc(C4CCCCCCC4)cc3)c3cc(-c4ccccc4)c4ccccc4c3)ccc2-c2c(-c3cccc4c3C(C)(C)c3ccccc3-4)cccc21 ; CC1(C)c2cc(N(c3ccc(-c4ccc5ccccc5c4-c4ccccc4)cc3)c3ccc(C4CCCCCCC4)cc3)ccc2-c2c(-c3cccc4c3C(C)(C)c3ccccc3-4)cccc21]

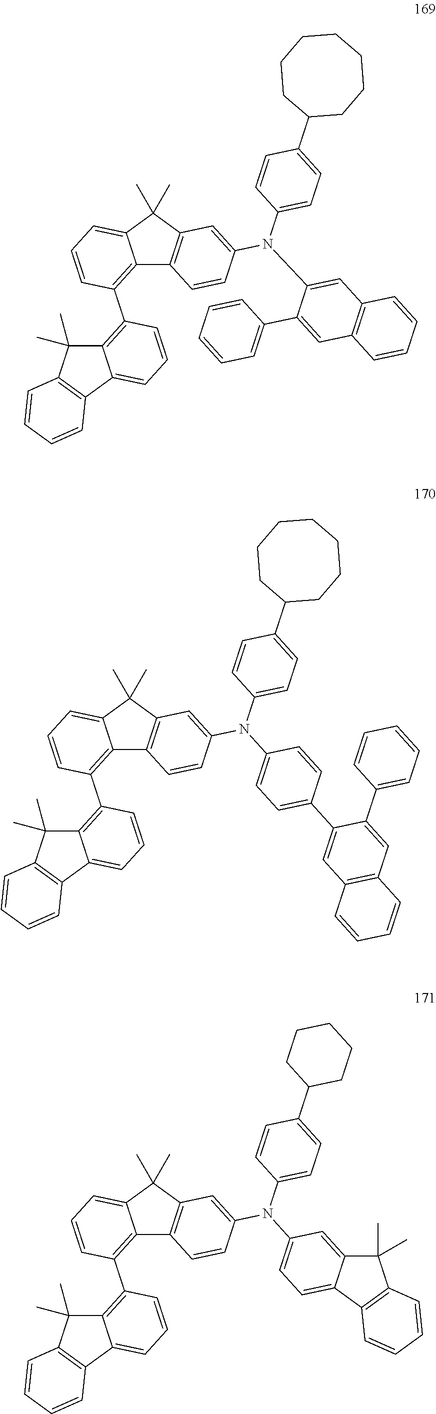 [SMILES: CC1(C)c2cc(N(c3ccc(C4CCCCCCC4)cc3)c3cc4ccccc4cc3-c3ccccc3)ccc2-c2c(-c3cccc4c3C(C)(C)c3ccccc3-4)cccc21 ; CC1(C)c2cc(N(c3ccc(-c4cc5ccccc5cc4-c4ccccc4)cc3)c3ccc(C4CCCCCCC4)cc3)ccc2-c2c(-c3cccc4c3C(C)(C)c3ccccc3-4)cccc21 ; CC1(C)c2ccccc2-c2ccc(N(c3ccc(C4CCCCC4)cc3)c3ccc4c(c3)C(C)(C)c3cccc(-c5cccc6c5C(C)(C)c5ccccc5-6)c3-4)cc21]

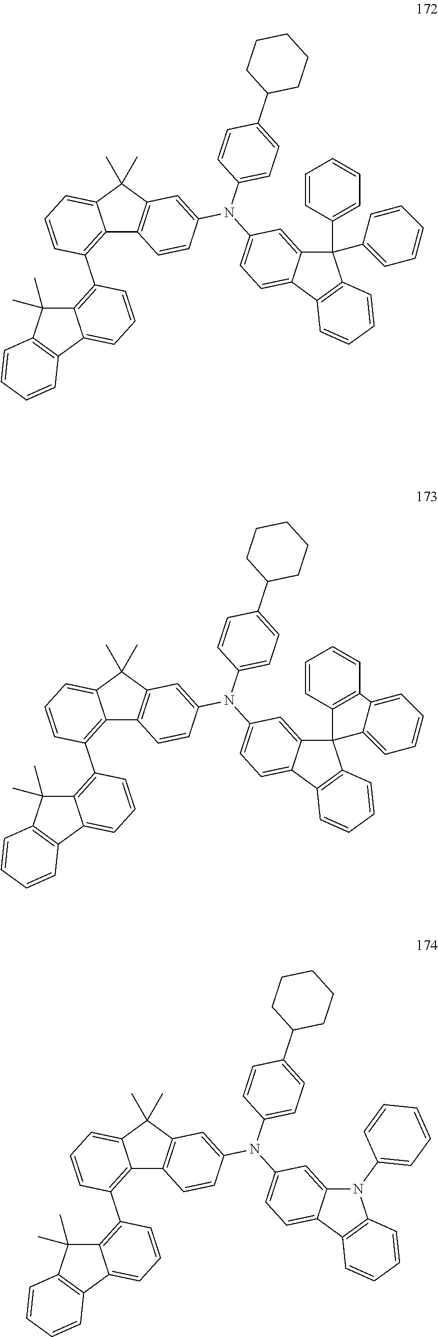 [SMILES: CC1(C)c2cc(N(c3ccc(C4CCCCC4)cc3)c3ccc4c(c3)C(c3ccccc3)(c3ccccc3)c3ccccc3-4)ccc2-c2c(-c3cccc4c3C(C)(C)c3ccccc3-4)cccc21 ; CC1(C)c2cc(N(c3ccc(C4CCCCC4)cc3)c3ccc4c(c3)C3(c5ccccc5-c5ccccc53)c3ccccc3-4)ccc2-c2c(-c3cccc4c3C(C)(C)c3ccccc3-4)cccc21 ; CC1(C)c2cc(N(c3ccc(C4CCCCC4)cc3)c3ccc4c5ccccc5n(-c5ccccc5)c4c3)ccc2-c2c(-c3cccc4c3C(C)(C)c3ccccc3-4)cccc21]

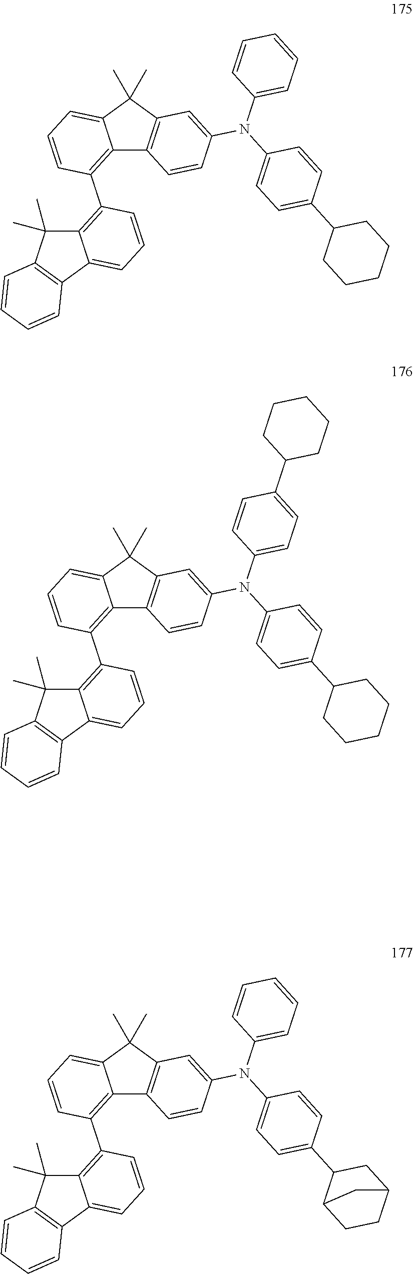 [SMILES: CC1(C)c2cc(N(c3ccccc3)c3ccc(C4CCCCC4)cc3)ccc2-c2c(-c3cccc4c3C(C)(C)c3ccccc3-4)cccc21 ; CC1(C)c2cc(N(c3ccc(C4CCCCC4)cc3)c3ccc(C4CCCCC4)cc3)ccc2-c2c(-c3cccc4c3C(C)(C)c3ccccc3-4)cccc21 ; CC1(C)c2cc(N(c3ccccc3)c3ccc(C4CC5CCC4C5)cc3)ccc2-c2c(-c3cccc4c3C(C)(C)c3ccccc3-4)cccc21]

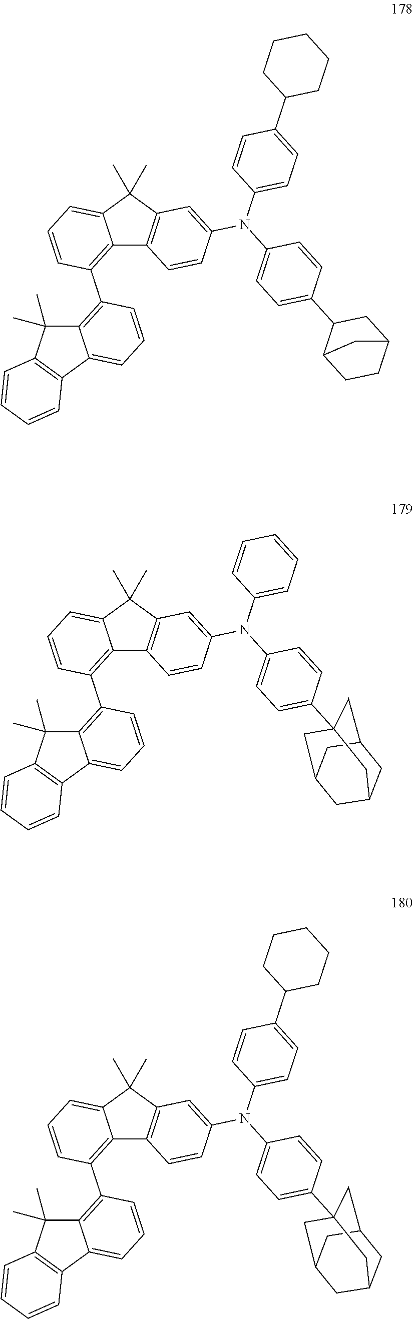 [SMILES: CC1(C)c2cc(N(c3ccc(C4CCCCC4)cc3)c3ccc(C4CC5CCC4C5)cc3)ccc2-c2c(-c3cccc4c3C(C)(C)c3ccccc3-4)cccc21 ; CC1(C)c2cc(N(c3ccccc3)c3ccc(C45CC6CC(CC(C6)C4)C5)cc3)ccc2-c2c(-c3cccc4c3C(C)(C)c3ccccc3-4)cccc21 ; CC1(C)c2cc(N(c3ccc(C4CCCCC4)cc3)c3ccc(C45CC6CC(CC(C6)C4)C5)cc3)ccc2-c2c(-c3cccc4c3C(C)(C)c3ccccc3-4)cccc21]

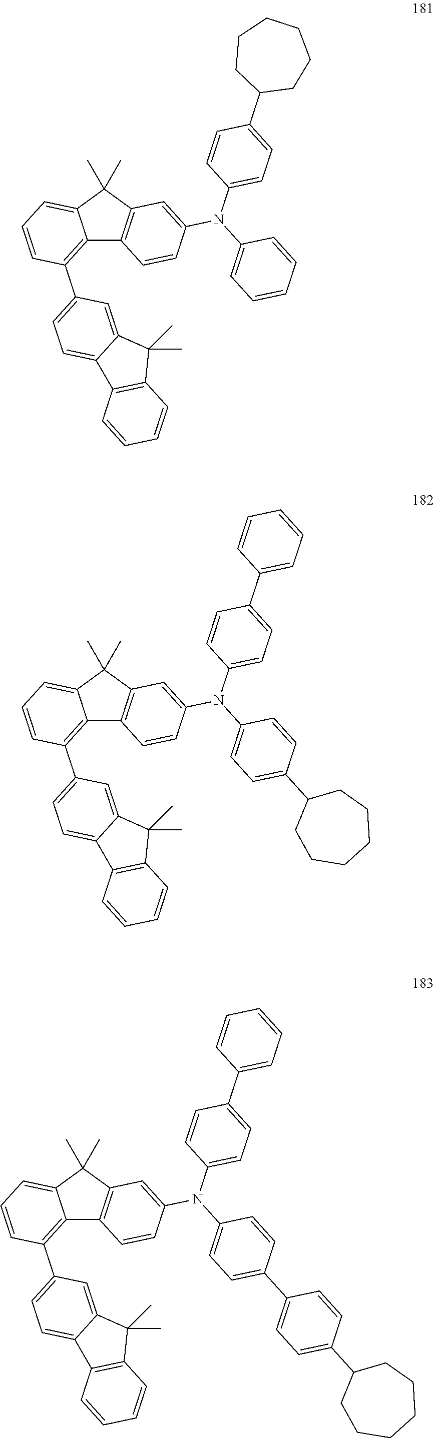 [SMILES: CC1(C)c2ccccc2-c2ccc(-c3cccc4c3-c3ccc(N(c5ccccc5)c5ccc(C6CCCCCC6)cc5)cc3C4(C)C)cc21 ; CC1(C)c2ccccc2-c2ccc(-c3cccc4c3-c3ccc(N(c5ccc(-c6ccccc6)cc5)c5ccc(C6CCCCCC6)cc5)cc3C4(C)C)cc21 ; CC1(C)c2ccccc2-c2ccc(-c3cccc4c3-c3ccc(N(c5ccc(-c6ccccc6)cc5)c5ccc(-c6ccc(C7CCCCCC7)cc6)cc5)cc3C4(C)C)cc21]

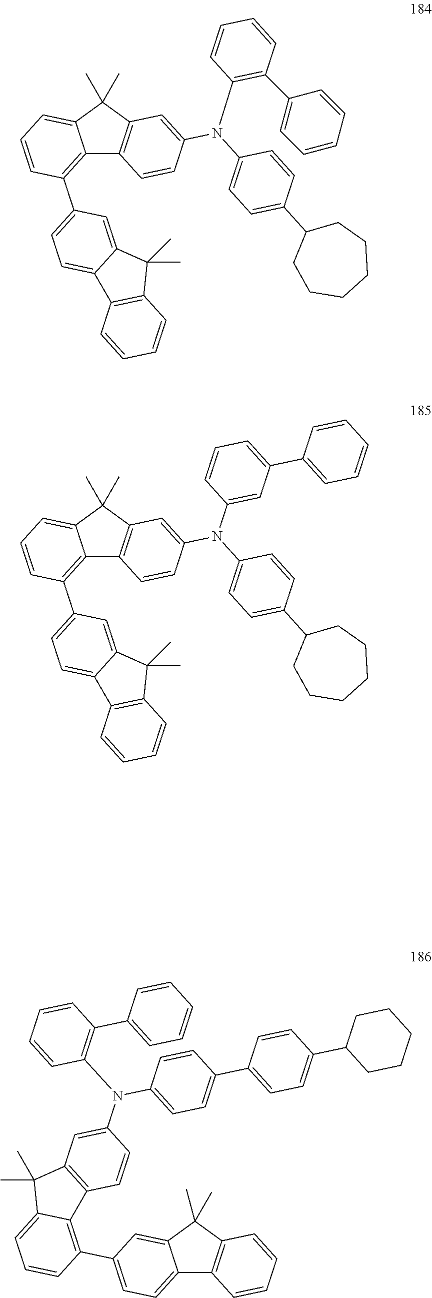 [SMILES: CC1(C)c2ccccc2-c2ccc(-c3cccc4c3-c3ccc(N(c5ccc(C6CCCCCC6)cc5)c5ccccc5-c5ccccc5)cc3C4(C)C)cc21 ; CC1(C)c2ccccc2-c2ccc(-c3cccc4c3-c3ccc(N(c5ccc(C6CCCCCC6)cc5)c5cccc(-c6ccccc6)c5)cc3C4(C)C)cc21 ; CC1(C)c2ccccc2-c2ccc(-c3cccc4c3-c3ccc(N(c5ccc(-c6ccc(C7CCCCC7)cc6)cc5)c5ccccc5-c5ccccc5)cc3C4(C)C)cc21]

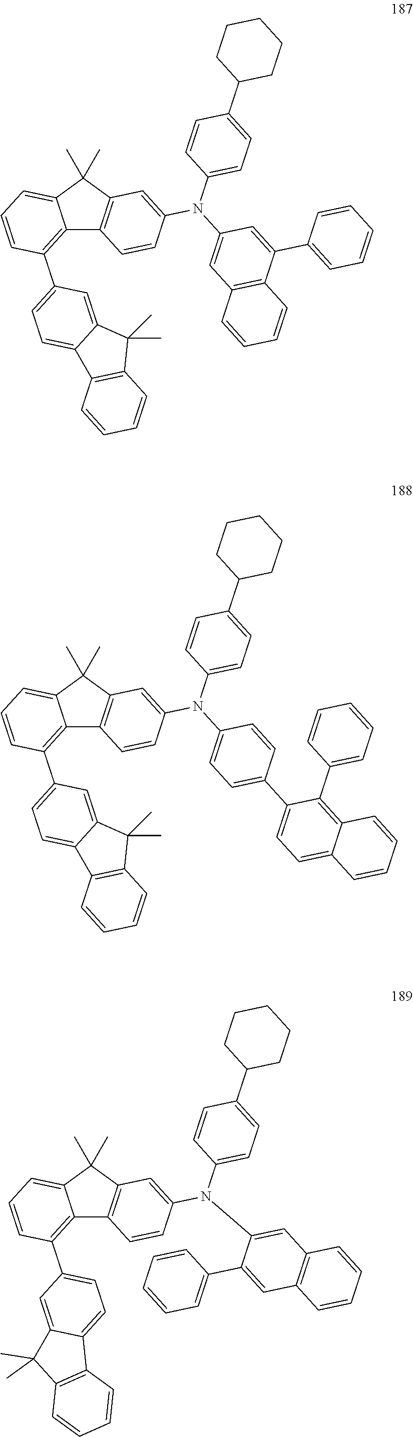 [SMILES: CC1(C)c2ccccc2-c2ccc(-c3cccc4c3-c3ccc(N(c5ccc(C6CCCCC6)cc5)c5cc(-c6ccccc6)c6ccccc6c5)cc3C4(C)C)cc21 ; CC1(C)c2ccccc2-c2ccc(-c3cccc4c3-c3ccc(N(c5ccc(-c6ccc7ccccc7c6-c6ccccc6)cc5)c5ccc(C6CCCCC6)cc5)cc3C4(C)C)cc21 ; CC1(C)c2ccccc2-c2ccc(-c3cccc4c3-c3ccc(N(c5ccc(C6CCCCC6)cc5)c5cc6ccccc6cc5-c5ccccc5)cc3C4(C)C)cc21]

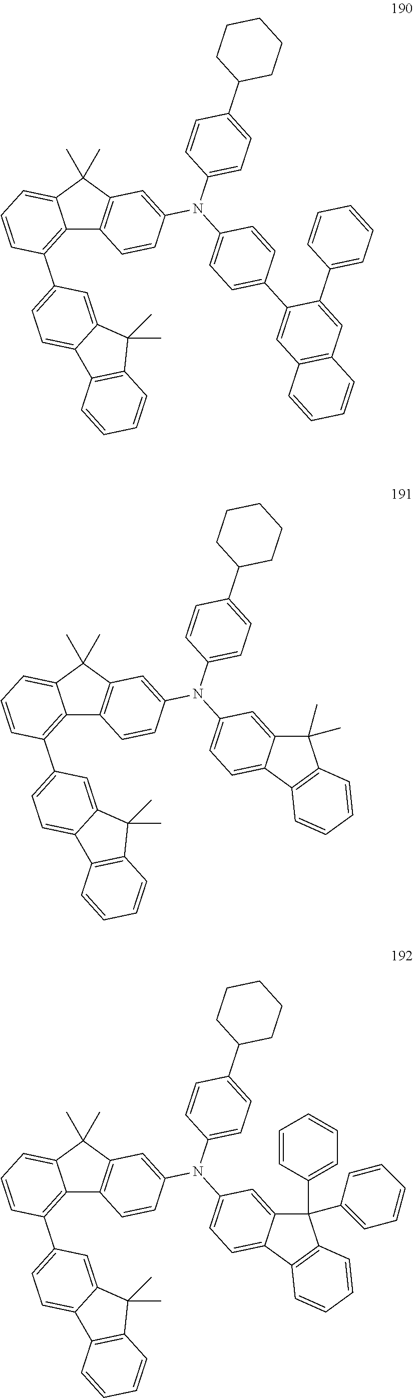 [SMILES: CC1(C)c2ccccc2-c2ccc(-c3cccc4c3-c3ccc(N(c5ccc(-c6cc7ccccc7cc6-c6ccccc6)cc5)c5ccc(C6CCCCC6)cc5)cc3C4(C)C)cc21 ; CC1(C)c2ccccc2-c2ccc(-c3cccc4c3-c3ccc(N(c5ccc(C6CCCCC6)cc5)c5ccc6c(c5)C(C)(C)c5ccccc5-6)cc3C4(C)C)cc21 ; CC1(C)c2ccccc2-c2ccc(-c3cccc4c3-c3ccc(N(c5ccc(C6CCCCC6)cc5)c5ccc6c(c5)C(c5ccccc5)(c5ccccc5)c5ccccc5-6)cc3C4(C)C)cc21]

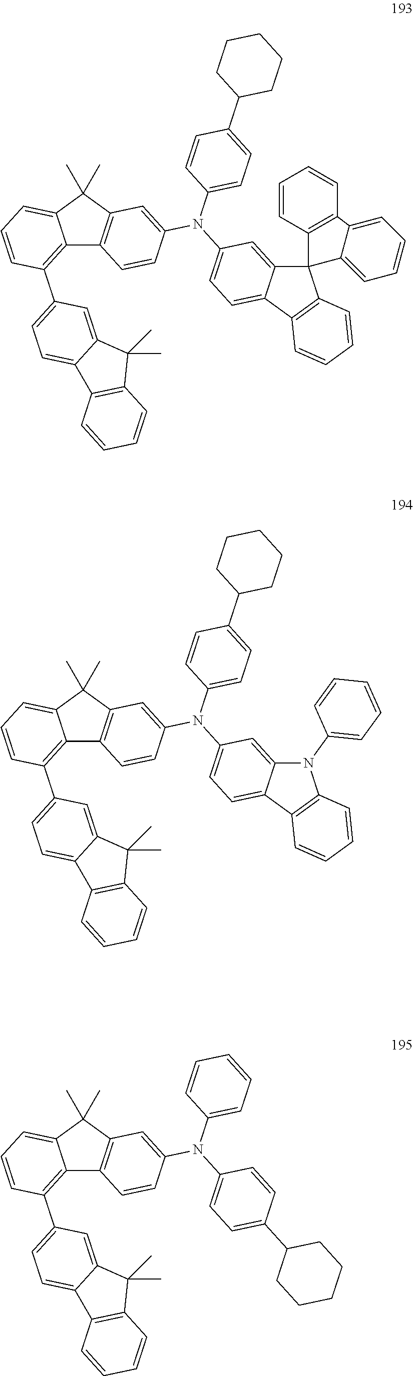 [SMILES: CC1(C)c2ccccc2-c2ccc(-c3cccc4c3-c3ccc(N(c5ccc(C6CCCCC6)cc5)c5ccc6c(c5)C5(c7ccccc7-c7ccccc75)c5ccccc5-6)cc3C4(C)C)cc21 ; CC1(C)c2ccccc2-c2ccc(-c3cccc4c3-c3ccc(N(c5ccc(C6CCCCC6)cc5)c5ccc6c7ccccc7n(-c7ccccc7)c6c5)cc3C4(C)C)cc21 ; CC1(C)c2ccccc2-c2ccc(-c3cccc4c3-c3ccc(N(c5ccccc5)c5ccc(C6CCCCC6)cc5)cc3C4(C)C)cc21]

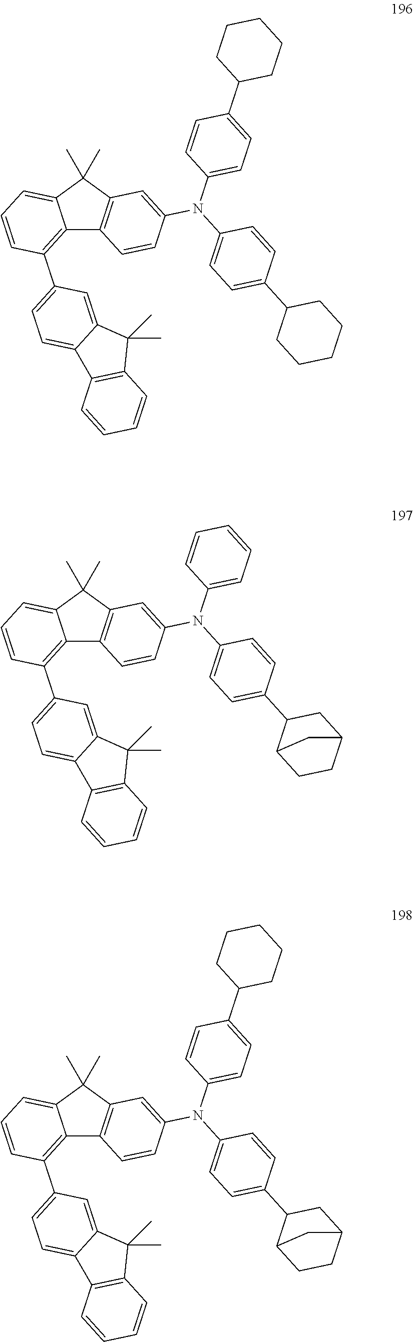 [SMILES: CC1(C)c2ccccc2-c2ccc(-c3cccc4c3-c3ccc(N(c5ccc(C6CCCCC6)cc5)c5ccc(C6CCCCC6)cc5)cc3C4(C)C)cc21 ; CC1(C)c2ccccc2-c2ccc(-c3cccc4c3-c3ccc(N(c5ccccc5)c5ccc(C6CC7CCC6C7)cc5)cc3C4(C)C)cc21 ; CC1(C)c2ccccc2-c2ccc(-c3cccc4c3-c3ccc(N(c5ccc(C6CCCCC6)cc5)c5ccc(C6CC7CCC6C7)cc5)cc3C4(C)C)cc21]

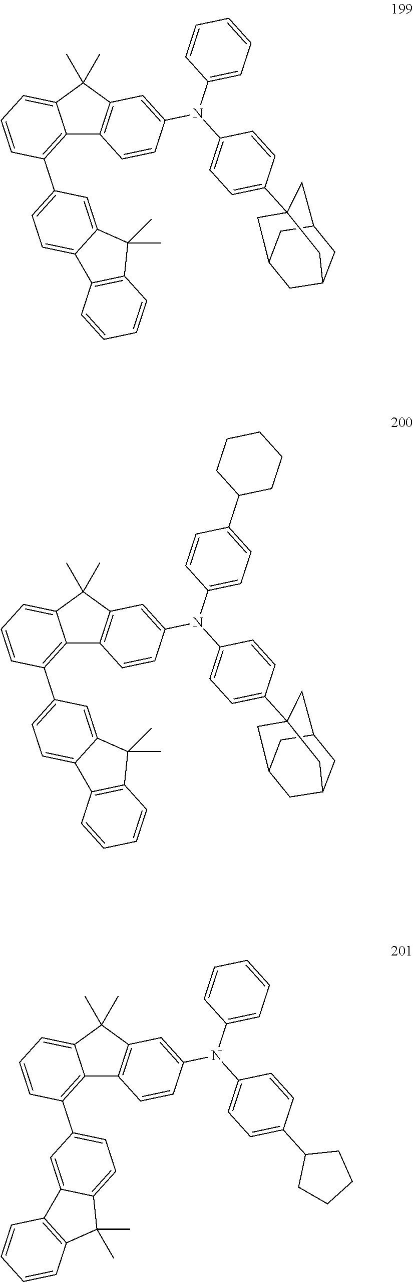 [SMILES: CC1(C)c2ccccc2-c2ccc(-c3cccc4c3-c3ccc(N(c5ccccc5)c5ccc(C67CC8CC(CC(C8)C6)C7)cc5)cc3C4(C)C)cc21 ; CC1(C)c2ccccc2-c2ccc(-c3cccc4c3-c3ccc(N(c5ccc(C6CCCCC6)cc5)c5ccc(C67CC8CC(CC(C8)C6)C7)cc5)cc3C4(C)C)cc21 ; CC1(C)c2ccccc2-c2cc(-c3cccc4c3-c3ccc(N(c5ccccc5)c5ccc(C6CCCC6)cc5)cc3C4(C)C)ccc21]

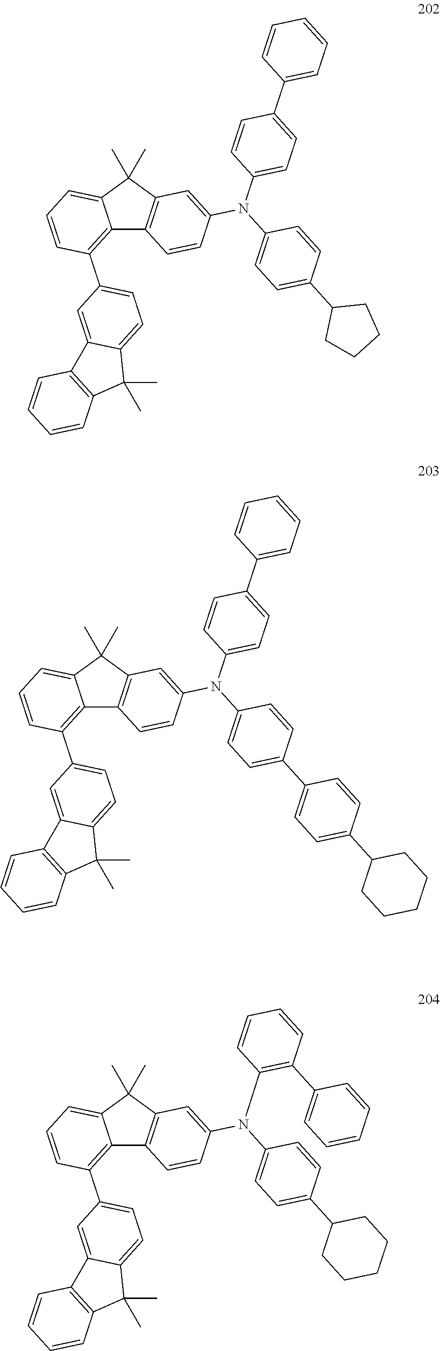 [SMILES: CC1(C)c2ccccc2-c2cc(-c3cccc4c3-c3ccc(N(c5ccc(-c6ccccc6)cc5)c5ccc(C6CCCC6)cc5)cc3C4(C)C)ccc21 ; CC1(C)c2ccccc2-c2cc(-c3cccc4c3-c3ccc(N(c5ccc(-c6ccccc6)cc5)c5ccc(-c6ccc(C7CCCCC7)cc6)cc5)cc3C4(C)C)ccc21 ; CC1(C)c2ccccc2-c2cc(-c3cccc4c3-c3ccc(N(c5ccc(C6CCCCC6)cc5)c5ccccc5-c5ccccc5)cc3C4(C)C)ccc21]

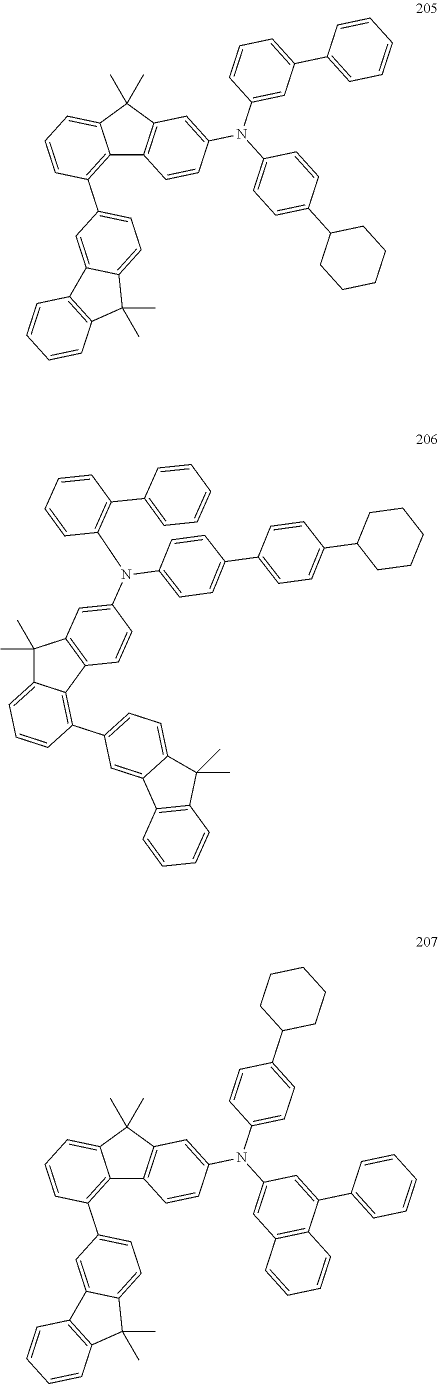 [SMILES: CC1(C)c2ccccc2-c2cc(-c3cccc4c3-c3ccc(N(c5ccc(C6CCCCC6)cc5)c5cccc(-c6ccccc6)c5)cc3C4(C)C)ccc21 ; CC1(C)c2ccccc2-c2cc(-c3cccc4c3-c3ccc(N(c5ccc(-c6ccc(C7CCCCC7)cc6)cc5)c5ccccc5-c5ccccc5)cc3C4(C)C)ccc21 ; CC1(C)c2ccccc2-c2cc(-c3cccc4c3-c3ccc(N(c5ccc(C6CCCCC6)cc5)c5cc(-c6ccccc6)c6ccccc6c5)cc3C4(C)C)ccc21]

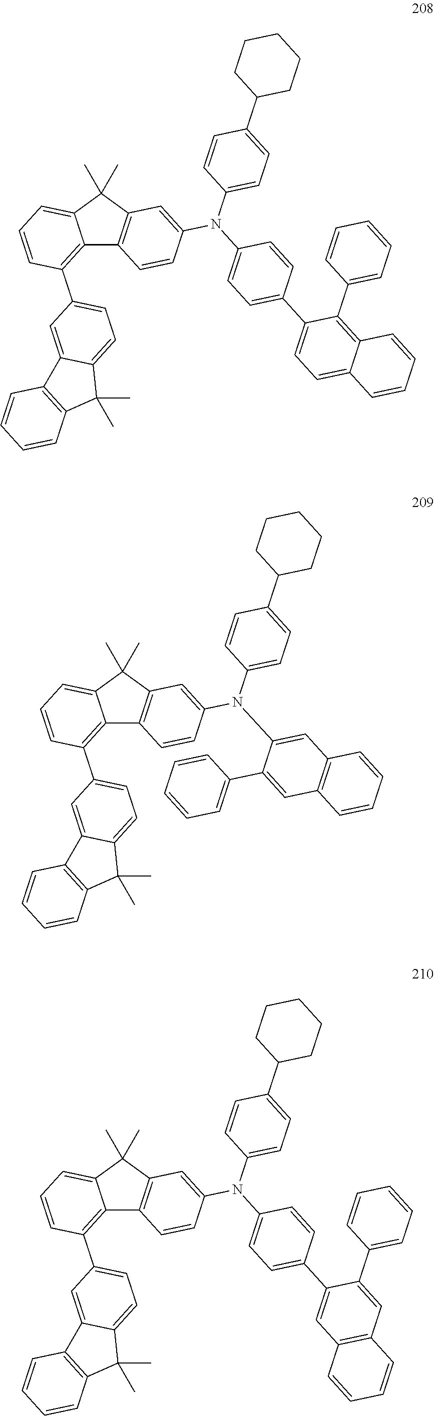 [SMILES: CC1(C)c2ccccc2-c2cc(-c3cccc4c3-c3ccc(N(c5ccc(-c6ccc7ccccc7c6-c6ccccc6)cc5)c5ccc(C6CCCCC6)cc5)cc3C4(C)C)ccc21 ; CC1(C)c2ccccc2-c2cc(-c3cccc4c3-c3ccc(N(c5ccc(C6CCCCC6)cc5)c5cc6ccccc6cc5-c5ccccc5)cc3C4(C)C)ccc21 ; CC1(C)c2ccccc2-c2cc(-c3cccc4c3-c3ccc(N(c5ccc(-c6cc7ccccc7cc6-c6ccccc6)cc5)c5ccc(C6CCCCC6)cc5)cc3C4(C)C)ccc21]

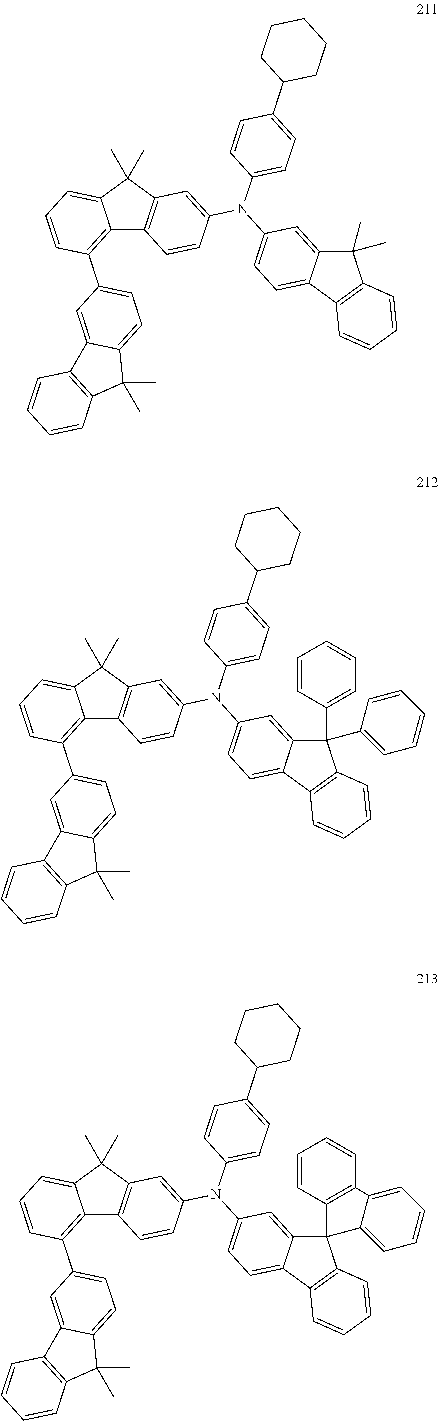 [SMILES: CC1(C)c2ccccc2-c2cc(-c3cccc4c3-c3ccc(N(c5ccc(C6CCCCC6)cc5)c5ccc6c(c5)C(C)(C)c5ccccc5-6)cc3C4(C)C)ccc21 ; CC1(C)c2ccccc2-c2cc(-c3cccc4c3-c3ccc(N(c5ccc(C6CCCCC6)cc5)c5ccc6c(c5)C(c5ccccc5)(c5ccccc5)c5ccccc5-6)cc3C4(C)C)ccc21 ; CC1(C)c2ccccc2-c2cc(-c3cccc4c3-c3ccc(N(c5ccc(C6CCCCC6)cc5)c5ccc6c(c5)C5(c7ccccc7-c7ccccc75)c5ccccc5-6)cc3C4(C)C)ccc21]

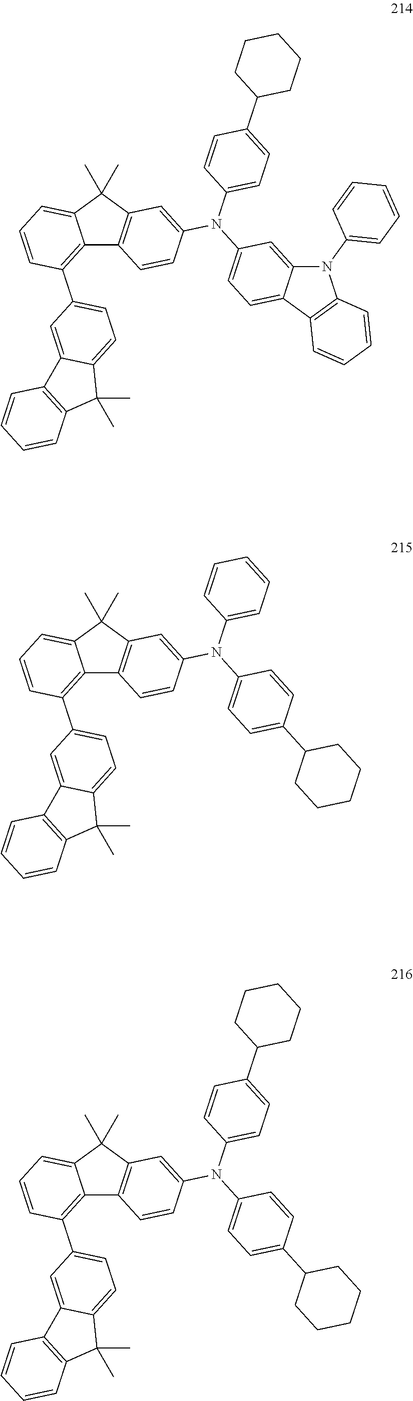 [SMILES: CC1(C)c2ccccc2-c2cc(-c3cccc4c3-c3ccc(N(c5ccc(C6CCCCC6)cc5)c5ccc6c7ccccc7n(-c7ccccc7)c6c5)cc3C4(C)C)ccc21 ; CC1(C)c2ccccc2-c2cc(-c3cccc4c3-c3ccc(N(c5ccccc5)c5ccc(C6CCCCC6)cc5)cc3C4(C)C)ccc21 ; CC1(C)c2ccccc2-c2cc(-c3cccc4c3-c3ccc(N(c5ccc(C6CCCCC6)cc5)c5ccc(C6CCCCC6)cc5)cc3C4(C)C)ccc21]

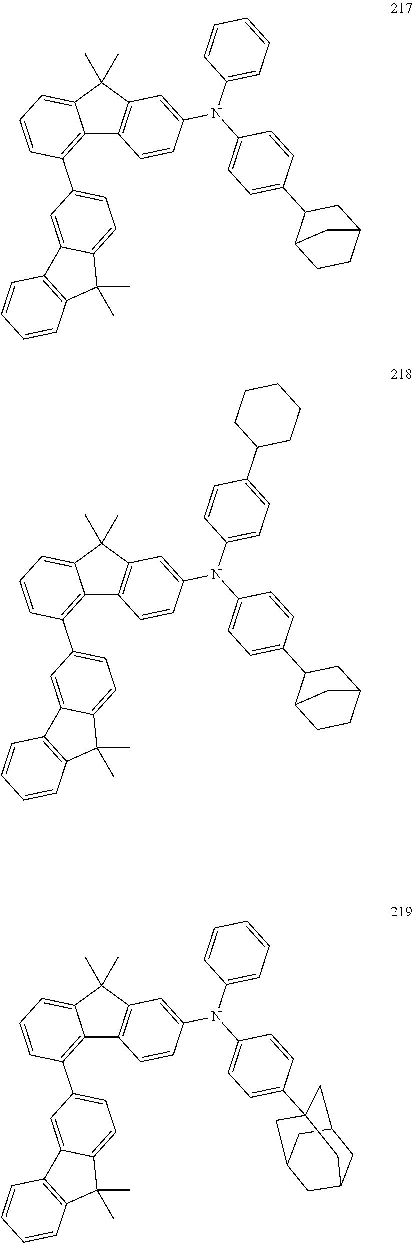 [SMILES: CC1(C)c2ccccc2-c2cc(-c3cccc4c3-c3ccc(N(c5ccccc5)c5ccc(C6CC7CCC6C7)cc5)cc3C4(C)C)ccc21 ; CC1(C)c2ccccc2-c2cc(-c3cccc4c3-c3ccc(N(c5ccc(C6CCCCC6)cc5)c5ccc(C6CC7CCC6C7)cc5)cc3C4(C)C)ccc21 ; CC1(C)c2ccccc2-c2cc(-c3cccc4c3-c3ccc(N(c5ccccc5)c5ccc(C67CC8CC(CC(C8)C6)C7)cc5)cc3C4(C)C)ccc21]

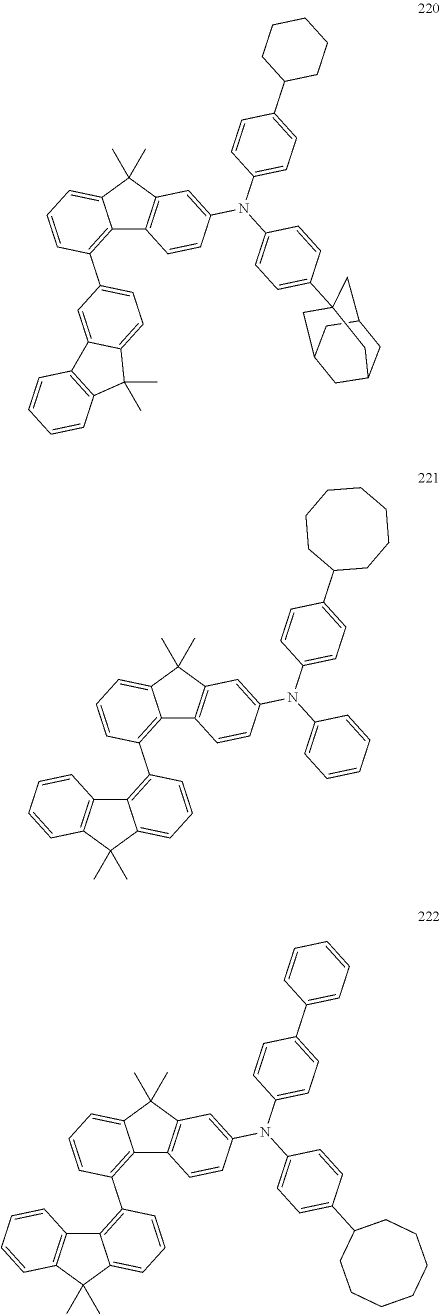 [SMILES: CC1(C)c2ccccc2-c2cc(-c3cccc4c3-c3ccc(N(c5ccc(C6CCCCC6)cc5)c5ccc(C67CC8CC(CC(C8)C6)C7)cc5)cc3C4(C)C)ccc21 ; CC1(C)c2ccccc2-c2c(-c3cccc4c3-c3ccc(N(c5ccccc5)c5ccc(C6CCCCCCC6)cc5)cc3C4(C)C)cccc21 ; CC1(C)c2ccccc2-c2c(-c3cccc4c3-c3ccc(N(c5ccc(-c6ccccc6)cc5)c5ccc(C6CCCCCCC6)cc5)cc3C4(C)C)cccc21]

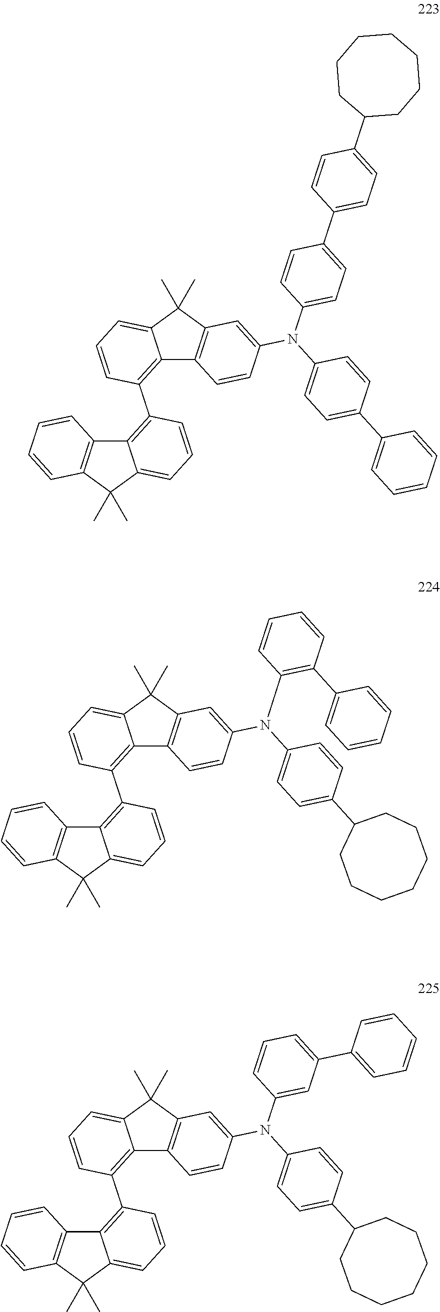 [SMILES: CC1(C)c2ccccc2-c2c(-c3cccc4c3-c3ccc(N(c5ccc(-c6ccccc6)cc5)c5ccc(-c6ccc(C7CCCCCCC7)cc6)cc5)cc3C4(C)C)cccc21 ; CC1(C)c2ccccc2-c2c(-c3cccc4c3-c3ccc(N(c5ccc(C6CCCCCCC6)cc5)c5ccccc5-c5ccccc5)cc3C4(C)C)cccc21 ; CC1(C)c2ccccc2-c2c(-c3cccc4c3-c3ccc(N(c5ccc(C6CCCCCCC6)cc5)c5cccc(-c6ccccc6)c5)cc3C4(C)C)cccc21]

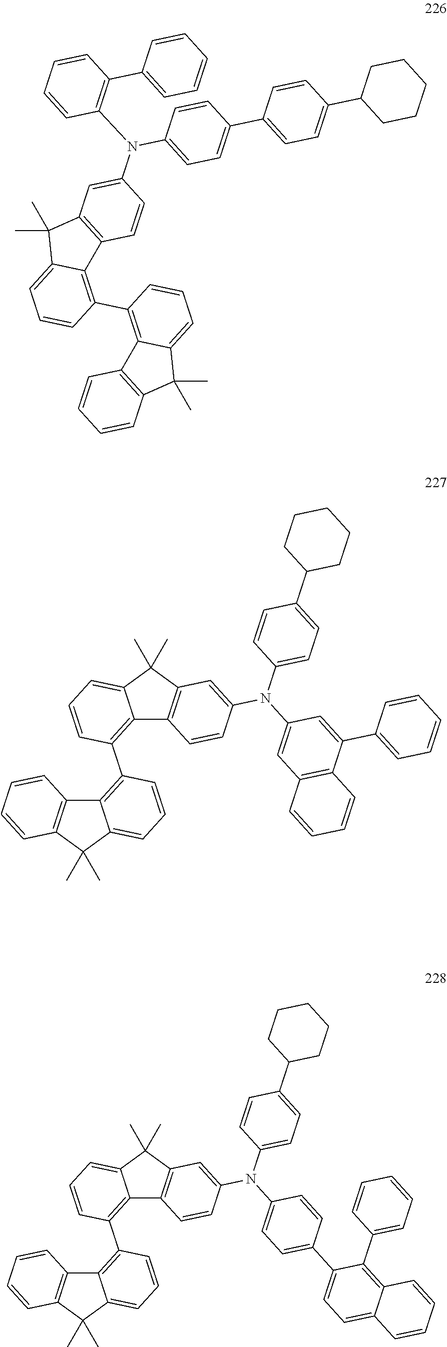 [SMILES: CC1(C)c2ccccc2-c2c(-c3cccc4c3-c3ccc(N(c5ccc(-c6ccc(C7CCCCC7)cc6)cc5)c5ccccc5-c5ccccc5)cc3C4(C)C)cccc21 ; CC1(C)c2ccccc2-c2c(-c3cccc4c3-c3ccc(N(c5ccc(C6CCCCC6)cc5)c5cc(-c6ccccc6)c6ccccc6c5)cc3C4(C)C)cccc21 ; CC1(C)c2ccccc2-c2c(-c3cccc4c3-c3ccc(N(c5ccc(-c6ccc7ccccc7c6-c6ccccc6)cc5)c5ccc(C6CCCCC6)cc5)cc3C4(C)C)cccc21]

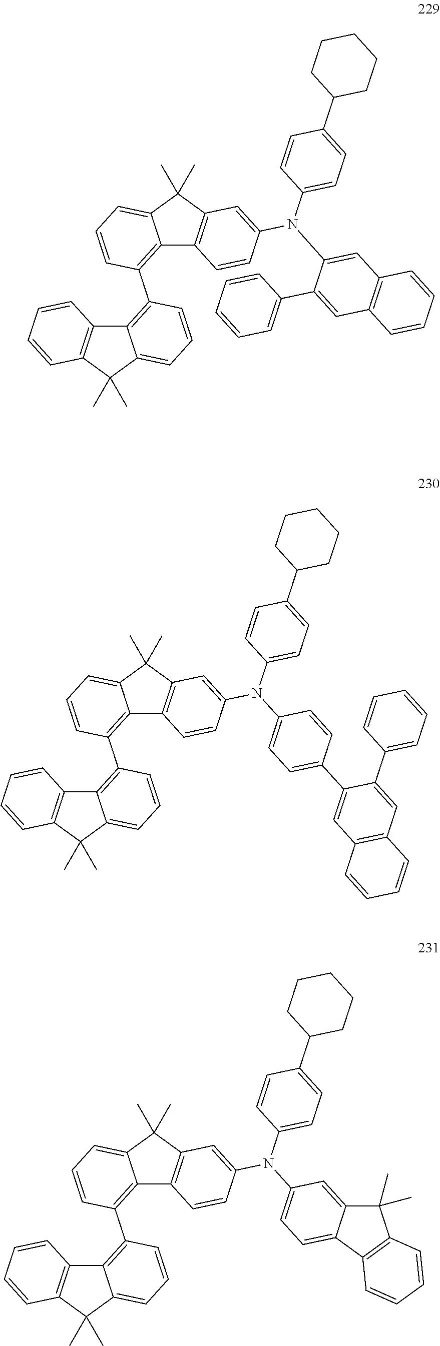 [SMILES: CC1(C)c2ccccc2-c2c(-c3cccc4c3-c3ccc(N(c5ccc(C6CCCCC6)cc5)c5cc6ccccc6cc5-c5ccccc5)cc3C4(C)C)cccc21 ; CC1(C)c2ccccc2-c2c(-c3cccc4c3-c3ccc(N(c5ccc(-c6cc7ccccc7cc6-c6ccccc6)cc5)c5ccc(C6CCCCC6)cc5)cc3C4(C)C)cccc21 ; CC1(C)c2ccccc2-c2ccc(N(c3ccc(C4CCCCC4)cc3)c3ccc4c(c3)C(C)(C)c3cccc(-c5cccc6c5-c5ccccc5C6(C)C)c3-4)cc21]

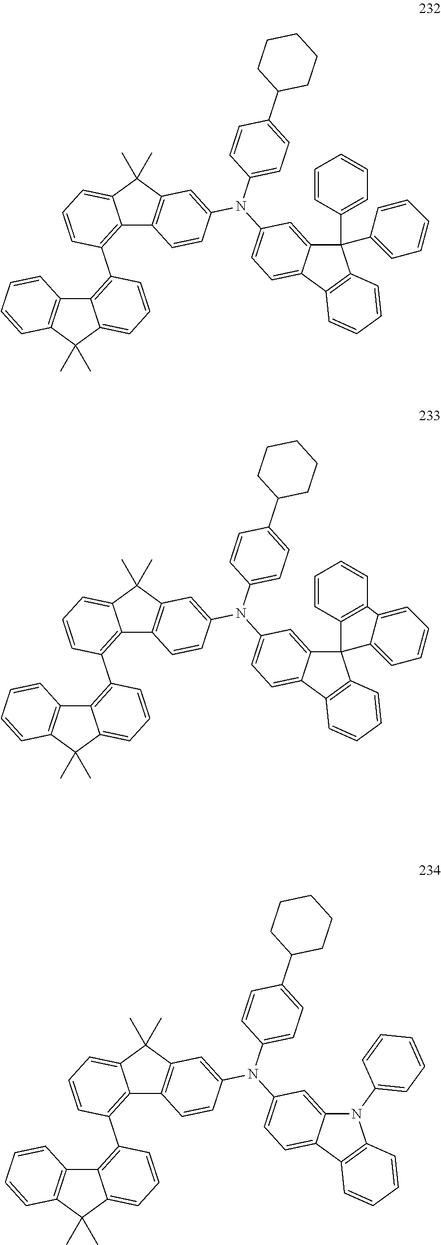 [SMILES: CC1(C)c2ccccc2-c2c(-c3cccc4c3-c3ccc(N(c5ccc(C6CCCCC6)cc5)c5ccc6c(c5)C(c5ccccc5)(c5ccccc5)c5ccccc5-6)cc3C4(C)C)cccc21 ; CC1(C)c2ccccc2-c2c(-c3cccc4c3-c3ccc(N(c5ccc(C6CCCCC6)cc5)c5ccc6c(c5)C5(c7ccccc7-c7ccccc75)c5ccccc5-6)cc3C4(C)C)cccc21 ; CC1(C)c2ccccc2-c2c(-c3cccc4c3-c3ccc(N(c5ccc(C6CCCCC6)cc5)c5ccc6c7ccccc7n(-c7ccccc7)c6c5)cc3C4(C)C)cccc21]

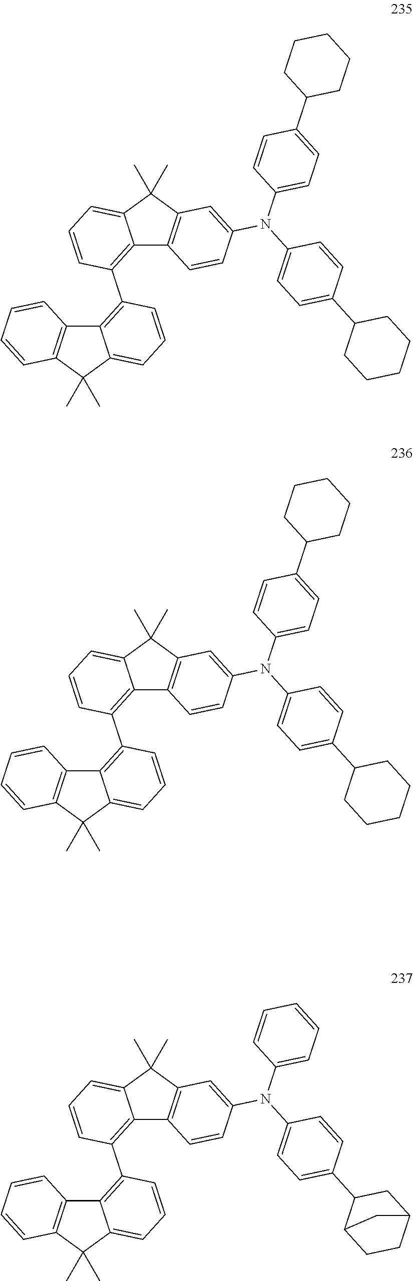 [SMILES: CC1(C)c2ccccc2-c2c(-c3cccc4c3-c3ccc(N(c5ccc(C6CCCCC6)cc5)c5ccc(C6CCCCC6)cc5)cc3C4(C)C)cccc21 ; CC1(C)c2ccccc2-c2c(-c3cccc4c3-c3ccc(N(c5ccc(C6CCCCC6)cc5)c5ccc(C6CCCCC6)cc5)cc3C4(C)C)cccc21 ; CC1(C)c2ccccc2-c2c(-c3cccc4c3-c3ccc(N(c5ccccc5)c5ccc(C6CC7CCC6C7)cc5)cc3C4(C)C)cccc21]

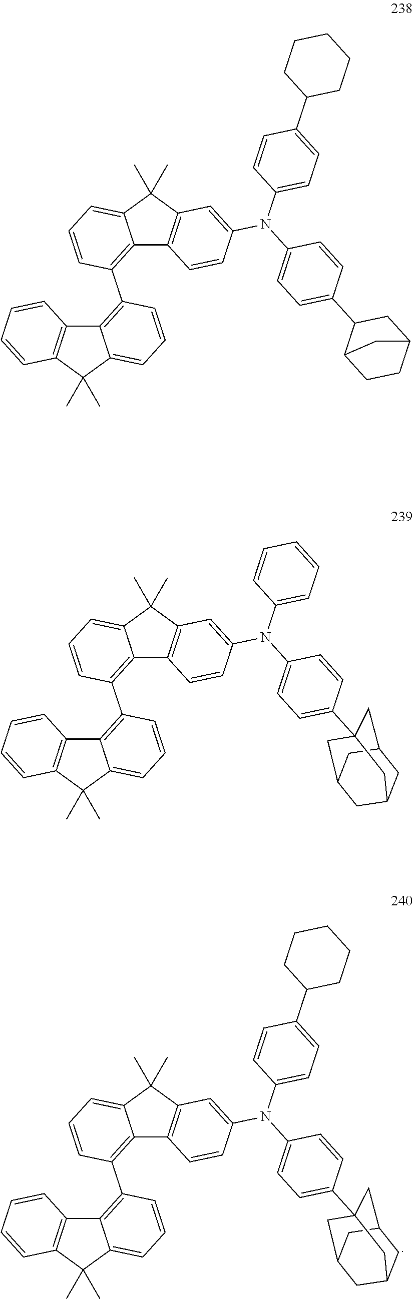 [SMILES: CC1(C)c2ccccc2-c2c(-c3cccc4c3-c3ccc(N(c5ccc(C6CCCCC6)cc5)c5ccc(C6CC7CCC6C7)cc5)cc3C4(C)C)cccc21 ; CC1(C)c2ccccc2-c2c(-c3cccc4c3-c3ccc(N(c5ccccc5)c5ccc(C67CC8CC(CC(C8)C6)C7)cc5)cc3C4(C)C)cccc21 ; CC1(C)c2ccccc2-c2c(-c3cccc4c3-c3ccc(N(c5ccc(C6CCCCC6)cc5)c5ccc(C67CC8CC(CC(C8)C6)C7)cc5)cc3C4(C)C)cccc21]

The amine-containing compound represented by Formula 1 may have a core structure of bifluorene. In some embodiments, at least one selected from among Ar₁ and Ar₂ may be a saturated C₃-C₃₀ cyclic group unsubstituted or substituted with at least one R₁₀ₐ. By extending a resonance length, the hole mobility may be increased, and a great molecular weight may significantly raise the glass transition temperature. In some embodiments, characteristics of low refractive index molecules may be exhibited by introducing a cycloalkyl group to amine. Moreover, by varying an aromatic condensed polycyclic derivative with extended conjugation, the highest occupied molecular orbital (HOMO) energy level of the amine-containing compound may be changed and adjusted variously. Accordingly, a hole injection barrier between ITO and the hole transport layer may be variously changed and adjusted, and an appropriate or suitable energy level may be maintained between the hole transport layer and the emission layer to increase exciton generation efficiency inside the emission layer.

In this manner, the amine-containing compound represented by Formula 1 may have excellent or suitable hole transport characteristics, and the light-emitting device including the amine-containing compound represented by Formula 1 may have a low driving voltage, high luminance, high luminescence efficiency and long lifespan.

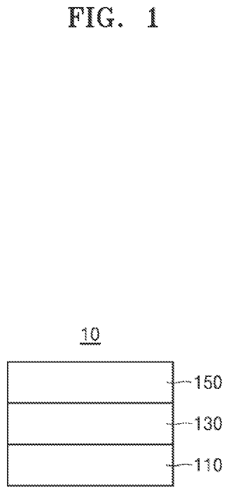

### Description of FIG. 1

FIG. 1 is a schematic diagram of a light-emitting device 10 according to one or more embodiments of the present disclosure. The light-emitting device 10 may include a first electrode 110, an interlayer 130, and a second electrode 150.

Hereinafter, a structure of the light-emitting device 10 according to one or more embodiments of the present disclosure and a method of manufacturing the light-emitting device 10 will be described with reference to FIG. 1.

### First Electrode 110

In FIG. 1, in some embodiments, a substrate may be additionally provided and disposed under the first electrode 110 and/or on the second electrode 150. The substrate may be a glass substrate or a plastic substrate. The substrate may be a flexible substrate. For example, in some embodiments, the flexible substrate may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by applying a material for forming the first electrode 110 onto the substrate by utilizing a deposition or sputtering method. When the first electrode 110 is an anode, a material for forming the first electrode 110 may be a high work function material that facilitates injection of holes.

The first electrode 110 may be a reflective electrode, a semi-transmissive electrode, or a transmissive electrode. In one or more embodiments, when the first electrode 110 is a transmissive electrode, the material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combinations thereof.

The first electrode 110 may have a single-layered structure including (e.g., consisting of) a single layer or a multi-layered structure including a plurality of layers. For example, in some embodiments, the first electrode 110 may have a three-layered structure of ITO/Ag/ITO.

### Interlayer 130

The interlayer 130 may be on the first electrode 110. The interlayer 130 may include an emission layer.

In one or more embodiments, the interlayer 130 may further include a hole transport region arranged between the first electrode 110 and the emission layer and an electron transport region arranged between the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer arranged between the two emitting units. When the interlayer 130 includes the two or more emitting units and the charge generation layer, the light-emitting device 10 may be a tandem light-emitting device.

### Hole Transport Region in Interlayer 130

The hole transport region may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The hole transport region may include a hole injection layer, a hole transport layer, an emission auxiliary layer, an electron-blocking layer, or any combination thereof.

For example, in some embodiments, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the layers of each structure being stacked sequentially from the first electrode 110 in the stated order.

The hole transport region may include a compound represented by Formula 201, a compound represented by Formula 202, or any combination thereof:

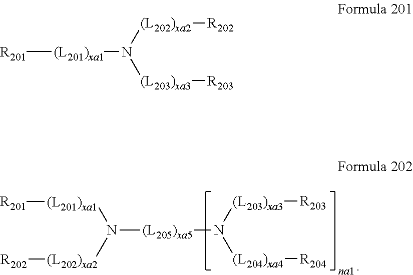

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)—*′, a C₁-C₆₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, see Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one selected from groups represented by Formulae CY201 to CY217:

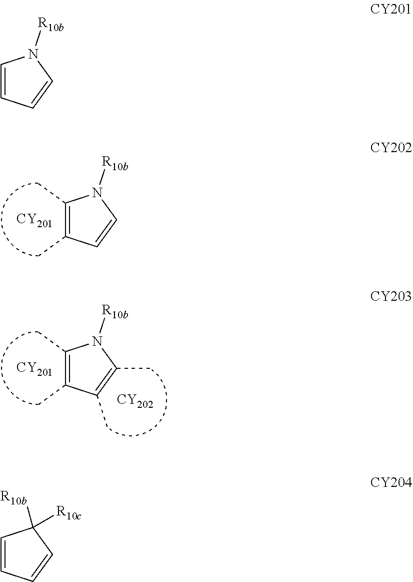

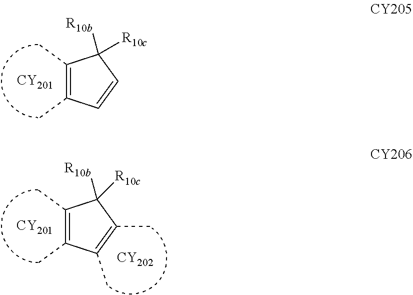

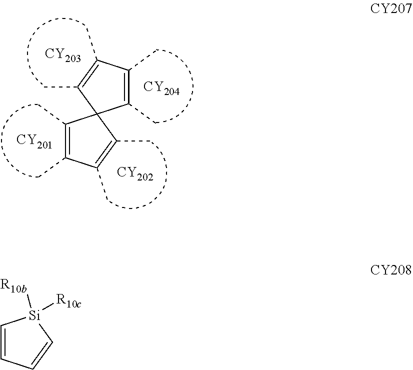

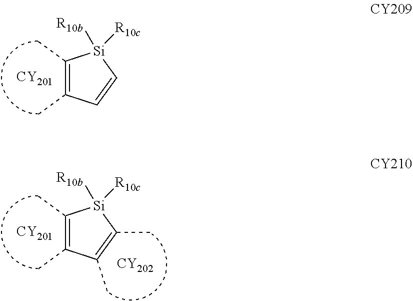

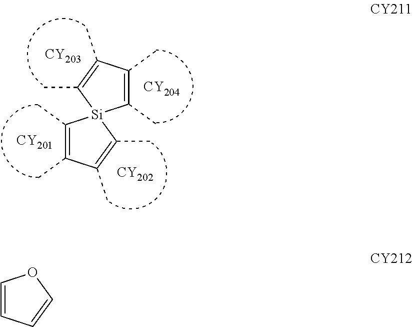

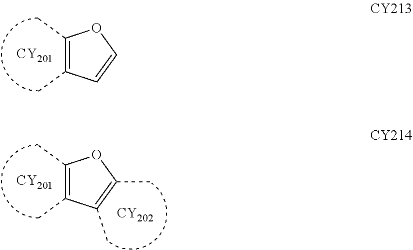

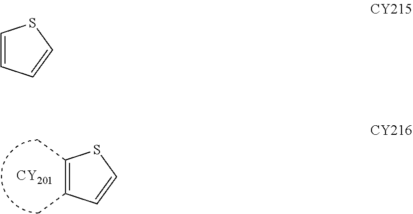

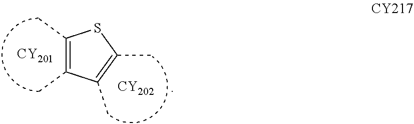

In Formulae CY201 to CY217, R_{10b} and R_{10c} may each be the same as described with respect to R₁₀ₐ, ring CY₂₀₁ to ring CY₂₀₄ may each independently be a C₃-C₂₀ carbocyclic group or a C₁-C₆₀ heterocyclic group, and at least one hydrogen in Formulae CY201 to CY217 may be unsubstituted or substituted with R₁₀ₐ as described above.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

According to one or more embodiments, Formula 201 may include at least one selected from the groups represented by Formulae CY201 to CY203 and at least one selected from groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include (e.g., may exclude) a group represented by one selected from Formulae CY201 to CY203.

In one or more embodiments, each of Formulae 201 and 202 may not include (e.g., may exclude) a group represented by one selected from Formulae CY201 to CY203, and may include at least one selected from the groups represented by Formulae CY204 to CY217.

In one or more embodiments, each of Formulae 201 and 202 may not include (e.g., may exclude) a group represented by one selected from Formulae CY201 to CY217.

In one or more embodiments, the hole transport region may include at least one selected from Compounds HT1 to HT46, 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), N,N′-di(1-naphthalene-1-yl)-N,N′-diphenyl-benzidine (NPB(NPD)), 8-NPB, N,N′-bis(3-methylphenyl)-N,N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

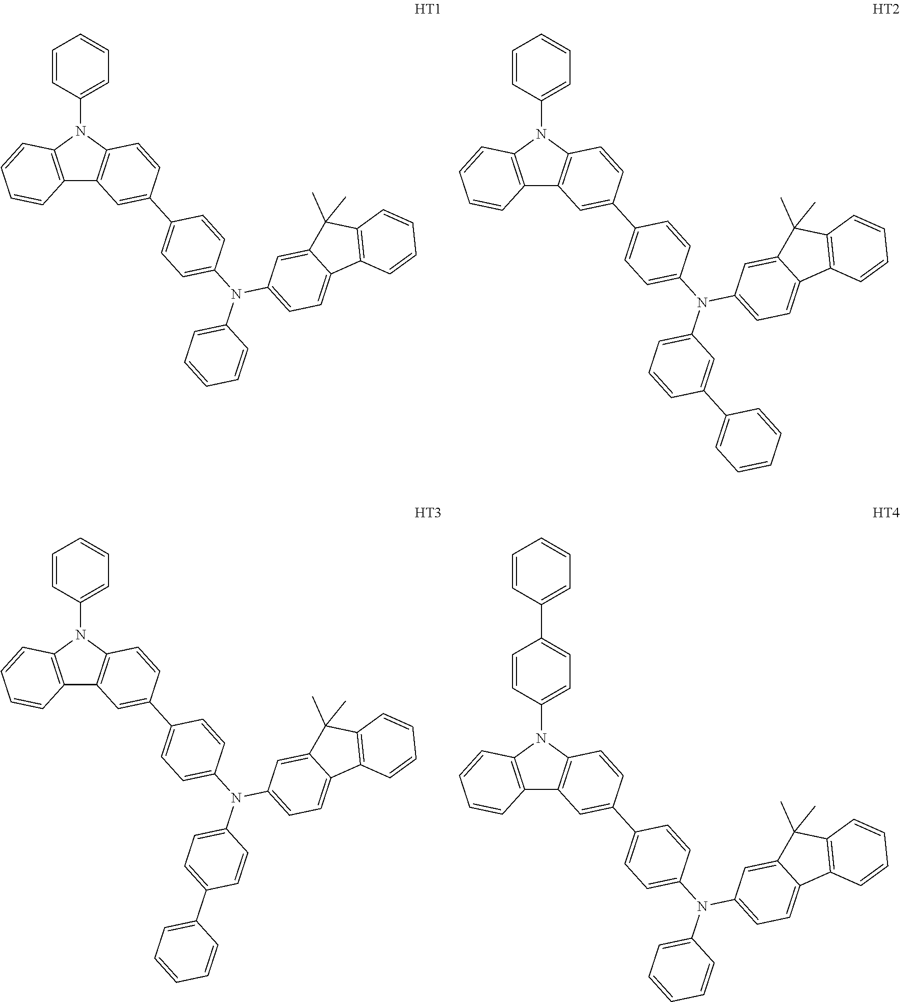

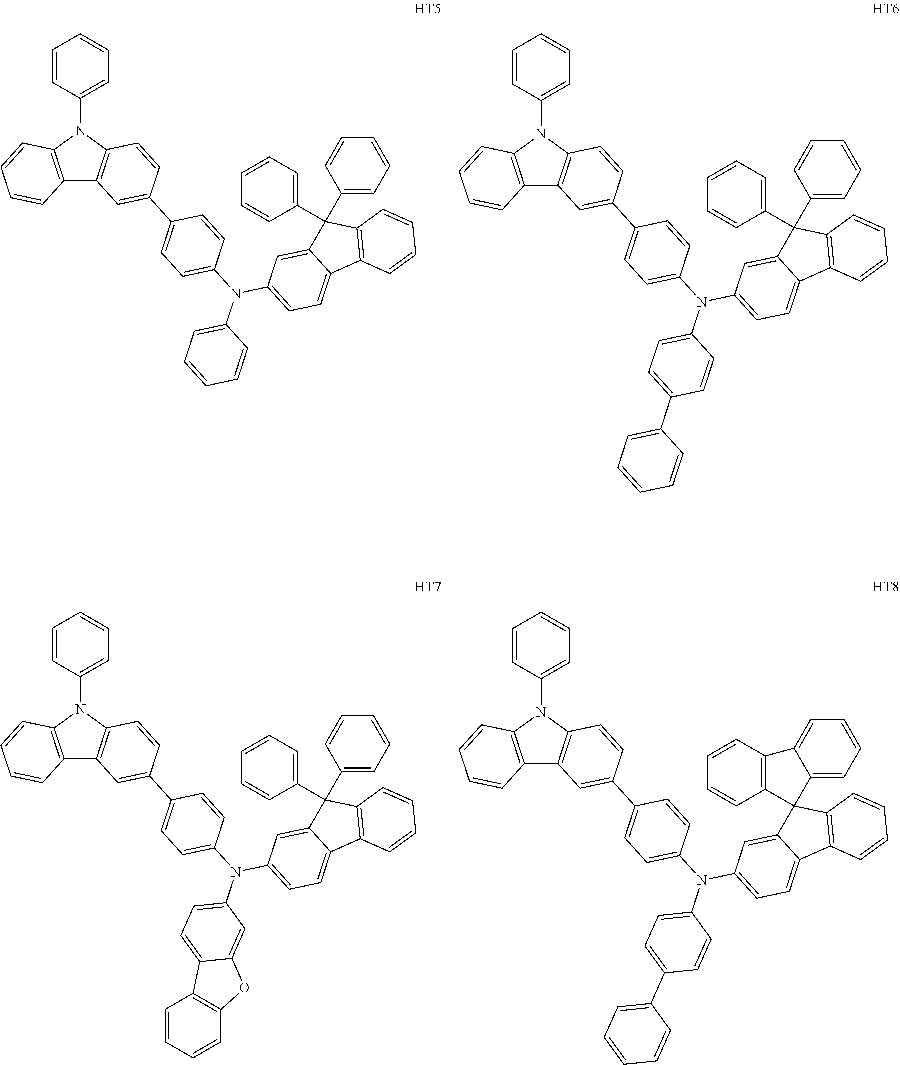 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

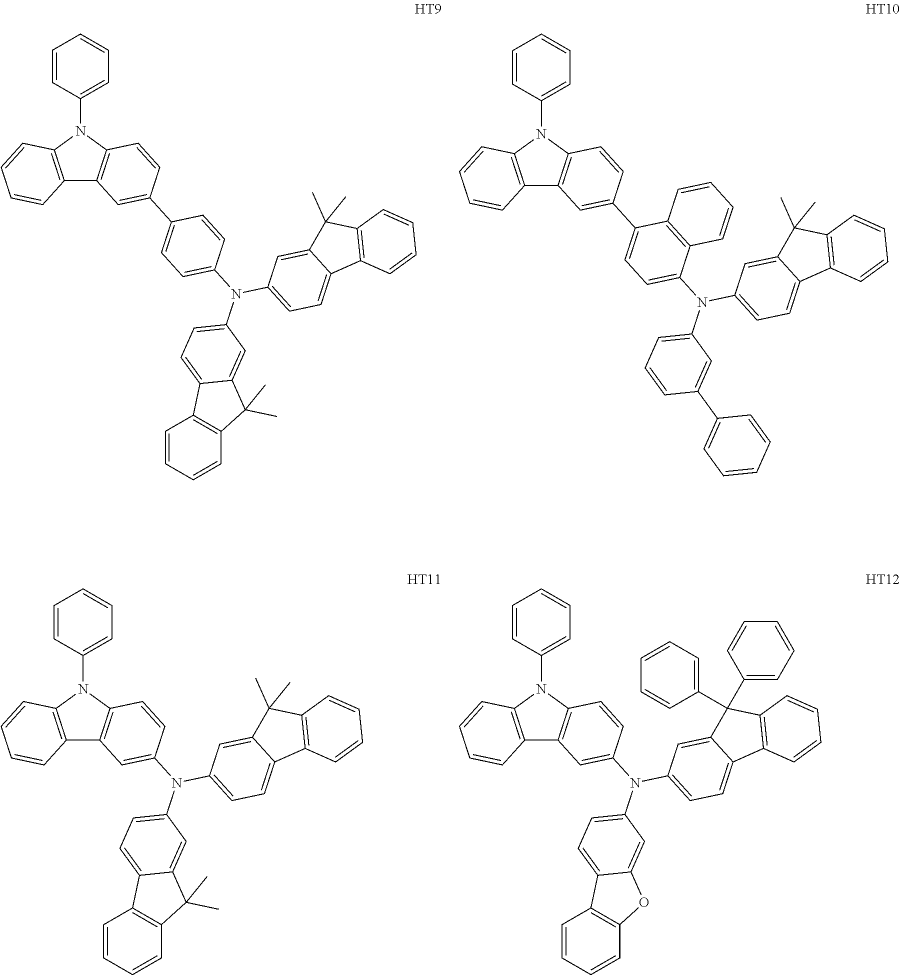 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

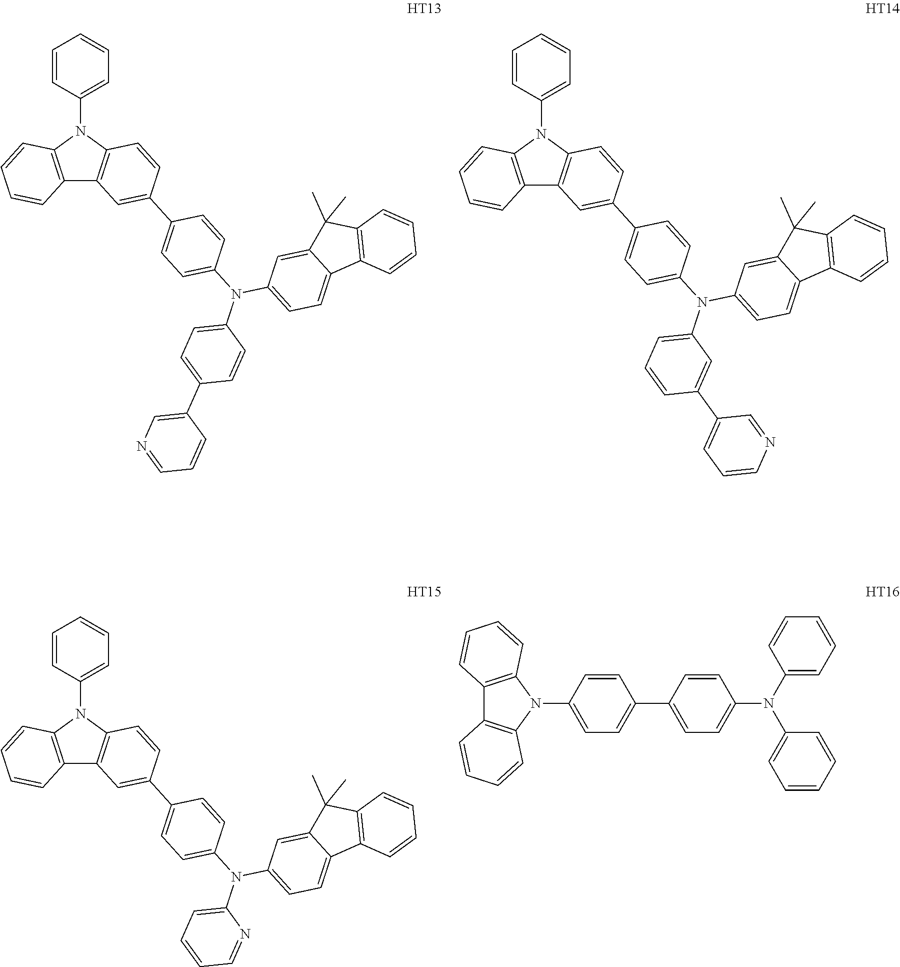 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

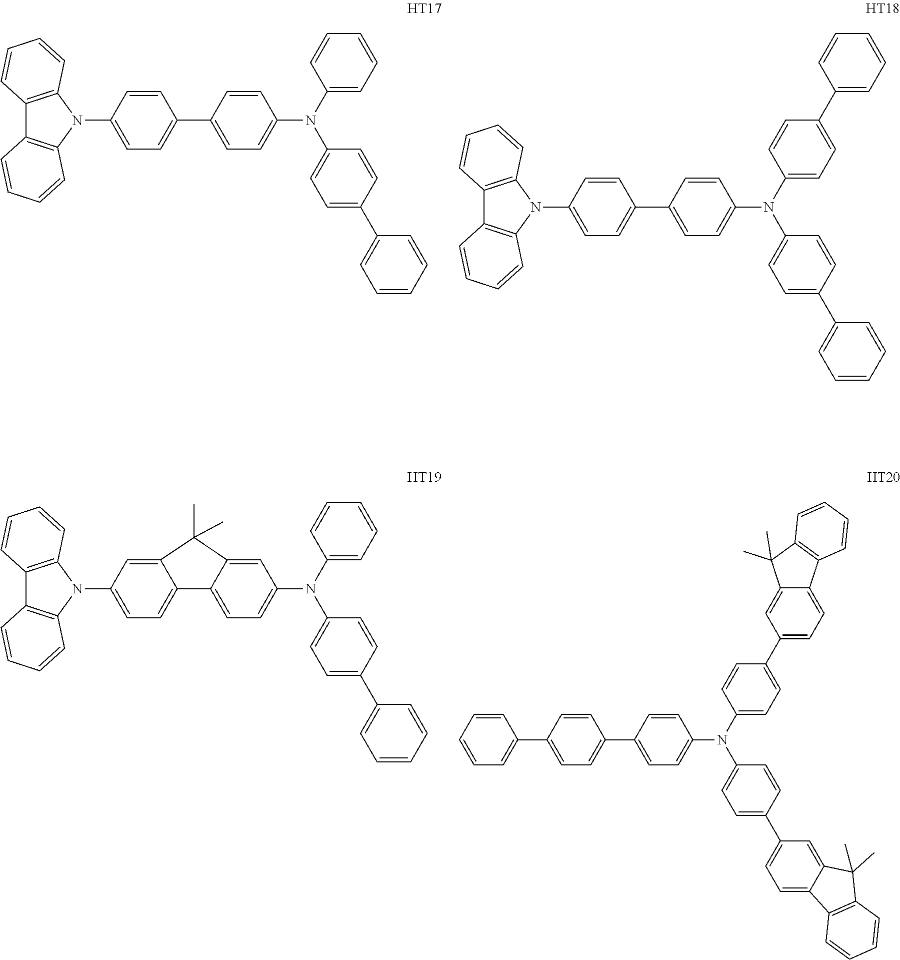

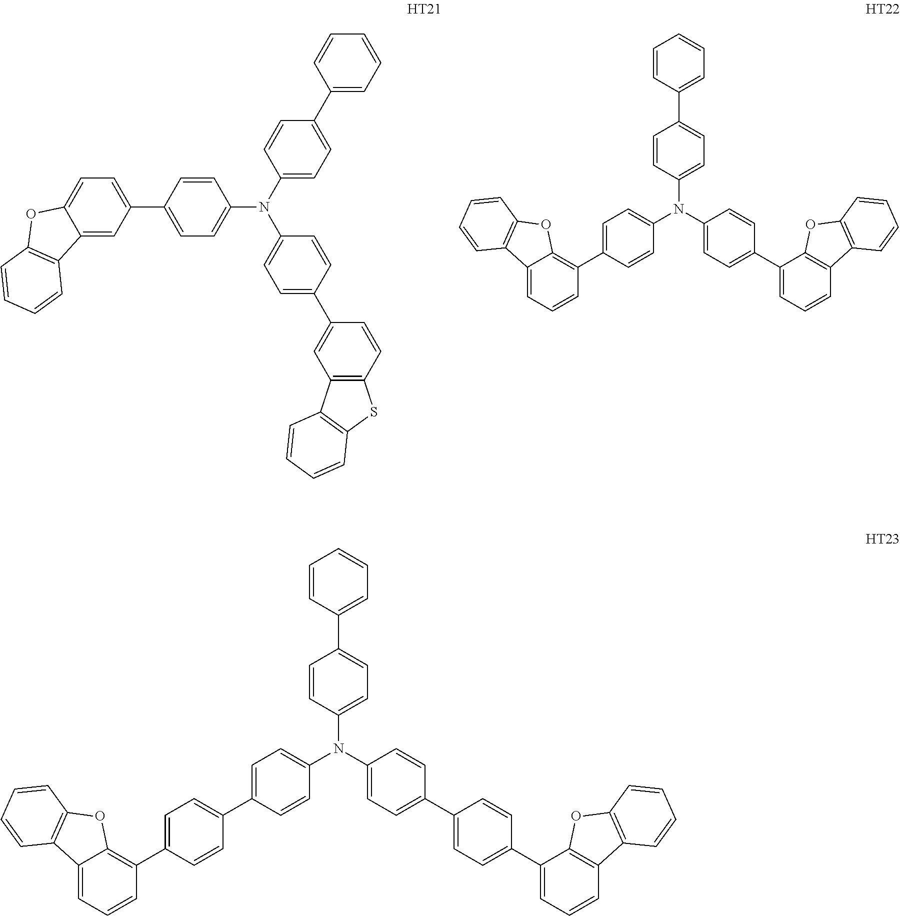 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

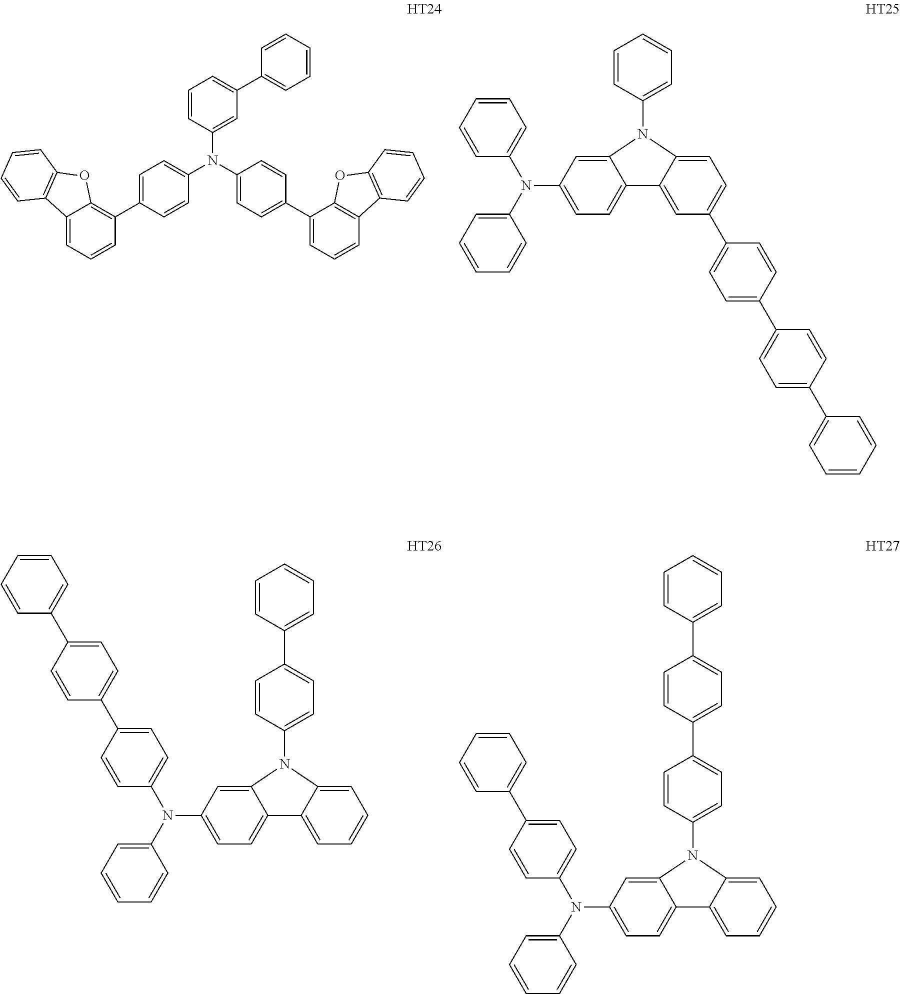 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

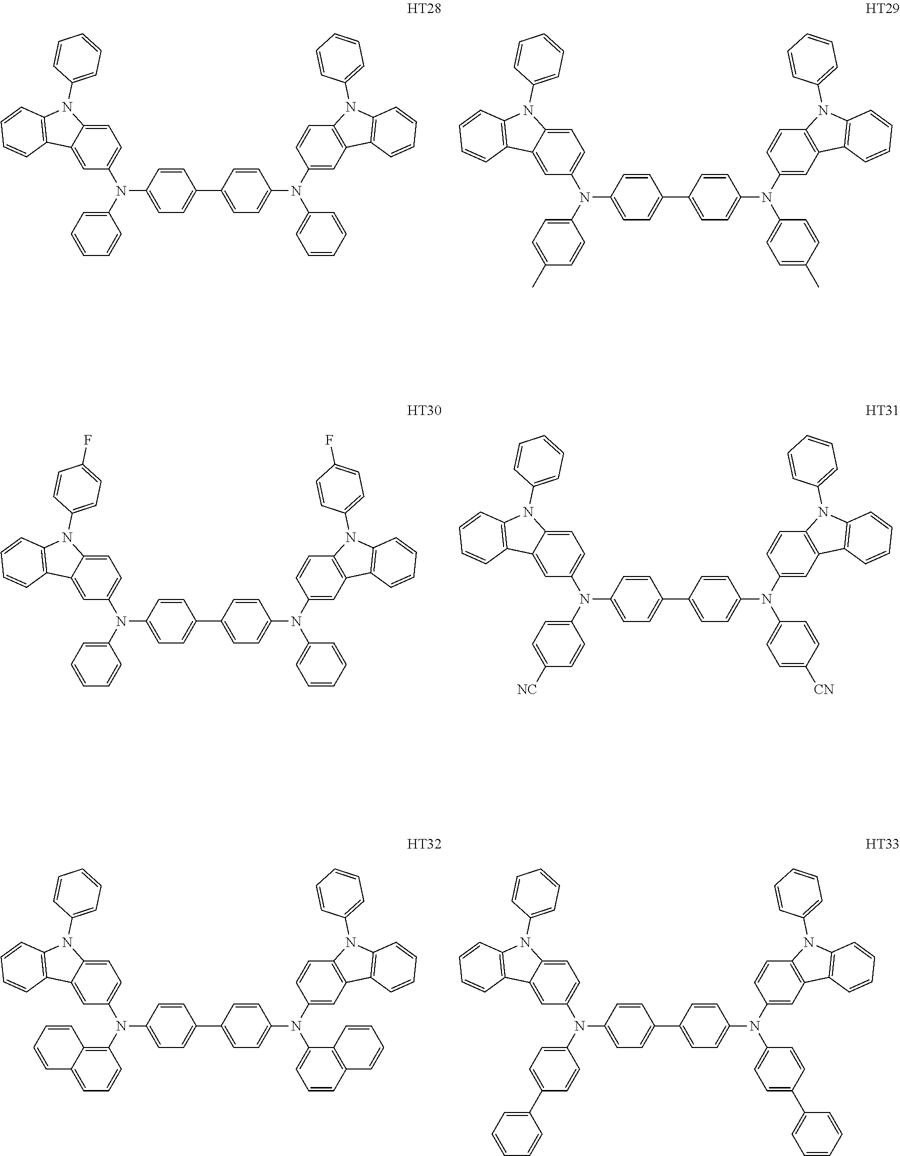

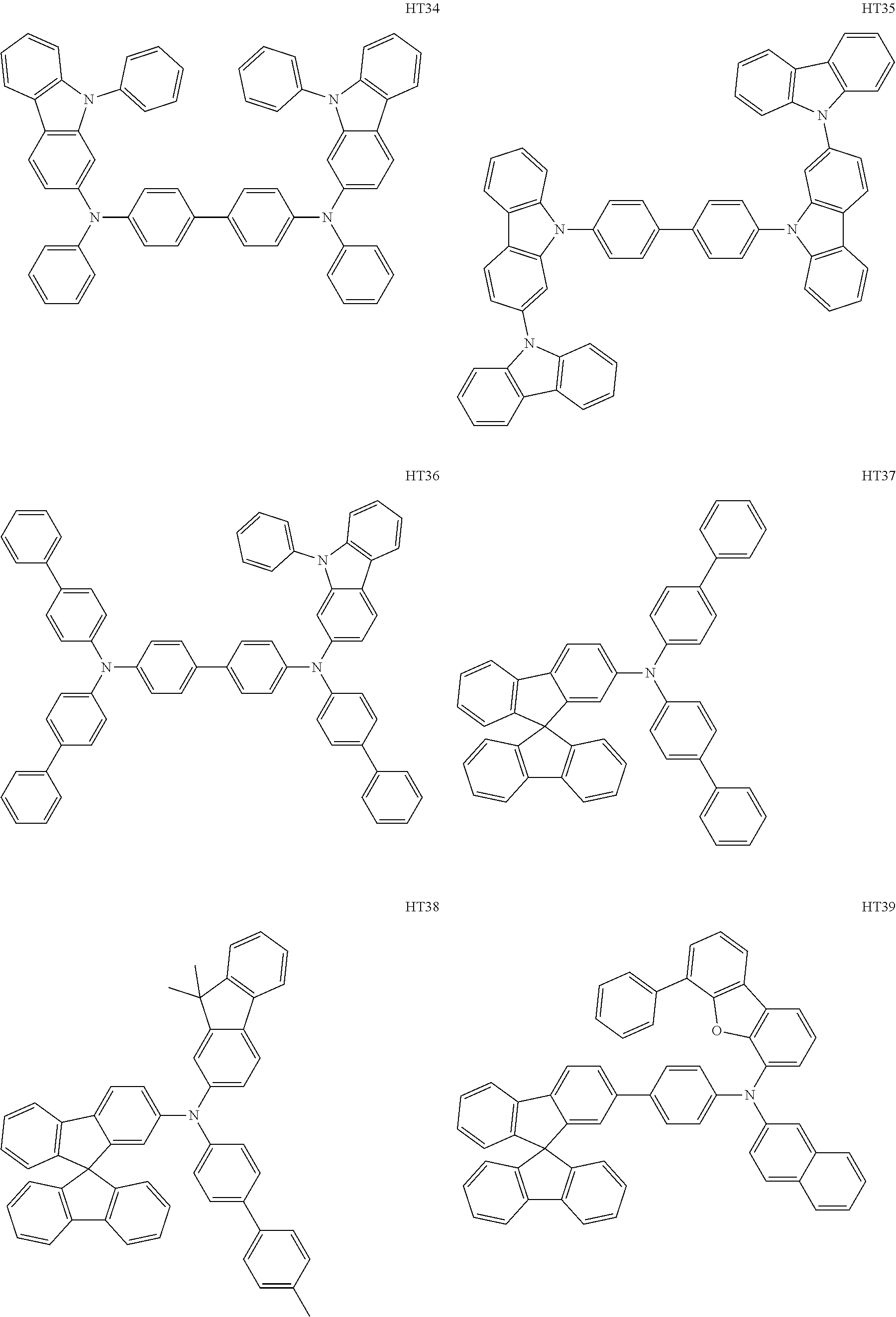

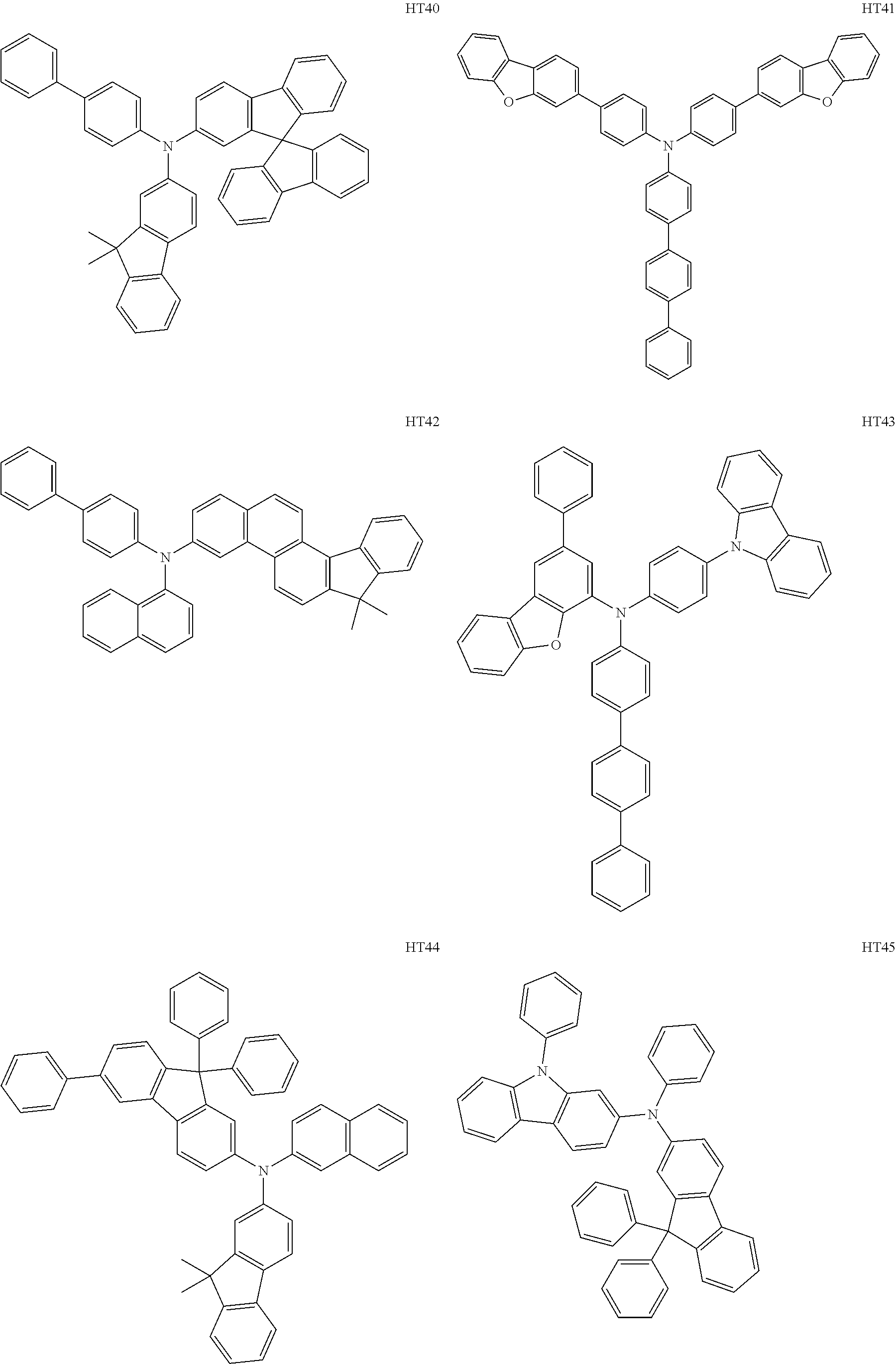

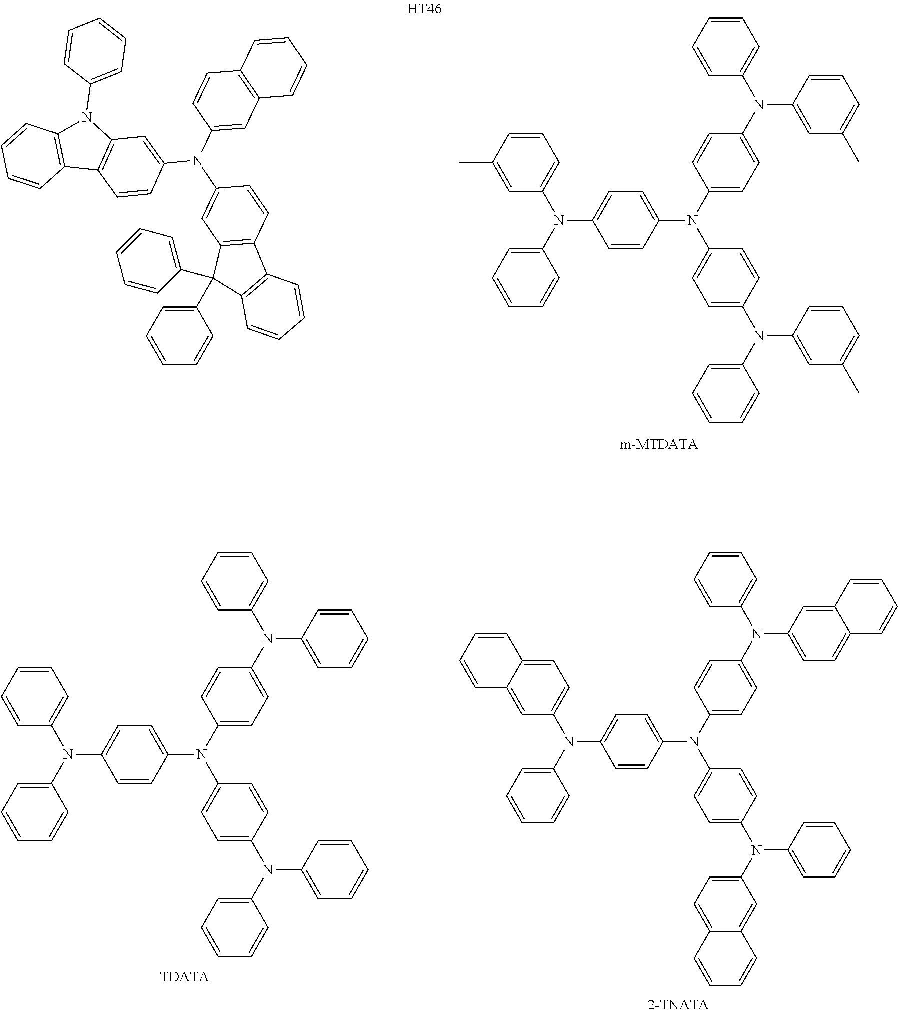

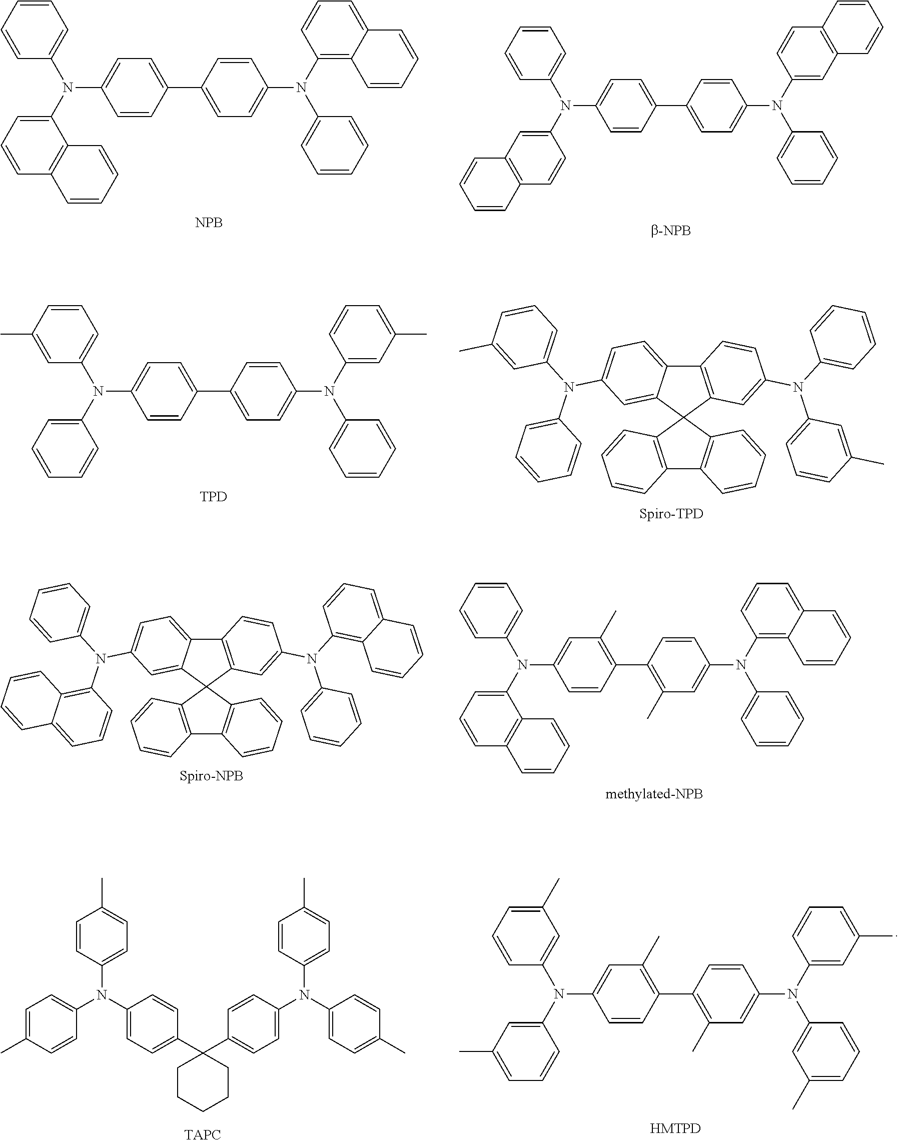

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may compensate for an optical resonance distance according to a wavelength of light emitted from the emission layer to improve the light emission efficiency. The electron blocking layer may be a layer that prevents electron leakage from the emission layer to the hole transport region. Materials that may be included in the hole transport region may be included in the emission auxiliary layer and the electron-blocking layer.

p-Dopant

In one or more embodiments, the hole transport region may further include, in addition to these materials, a charge-generation material for the improvement of conductive properties. The charge generation material may be uniformly or non-uniformly dispersed in the hole transport region (for example, in the form of a single layer including (e.g., consisting of) a charge generation material).

The charge-generation material may be, for example, a p-dopant.

For example, a lowest unoccupied molecular orbital (LUMO) energy level of the p-dopant may be about −3.5 eV or less.

In one or more embodiments, the p-dopant may include a quinone derivative, a cyano group-containing compound, a compound including element EL1 and element EL2, or any combination thereof.

Non-limiting examples of the quinone derivative may be tetracyanoquinodimethane (TCNQ), 2,3,5,6-tetrafluoro-7,7,8,8-tetracyanoquinodimethane (F4-TCNQ), etc.

Non-limiting examples of the cyano group-containing compound may be dipyrazino[2,3-f:2′,3′-h] quinoxaline-2,3,6,7,10,11-hexacarbonitrile (HAT-CN), and a compound represented by Formula 221:

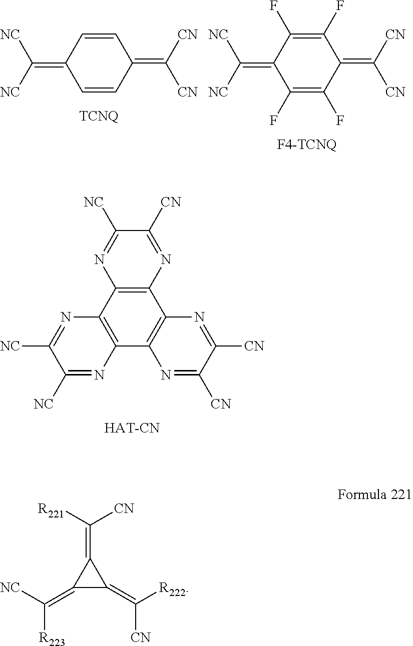

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be: a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Non-limiting examples of the metal may be an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and/or a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Non-limiting examples of the metalloid may be silicon (Si), antimony (Sb), and/or tellurium (Te).

Non-limiting examples of the non-metal may be oxygen (O) and/or halogen (for example, F, Cl, Br, I, etc.).

Non-limiting examples of the compound including element EL1 and element EL2 may be metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, and/or any combination thereof.

Non-limiting examples of the metal oxide may be tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and/or rhenium oxide (for example, ReO₃, etc.).

Non-limiting examples of the metal halide may be alkali metal halide, alkaline earth metal halide, transition metal halide, post-transition metal halide, and/or lanthanide metal halide.

Non-limiting examples of the alkali metal halide may be LiF, NaF, KF, RbF, CsF, LiCl, NaCl, KCl, RbCl, CsCl, LiBr, NaBr, KBr, RbBr, CsBr, LiI, NaI, KI, RbI, and/or CsI.

Non-limiting examples of the alkaline earth metal halide may be BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and/or Bale.

Non-limiting examples of the transition metal halide may be titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr3, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, Felt, etc.), ruthenium halide (for example, RuF₂, RuCl₂, RuBr₂, Rule, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, Nile, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and/or gold halide (for example, AuF, AuCl, AuBr, AuI, etc.).

Non-limiting examples of the post-transition metal halide may be zinc halide (for example, ZnF₂, ZnCl₂, ZnBr₂, ZnI₂, etc.), indium halide (for example, InI₃, etc.), and/or tin halide (for example, SnI₂, etc.).

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

A non-limiting example of the metalloid halide may be antimony halide (for example, SbCl₅, etc.).

Non-limiting examples of the metal telluride may be alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and/or lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers selected from a red emission layer, a green emission layer, and a blue emission layer, in which the two or more layers contact each other or are separated from each other to emit white light (e.g., combined white light). In one or more embodiments, the emission layer may include two or more materials selected from a red light-emitting material, a green light-emitting material, and a blue light-emitting material, in which the two or more materials are mixed with each other in a single layer to emit white light (e.g., combined white light).

The emission layer may include a host and a dopant. The dopant may include a phosphorescent dopant, a fluorescent dopant, or any combination thereof.

The amount of the dopant in the emission layer may be from about 0.01 part by weight to about 15 parts by weight based on 100 parts by weight of the host.

In one or more embodiments, the emission layer may include a quantum dot.

In some embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent or suitable light-emission characteristics may be obtained without a substantial increase in driving voltage.

### Host

In one or more embodiments, the host may include a compound represented by Formula 301:

[Ar₃₀₁]xm₁₁-[(L₃₀₁)xb₁-R₃₀₁]xb₂₁ Formula 301

In Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be the same as described herein with respect to Q₁.

For example, in some embodiments, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

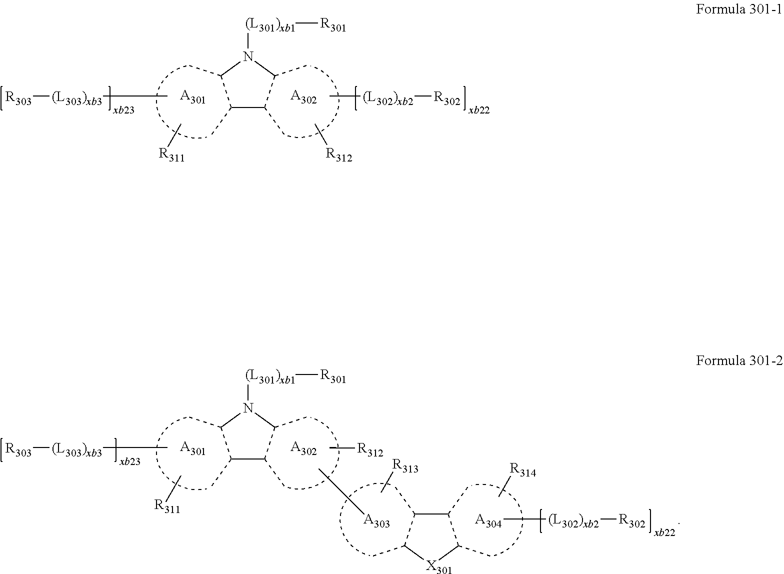

In Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₆), or Si(R₃₀₄)(R₃₀₆),
xb22 and xb23 may each independently be 0, 1, or 2,
L₃₀₁, xb1, and R₃₀₁ may each be the same as described herein,
L₃₀₂ to L₃₀₄ may each independently be the same as described herein with respect to with L₃₀₁,
xb2 to xb4 may each independently be the same as described herein with respect to xb1, and
R₃₀₂ to R₃₀₅ and R₃₁₁ to R₃₁₄ may each be the same as described herein with respect to R₃₀₁.

In one or more embodiments, the host may include an alkaline earth metal complex, a post-transition metal complex, or any combination thereof. For example, in some embodiments, the host may include a Be complex (for example, Compound H55), an Mg complex, a Zn complex, or any combination thereof.

In one or more embodiments, the host may include at least one selected from among Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), and/or any combination thereof:

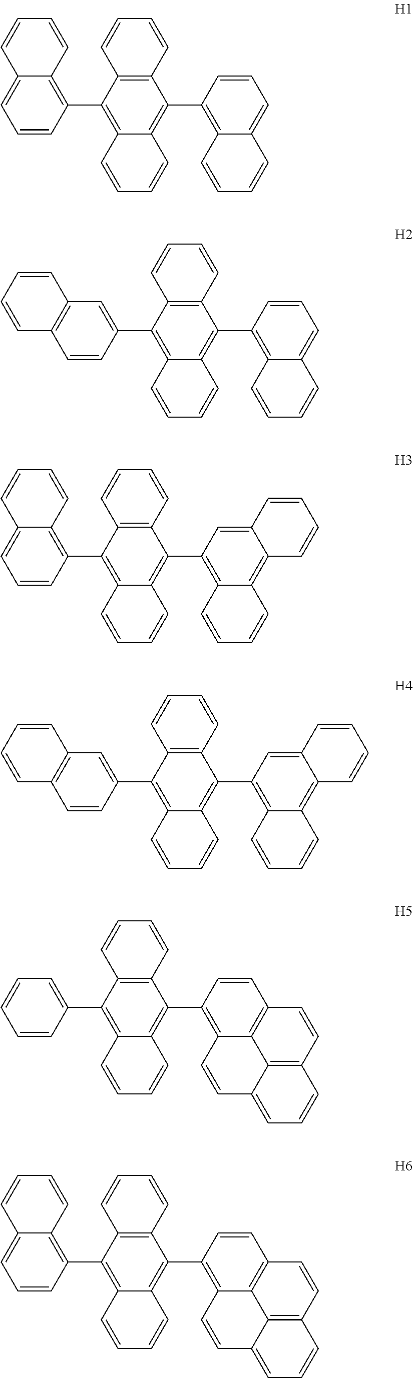

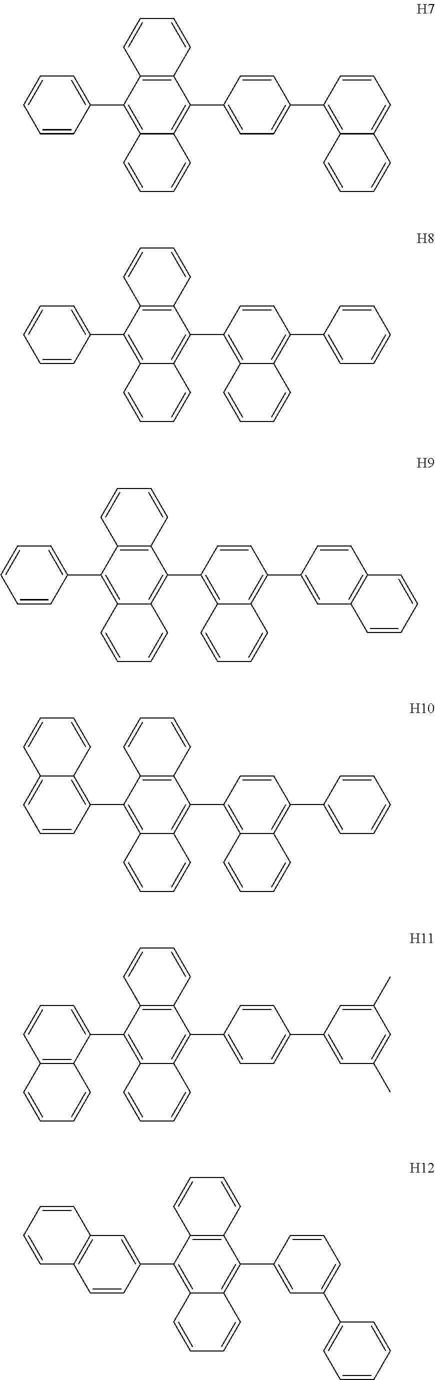

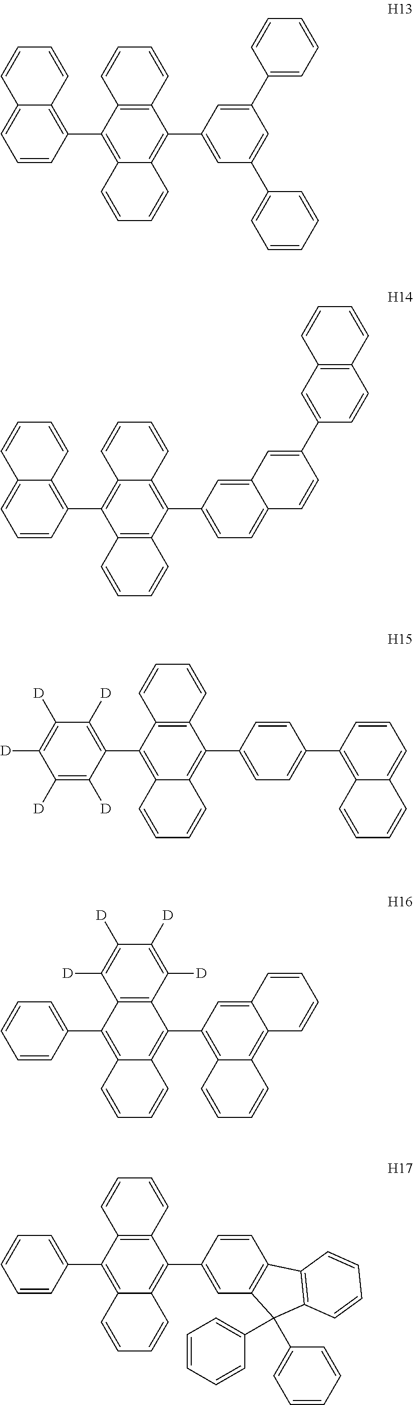

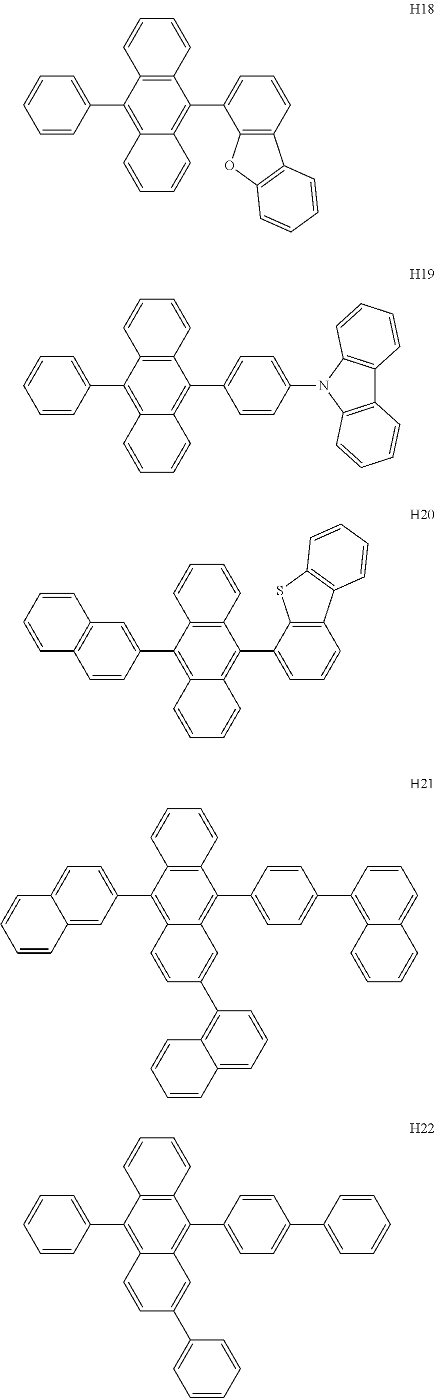

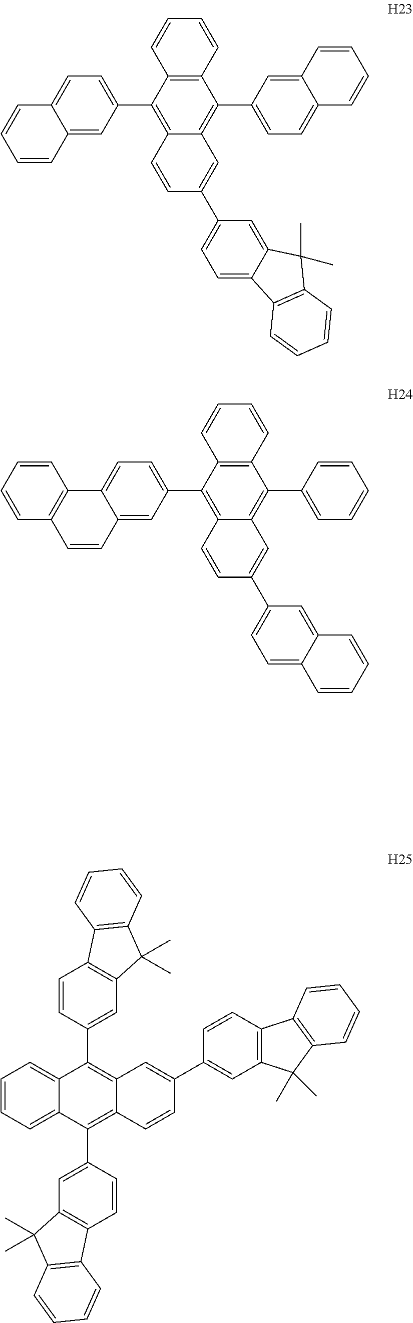

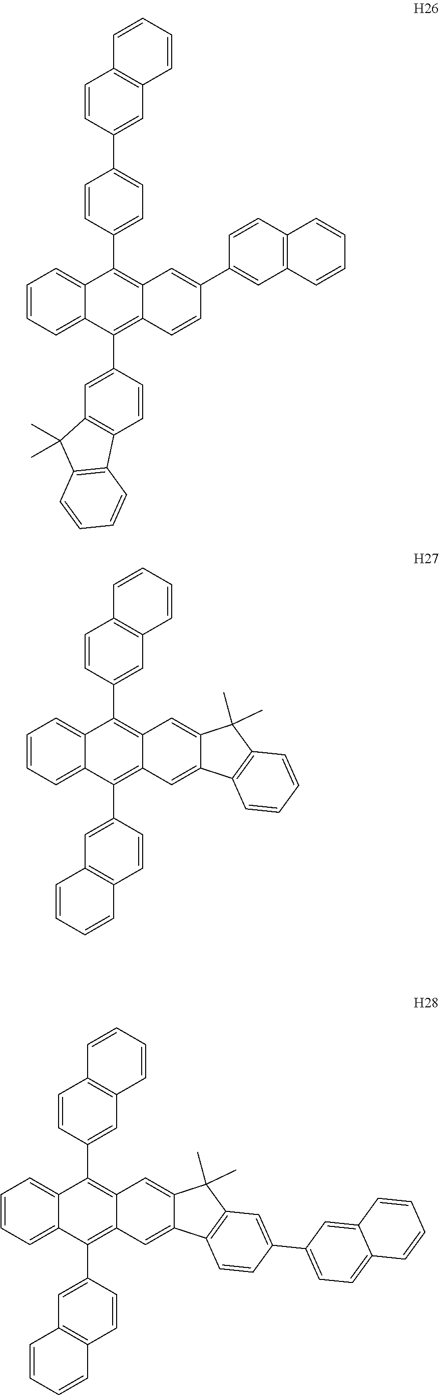

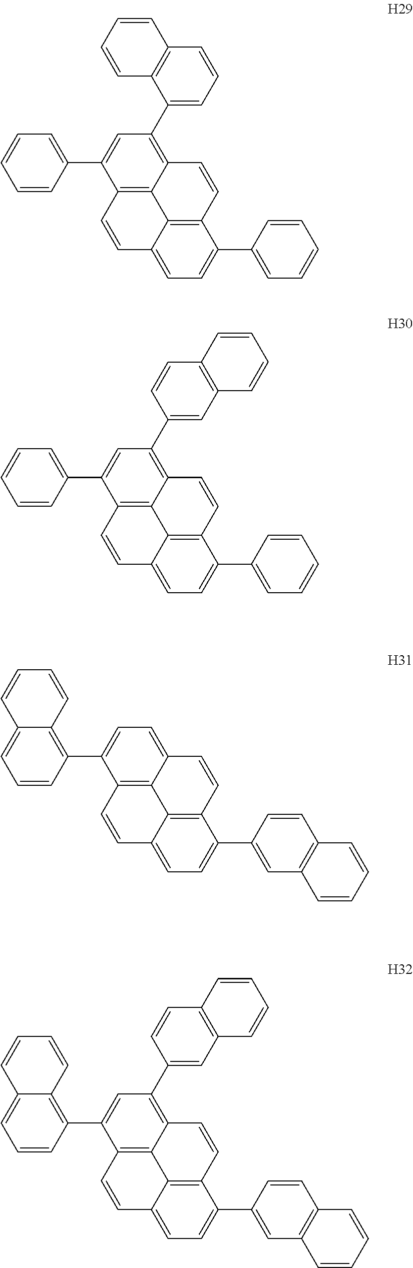

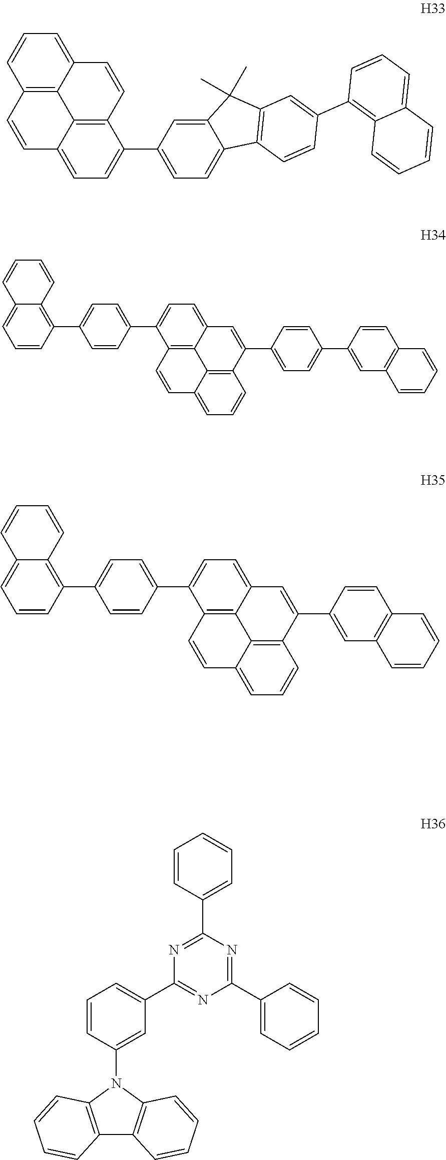

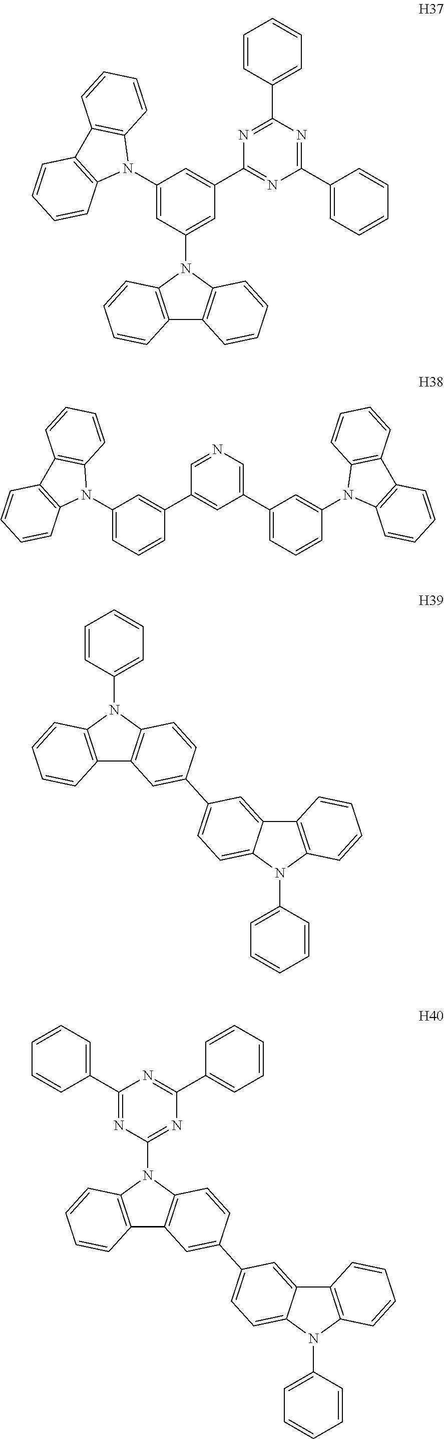 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

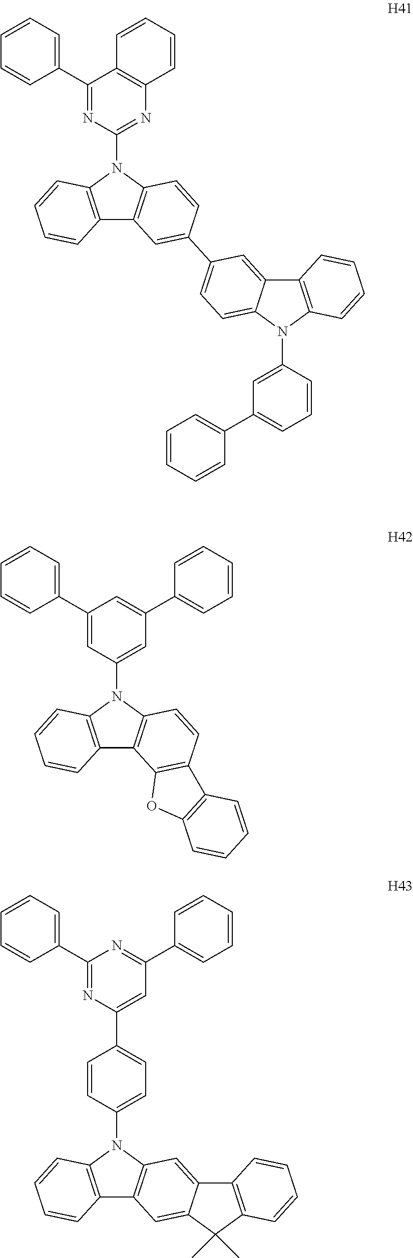

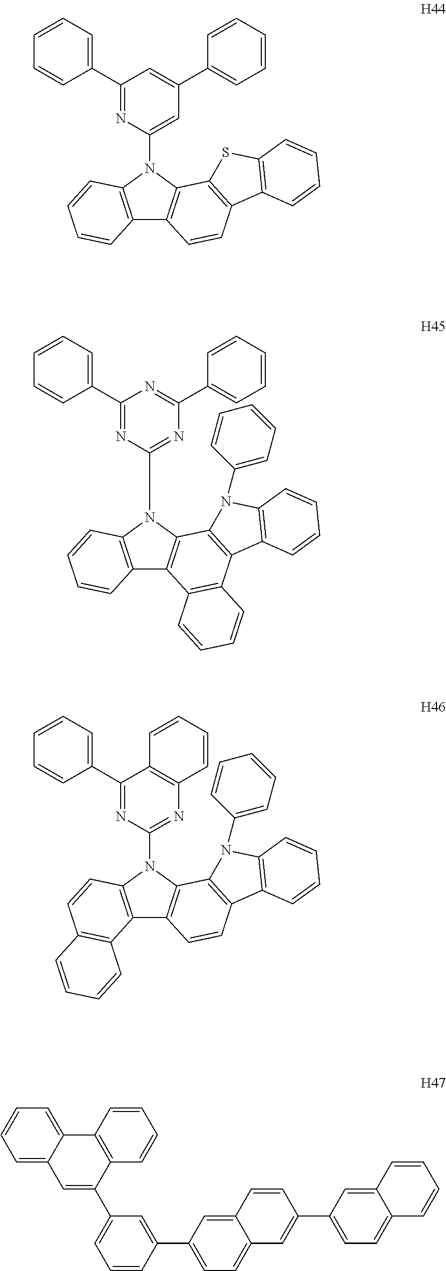

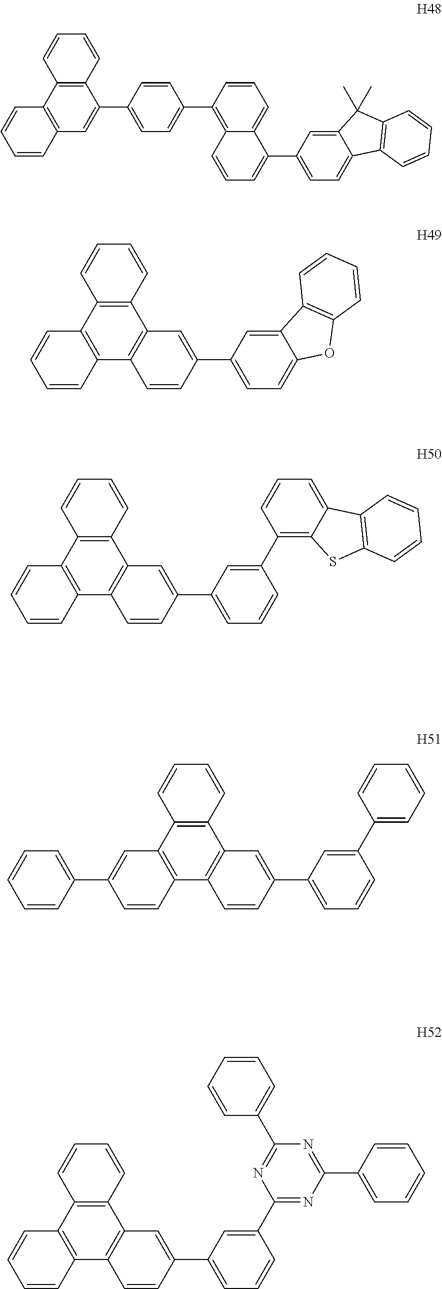

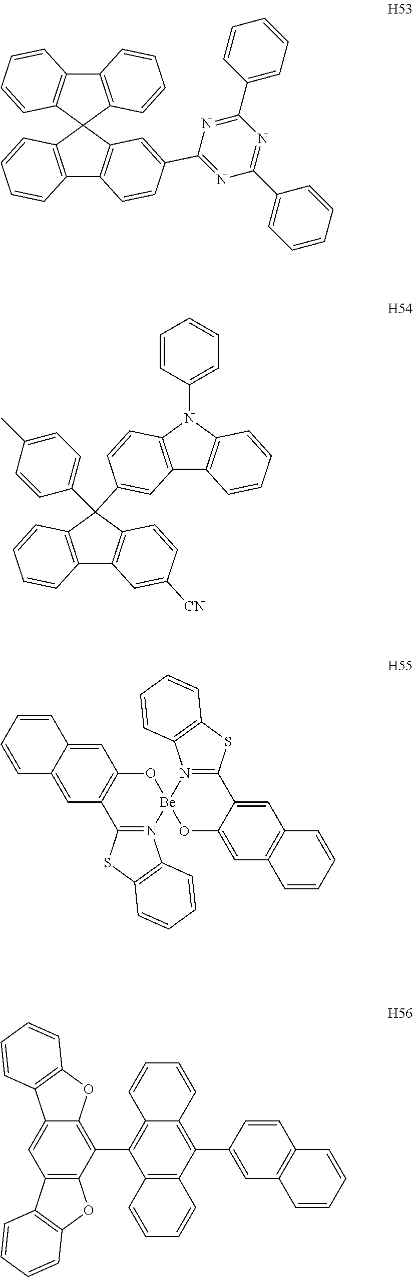

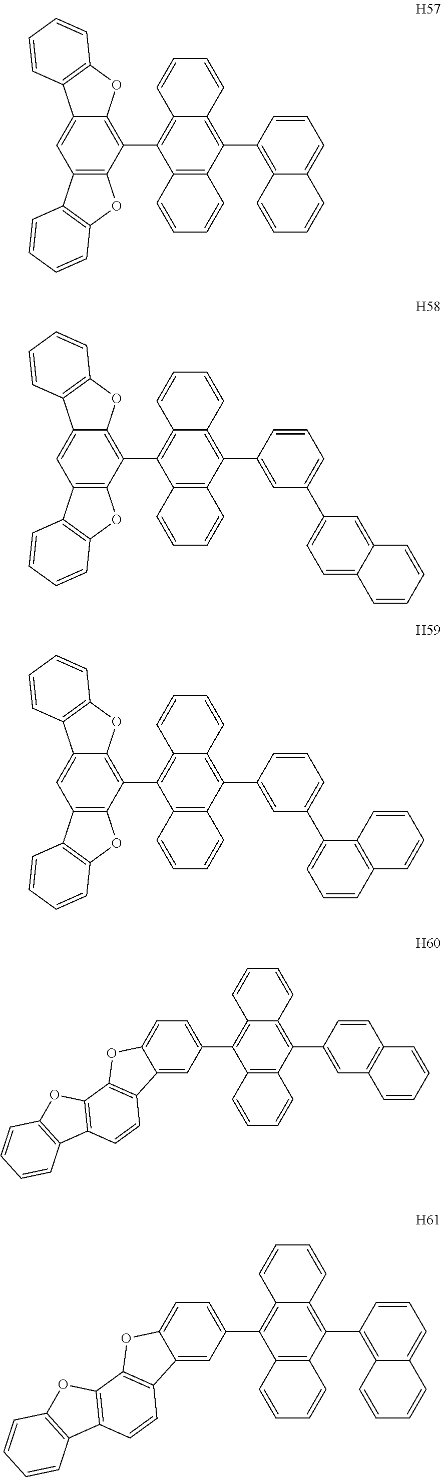 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1]

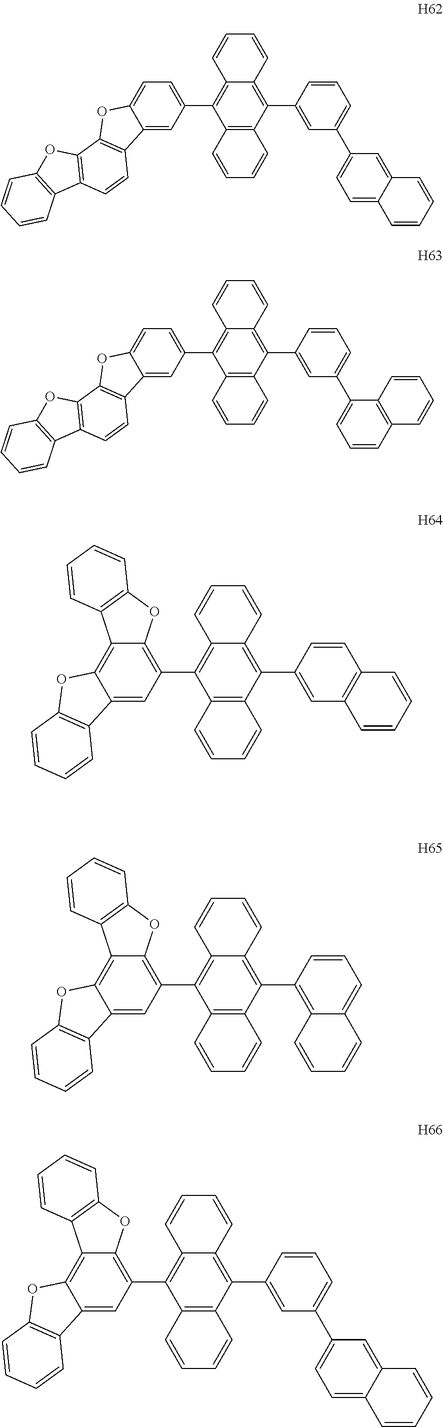 [SMILES: c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1]

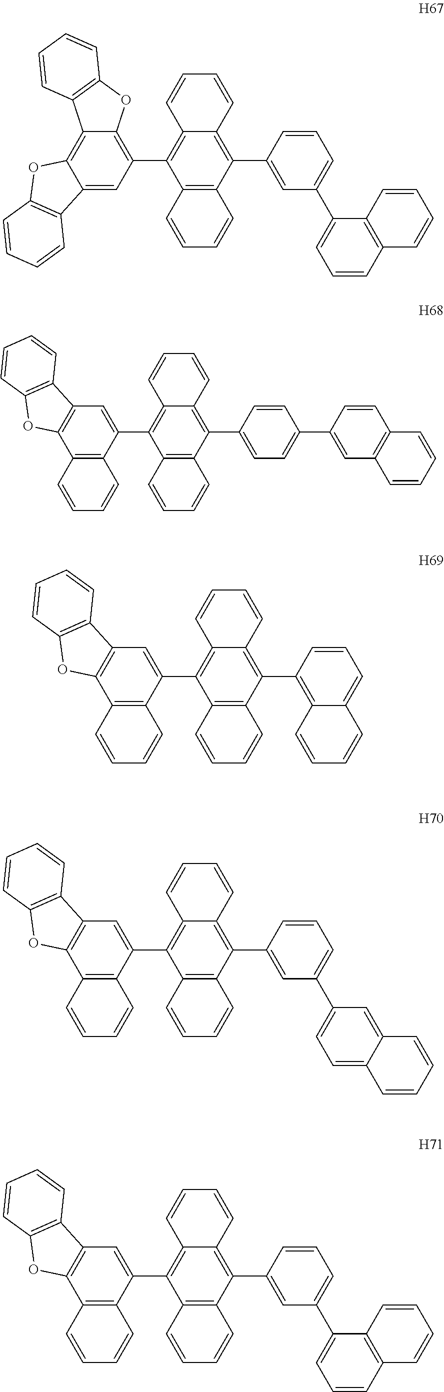 [SMILES: c1cc(-c2cccc3ccccc23)cc(-c2c3ccccc3c(-c3cc4c5ccccc5oc4c4c3oc3ccccc34)c3ccccc23)c1 ; c1ccc2cc(-c3ccc(-c4c5ccccc5c(-c5cc6c7ccccc7oc6c6ccccc56)c5ccccc45)cc3)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1]

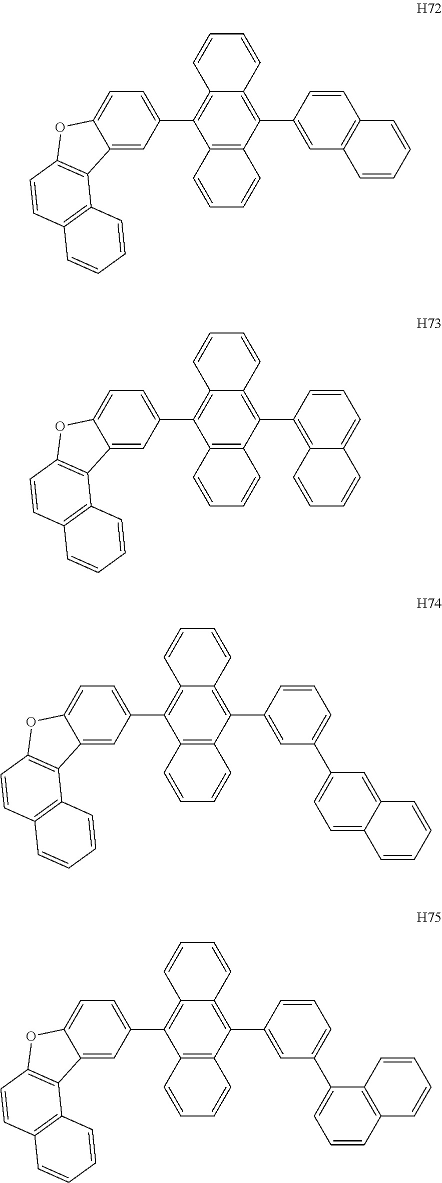 [SMILES: c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1]

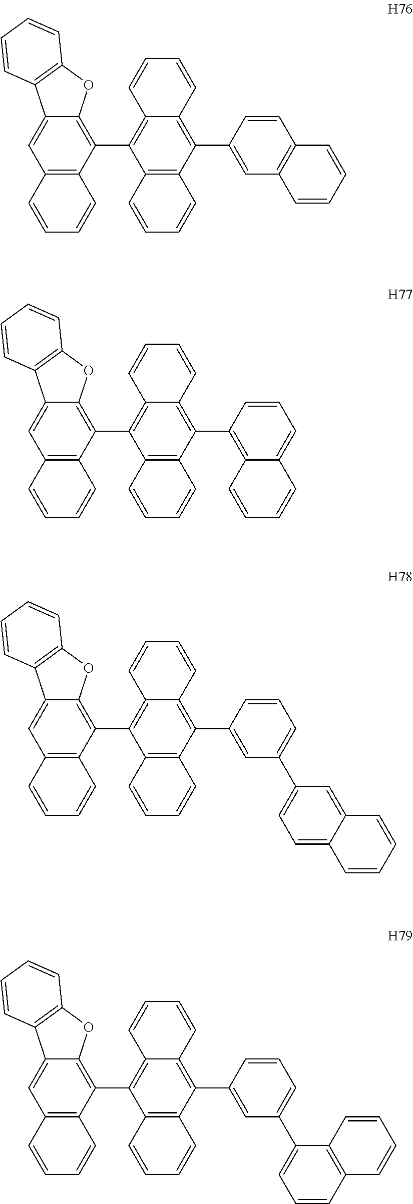 [SMILES: c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1]

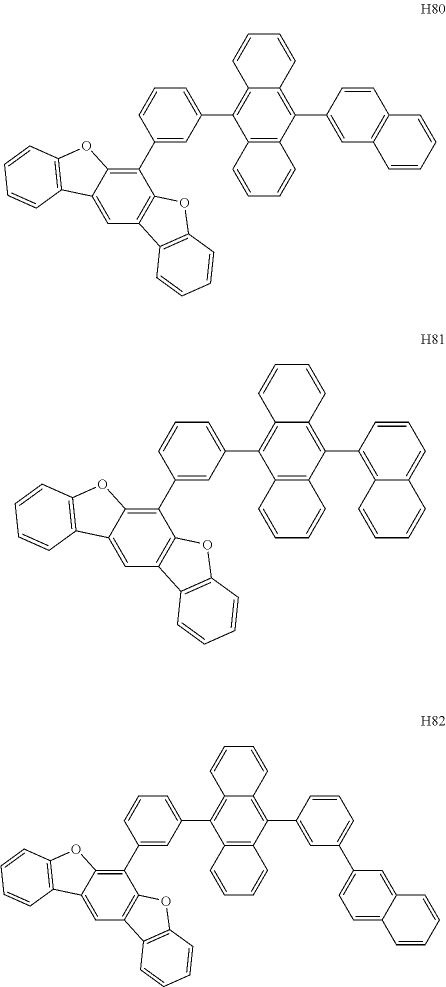 [SMILES: c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1]

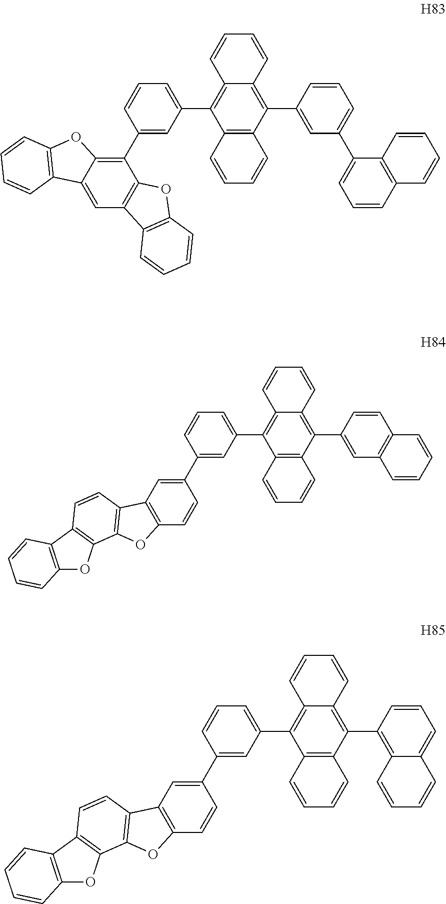 [SMILES: c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1]

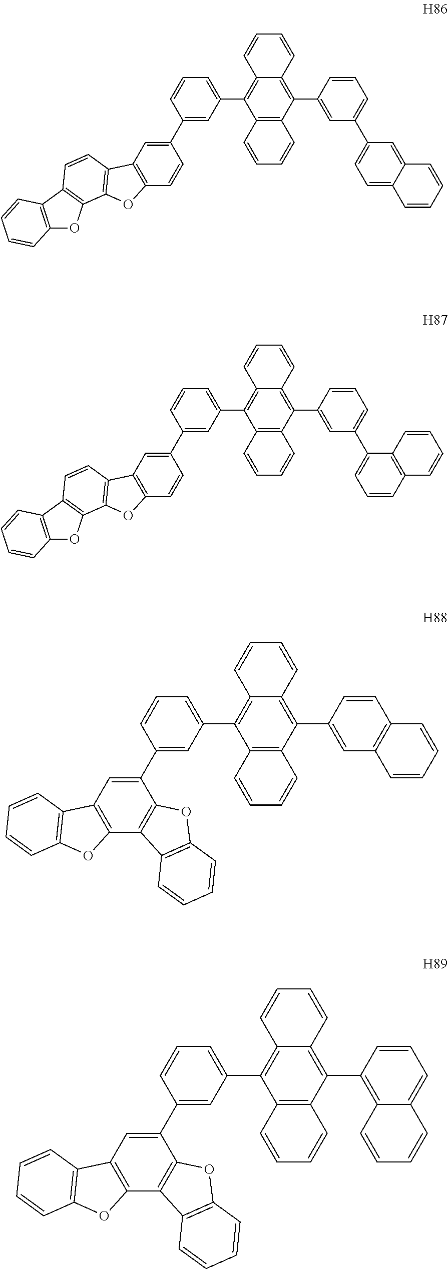 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

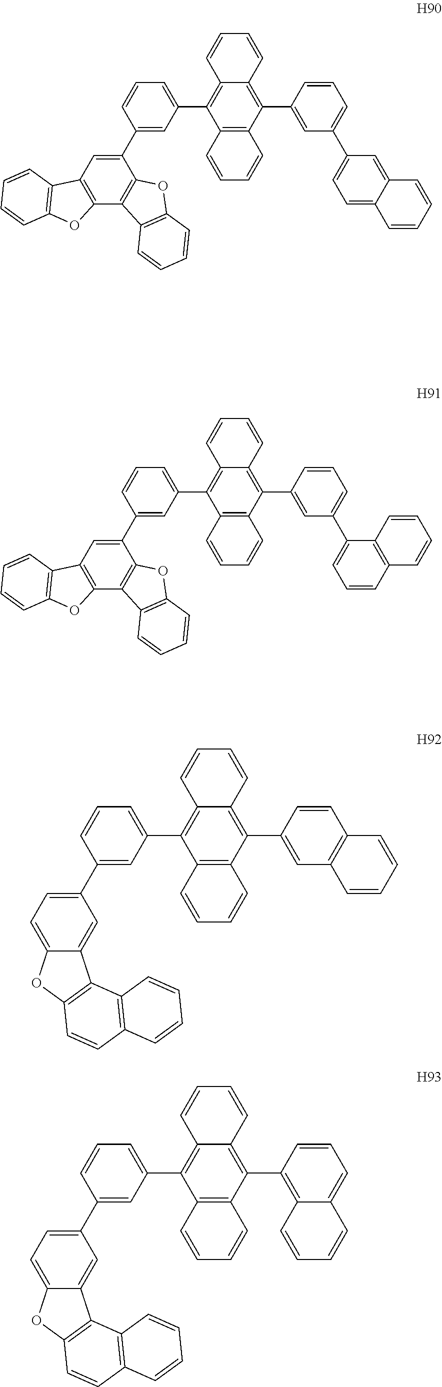 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

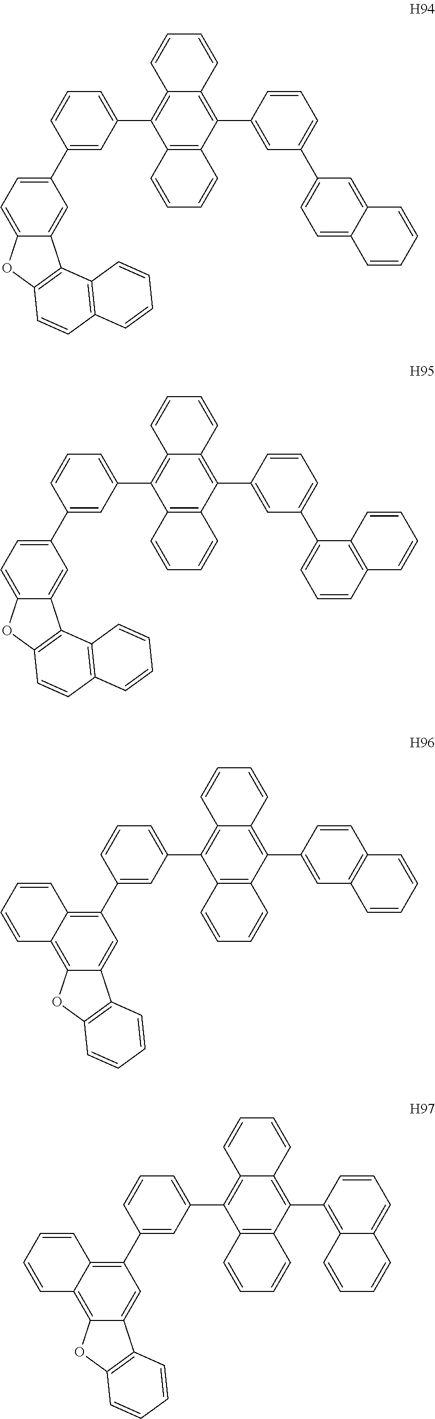 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

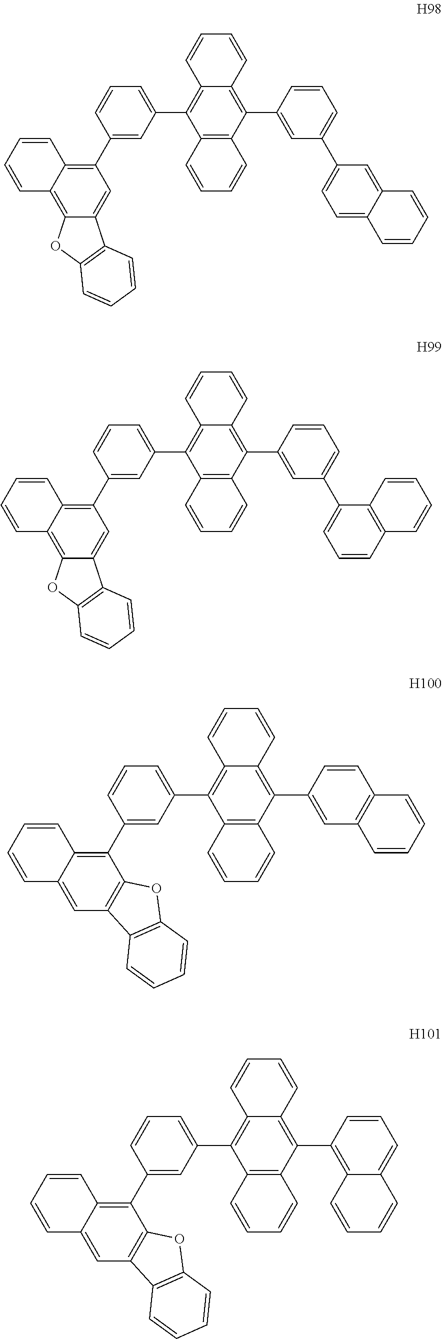 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

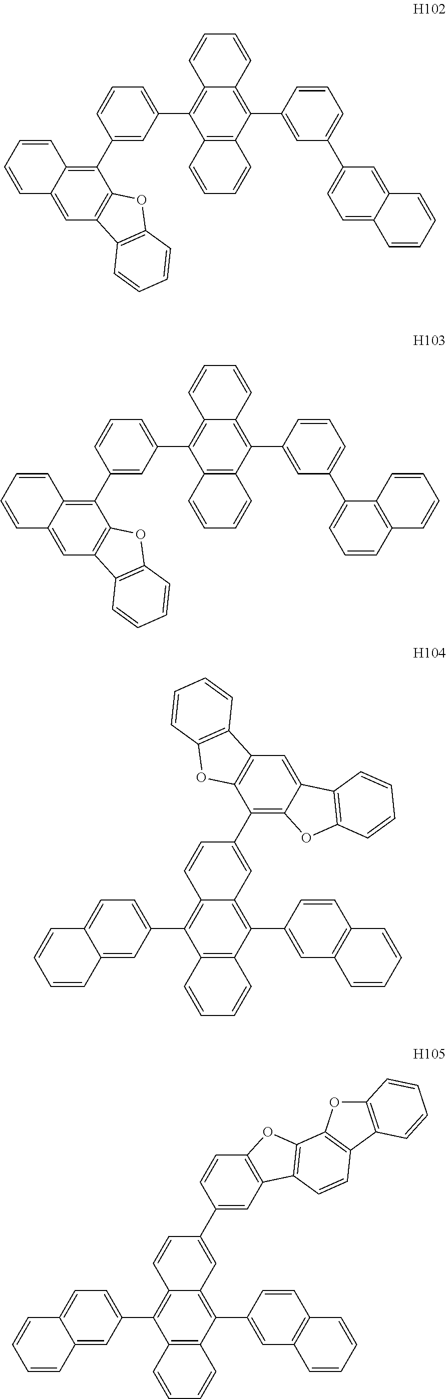 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1]

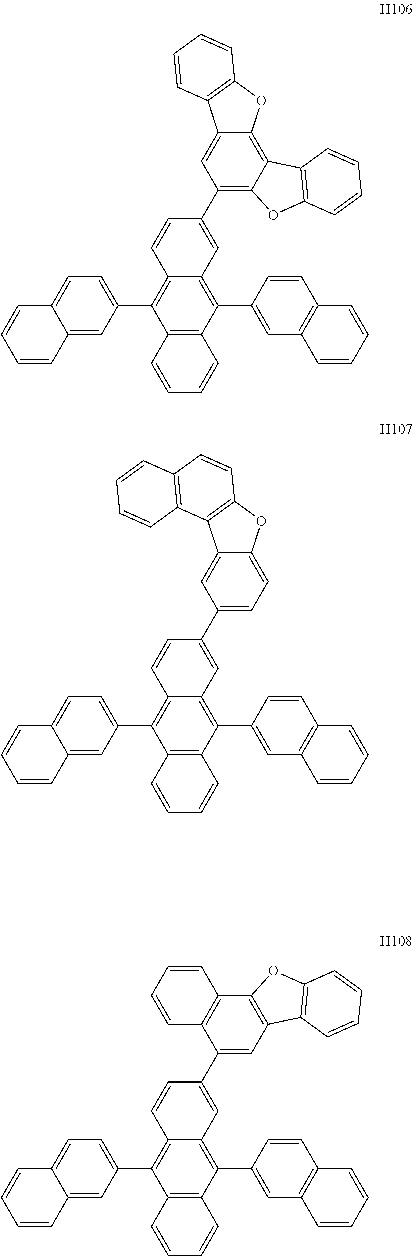 [SMILES: c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

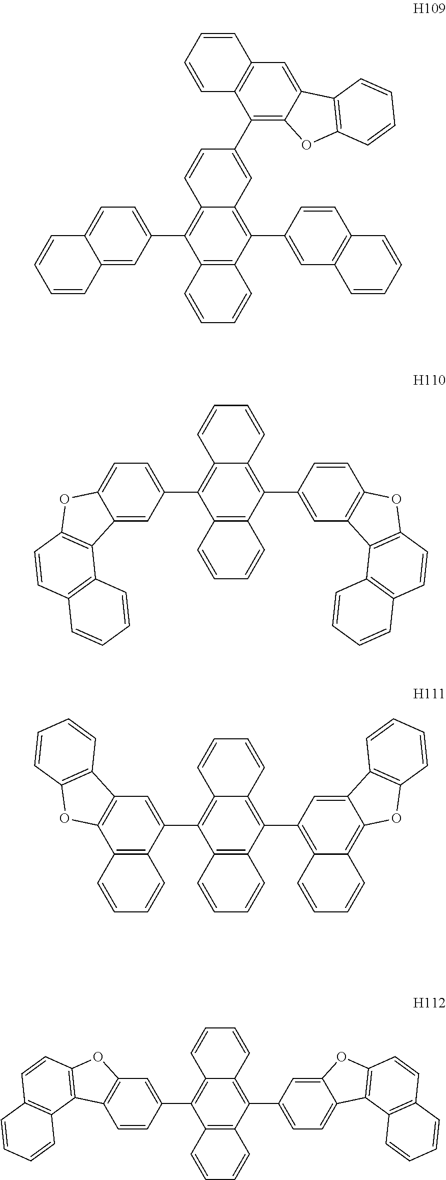 [SMILES: c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12]

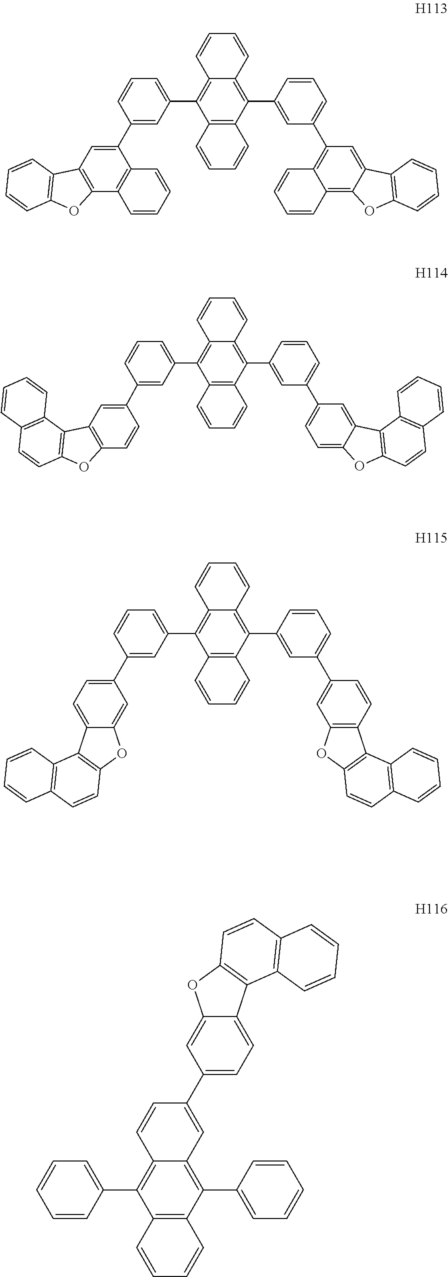 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

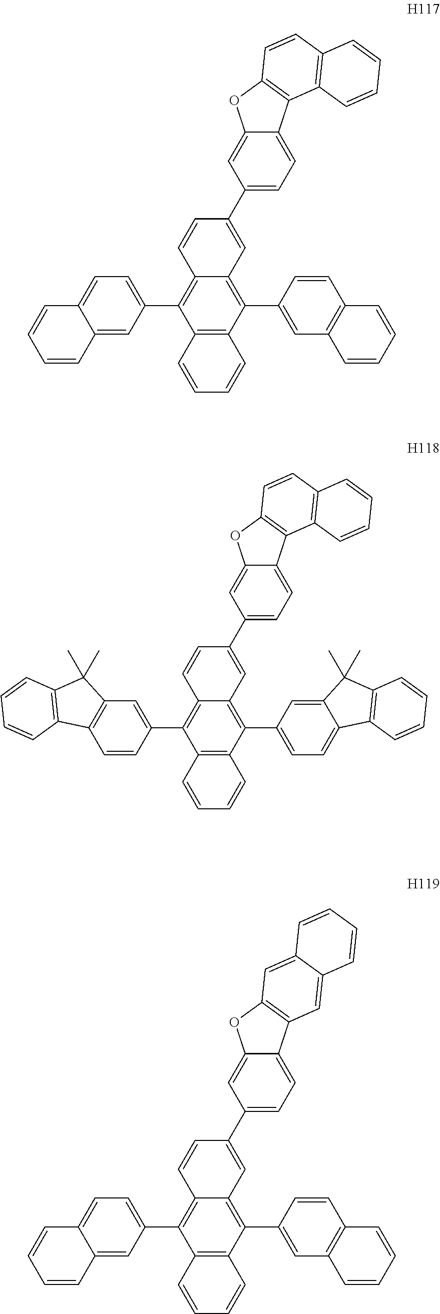 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1]

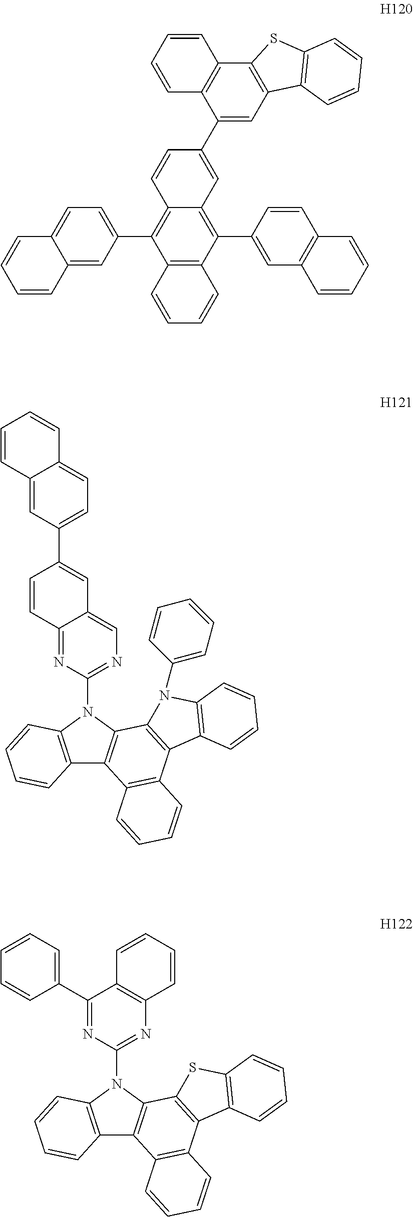 [SMILES: c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1]

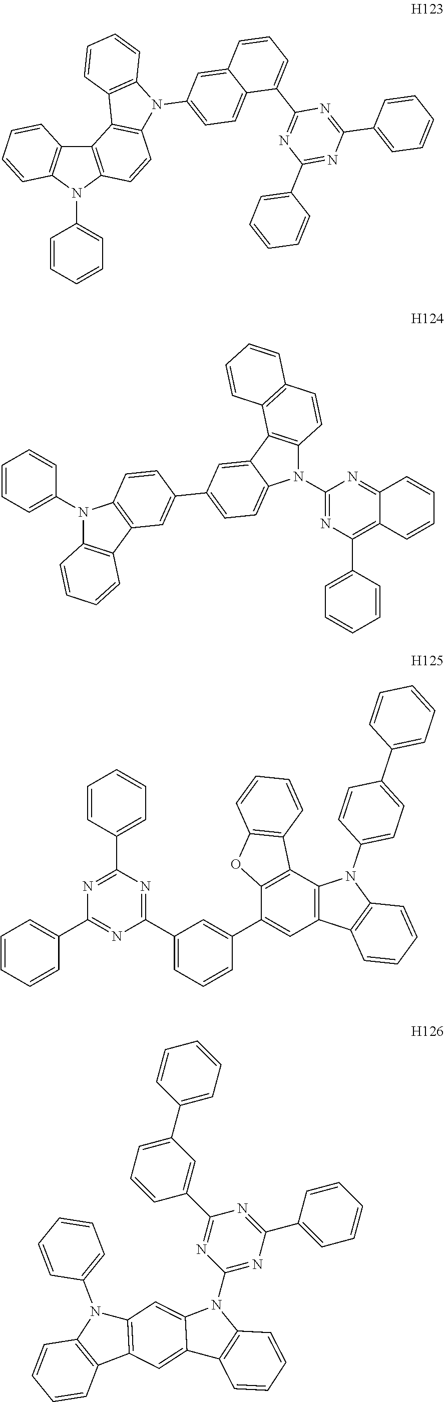 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1]

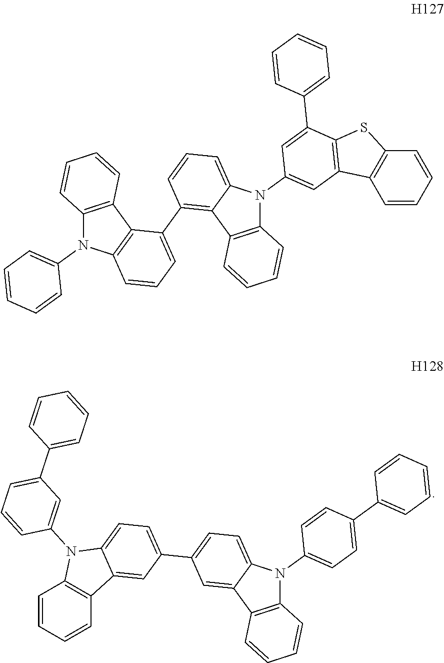 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

In some embodiments, the phosphorescent dopant may be electrically neutral.

For example, in some embodiments, the phosphorescent dopant may include an organometallic compound represented by Formula 401:

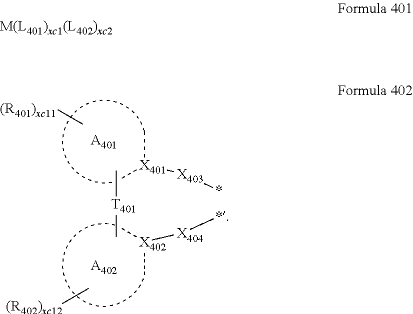

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)—*′, *—C(Q₄₁₁)(Q₄₁₂)—*′, *—C(Q₄₁₁)═C(Q₄₁₂)—*′, *—C(Q₄₁₁)═*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be the same as described herein with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be the same as described herein with respect to Q₁,
xc11 and xc12 may each independently be an integer from 0 to 10, and
* and *′ in Formula 402 may each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁(s) in two or more of L₄₀₁(s) may optionally be linked to each other via T₄₀₂, which is a linking group, and/or two ring A₄₀₂(s) may optionally be linked to each other via T₄₀₃, which is a linking group (see Compounds PD1 to PD4 and PD7). T₄₀₂ and T₄₀₃ may each be the same as described herein with respect to T₄₀₁.

L₄₀₂ in Formula 401 may be an organic ligand. In one or more embodiments, L₄₀₂ may include a halogen, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

In one or more embodiments, the phosphorescent dopant may include, for example, at least one selected from among compounds PD1 to PD39, and/or any combination thereof:

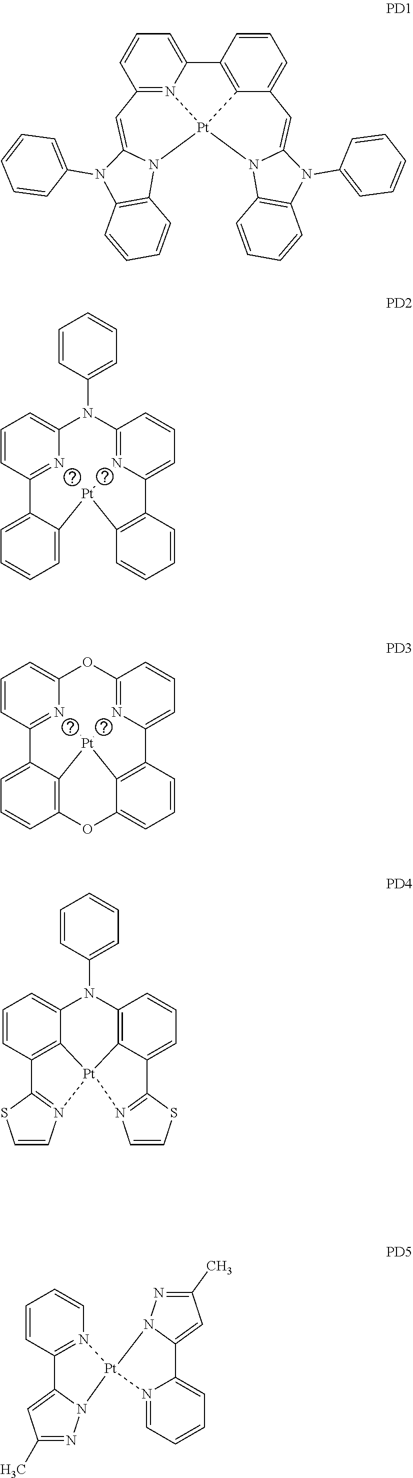

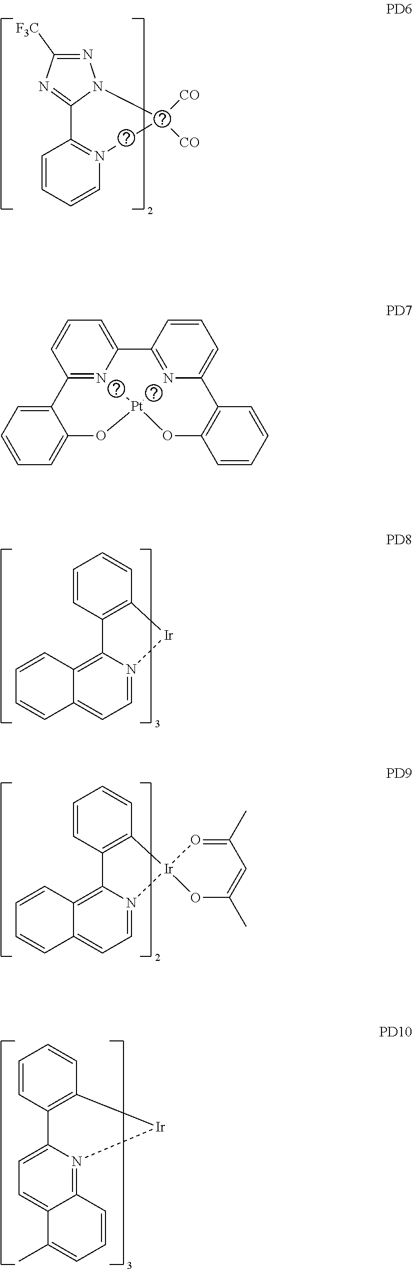

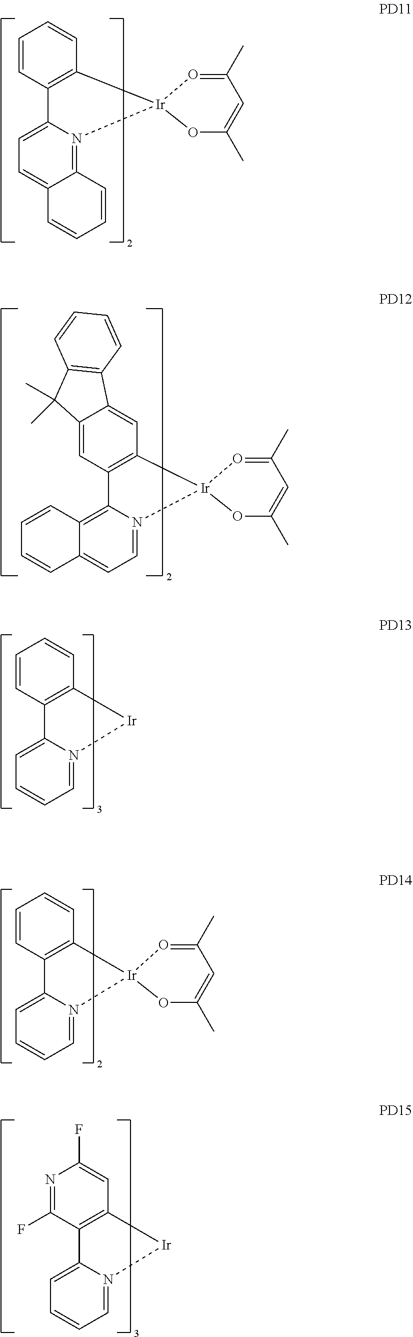

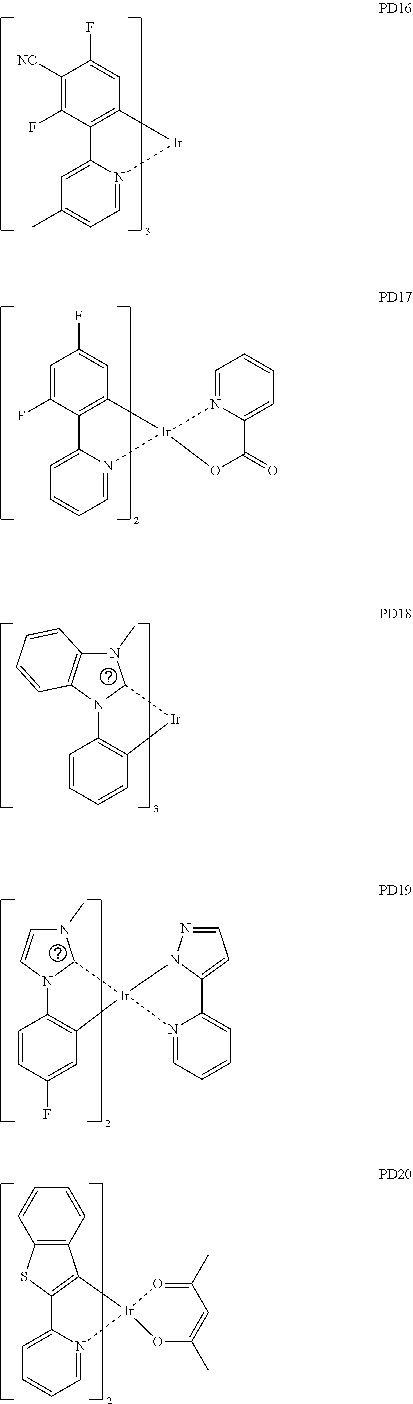

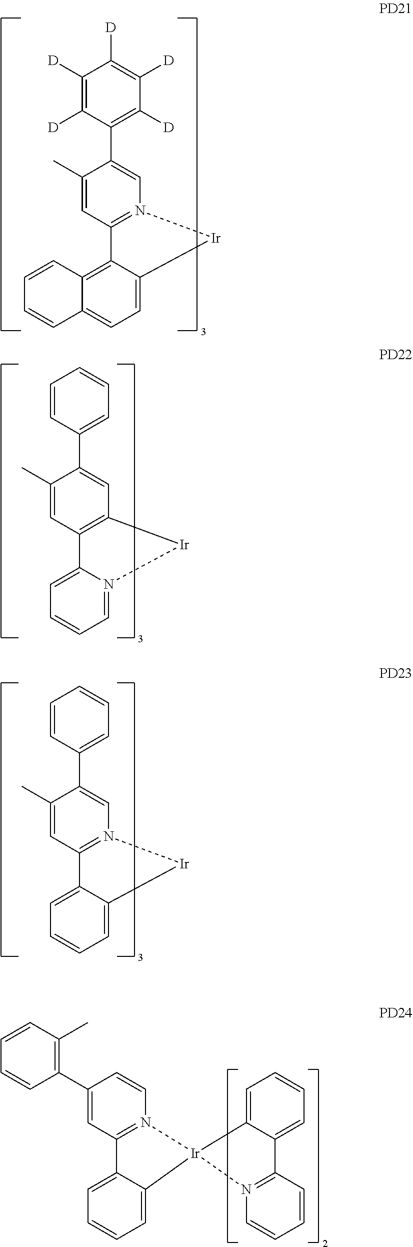

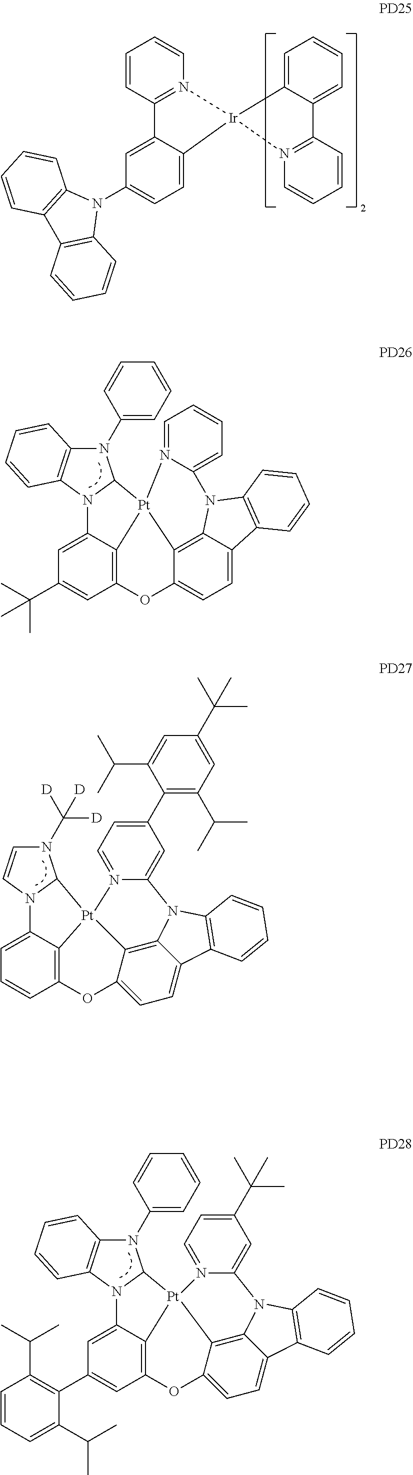

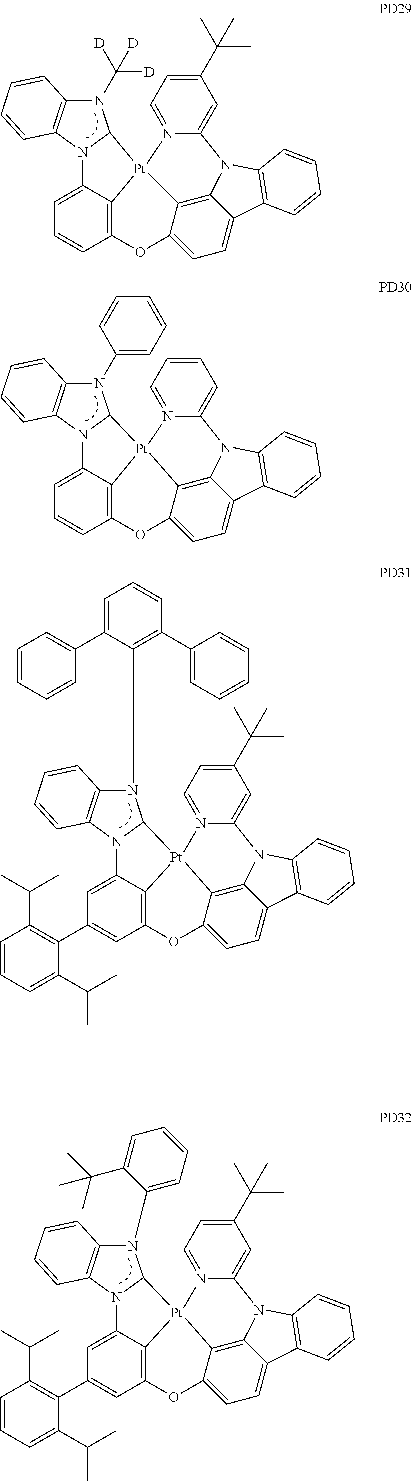

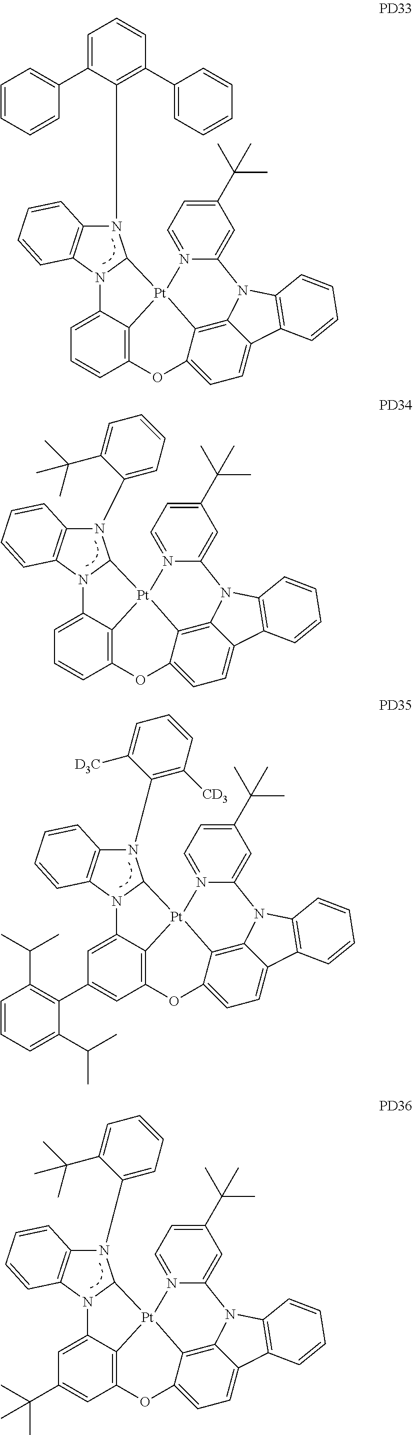

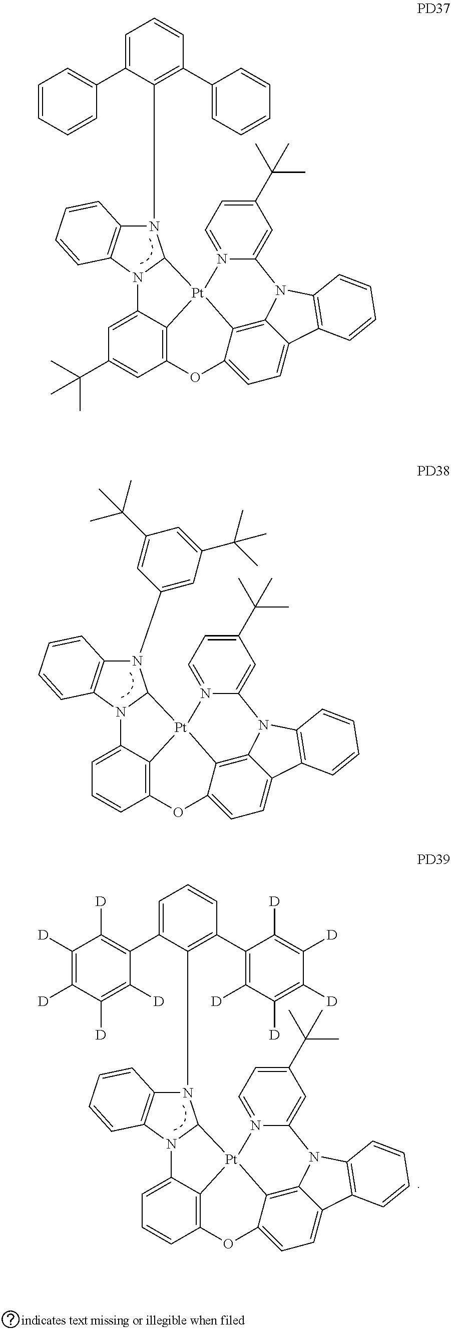

### Fluorescent Dopant

In one or more embodiments, the fluorescent dopant may include an amine group-containing compound, a styryl group-containing compound, or any combination thereof.

For example, in some embodiments, the fluorescent dopant may include a compound represented by Formula 501:

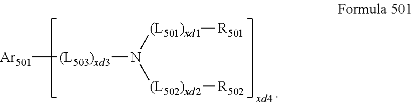

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, in some embodiments, Ar₅₀₁ in Formula 501 may be a condensed cyclic group (for example, an anthracene group, a chrysene group, or a pyrene group) in which three or more monocyclic groups are condensed together. In one or more embodiments, xd4 in Formula 501 may be 2.

In one or more embodiments, the fluorescent dopant may include: at least one selected from Compounds FD1 to FD37; 4,4′-bis(2,2-diphenylvinyl)-1,1′-biphenyl (DPVBi); 4,4′-bis[4-(diphenylamino)styryl]biphenyl (DPAVBi); and/or any combination thereof:

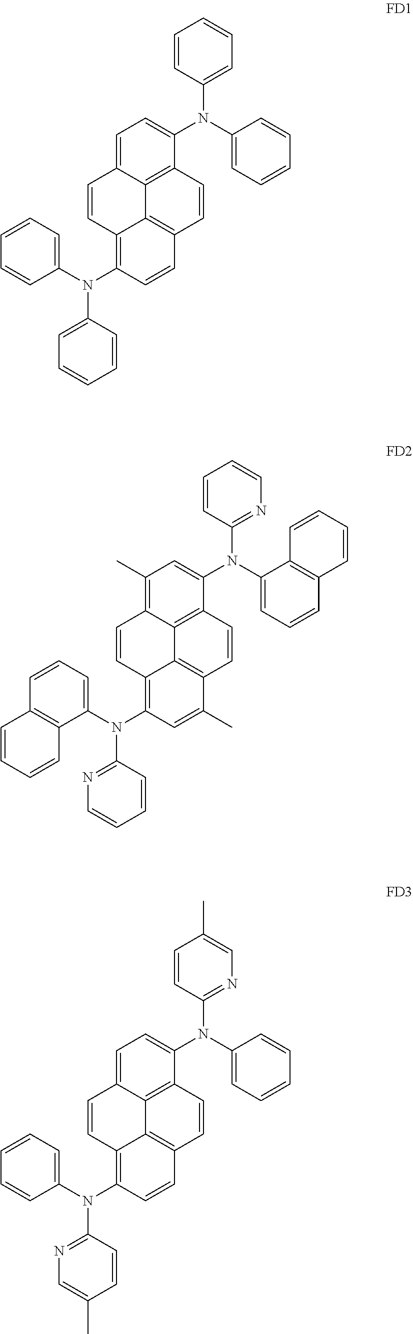 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

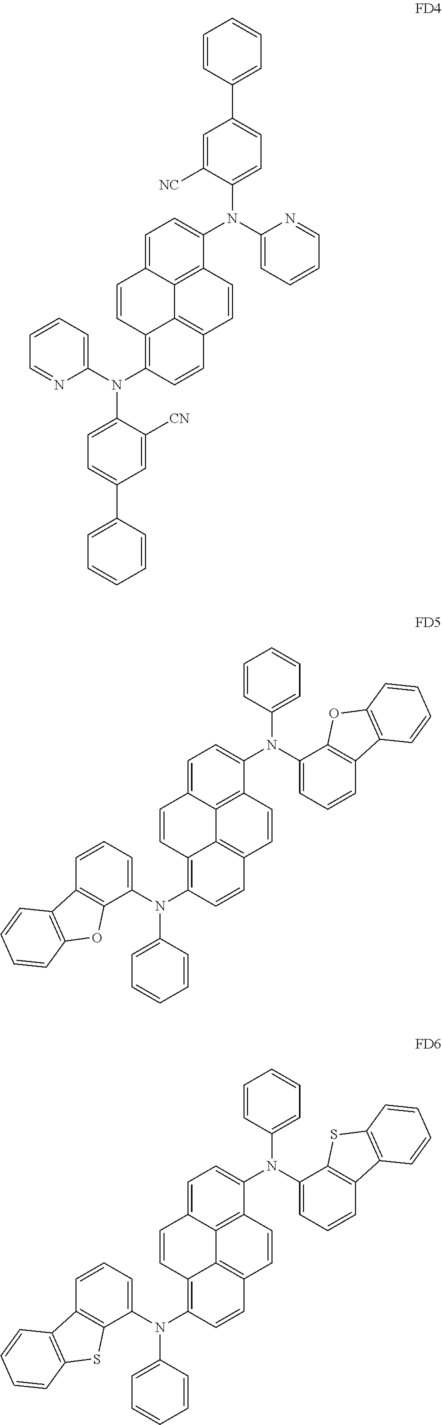 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

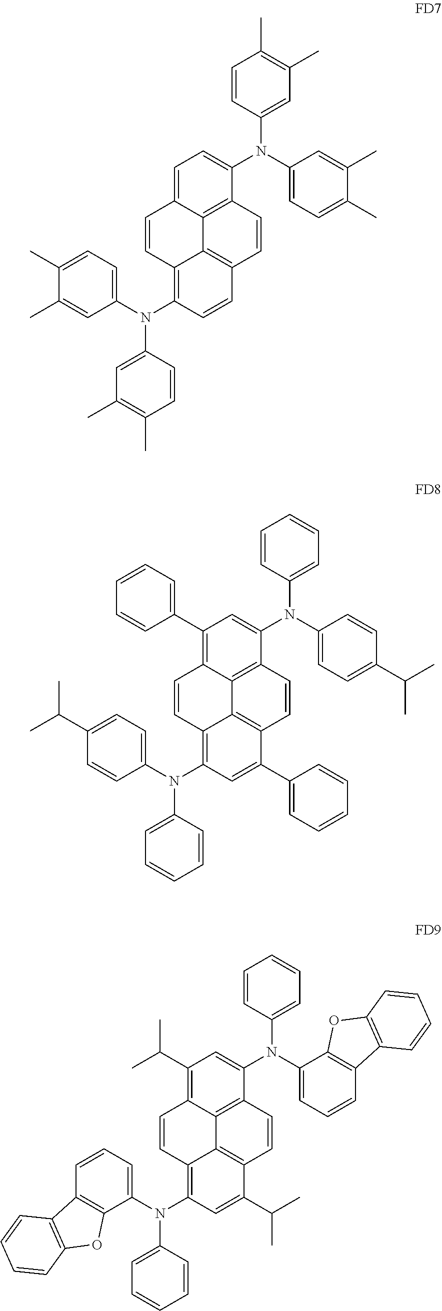

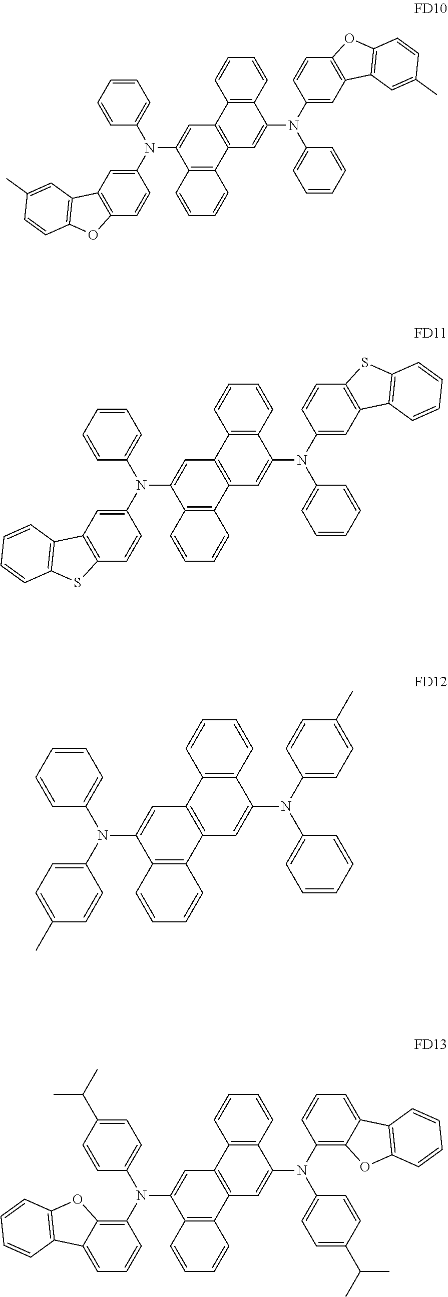

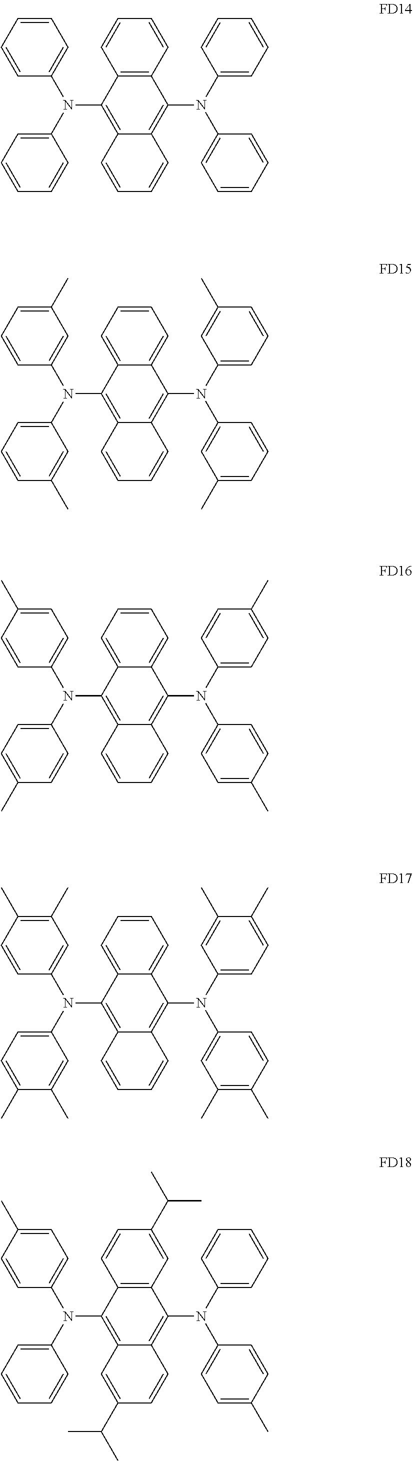 [SMILES: c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

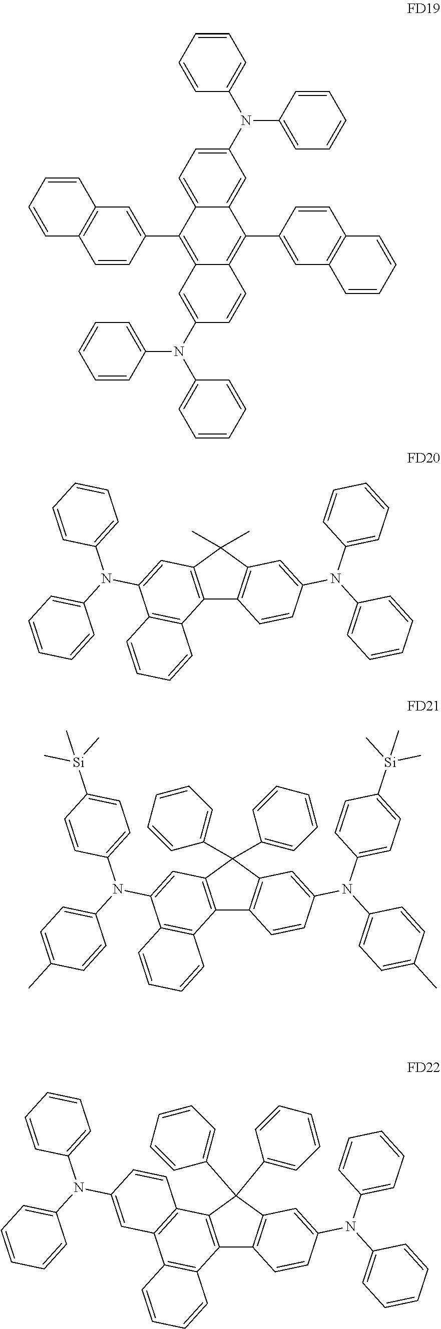

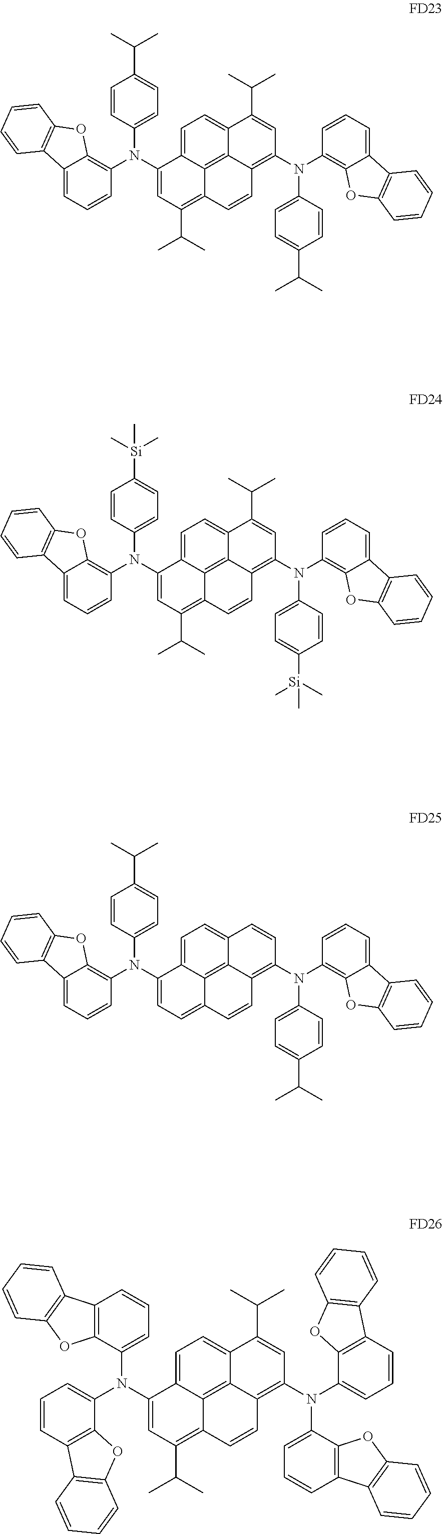 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

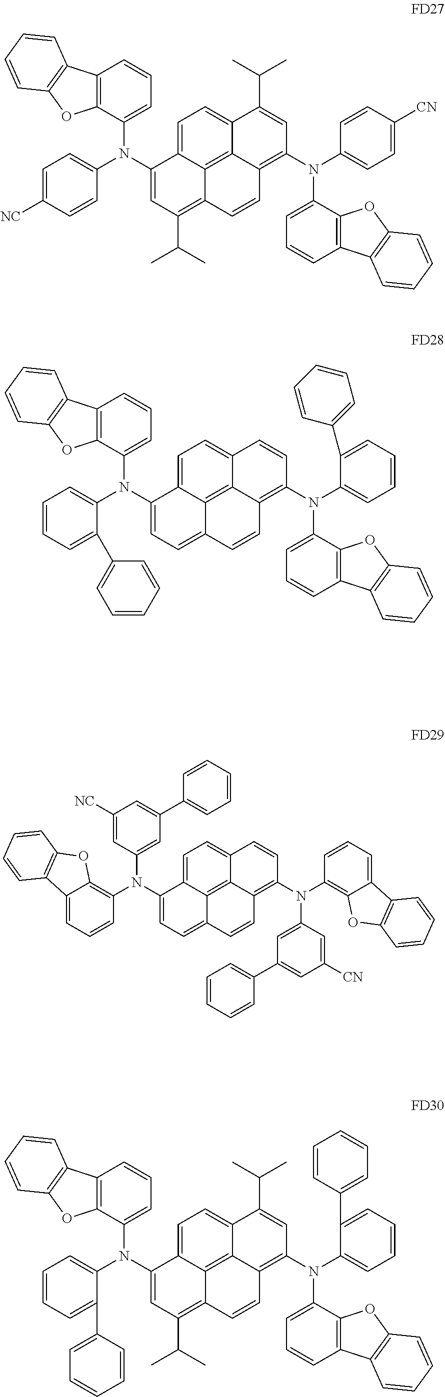 [SMILES: CC(C)c1cc(N(c2ccc(C#N)cc2)c2cccc3c2oc2ccccc23)c2ccc3c(C(C)C)cc(N(c4ccc(C#N)cc4)c4cccc5c4oc4ccccc45)c4ccc1c2c34 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; N#Cc1cc(-c2ccccc2)cc(N(c2ccc3ccc4c(N(c5cc(C#N)cc(-c6ccccc6)c5)c5cccc6c5oc5ccccc56)ccc5ccc2c3c54)c2cccc3c2oc2ccccc23)c1 ; CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34]

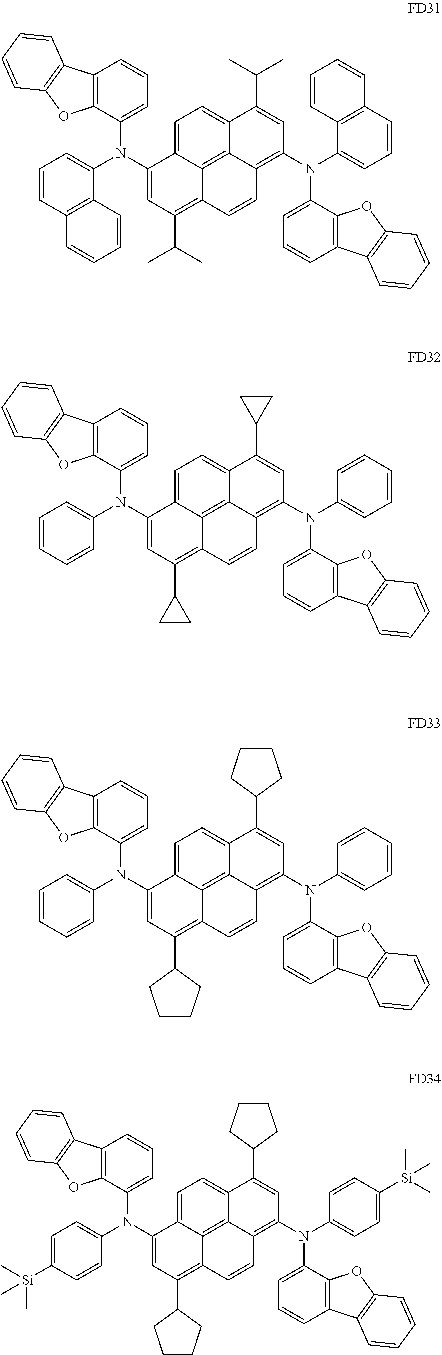

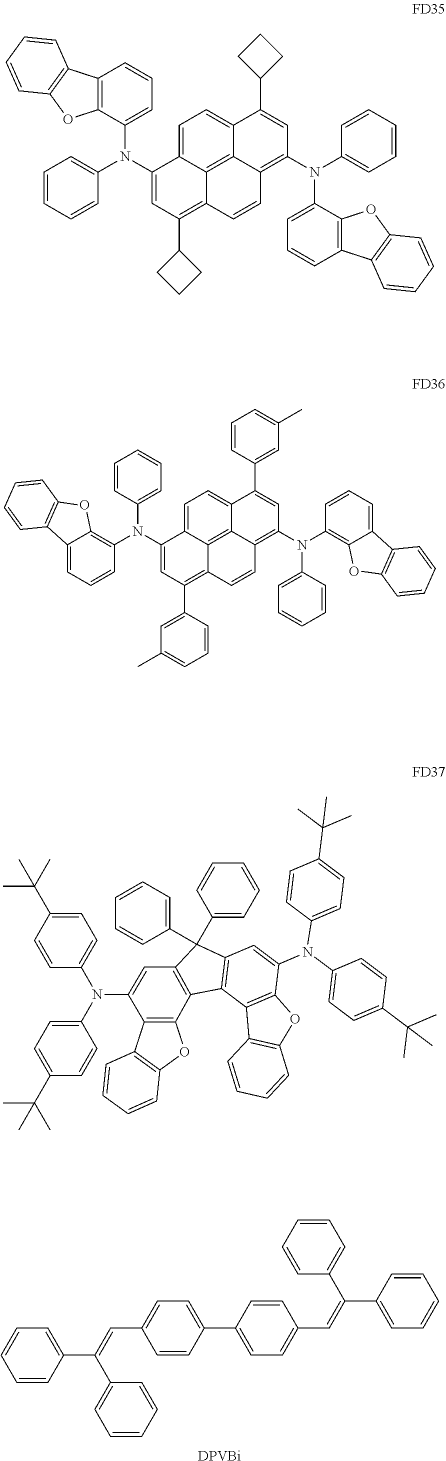

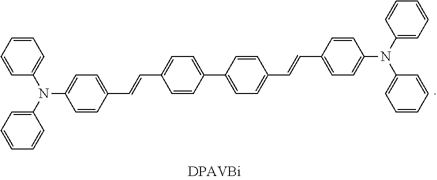 [SMILES: C(=C\c1ccc(N(c2ccccc2)c2ccccc2)cc1)/c1ccc(-c2ccc(/C=C/c3ccc(N(c4ccccc4)c4ccccc4)cc3)cc2)cc1]

### Delayed Fluorescence Material

In one or more embodiments, the emission layer may include a delayed fluorescence material.

In the present disclosure, the delayed fluorescence material may be selected from compounds capable of emitting delayed fluorescent light based on a delayed fluorescence emission mechanism.

The delayed fluorescence material included in the emission layer may act as a host or a dopant depending on the type or kind of other materials included in the emission layer.

According to one or more embodiments, the difference between the triplet energy level of the delayed fluorescence material and the singlet energy level of the delayed fluorescence material may be greater than or equal to about 0 eV and less than or equal to about 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, in some embodiments, the delayed fluorescence material may include: i) a material including at least one electron donor (for example, a π electron-rich C₃-C₆₀ cyclic group and/or the like, such as a carbazole group) and at least one electron acceptor (for example, a sulfoxide group, a cyano group, a π electron-deficient nitrogen-containing C₁-C₆₀ cyclic group, and/or the like), ii) a material including a C₈-C₆₀ polycyclic group including at least two cyclic groups condensed to each other while sharing boron (B), and/or the like.

Non-limiting examples of the delayed fluorescence material may include at least one selected from among Compounds DF1 to DF14:

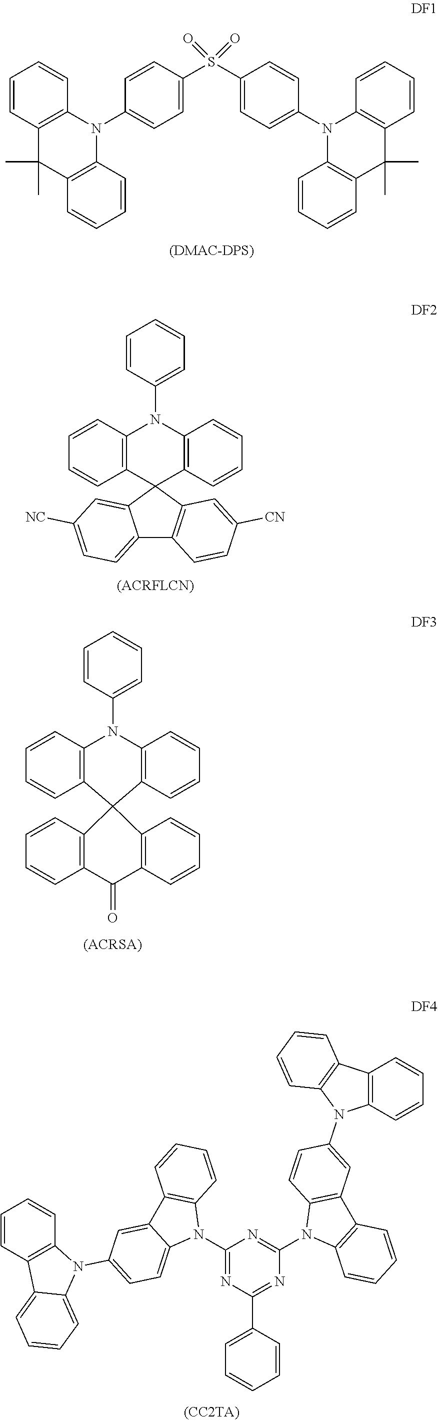

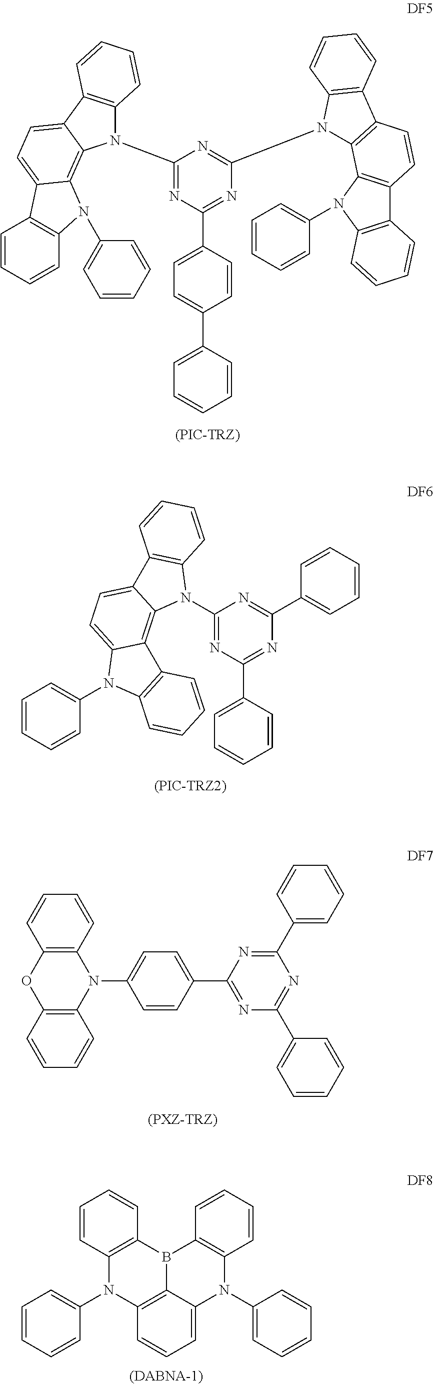

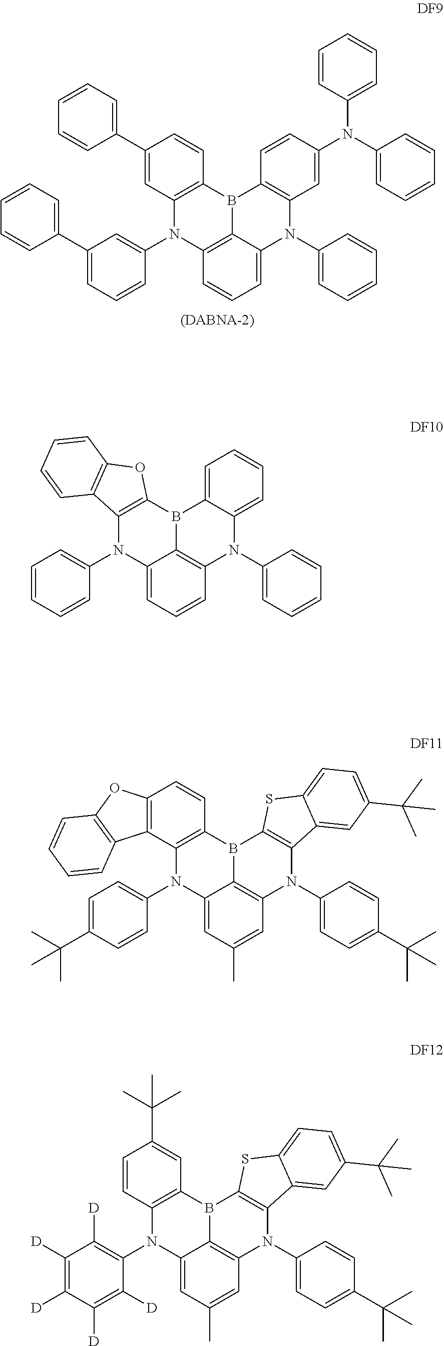

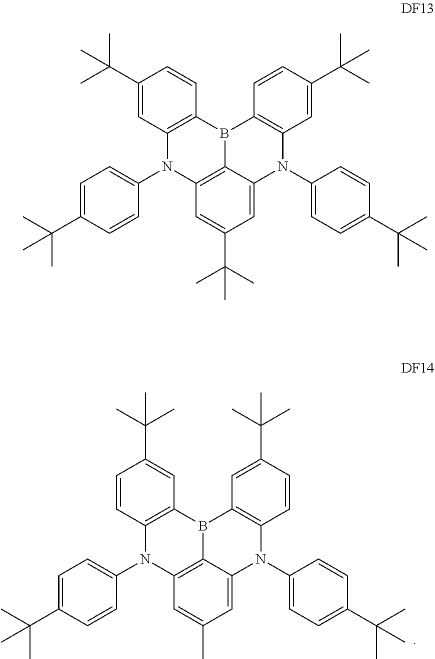

### Quantum Dot

In one or more embodiments, the emission layer may include a quantum dot.

The term “quantum dot” as utilized herein may refer to a crystal of a semiconductor compound, and may include any material capable of emitting light of one or more suitable emission wavelengths according to the size of the crystal.

A diameter of the quantum dots may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE),

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element or compound, or any combination thereof.

Non-limiting examples of the Group II-VI semiconductor compound may be a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Non-limiting examples of the Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. In some embodiments, the Group III-V semiconductor compound may further include a Group II element. Non-limiting examples of the Group III-V semiconductor compound further including a Group II element may be InZnP, InGaZnP, InAlZnP, etc.

Non-limiting examples of the Group III-VI semiconductor compound may be: a binary compound, such as GaS, GaSe, Ga₂Se₃, GaTe, InS, InSe, In₂S₃, In₂Se₃, or InTe; a ternary compound, such as InGaS₃, or InGaSe₃; and/or any combination thereof.

Non-limiting examples of the Group I-III-VI semiconductor compound may be: a ternary compound, such as AgInS, AgInS₂, CuInS, CuInS₂, CuGaO₂, AgGaO₂, or AgAlO₂; or any combination thereof.

Non-limiting examples of the Group IV-VI semiconductor compound may be: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

The Group IV element or compound may include: a single element compound, such as Si or Ge; a binary compound, such as SiC or SiGe; or any combination thereof.

Each element included in a multi-element compound such as the binary compound, the ternary compound, and the quaternary compound may be present at a substantially uniform concentration or non-substantially uniform concentration in a particle.

In some embodiments, the quantum dot may have a single structure in which the concentration of each element in the quantum dot is substantially uniform, or a core-shell dual structure. For example, the material included in the core and the material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Non-limiting examples of the shell of the quantum dot may be an oxide of metal, metalloid, or non-metal, a semiconductor compound, and/or any combination thereof. Non-limiting examples of the oxide of metal, metalloid, or non-metal may be a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and/or any combination thereof. Non-limiting examples of the semiconductor compound may be, as described herein, a Group II-VI semiconductor compound; a Group III-V semiconductor compound; a Group III-VI semiconductor compound; a Group I-III-VI semiconductor compound; a Group IV-VI semiconductor compound; and/or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be about 45 nm or less, for example, about 40 nm or less, for example, about 30 nm or less, and within these ranges, color purity or color reproducibility may be increased. In some embodiments, because the light emitted through the quantum dot is emitted in all directions, the wide viewing angle may be improved.

In some embodiments, the quantum dot may be in the form of a substantially spherical particle, a pyramidal particle, a multi-arm particle, a cubic nanoparticle, a nanotube particle, a nanowire particle, a nanofiber particle, or a nanoplate particle.

Because the energy band gap may be adjusted by controlling the size of the quantum dot, light having one or more suitable wavelength bands may be obtained from the quantum dot emission layer. Accordingly, by utilizing quantum dots of different sizes, a light-emitting device that emits light of one or more suitable wavelengths may be implemented. In one or more embodiments, the size of the quantum dot may be selected to emit red, green, and/or blue light. In some embodiments, the size of the quantum dot may be configured to emit white light by combination of light of one or more suitable colors.

### Electron Transport Region in Interlayer 130

The electron transport region may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron transport region may include a buffer layer, a hole-blocking layer, an electron control layer, an electron-transport layer, an electron injection layer, or any combination thereof.

For example, the electron transport region may include an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the constituting layers of each structure being sequentially stacked from an emission layer in the stated order.

In one or more embodiments, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, in some embodiments, the electron transport region may include a compound represented by Formula 601:

[Ar₆₀₁]ₓₑ₁₁-[(L₆₀₁)ₓₑ₁-R₆₀₁]ₓₑ₂₁ Formula 601

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described herein with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In some embodiments, Ar₆₀₁ in Formula 601 may be an anthracene group unsubstituted or substituted with at least one R₁₀ₐ.

In some embodiments, the electron transport region may include a compound represented by Formula 601-1:

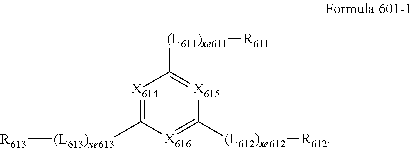

In Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as described herein with respect to L₆₀₁,
xe611 to xe613 may each be the same as described herein with respect to xe1,
R₆₁₁ to R₆₁₃ may each be the same as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₆₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), Alq3, bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), and/or any combination thereof:

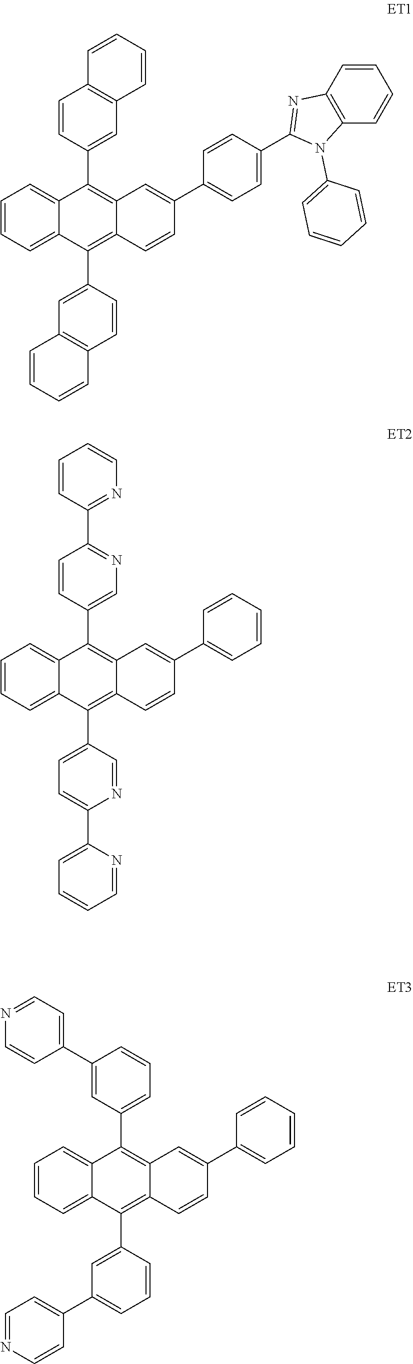 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

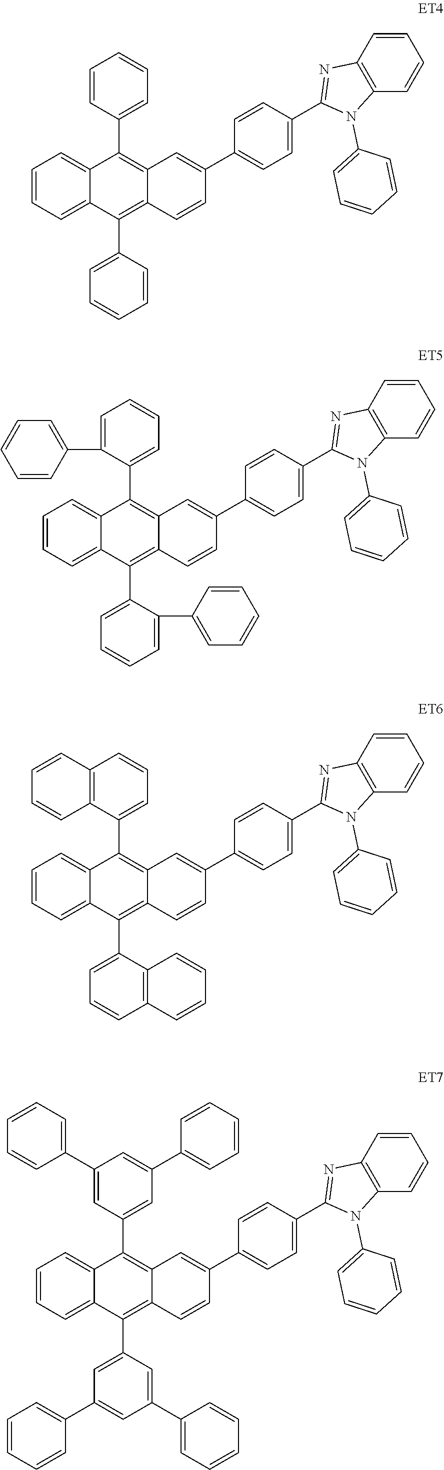 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

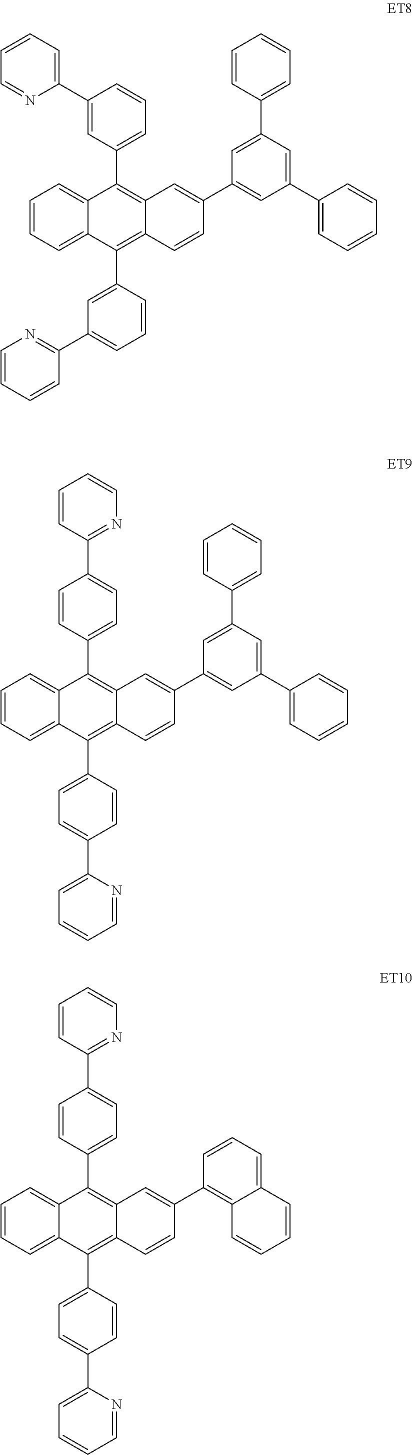 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

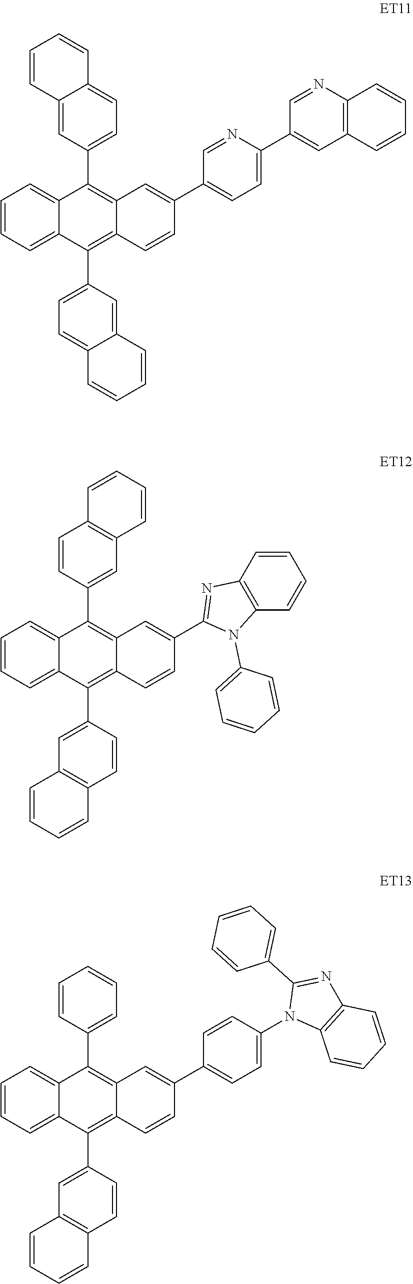 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

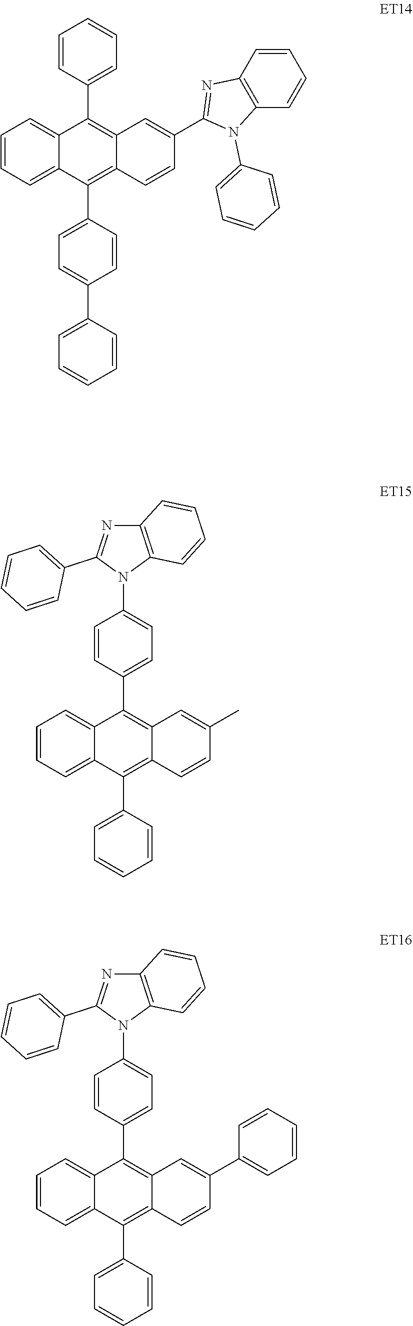 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

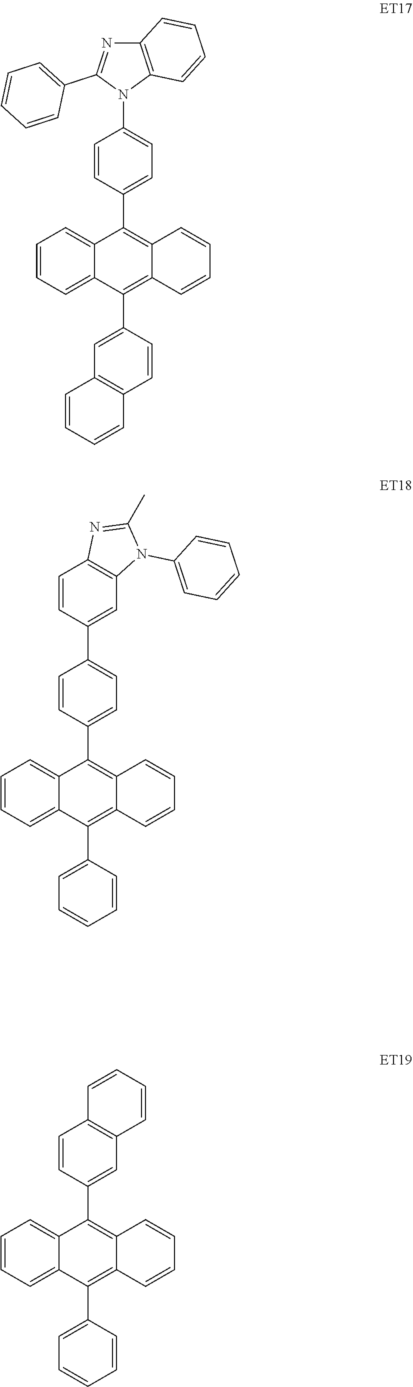

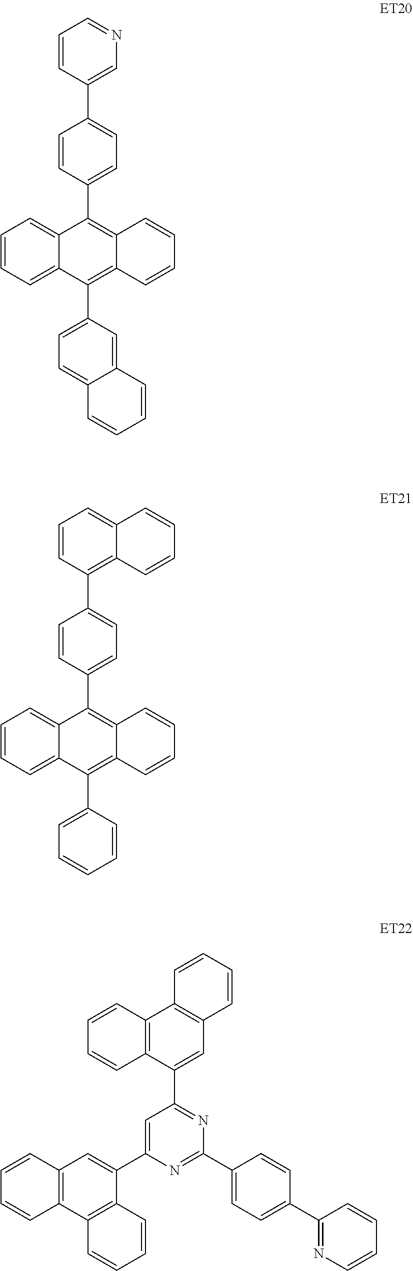

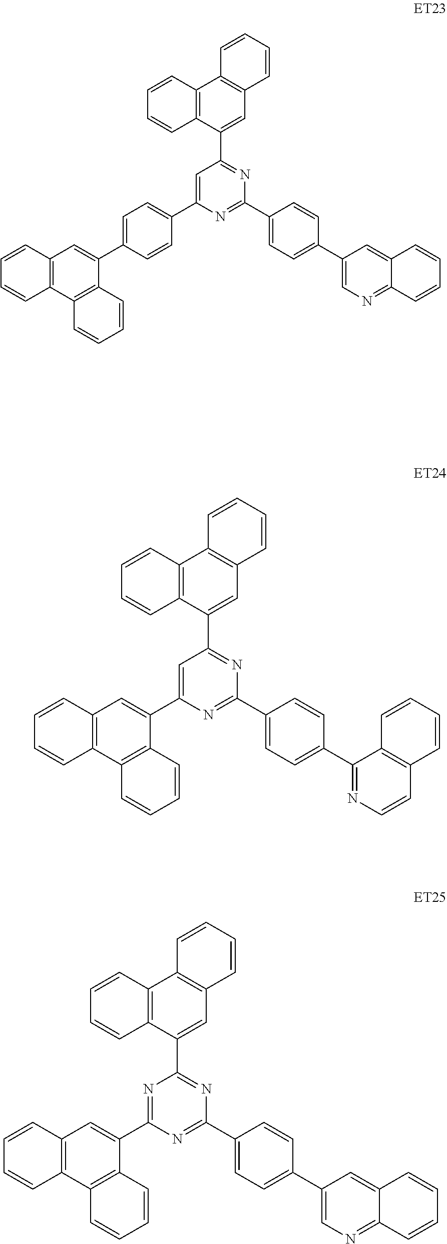 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

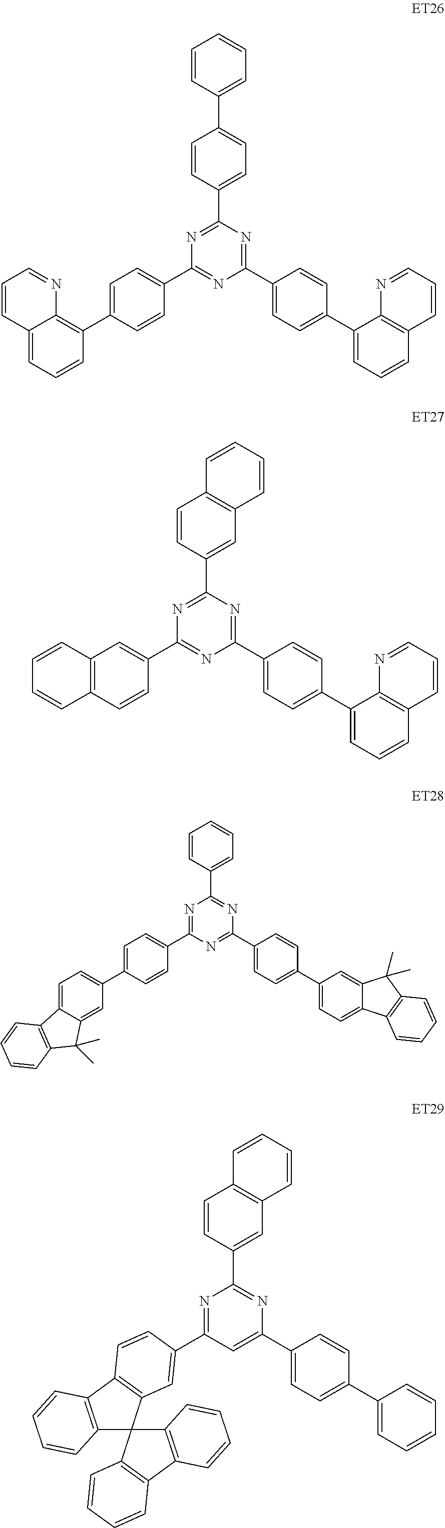 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

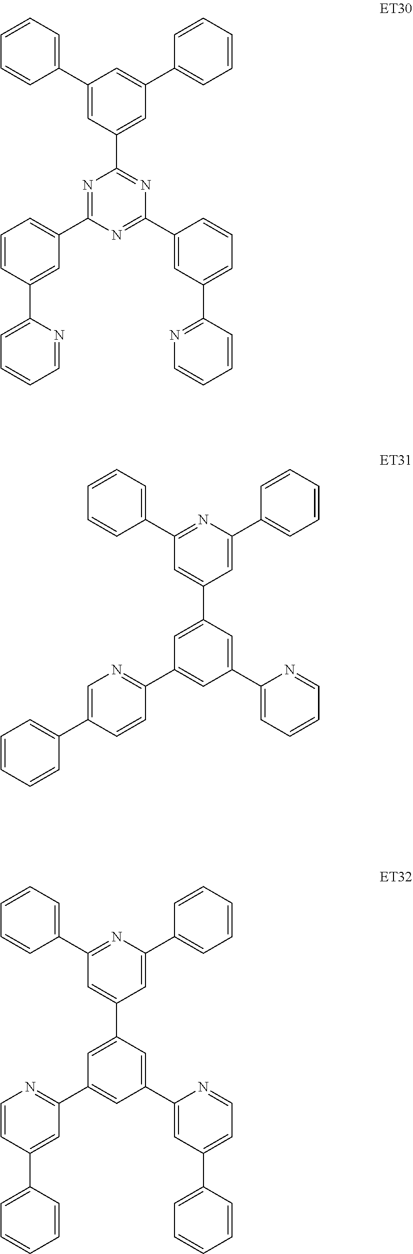 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

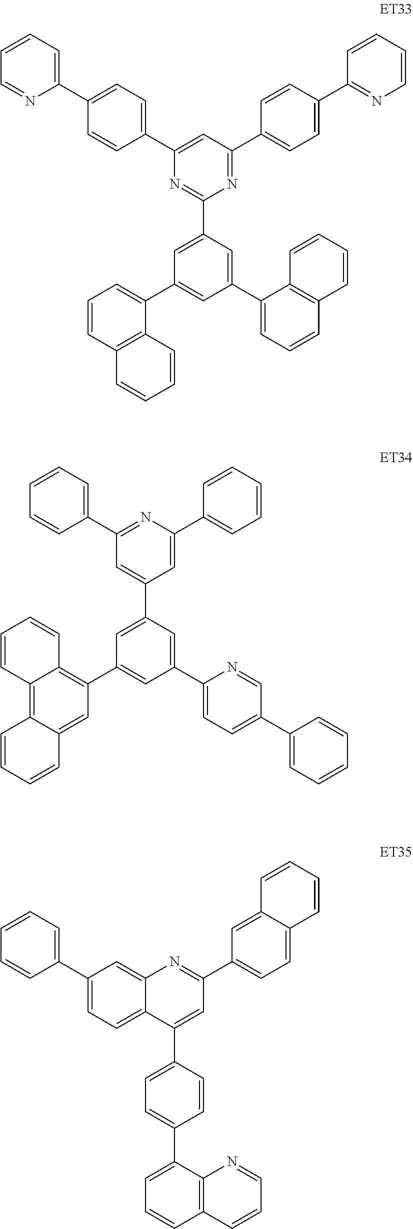 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

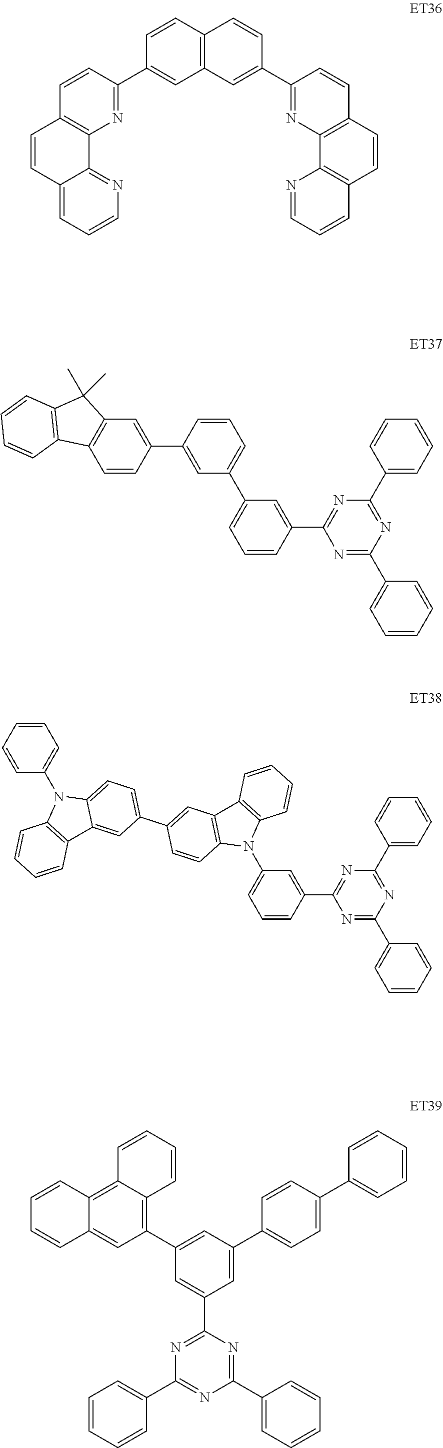 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

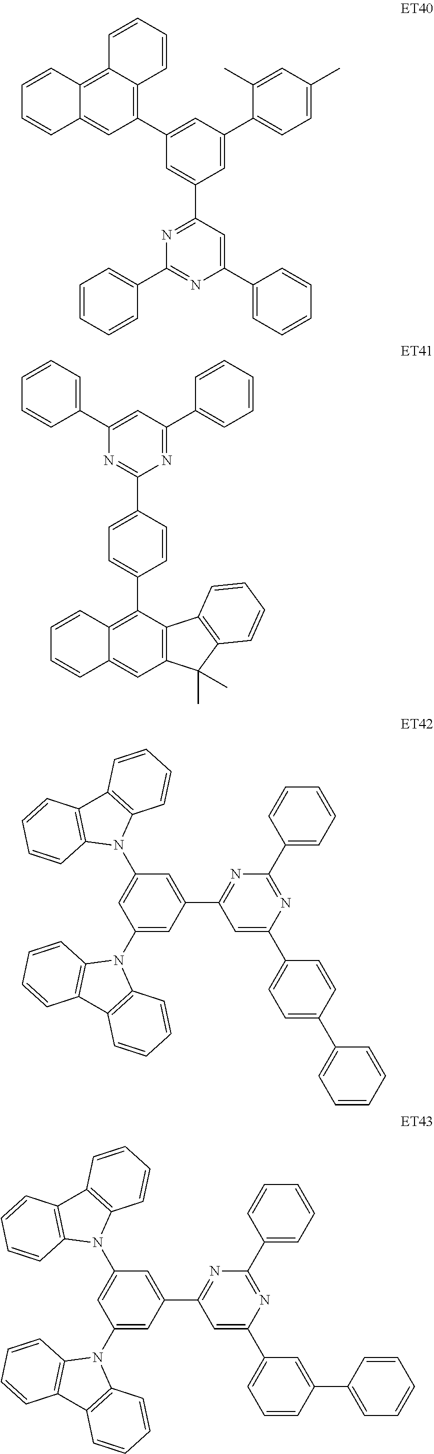 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

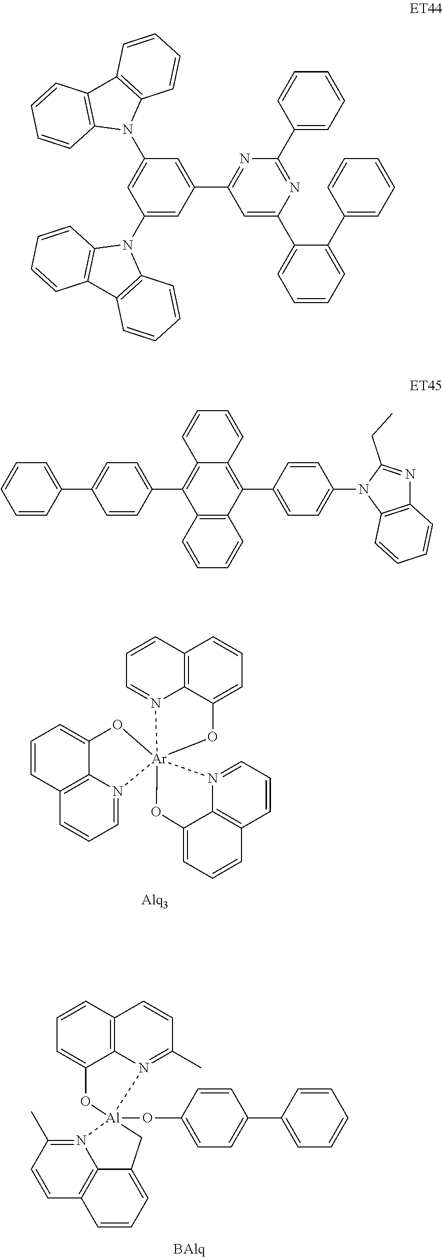

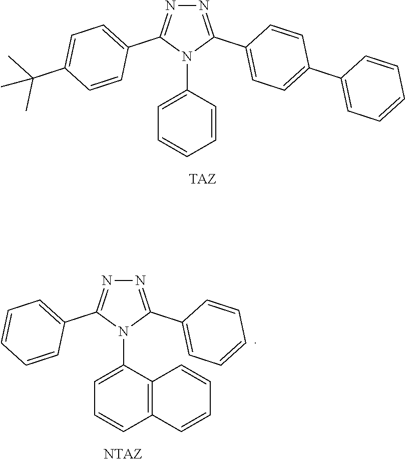 [SMILES: CC(C)(C)c1ccc(-c2nnc(-c3ccc(-c4ccccc4)cc3)n2-c2ccccc2)cc1 ; c1ccc(-c2nnc(-c3ccccc3)n2-c2cccc3ccccc23)cc1]

A thickness of the electron transport region may be from about 100 Å to about 5,000 Å, for example, about 160 Å to about 4,000 Å. When the electron transport region includes a buffer layer, a hole-blocking layer, an electron control layer, an electron transport layer, or any combination thereof, the thickness of the buffer layer, the hole-blocking layer, or the electron control layer may each independently be from about 20 Å to about 1,000 Å, for example, about 30 Å to about 300 Å, and the thickness of the electron transport layer may be from about 100 Å to about 1,000 Å, for example, about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

In one or more embodiments, the electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, in some embodiments, the metal-containing material may include a Li complex. The Li complex may include, for example, Compound ET-D1 (LiQ) or ET-D2:

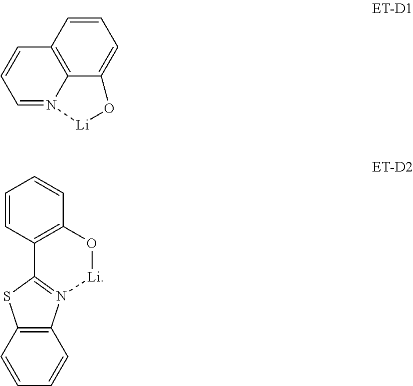

In one or more embodiments, the electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaxSr₁₋ₓO (wherein x is a real number satisfying the condition of 0<x<1), BaxCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Non-limiting examples of the lanthanide metal telluride may be LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) one of ions of the alkali metal, the alkaline earth metal, and the rare earth metal, respectively, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

### Second Electrode 150

The second electrode 150 may be on the interlayer 130. The second electrode 150 may be a cathode which is an electron injection electrode. A material for forming the second electrode 150 may include metal, an alloy, an electrically conductive compound, or any combination thereof, each of which has a low work function.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be located outside the first electrode 110, and/or a second capping layer may be located outside the second electrode 150. In some embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

For example, in some embodiments, light generated by the emission layer in the interlayer 130 of the light-emitting device 10 may be extracted to the outside through the first electrode 110, which is a semi-transmissive electrode or a transmissive electrode, and the first capping layer. For example, in some embodiments, light generated by the emission layer in the interlayer 130 of the light-emitting device 10 may be extracted to the outside through the second electrode 150, which is a semi-transmissive electrode or a transmissive electrode, and the second capping layer.

The first capping layer and the second capping layer may increase external emission efficiency according to the principle of constructive interference. Consequently, the light extraction efficiency of the light-emitting device 10 may be increased, so that the luminescence efficiency of the light-emitting device 10 may be improved.

The first capping layer and the second capping layer may each include a material having a refractive index of about 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may each independently include carbocyclic compounds, heterocyclic compounds, amine group-containing compounds, porphine derivatives, phthalocyanine derivatives, naphthalocyanine derivatives, alkali metal complexes, alkaline earth metal complexes, or any combination thereof. In some embodiments, the carbocyclic compound, the heterocyclic compound, and the amine group-containing compound may be substituted with a substituent including O, N, S, Se, Si, F, Cl, Br, I, or any combination thereof. In one or more embodiments, at least one selected from among the first capping layer and the second capping layer may each independently include an amine group-containing compound.

For example, in some embodiments, at least one selected from among the first capping layer and the second capping layer may each independently include a compound represented by Formula 201, a compound represented by Formula 202, or any combination thereof.

According to one or more embodiments, at least one selected from among the first capping layer and the second capping layer may each independently include at least one selected from among Compounds CP1 to CP6, β-NPB, and/or any combination thereof:

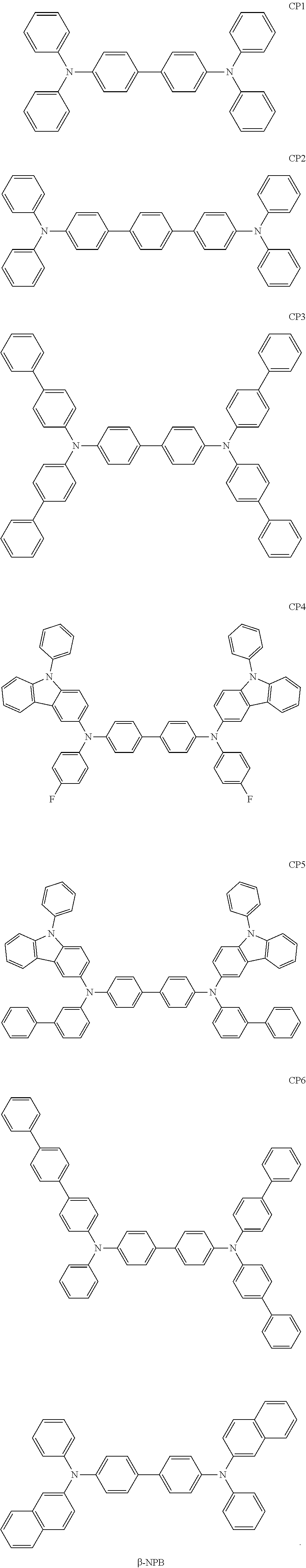

### Film

The amine-containing compound represented by Formula 1 may be included in one or more suitable films. Accordingly, one or more aspects of embodiments of the present disclosure are directed toward a film including the amine-containing compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, and/or like), a light-blocking member (for example, a light reflective layer, a light absorbing layer, and/or the like), and/or a protective member (for example, an insulating layer, a dielectric layer, and/or the like).

### Electronic Device

The light-emitting device may be included in one or more suitable electronic devices. For example, in one or more embodiments, the electronic device including the light-emitting device may be a light-emitting apparatus, an authentication apparatus, and/or the like.

In one or more embodiments, the electronic device (for example, a light-emitting apparatus) may further include, in addition to the light-emitting device, i) a color filter, ii) a color conversion layer, or iii) a color filter and a color conversion layer. The color filter and/or the color conversion layer may be located in at least one direction in which light emitted from the light-emitting device travels. For example, in some embodiments, the light emitted from the light-emitting device may be blue light or white light (e.g., combined white light). For details on the light-emitting device, related description provided above may be referred to. In one or more embodiments, the color conversion layer may include a quantum dot. The quantum dot may be, for example, a quantum dot as described herein.

The electronic device may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or a plurality of color conversion areas) may include a first area emitting first-color light, a second area emitting second-color light, and/or a third area emitting third-color light. The first-color light, the second-color light, and/or the third-color light may have different maximum luminescence wavelengths from one another. For example, in some embodiments, the first color light may be red light, the second color light may be green light, and the third color light may be blue light. For example, in one or more embodiments, the plurality of color filter areas (or the plurality of color conversion areas) may include quantum dots. In some embodiments, the first area may include a red quantum dot to emit red light, the second area may include a green quantum dot to emit green light, and the third area may not include (e.g., may exclude) a quantum dot. For details on the quantum dot, related descriptions provided herein may be referred to. The first area, the second area, and/or the third area may each include a scatter.

For example, in one or more embodiments, the light-emitting device may be to emit first light, the first area may be to absorb the first light to emit first-first color light, the second area may be to absorb the first light to emit second-first color light, and the third area may be to absorb the first light to emit third-first color light. The first-first color light, the second-first color light, and the third-first color light may have different maximum emission wavelengths from each other. In some embodiments, the first light may be blue light, the first-first color light may be red light, the second-first color light may be green light, and the third-first color light may be blue light.

In one or more embodiments, the electronic device may further include a thin-film transistor, in addition to the light-emitting device as described above. The thin-film transistor may include a source electrode, a drain electrode, and an activation layer, wherein one of the source electrode or the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic device may further include a sealing portion for sealing the light-emitting device. The sealing portion may be located between the color filter and/or the color conversion layer and the light-emitting device. The sealing portion allows light from the light-emitting device to be extracted to the outside, and concurrently (e.g., simultaneously) prevents or reduces ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic device may be flexible.

Various functional layers may be additionally located on the sealing portion, in addition to the color filter and/or the color conversion layer, according to the utilization of the electronic device. The functional layers may include a touch screen layer, a polarizing layer, and/or the like. The touch screen layer may be a pressure-sensitive touch screen layer, a capacitive touch screen layer, or an infrared touch screen layer.

The authentication apparatus may be, for example, a biometric authentication apparatus that authenticates an individual by utilizing biometric information of a living body (for example, fingertips, pupils, etc.).

The authentication apparatus may further include, in addition to the light-emitting device as described above, a biometric information collector.

The electronic device may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Apparatus

The light-emitting device may be included in one or more suitable electronic apparatuses.

For example, the electronic apparatus including the light-emitting device may be one selected from a flat panel display, a curved display, a computer monitor, a medical monitor, a TV, a billboard, indoor or outdoor illuminations and/or signal light, a head-up display, a fully or partially transparent display, a flexible display, a rollable display, a foldable display, a stretchable display, a laser printer, a phone, a cell phone, a tablet, a phablet, a personal digital assistant (PDA), a wearable device, laptop computers, digital cameras, camcorders, viewfinders, micro displays, 3D displays, virtual or augmented reality displays, vehicles, a video wall including multiple displays tiled together, a theater or stadium screen, a phototherapy device, and a signage.

As the light-emitting device has the characteristics of excellent or suitable light emission efficiency, long lifespan, etc., the electronic apparatus including the light-emitting device may have the characteristics of high luminance, high resolution, low power consumption, etc.

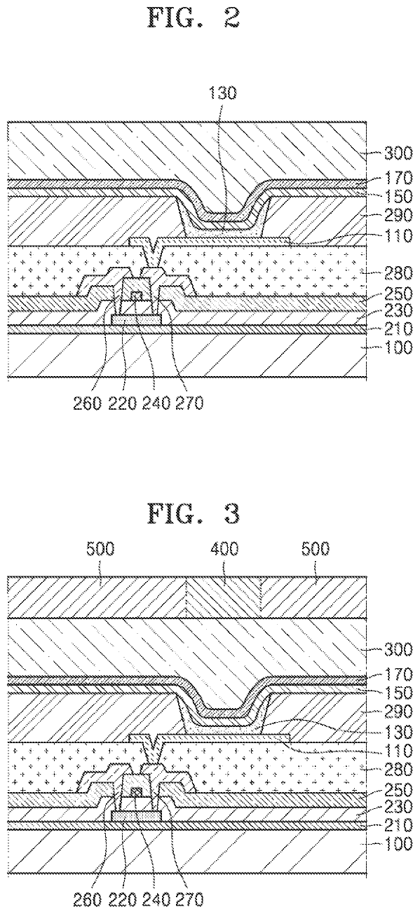

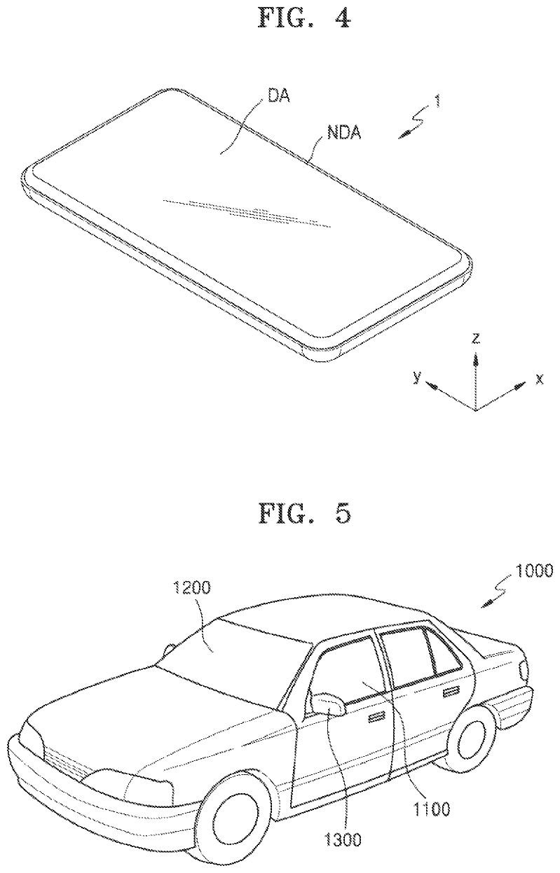

### Descriptions of FIGS. 2 and 3

FIG. 2 is a schematic diagram of an electronic device according to one or more embodiments of the present disclosure.

The electronic device of FIG. 2 (e.g., a light-emitting apparatus) may include a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located on the substrate 100. The buffer layer 210 may prevent or reduce infiltration of impurities through the substrate 100. The buffer layer 210 may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor. The activation layer 220 may include a source region, a drain region, and a channel region.

A gate insulating film 230 for insulating the activation layer 220 from the gate electrode 240 may be located on the activation layer 220, and the gate electrode 240 may be located on the gate insulating film 230.

An interlayer insulating film 250 may be located on the gate electrode 240. The interlayer insulating film 250 may be located between the gate electrode 240 and the source electrode 260 and between the gate electrode 240 and the drain electrode 270, to insulate from one another.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220, respectively.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include a first electrode 110, an interlayer 130, and a second electrode 150.

The first electrode 110 may be located on the passivation layer 280. The passivation layer 280 may not entirely cover the drain electrode 270. The passivation layer 280 may be arranged to expose a certain region of the drain electrode 270. The first electrode 110 may be arranged to be connected with the exposed drain electrode 270.

A pixel defining layer 290 including an insulating material may be located on the first electrode 110. The pixel defining layer 290 may expose a certain region of the first electrode 110. The interlayer 130 may be formed in the exposed region. The pixel defining layer 290 may be a polyimide or polyacrylic organic film. In some embodiments, at least some layers of the interlayer 130 may extend beyond the upper portion of the pixel defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 is a schematic diagram of an electronic device according to one or more embodiments of the present disclosure.

The electronic device of FIG. 3 (e.g., the light-emitting apparatus) may be substantially the same as the electronic device of FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic device of FIG. 3 may be a tandem light-emitting device.

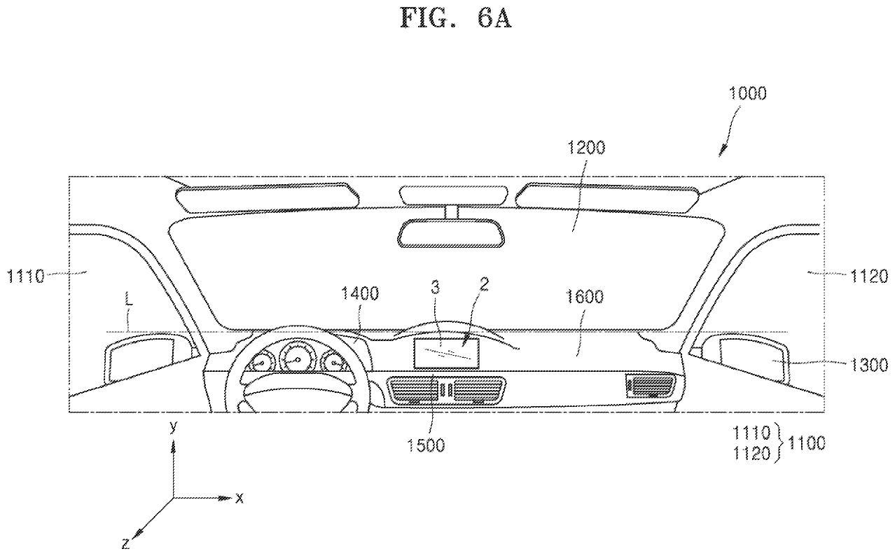

### Description of FIG. 4

FIG. 4 is a schematic diagram of an electronic apparatus 1 including a light-emitting device according to one or more embodiments of the present disclosure.

The electronic apparatus 1 may be an apparatus displaying a video or a still image and may include a portable electronic apparatus, such as a mobile phone, a smartphone, a tablet personal computer (PC), a mobile communication terminal, an electronic notebook, an electronic book, a portable multimedia player (PMP), a navigation, an ultra mobile PC, etc. and/or one or more suitable products including a television, a laptop, a monitor, a signboard, internet of things (IOT), or a part thereof. In some embodiments, the electronic apparatus 1 may be a wearable device such as a smart watch, a watch phone, a glasses-type or kind display, a head mounted display (HMD), or a part thereof. However, embodiments of the present disclosure are not limited thereto. For example, in some embodiments, the electronic apparatus 1 may be a dashboard of a vehicle, a center information display (CID) provided at a center fascia or a dashboard of a vehicle, a room mirror display functioning as a side mirror of a vehicle, a display provided at a backseat entertainment system or at the back of the front seat of a vehicle, a head up display (HUD) arranged at the front of a vehicle or projected on a front window of a vehicle, or a computer generated hologram augmented reality HUD (CGH AR HUD). FIG. 4 illustrates an embodiment where the electronic apparatus 1 is a smartphone for convenience in explanation.

The electronic apparatus 1 may include a display area DA and a non-display area NDA outside the display area DA. A display device of the electronic apparatus 1 may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices arranged on the display area DA may be arranged. On the non-display area NDA, a pad, which is an area to which an electronic element or a printing circuit board may be electrically connected, may be arranged.

In the electronic apparatus 1, a length in the x-axis direction and a length in the y-axis direction may be different from each other. For example, as shown in FIG. 4, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

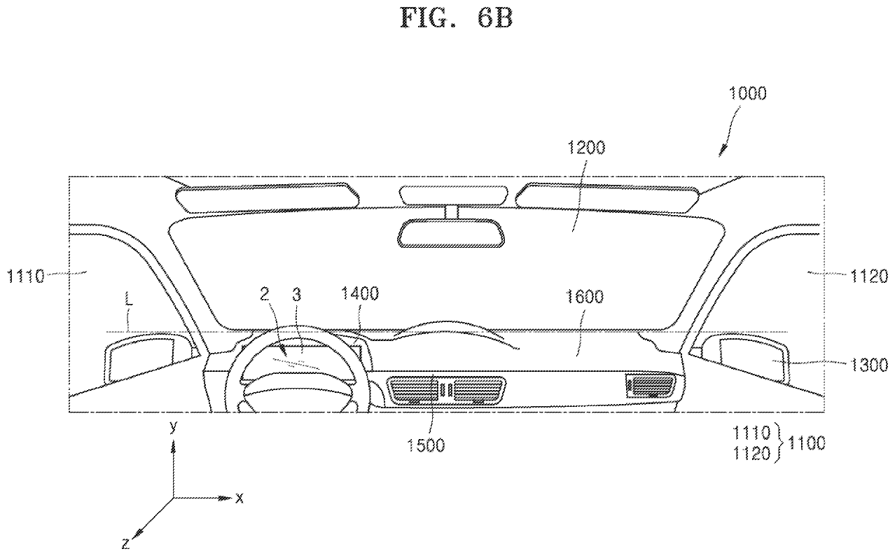

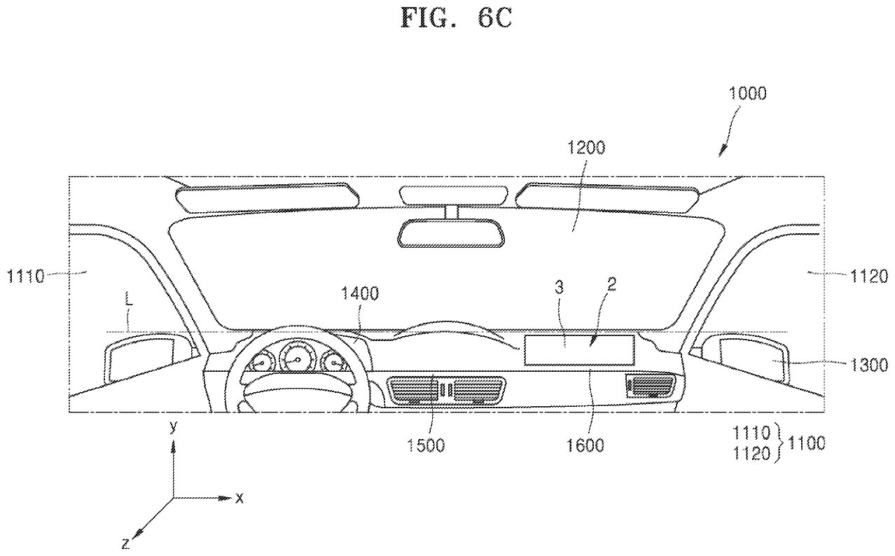

### Descriptions of FIGS. 5 and 6A to 6C

FIG. 5 is a schematic diagram of an exterior of a vehicle 1000 as an electronic apparatus including a light-emitting device according to one or more embodiments of the present disclosure. FIGS. 6A to 6C are schematic diagrams of an interior of the vehicle 1000 according to one or more suitable embodiments of the present disclosure.

Referring to FIGS. 5, 6A, 6B, and 6C, the vehicle 1000 may refer to one or more suitable apparatuses for moving an object to be transported, such as a human, an object, or an animal, from a departure point to a destination. The vehicle 1000 may include a vehicle traveling on a road or a track, a vessel moving over a sea or a river, an airplane flying in the sky utilizing the action of air, and/or the like.

In one or more embodiments, the vehicle 1000 may travel on a road or a track. The vehicle 1000 may move in a set or predetermined direction according to the rotation of at least one wheel. For example, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body. The exterior of the vehicle body may include a front panel, a bonnet, a roof panel, a rear panel, a trunk, a filler/pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a filler/pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced apart from each other in the x-direction or the −x-direction. For example, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, a hodometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and/or a heater of a seat are disposed. The center fascia 1500 may be arranged on one side of the cluster 1400.

A passenger seat dashboard 1600 may be spaced apart from the cluster 1400 with the center fascia 1500 arranged therebetween. In one or more embodiments, the cluster 1400 may be arranged to correspond to a driver seat, and the passenger seat dashboard 1600 may be disposed to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In one or more embodiments, the display device 2 may be arranged between the side window glasses 1100 facing each other. The display device 2 may be arranged on at least one selected from among the cluster 1400, the center fascia 1500, and the passenger seat dashboard 1600.

The display device 2 may include an organic light-emitting display device, an inorganic electroluminescent (EL) display device, a quantum dot display device, and/or the like. Hereinafter, as the display device 2 according to one or more embodiments of the present disclosure, an organic light-emitting display device including the light-emitting device according to the present disclosure will be described as an example, but one or more suitable types (kinds) of display devices as described above may be utilized in embodiments of the present disclosure.

Referring to FIG. 6A, the display device 2 may be arranged on the center fascia 1500. In one embodiment, the display device 2 may display navigation information. In one embodiment, the display device 2 may display audio, video, or information regarding vehicle settings.

Referring to FIG. 6B, the display device 2 may be arranged on the cluster 1400. When the display device 2 is arranged on the cluster 1400, the cluster 1400 may display driving information and/or the like through the display device 2. For example, in some embodiments, the cluster 1400 may be implemented digitally. The digital cluster 1400 may display vehicle information and driving information as images. For example, a needle and a gauge of a tachometer and one or more suitable warning light icons may be displayed by a digital signal.

Referring to FIG. 6C, the display device 2 may be arranged on the dashboard 1600 of the passenger seat. The display device 2 may be embedded in the passenger seat dashboard 1600 or arranged on the passenger seat dashboard 1600. In one or more embodiments, the display device 2 arranged on the dashboard 1600 of the passenger seat may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in a certain region by utilizing one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### DEFINITION OF TERMS

The term “C₃-C₆₀ carbocyclic group” as utilized herein refers to a cyclic group that includes only carbon as a ring-forming atom and includes (e.g., consists of) 3 to 60 carbon atoms. The term “C₁-C₆₀ carbocyclic group” as utilized herein refers to a cyclic group that includes hetero atoms as a ring-forming atom, in addition to carbon, and includes (e.g., consists of) 1 to 60 carbon atoms. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The “cyclic group” as utilized herein may include the C₃-C₆₀ carbocyclic group, and the C₁-C₆₀ heterocyclic group.

The term “π electron-rich C₃-C₆₀ cyclic group” utilized herein refers to a cyclic group that does not include *—N═*′ as a ring-forming moiety and includes (e.g., consists of) 3 to 60 carbon atoms. The term “π electron-deficient nitrogen-containing C₁-C₆₀ cyclic group” utilized herein refers to a heterocyclic group that includes *—N═*′ as a ring-forming moiety and includes (e.g., consists of) 1 to 60 carbon atoms.

For example, the C₃-C₆₀ carbocyclic group may be i) a T1 group or ii) a condensed cyclic group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group).

The C₁-C₆₀ heterocyclic group may be i) a T2 group, ii) a condensed cyclic group in which at least two T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like.)

The π electron-rich C₃-C₆₀ cyclic group may be i) a T1 group, ii) a condensed cyclic group in which at least two T1 groups are condensed with each other, iii) a T3 group, iv) a condensed cyclic group in which at least two T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like.)

The π electron-deficient nitrogen-containing C₁-C₆₀ cyclic group may be i) a T4 group, ii) a condensed cyclic group in which at least two T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like.)

The T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group.

The T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The T3 group may be a furan group, a thiophene group, a 1H-pyrrole group, a silole group, or a borole group.

The T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as utilized herein refer to a group condensed to any cyclic group, a monovalent group, or a polyvalent group (for example, a divalent group, a trivalent group, a tetravalent group, and/or the like) according to the structure of a formula for which the corresponding term is utilized. In one or more embodiments, “a benzene group” may be a benzo group, a phenyl group, a phenylene group, and/or the like, which may be easily understood by one of ordinary skill in the art according to the structure of a formula including the “benzene group.”

Non-limiting examples of a monovalent C₃-C₆₀ carbocyclic group and a monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Non-limiting examples of a divalent C₃-C₆₀ carbocyclic group and a monovalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as utilized herein may refer to a linear or branched aliphatic hydrocarbon monovalent group with 1 to 60 carbon atoms. For example, a C₁-C₆₀ alkyl group may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

The term “C₁-C₆₀ alkylene group” as utilized herein refers to a divalent group having the same structure as the C₁-C₆₀ alkyl group.

The term “C₂-C₆₀ alkenyl group” as utilized herein may refer to a monovalent hydrocarbon group having at least one carbon-carbon double bond in the middle or at the terminus of the C₂-C₆₀ alkyl group. For example, a C₂-C₆₀ alkenyl group may include an ethenyl group, a propenyl group, a butenyl group, etc.

The term “C₂-C₆₀ alkenylene group” as utilized herein refers to a divalent group having the same structure as the C₂-C₆₀ alkenyl group.

The term “C₂-C₆₀ alkynyl group” as utilized herein may refer to a monovalent hydrocarbon group having at least one carbon-carbon triple bond in the middle or at the terminus of the C₂-C₆₀ alkyl group. For example, a C₂-C₆₀ alkynyl group may include an ethynyl group, a propynyl group, etc.

The term “C₂-C₆₀ alkynylene group” as utilized herein refers to a divalent group having the same structure as the C₂-C₆₀ alkynyl group.

The term “C₁-C₆₀ alkoxy group” as utilized herein may refer to a monovalent group having the formula of —OA₁₀₁ (where A₁₀₁ is the C₁-C₆₀ alkyl group). For example, a C₁-C₆₀ alkoxy group may include a methoxy group, an ethoxy group, an isopropyloxy group, etc.

The term “C₃-C₁₀ cycloalkyl group” as utilized herein refers to a monovalent saturated hydrocarbon cyclic group with 3 to 10 carbon atoms. The C₃-C₁₀ cycloalkyl group may include a cyclopropyl group, a cyclobutyl group, a cyclopentyl, cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and/or the like.

The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group that includes, in addition to carbon atoms, at least one hetero atom as a ring-forming atom, and includes (e.g., consists of) 1 to 10 carbon atoms. For example, the C₁-C₁₀ heterocycloalkyl group may include a 1,2,3,4-oxatriazolidinyl, a tetrahydrofuranyl group, a tetrahydrothiophenyl group, etc.

The term “C₁-C₁₀ heterocycloalkylene group” as utilized herein refers to a divalent group having the same structure as the C₁-C₁₀ heterocycloalkyl group.

The term “C₃-C₁₀ cycloalkenyl group” as utilized herein refers to a monovalent cyclic group that has 3 to 10 carbon atoms and at least one carbon-carbon double bond in its ring, but no aromaticity. For example, the C₃-C₁₀ cycloalkenyl group may include a cyclopentenyl group, a cyclohexenyl group, a cycloheptenyl group, etc.

The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. For example, the C₁-C₁₀ heterocycloalkenyl group may include a 4,5-dihydro-1,2,3,4-oxatriazolyl group, a 2,3-dihydrofuranyl group, a 2,3-dihydrothiophenyl group, etc.

The term “C₁-C₁₀ heterocycloalkenylene group” as utilized herein refers to a divalent group having the same structure as the C₁-C₁₀ heterocycloalkenyl group.

The term “C₆-C₆₀ aryl group” utilized herein refers to a monovalent group having a carbocyclic aromatic system with 6 to 60 carbon atoms. For example, the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, etc.

The term “C₆-C₆₀ arylene group” utilized herein refers to a divalent group having a carbocyclic aromatic system with 6 to 60 carbon atoms.

When the C₆-C₆₀ aryl group and the C₆-C₆₀ arylene group each include two or more rings, the rings may be condensed with each other.

The term “C₁-C₆₀ heteroaryl group” utilized herein may further include at least one hetero atom as a ring-forming atom, in addition to carbon atoms and refers to a monovalent group having a heterocyclic aromatic system with 1 to 60 carbon atoms. For example, the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, etc.

The term “C₁-C₆₀ heteroarylene group” utilized herein may further include at least one hetero atom as a ring-forming atom, in addition to carbon atoms and refers to a divalent group having a heterocyclic aromatic system with 1 to 60 carbon atoms.

When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure as a whole. For example, the monovalent non-aromatic condensed polycyclic group may include an indenyl group, a fluorenyl group, a spiro-bifluorenyl group, a benzofluorenyl group, an indenophenanthrenyl group, an indenoanthracenyl group, etc.

The term “divalent non-aromatic condensed polycyclic group” as utilized herein refers to a divalent group having the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure as a whole. For example, the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and/or the like.

The term “divalent non-aromatic condensed heteropolycyclic group” as utilized herein refers to a divalent group having the same structure as the monovalent non-aromatic condensed heteropolycyclic group described above.

The term “C₆-C₆₀ aryloxy group” utilized herein refers to —OA₁₀₂ (here, A₁₀₂ is the C₆-C₆₀ aryl group.)

The term “C₆-C₆₀ arylthio group” utilized herein refers to —SA₁₀₃ (here, A₁₀₃ is the C₆-C₆₀ aryl group.)

The term “C₇-C₆₀ arylalkyl group” utilized herein refers to -A₁₀₄A₁₀₅ (here, A₁₀₄ is a C₁-C₅₄ alkylene group, and A₁₀₅ is a C₆-C₅₉ aryl group.)

The term “C₂-C₆₀ heteroarylalkyl group” utilized herein refers to -A₁₀₆A₁₀₇ (here, A₁₀₆ is a C₁-C₆₀ alkylene group, and A₁₀₇ is a C₁-C₆₀ heteroaryl group.)

The term “R₁₀ₐ” as utilized herein refers to:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ utilized herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom” as utilized herein refers to any atom other than a carbon atom. Non-limiting examples of the heteroatom may be O, S, N, P, Si, B, Ge, Se, and any combinations thereof.

The term “third-row transition metal” utilized herein includes hafnium (Hf), tantalum (Ta), tungsten (W), rhenium (Re), osmium (Os), iridium (Ir), platinum (Pt), gold (Au), and/or the like.

The term “Ph” as utilized herein refers to a phenyl group, the term “Me” as utilized herein refers to a methyl group, the term “Et” as utilized herein refers to an ethyl group, the term “tert-Bu” or “But” as utilized herein refers to a tert-butyl group, and the term “OMe” as utilized herein refers to a methoxy group.

The term “biphenyl group” as utilized herein refers to “a phenyl group substituted with a phenyl group.” In other words, the “biphenyl group” is a substituted phenyl group having a C₆-C₆₀ aryl group as a substituent.

The term “terphenyl group” as utilized herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as utilized herein, unless defined otherwise, each refer to a binding site to a neighboring atom in a corresponding formula or moiety.

In the present disclosure, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in more detail with reference to the following synthesis examples and examples. The wording “B was utilized instead of A” utilized in describing Synthesis Examples refers to that an identical molar equivalent of B was utilized in place of A.

### SYNTHESIS Examples 1 to 10 (Synthesis of Compounds 3, 16, 34, 60, 83, 111, 130, 163, 183, and 227)

### Synthesis Example 1: Synthesis of Compound 3

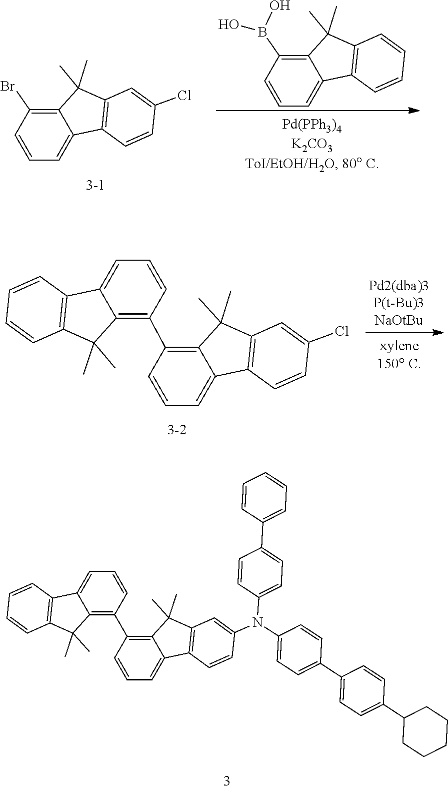 [SMILES: CC1(C)c2cc(Cl)ccc2-c2cccc(Br)c21 ; CC1(C)c2ccccc2-c2cccc(B(O)O)c21 ; CC1(C)c2ccccc2-c2cccc(-c3cccc4c3C(C)(C)c3cc(Cl)ccc3-4)c21 ; CC1(C)c2ccccc2-c2cccc(-c3cccc4c3C(C)(C)c3cc(N(c5ccc(-c6ccccc6)cc5)c5ccc(-c6ccc(C7CCCCC7)cc6)cc5)ccc3-4)c21]

### Synthesis of Intermediate 3-2

Intermediate 3-1 (3.07 g), Pd(PPh₃)₄ (0.56 g), K₂CO₃ (3.45 g), and (9,9-dimethyl-9H-fluoren-1-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 3-2 (2.56 g, yield: 61%).

### Synthesis of Compound 3

Intermediate 3-2 (4.20 g), N-([1,1′-biphenyl]-4-yl)-4′-cyclohexyl-[1,1′-biphenyl]-4-amine (4.03 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 3 (5.75 g, yield: 73%).

### Synthesis Example 2: Synthesis of Compound 16

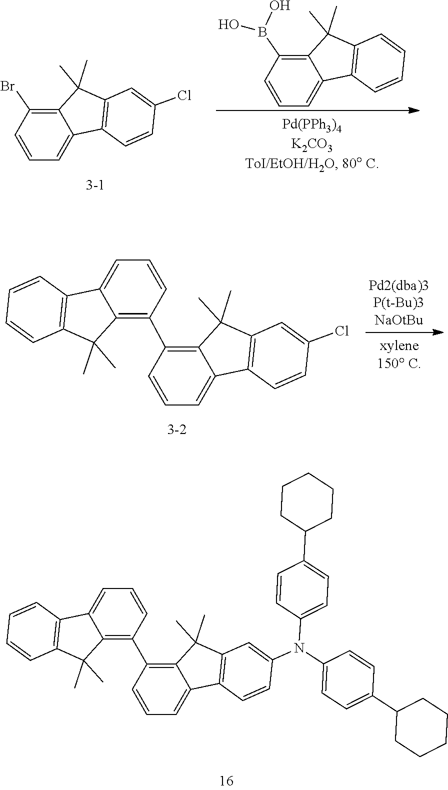 [SMILES: CC1(C)c2cc(Cl)ccc2-c2cccc(Br)c21 ; CC1(C)c2ccccc2-c2cccc(B(O)O)c21 ; CC1(C)c2ccccc2-c2cccc(-c3cccc4c3C(C)(C)c3cc(Cl)ccc3-4)c21 ; CC1(C)c2ccccc2-c2cccc(-c3cccc4c3C(C)(C)c3cc(N(c5ccc(C6CCCCC6)cc5)c5ccc(C6CCCCC6)cc5)ccc3-4)c21]

### Synthesis of Compound 16

Intermediate 3-2 (4.20 g), bis(4-cyclohexylphenyl)amine (3.33 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 16 (4.95 g, yield: 69%).

### Synthesis Example 3: Synthesis of Compound 34

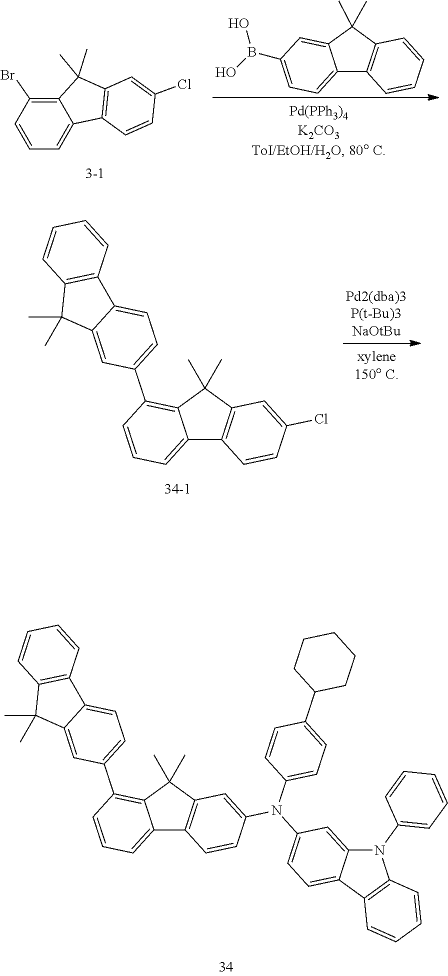 [SMILES: CC1(C)c2cc(Cl)ccc2-c2cccc(Br)c21 ; CC1(C)c2ccccc2-c2ccc(B(O)O)cc21 ; CC1(C)c2ccccc2-c2ccc(-c3cccc4c3C(C)(C)c3cc(Cl)ccc3-4)cc21 ; CC1(C)c2ccccc2-c2ccc(-c3cccc4c3C(C)(C)c3cc(N(c5ccc(C6CCCCC6)cc5)c5ccc6c7ccccc7n(-c7ccccc7)c6c5)ccc3-4)cc21]

### Synthesis of Intermediate 34-1

Intermediate 3-1 (3.07 g), Pd(PPh₃)₄ (0.56 g), K₂CO₃ (3.45 g), and (9,9-dimethyl-9H-fluoren-2-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 34-1 (2.52 g, yield: 60%).

### Synthesis of Compound 34

Intermediate 34-1 (4.20 g), N-(4-cyclohexylphenyl)-9-phenyl-9H-carbazol-2-amine (4.16 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 34 (5.84 g, yield: 73%).

### Synthesis Example 4: Synthesis of Compound 60

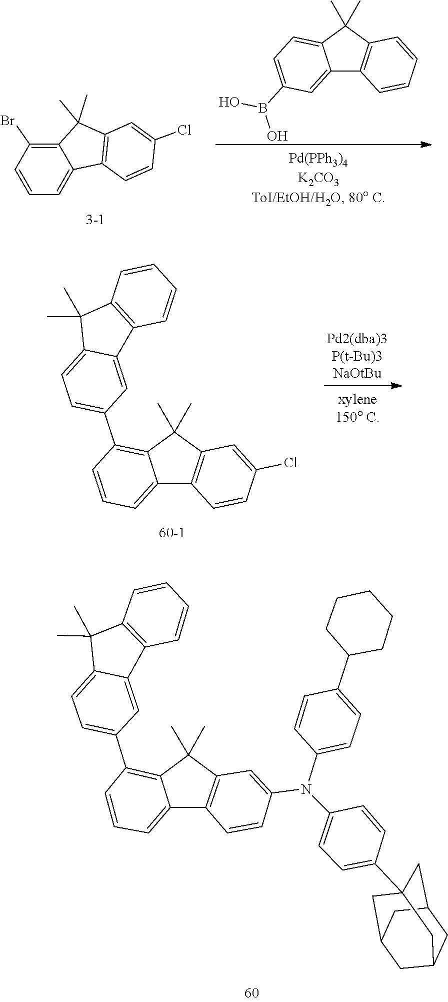 [SMILES: CC1(C)c2cc(Cl)ccc2-c2cccc(Br)c21 ; CC1(C)c2ccccc2-c2cc(B(O)O)ccc21 ; CC1(C)c2ccccc2-c2cc(-c3cccc4c3C(C)(C)c3cc(Cl)ccc3-4)ccc21 ; CC1(C)c2ccccc2-c2cc(-c3cccc4c3C(C)(C)c3cc(N(c5ccc(C6CCCCC6)cc5)c5ccc(C67CC8CC(CC(C8)C6)C7)cc5)ccc3-4)ccc21]

### Synthesis of Intermediate 60-1

Intermediate 3-1 (3.07 g), Pd(PPh₃)₄ (0.56 g), K₂CO₃ (3.45 g), and (9,9-dimethyl-9H-fluoren-3-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 60-1 (2.43 g, yield: 58%).

### Synthesis of Compound 60

Intermediate 60-1 (4.20 g), 4-((3R,5R)-adamantan-1-yl)-N-(4-cyclohexylphenyl)aniline (3.85 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 60 (5.77 g, yield: 75%).

### Synthesis Example 5: Synthesis of Compound 83

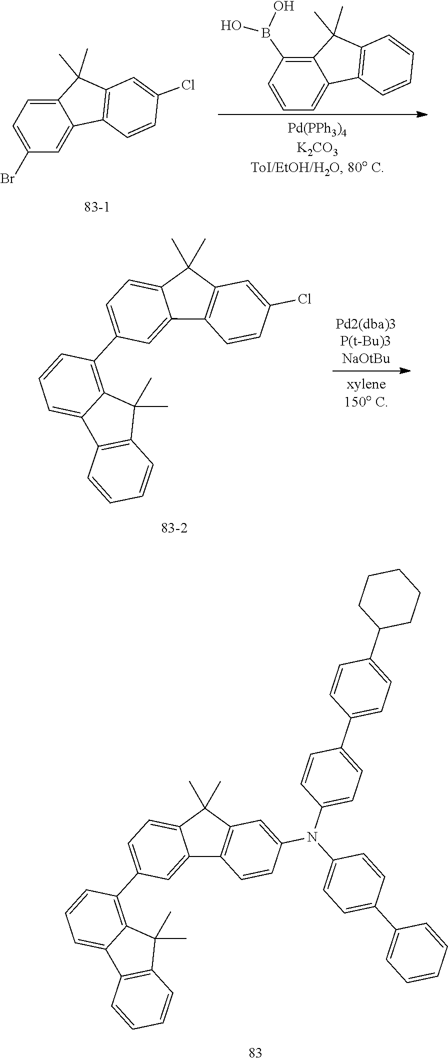 [SMILES: CC1(C)c2ccc(Br)cc2-c2ccc(Cl)cc21 ; CC1(C)c2ccccc2-c2cccc(B(O)O)c21 ; CC1(C)c2ccc(-c3cccc4c3C(C)(C)c3ccccc3-4)cc2-c2ccc(Cl)cc21 ; CC1(C)c2ccc(-c3cccc4c3C(C)(C)c3ccccc3-4)cc2-c2ccc(N(c3ccc(-c4ccccc4)cc3)c3ccc(-c4ccc(C5CCCCC5)cc4)cc3)cc21]

### Synthesis of Intermediate 83-2

Intermediate 83-1 (3.07 g), Pd(PPh₃)₄ (0.56 g), K₂CO₃ (3.45 g), and (9,9-dimethyl-9H-fluoren-1-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 83-2 (2.48 g, yield: 59%).

### Synthesis of Compound 83

Intermediate 83-2 (4.20 g), N-([1,1′-biphenyl]-4-yl)-4′-cyclohexyl-[1,1′-biphenyl]-4-amine (3.21 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 83 (5.36 g, yield: 76%).

### Synthesis Example 6: Synthesis of Compound 111

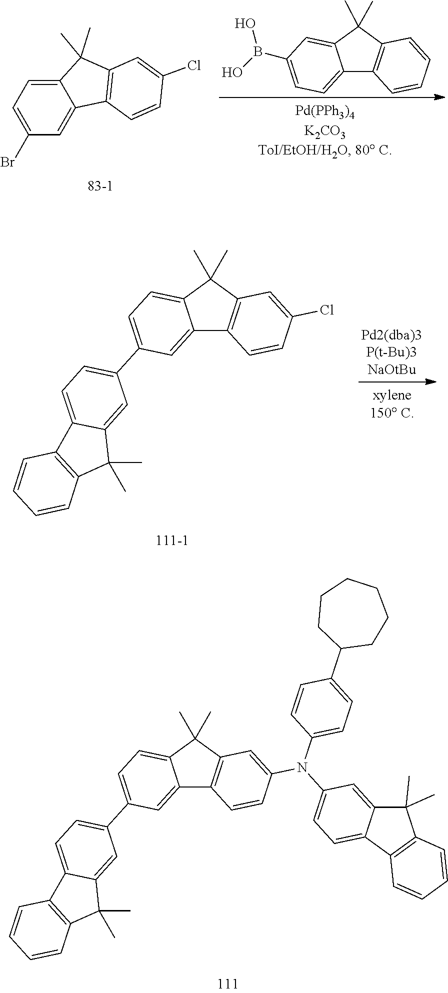 [SMILES: CC1(C)c2ccc(Br)cc2-c2ccc(Cl)cc21 ; CC1(C)c2ccccc2-c2ccc(B(O)O)cc21 ; CC1(C)c2ccc(-c3ccc4c(c3)C(C)(C)c3ccccc3-4)cc2-c2ccc(Cl)cc21 ; CC1(C)c2ccccc2-c2ccc(-c3ccc4c(c3)-c3ccc(N(c5ccc(C6CCCCCC6)cc5)c5ccc6c(c5)C(C)(C)c5ccccc5-6)cc3C4(C)C)cc21]

### Synthesis of Intermediate 111-1

Intermediate 83-1 (3.07 g), Pd(PPh₃)₄ (0.56 g), K₂CO₃ (3.45 g), and (9,9-dimethyl-9H-fluoren-2-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 111-1 (2.27 g, yield: 54%).

### Synthesis of Compound 111

Intermediate 111-1 (4.20 g), N-(4-cycloheptylphenyl)-9,9-dimethyl-9H-fluoren-2-amine (3.81 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 111 (6.12 g, yield: 80%).

### Synthesis Example 7: Synthesis of Compound 130

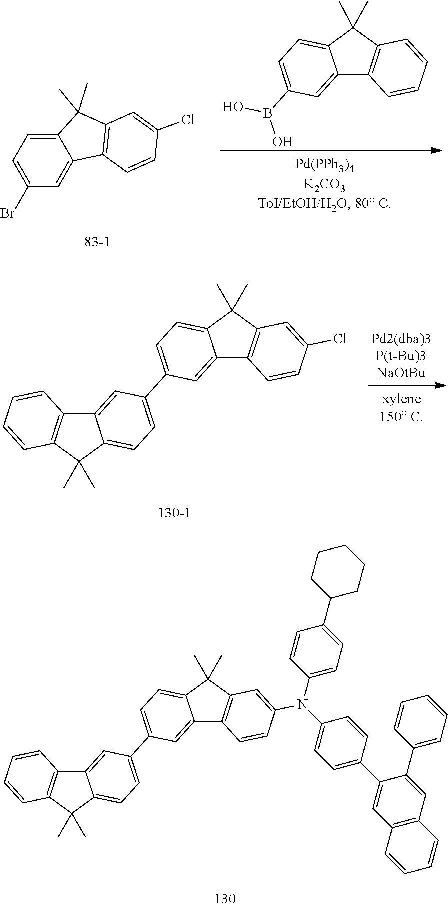 [SMILES: CC1(C)c2ccc(Br)cc2-c2ccc(Cl)cc21 ; CC1(C)c2ccccc2-c2cc(B(O)O)ccc21 ; CC1(C)c2ccccc2-c2cc(-c3ccc4c(c3)-c3ccc(Cl)cc3C4(C)C)ccc21 ; CC1(C)c2ccccc2-c2cc(-c3ccc4c(c3)-c3ccc(N(c5ccc(-c6cc7ccccc7cc6-c6ccccc6)cc5)c5ccc(C6CCCCC6)cc5)cc3C4(C)C)ccc21]

### Synthesis of Intermediate 130-1

Intermediate 83-1 (3.07 g), Pd(PPh₃)₄ (0.56 g), K₂CO₃ (3.45 g), and (9,9-dimethyl-9H-fluoren-3-yl)boronic acid (2.38 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 130-1 (2.35 g, yield: 56%).

### Synthesis of Compound 130

Intermediate 130-1 (4.20 g), 4-cyclohexyl-N-(4-(3-phenylnaphthalen-2-yl)phenyl)aniline (4.53 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 130 (6.36 g, yield: 71%).

### Synthesis Example 8: Synthesis of Compound 163

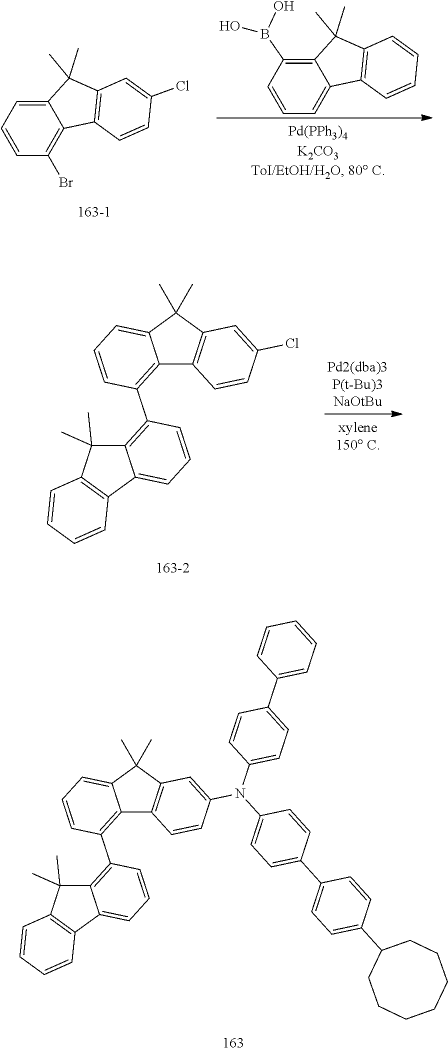 [SMILES: CC1(C)c2cc(Cl)ccc2-c2c(Br)cccc21 ; CC1(C)c2ccccc2-c2cccc(B(O)O)c21 ; CC1(C)c2cc(Cl)ccc2-c2c(-c3cccc4c3C(C)(C)c3ccccc3-4)cccc21 ; CC1(C)c2cc(N(c3ccc(-c4ccccc4)cc3)c3ccc(-c4ccc(C5CCCCCCC5)cc4)cc3)ccc2-c2c(-c3cccc4c3C(C)(C)c3ccccc3-4)cccc21]

### Synthesis of Intermediate 163-2

Intermediate 163-1 (3.07 g), Pd(PPh₃)₄ (0.56 g), K₂CO₃ (3.45 g), and (9,9-dimethyl-9H-fluoren-1-yl)boronic acid (4.53 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 163-2 (2.77 g, yield: 66%).

### Synthesis of Compound 163

Intermediate 163-2 (4.20 g), N-([1,1′-biphenyl]-4-yl)-4′-cyclooctyl-[1,1′-biphenyl]-4-amine (4.31 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 163 (6.12 g, yield: 75%).

### Synthesis Example 9: Synthesis of Compound 183

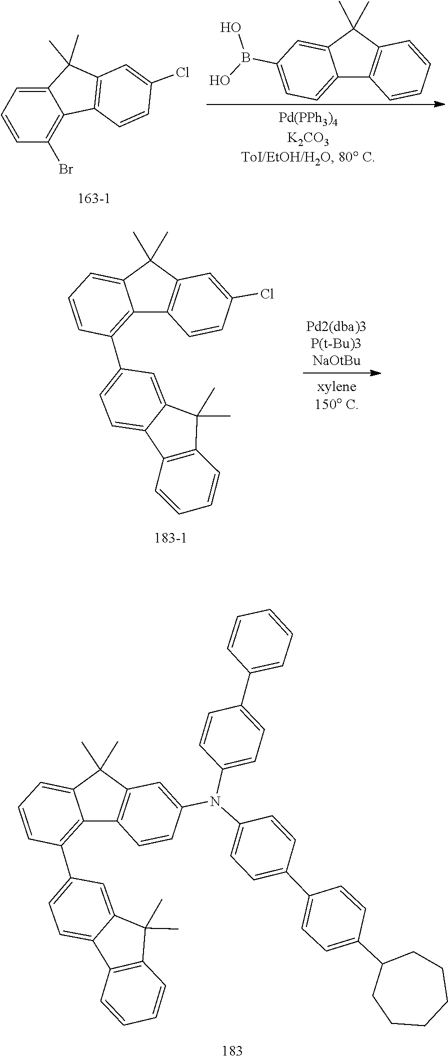

### Synthesis of Intermediate 183-1

Intermediate 163-1 (3.07 g), Pd(PPh₃)₄ (0.56 g), K₂CO₃ (3.45 g), and (9,9-dimethyl-9H-fluoren-2-yl)boronic acid (4.53 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 183-1 (2.77 g, yield: 66%).

### Synthesis of Compound 183

Intermediate 183-1 (4.20 g), N-([1,1′-biphenyl]-4-yl)-4′-cycloheptyl-[1,1′-biphenyl]-4-amine (4.17 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 183 (5.93 g, yield: 74%).

### Synthesis Example 10: Synthesis of Compound 227

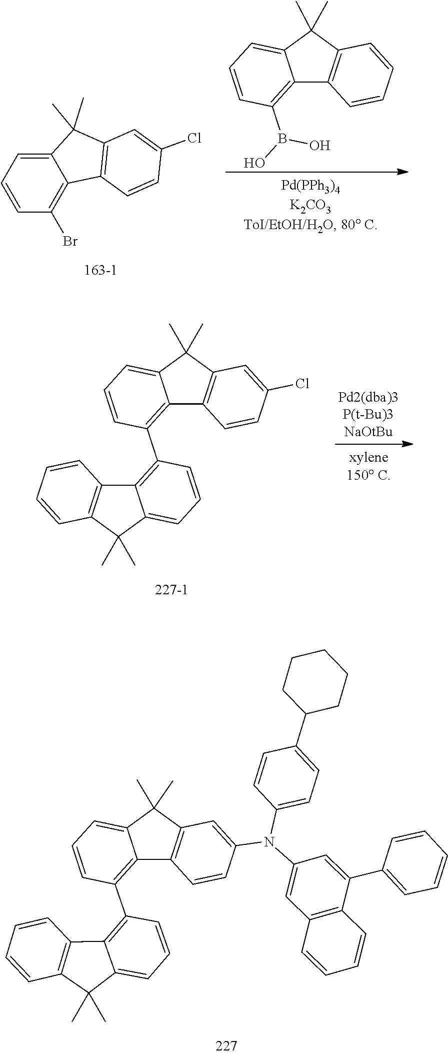 [SMILES: CC1(C)c2cc(Cl)ccc2-c2c(Br)cccc21 ; CC1(C)c2ccccc2-c2c(B(O)O)cccc21 ; CC1(C)c2ccccc2-c2c(-c3cccc4c3-c3ccc(Cl)cc3C4(C)C)cccc21 ; CC1(C)c2ccccc2-c2c(-c3cccc4c3-c3ccc(N(c5ccc(C6CCCCC6)cc5)c5cc(-c6ccccc6)c6ccccc6c5)cc3C4(C)C)cccc21]

### Synthesis of Intermediate 227-1

Intermediate 163-1 (3.07 g), Pd(PPh₃)₄ (0.56 g), K₂CO₃ (3.45 g), and (9,9-dimethyl-9H-fluoren-4-yl)boronic acid (4.53 g) were dissolved in Toluene/EtOH/H₂O (100 mL/25 mL/25 mL) and stirred at 80° C. for 12 hours. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 227-1 (2.94 g, yield: 70%).

### Synthesis of Compound 227

Intermediate 227-1 (4.20 g), N-(4-cyclohexylphenyl)-4-phenylnaphthalen-2-amine (3.77 g), Pd₂(dba)₃ (0.46 g), P(t-Bu)₃ (0.21 g), and NaOtBu (2.44 g) were dissolved in o-xylene (50 mL) and stirred at 150° C. for 1 hour. The reaction temperature was lowered to room temperature, and the reaction was terminated by utilizing water. Then, an extraction process was performed thereon three times by utilizing ethylether. An organic layer extracted therefrom was dried by utilizing anhydrous magnesium sulfate and distilled under reduced pressure, and a residue thus obtained was separated and purified by column chromatography, thereby obtaining Compound 227 (5.56 g, yield: 73%).

### Comparative Example 1

As an anode, a glass substrate with 15 Ω/cm 2 (1,200 Å) ITO thereon, which was manufactured by Corning Inc., was cut to a size of 50 mm×50 mm×0.7 mm, and the glass substrate was sonicated in isopropyl alcohol and pure water for 5 minutes each, and then ultraviolet (UV) light was irradiated for 30 minutes thereto, and ozone was exposed thereto for cleaning. Then, the resultant ITO glass substrate was loaded onto a vacuum deposition apparatus.

On the glass substrate, first, 2-TNATA which is a suitable material in the art was vacuum-deposited to form a hole injection layer having a thickness of 600 Å, and 4,4′-bis[N-(1-naphthyl)-N-phenylamino]piphenyl (hereinafter, referred to as NPB) which is a hole transport material was vacuum-deposited as a hole transport compound to form a hole transport layer having a thickness of 300 Å.

9,10-di(naphthalen-2-yl)anthracene (hereinafter, referred to as DNA) which is a suitable blue fluorescence host in the art and 4,4′-bis[2-(4-(N,N-diphenylamino)phenyl)vinyl]biphenyl (hereinafter, referred to as DPAVBi) which is a suitable blue phosphorescence dopant compound in the art were co-deposited on the hole transport layer at a weight ratio of 98:2 to form an emission layer having a thickness of 300 Å.

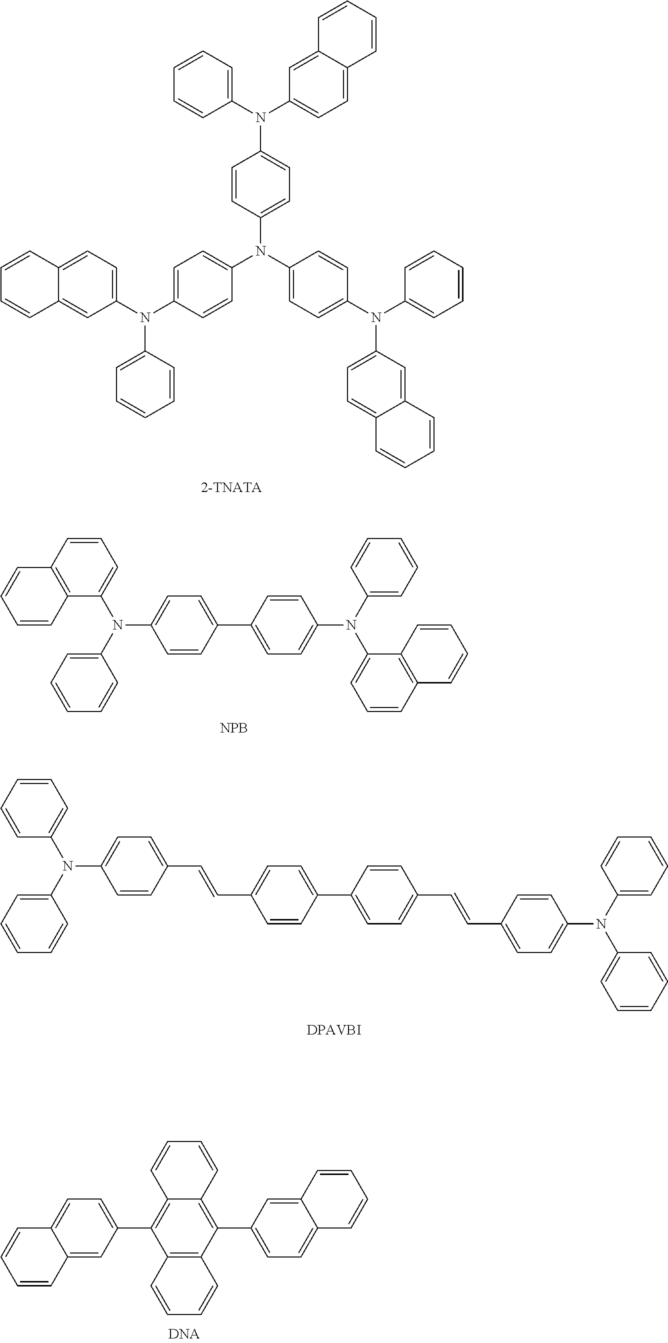

Then, Alq3 was deposited on the emission layer to form an electron transport layer having a thickness of 300 Å, and then, LiF, which is a halogenated alkali metal, was deposited on the electron transport layer to form an electron injection layer having a thickness of 10 Å and Al was vacuum-deposited thereon to form an Al electrode having a thickness of 3,000 Å (cathode) to form an LiF/AI electrode, thereby completing the manufacture of an organic electroluminescent device.

### Comparative Examples 2 to 5 and Examples 1 to 10

Organic light-emitting devices were manufactured in substantially the same manner as in Comparative Example 1, except that, in forming a hole transport layer, corresponding compounds shown in Table 1 were utilized instead of NPB.

### Evaluation Example 1

To evaluate characteristics of each of the organic light-emitting devices manufactured according to Comparative Examples 1 to 5 and Examples 1 to 10, driving voltage, luminance, luminescence efficiency, and lifespan were measured, and results thereof are shown in Table 1.

The driving voltage at the current density of 50 mA/cm 2 was measured utilizing a source meter (2400 series, Keithley Instrument Inc.)

Power was supplied from a current-voltage source-measure unit (Keithley SMU 236), and the luminance and the luminescence efficiency were measured by utilizing a luminance meter PR650.

TABLE 1

| | | | | | | | Half |
|---|---|---|---|---|---|---|---|
| | Hole | Driving | Current | | | | lifespan |
| | transport | voltage | density | Luminance | Efficiency | Emission | (hr @100 |
| | material | (V) | (mA/cm²) | (cd/m²) | (cd/A) | color | mA/cm²) |
| Example 1 | Compound 3 | 4.40 | 50 | 4055 | 8.11 | Blue | 463 |
| Example 2 | Compound 16 | 4.48 | 50 | 4255 | 8.51 | Blue | 468 |
| Example 3 | Compound 34 | 4.32 | 50 | 4240 | 8.48 | Blue | 417 |
| Example 4 | Compound 60 | 4.48 | 50 | 4060 | 8.12 | Blue | 474 |
| Example 5 | Compound 83 | 4.69 | 50 | 4280 | 8.56 | Blue | 497 |
| Example 6 | Compound 111 | 4.67 | 50 | 4370 | 8.74 | Blue | 445 |
| Example 7 | Compound 130 | 4.78 | 50 | 4230 | 8.46 | Blue | 443 |
| Example 8 | Compound 163 | 4.49 | 50 | 4050 | 8.10 | Blue | 483 |
| Example 9 | Compound 183 | 4.60 | 50 | 4170 | 8.34 | Blue | 442 |
| Example 10 | Compound 227 | 4.30 | 50 | 4325 | 8.65 | Blue | 455 |
| Comparative | NPB | 7.01 | 50 | 2645 | 5.29 | Blue | 258 |
| Example 1 | | | | | | | |
| Comparative | A | 5.43 | 50 | 3780 | 7.56 | Blue | 315 |
| Example 2 | | | | | | | |
| Comparative | B | 6.41 | 50 | 3385 | 6.77 | Blue | 294 |
| Example 3 | | | | | | | |
| Comparative | C | 5.21 | 50 | 3730 | 7.46 | Blue | 346 |
| Example 4 | | | | | | | |
| Comparative | D | 4.98 | 50 | 3915 | 7.83 | Blue | 371 |
| Example 5 | | | | | | | |

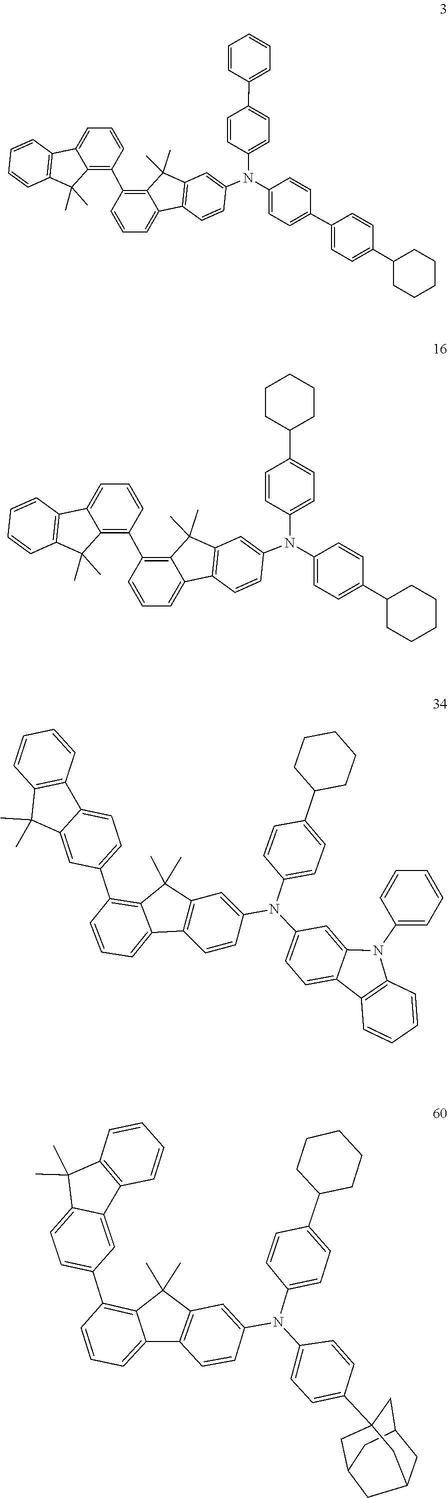 [SMILES: CC1(C)c2ccccc2-c2cccc(-c3cccc4c3C(C)(C)c3cc(N(c5ccc(-c6ccccc6)cc5)c5ccc(-c6ccc(C7CCCCC7)cc6)cc5)ccc3-4)c21 ; CC1(C)c2ccccc2-c2cccc(-c3cccc4c3C(C)(C)c3cc(N(c5ccc(C6CCCCC6)cc5)c5ccc(C6CCCCC6)cc5)ccc3-4)c21 ; CC1(C)c2ccccc2-c2ccc(-c3cccc4c3C(C)(C)c3cc(N(c5ccc(C6CCCCC6)cc5)c5ccc6c7ccccc7n(-c7ccccc7)c6c5)ccc3-4)cc21 ; CC1(C)c2ccccc2-c2cc(-c3cccc4c3C(C)(C)c3cc(N(c5ccc(C6CCCCC6)cc5)c5ccc(C67CC8CC(CC(C8)C6)C7)cc5)ccc3-4)ccc21]

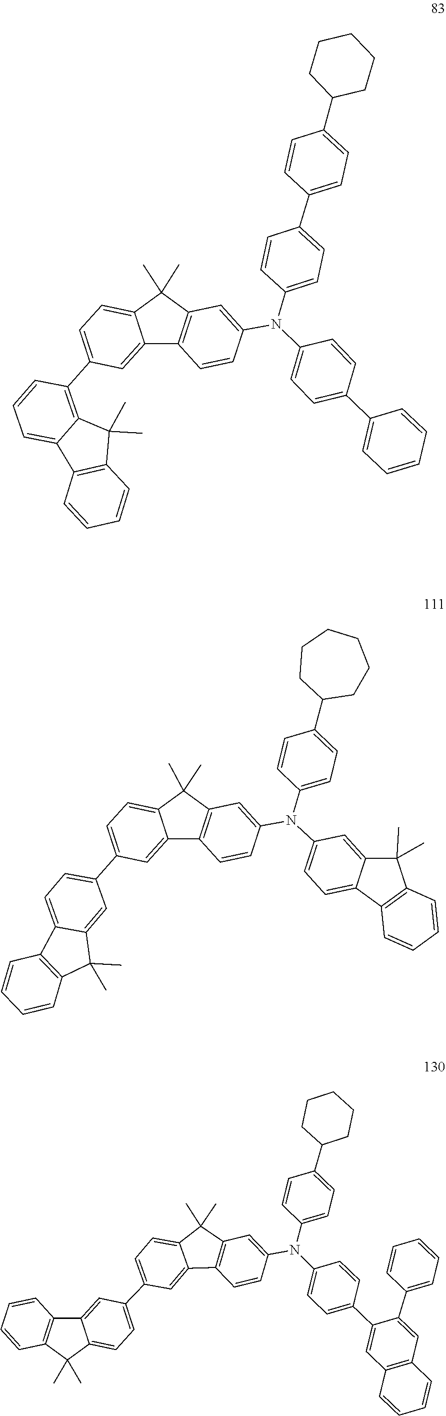 [SMILES: CC1(C)c2ccc(-c3cccc4c3C(C)(C)c3ccccc3-4)cc2-c2ccc(N(c3ccc(-c4ccccc4)cc3)c3ccc(-c4ccc(C5CCCCC5)cc4)cc3)cc21 ; CC1(C)c2ccccc2-c2ccc(-c3ccc4c(c3)-c3ccc(N(c5ccc(C6CCCCCC6)cc5)c5ccc6c(c5)C(C)(C)c5ccccc5-6)cc3C4(C)C)cc21 ; CC1(C)c2ccccc2-c2cc(-c3ccc4c(c3)-c3ccc(N(c5ccc(-c6cc7ccccc7cc6-c6ccccc6)cc5)c5ccc(C6CCCCC6)cc5)cc3C4(C)C)ccc21]

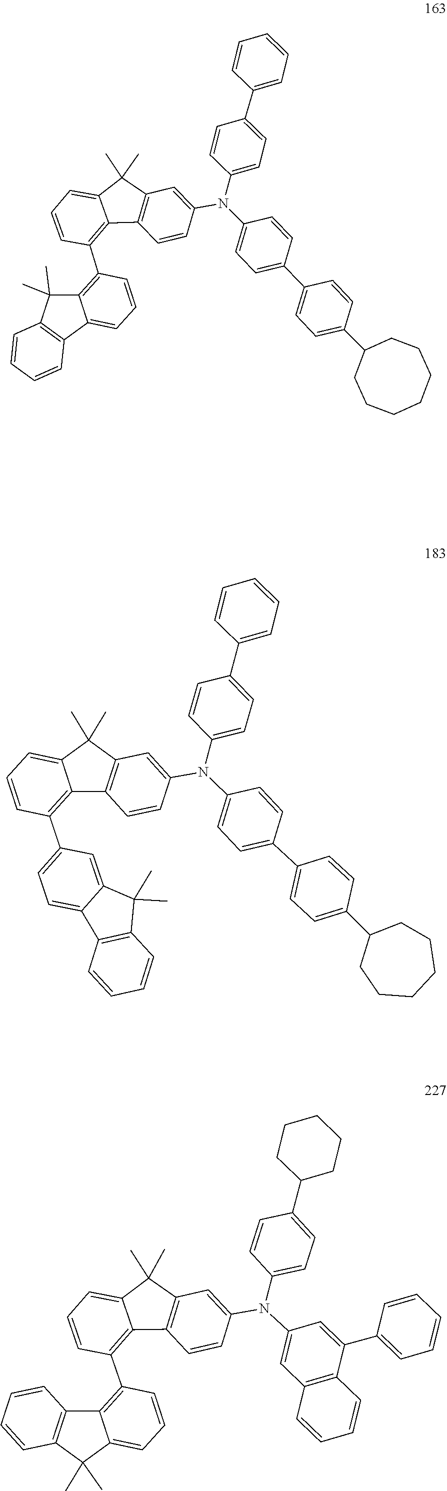 [SMILES: CC1(C)c2cc(N(c3ccc(-c4ccccc4)cc3)c3ccc(-c4ccc(C5CCCCCCC5)cc4)cc3)ccc2-c2c(-c3cccc4c3C(C)(C)c3ccccc3-4)cccc21 ; CC1(C)c2ccccc2-c2ccc(-c3cccc4c3-c3ccc(N(c5ccc(-c6ccccc6)cc5)c5ccc(-c6ccc(C7CCCCCC7)cc6)cc5)cc3C4(C)C)cc21 ; CC1(C)c2ccccc2-c2c(-c3cccc4c3-c3ccc(N(c5ccc(C6CCCCC6)cc5)c5cc(-c6ccccc6)c6ccccc6c5)cc3C4(C)C)cccc21]

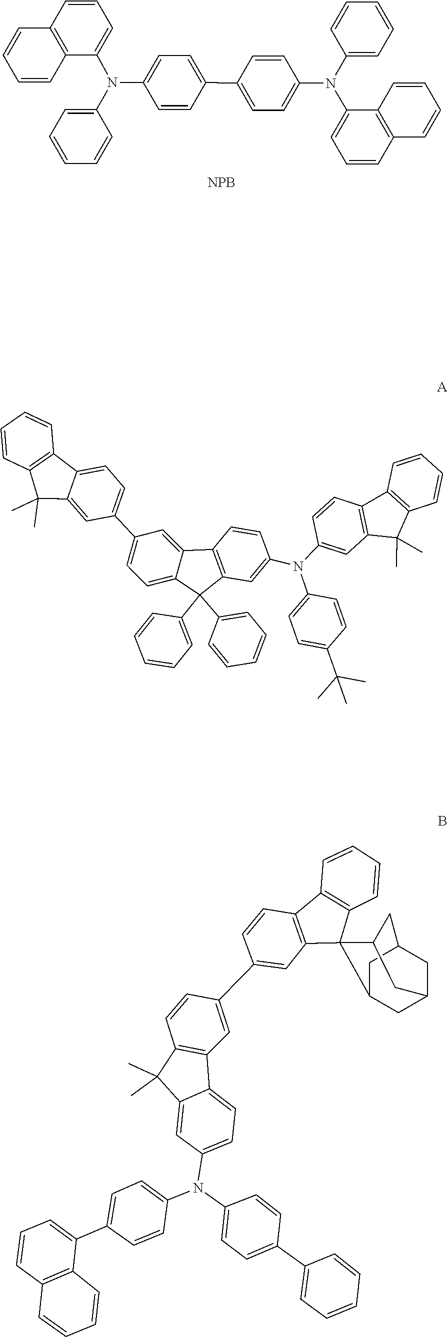 [SMILES: c1ccc(N(c2ccc(-c3ccc(N(c4ccccc4)c4cccc5ccccc45)cc3)cc2)c2cccc3ccccc23)cc1 ; "NPB" ; CC(C)(C)c1ccc(N(c2ccc3c(c2)C(C)(C)c2ccccc2-3)c2ccc3c(c2)C(c2ccccc2)(c2ccccc2)c2ccc(-c4ccc5c(c4)C(C)(C)c4ccccc4-5)cc2-3)cc1 ; CC1(C)c2ccc(-c3ccc4c(c3)C3(c5ccccc5-4)C4CC5CC(C4)CC3C5)cc2-c2ccc(N(c3ccc(-c4ccccc4)cc3)c3ccc(-c4cccc5ccccc45)cc3)cc21]

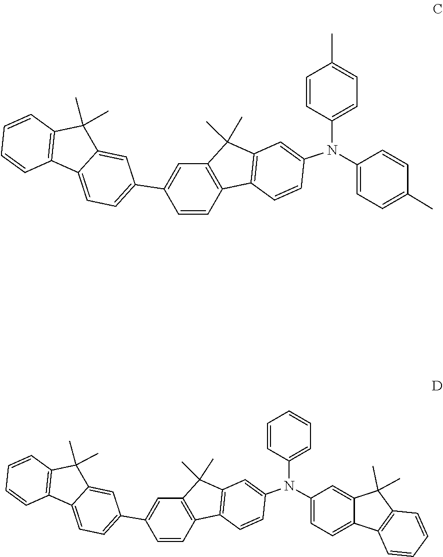 [SMILES: Cc1ccc(N(c2ccc(C)cc2)c2ccc3c(c2)C(C)(C)c2cc(-c4ccc5c(c4)C(C)(C)c4ccccc4-5)ccc2-3)cc1 ; CC1(C)c2ccccc2-c2ccc(-c3ccc4c(c3)C(C)(C)c3cc(N(c5ccccc5)c5ccc6c(c5)C(C)(C)c5ccccc5-6)ccc3-4)cc21]

From Table 1, it is confirmed that the organic light-emitting devices according to Examples 1 to 10 had a relatively lower driving voltage, a relatively higher luminance, relatively higher luminescence efficiency, and a relatively longer lifespan than the organic light-emitting devices according to Comparative Examples 1 to 5.

According to the one or more embodiments, an amine-containing compound represented by Formula 1 may have excellent or suitable hole transport characteristics. A light-emitting device including the amine-containing compound may have a low driving voltage, a high luminance, high luminescence efficiency, and a long lifespan. The display quality of an electronic device including the light-emitting device and an electronic apparatus utilizing the electronic device may be improved.

In the present disclosure, singular expressions may include plural expressions unless the context clearly indicates otherwise. It will be further understood that the terms “comprise,” “include,” or “have” when utilized in the present disclosure, specify the presence of stated features, integers, steps, operations, elements, and/or components, but do not preclude the presence or addition of one or more other features, integers, steps, operations, elements, components, and/or groups thereof. The “/” utilized below may be interpreted as “and” or as “or” depending on the situation.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” and “the” are intended to include the plural forms as well, unless the context clearly indicates otherwise. Further, the use of “may” when describing embodiments of the present disclosure refers to “one or more embodiments of the present disclosure”.

In the present disclosure, when particles are spherical, “diameter” indicates a particle diameter or an average particle diameter, and when the particles are non-spherical, the “diameter” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting device, the display device, the electronic apparatus, the electronic device, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode;
an interlayer between the first electrode and the second electrode and comprising an emission layer; and
an amine-containing compound represented by Formula 1: 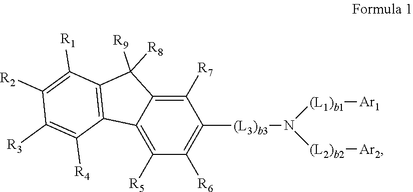
wherein, in Formula 1,
R₁ to R₉ are each independently a group represented by Formula 2,
hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one selected from among R₁ to R₄ is the group represented by Formula 2,
R₈ and R₉ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
at least one selected from among Ar₁ and Ar₂ is a saturated C₃-C₆₀ cyclic group unsubstituted or substituted with at least one R₁₀ₐ,
L₁ to L₃ are each independently a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b1 to b3 are each independently an integer from 0 to 5, 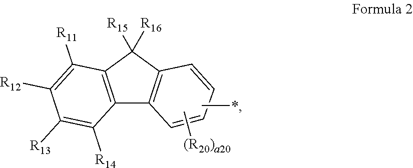
wherein, in Formula 2,
R₁₁ to R₁₆ and R₂₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
* indicates a binding site to a neighboring atom,
a20 is an integer from 0 to 3,
R₁₅ and R₁₆ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a buffer layer, a hole-blocking layer, an electron control layer, an electron transport layer, an electron injection layer, or any combination thereof.

3. The light-emitting device of claim 1, wherein the amine-containing compound is in the interlayer.

4. The light-emitting device of claim 2, wherein the amine-containing compound is in the hole transport region.

5. The light-emitting device of claim 2, wherein the amine-containing compound is in the hole transport layer, and
the hole transport layer is in direct contact with the emission layer.

6. The light-emitting device of claim 1, further comprising a capping layer located outside the first electrode,
wherein the amine-containing compound is in the capping layer.

7. The light-emitting device of claim 1, further comprising:
a first capping layer located outside the first electrode; and
a second capping layer located outside the second electrode,
wherein the amine-containing compound is in the first capping layer or the second capping layer.

8. An electronic device comprising the light-emitting device of claim 1.

9. The electronic device of claim 8, further comprising:
a thin-film transistor electrically connected with the light-emitting device; and
a color filter, a color conversion layer, a touch screen layer, a polarizing layer, or any combination thereof.

10. An electronic apparatus comprising the electronic device of claim 8, wherein the electronic apparatus is one selected from a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and a signboard.

11. An amine-containing compound represented by Formula 1: 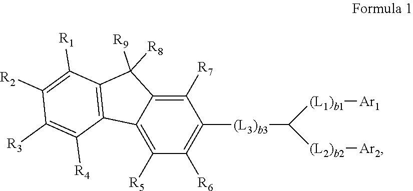
wherein, in Formula 1,
R₁ to R₉ are each independently a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₁-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
at least one selected from among R₁ to R₄ is the group represented by Formula 2,
R₈ and R₉ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
Ar₁ and Ar₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
at least one selected from among Ar₁ and Ar₂ is a saturated C₃-C₆₀ cyclic group unsubstituted or substituted with at least one R₁₀ₐ,
L₁ to L₃ are each independently a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b₁ to b₃ are each independently an integer from 0 to 5, 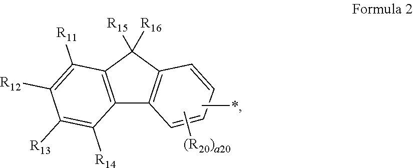
wherein, in Formula 2,
R₁₁ to R₁₆ and R₂₀ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
* indicates a binding site to a neighboring atom,
a20 is an integer from 0 to 3,
R₁₅ and R₁₆ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

12. The amine-containing compound of claim 11, wherein at least one selected from among Ar₁ and Ar₂ is:
a cyclohexyl group, a cycloheptyl group, or a cyclooctyl group, each unsubstituted or substituted with at least one R₁₀ₐ; or
a group represented by any one selected from among Formulae 3-1 to 3-5: 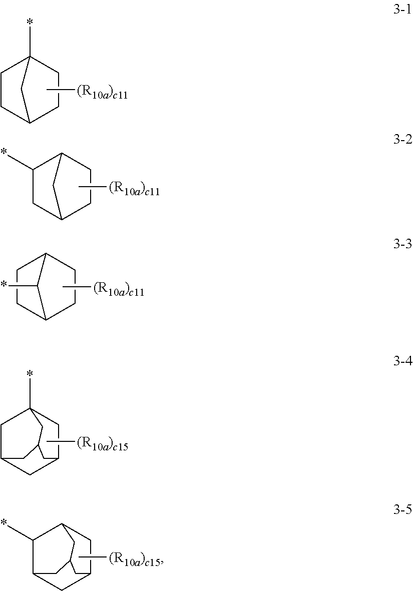
wherein, in Formulae 3-1 to 3-5,
R₁₀ₐ is as described in Formula 1,
c11 is an integer from 0 to 11,
c15 is an integer from 0 to 15, and
* indicates a binding site to a neighboring atom.

13. The amine-containing compound of claim 11, wherein Ar₁ and Ar₂ are each independently:
a cyclohexyl group, a cycloheptyl group, a cyclooctyl group, or a phenyl group, each unsubstituted or substituted with at least one R₁₀ₐ; or
a group represented by any one selected from among Formulae 4-1 to 4-15: 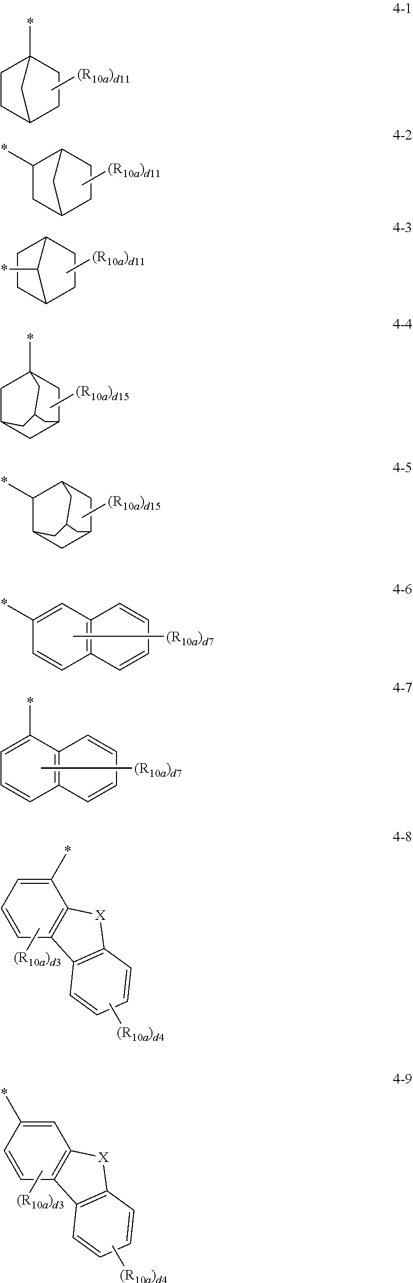 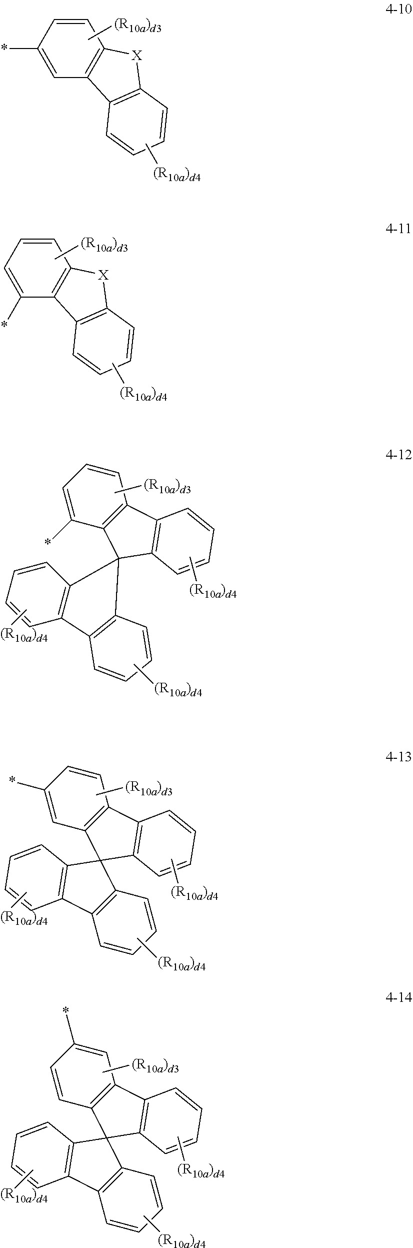 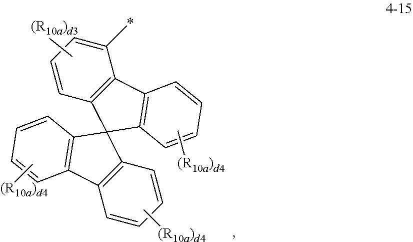
wherein, in Formulae 4-1 to 4-15,
R₁₀ₐ is as described in Formula 1,
X is C(R_{10b})(R_{10c}), N(R₁₀ₐ), O, or S,
R_{10b}, R_{10c}, and R_{10d} are each as described in Formula 1 with respect to R₁₀ₐ,
d3 is an integer from 0 to 3,
d4 is an integer from 0 to 4,
d7 is an integer from 0 to 7,
d11 is an integer from 0 to 11,
d15 is an integer from 0 to 15, and
* indicates a binding site to a neighboring atom.

14. The amine-containing compound of claim 11, wherein L₁ to L₃ are each independently:
a single bond; or
a benzene group, a naphthalene group, an anthracene group, a phenanthrene group, a triphenylene group, a pyrene group, or a chrysene group, each unsubstituted or substituted with at least one R₁₀ₐ, and
R₁₀ₐ is as described in Formula 1.

15. The amine-containing compound of claim 11, wherein L₁ to L₃ are each independently:
a single bond; or
a group represented by any one selected from among Formulae 5-1 to 5-13: 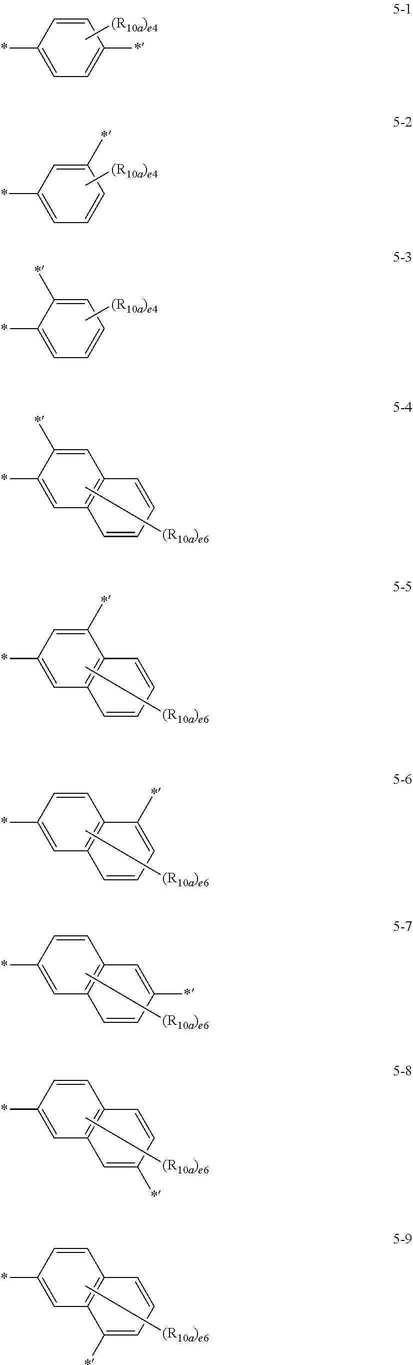 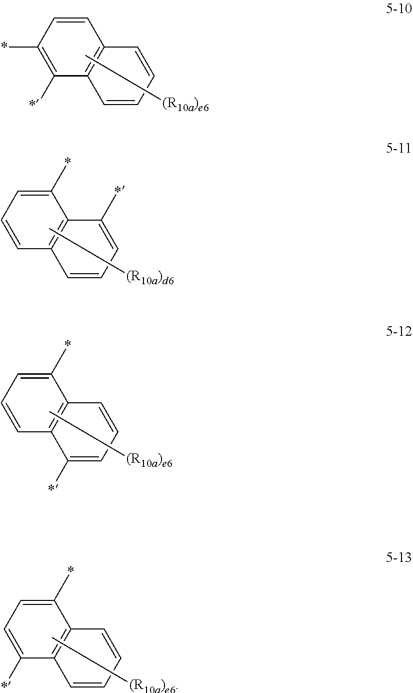
wherein, in Formulae 5-1 to 5-13,
R₁₀ₐ is as described in Formula 1,
e4 is an integer from 0 to 4,
e6 is an integer from 0 to 6, and
* and *′ each indicate a binding site to a neighboring atom.

16. The amine-containing compound of claim 11, wherein b3 is 0.

17. The amine-containing compound of claim 11, wherein any one selected from among R₁, R₃, and R₄ is the group represented by Formula 2.

18. The amine-containing compound of claim 11, wherein the group represented by Formula 2 is a group represented by any one selected from among Formulae 2-1 to 2-4: 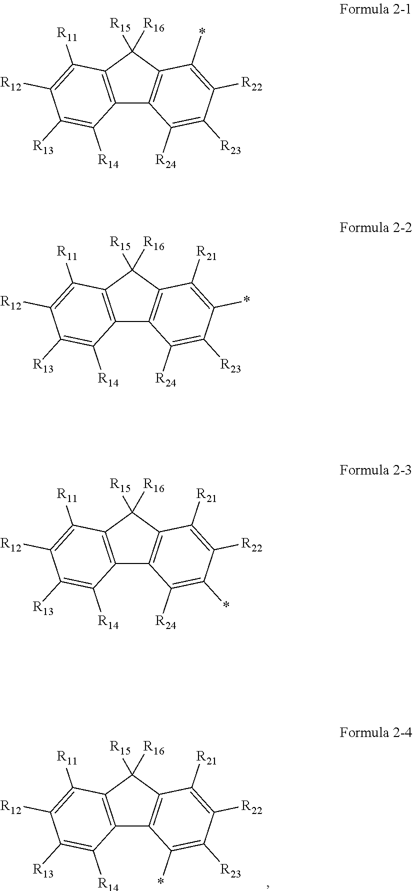
wherein, in Formulae 2-1 to 2-4,
R₁₁ to R₁₆ are each as described in Formula 1,
R₂₁ to R₂₄ are each independently as described in Formula 1 with respect to R₂₀, and * indicates a binding site to a neighboring atom.

19. The amine-containing compound of claim 11, wherein Formula 1 is represented by any one selected from among Formulae 1-1 to 1-16: 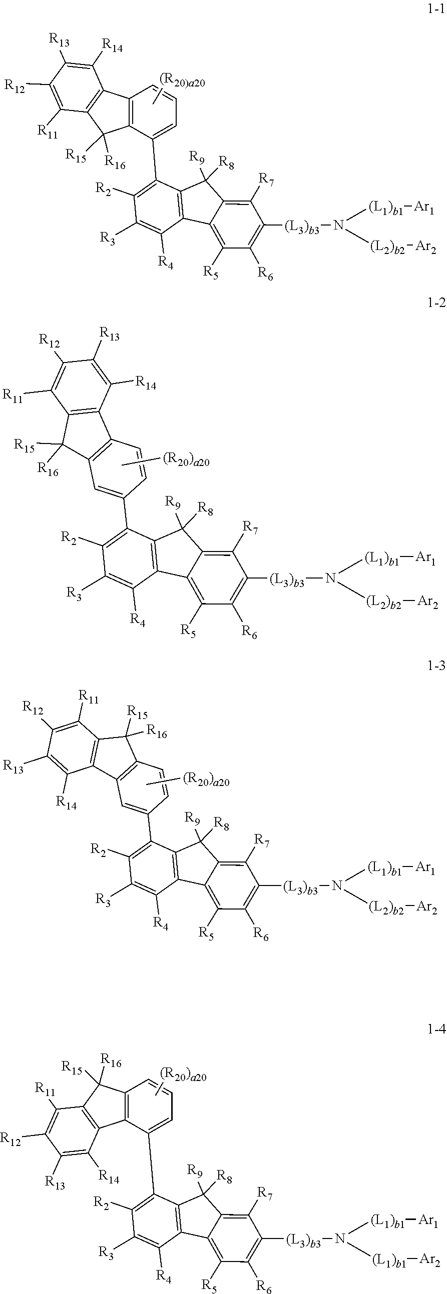 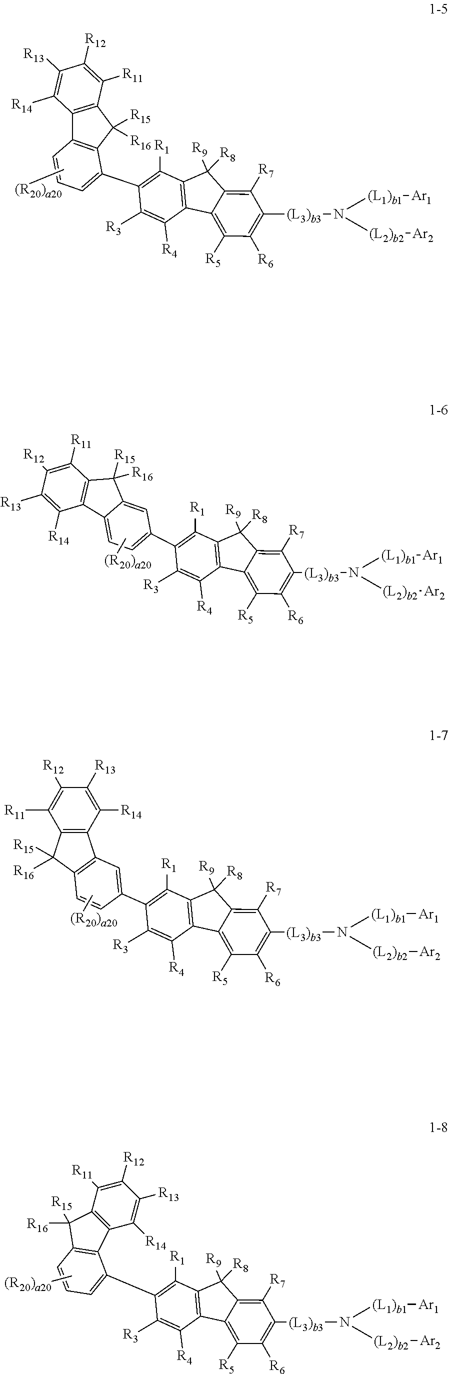 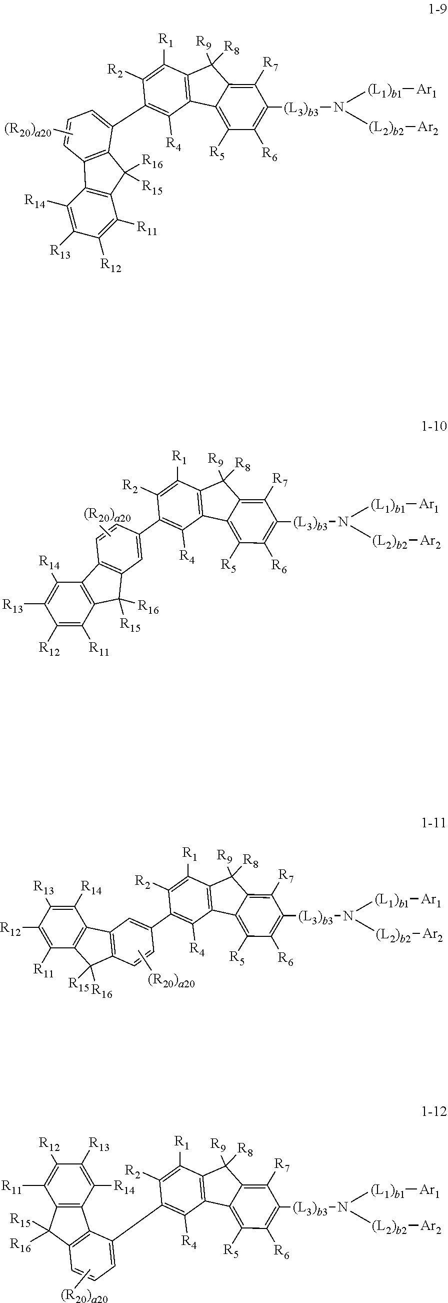 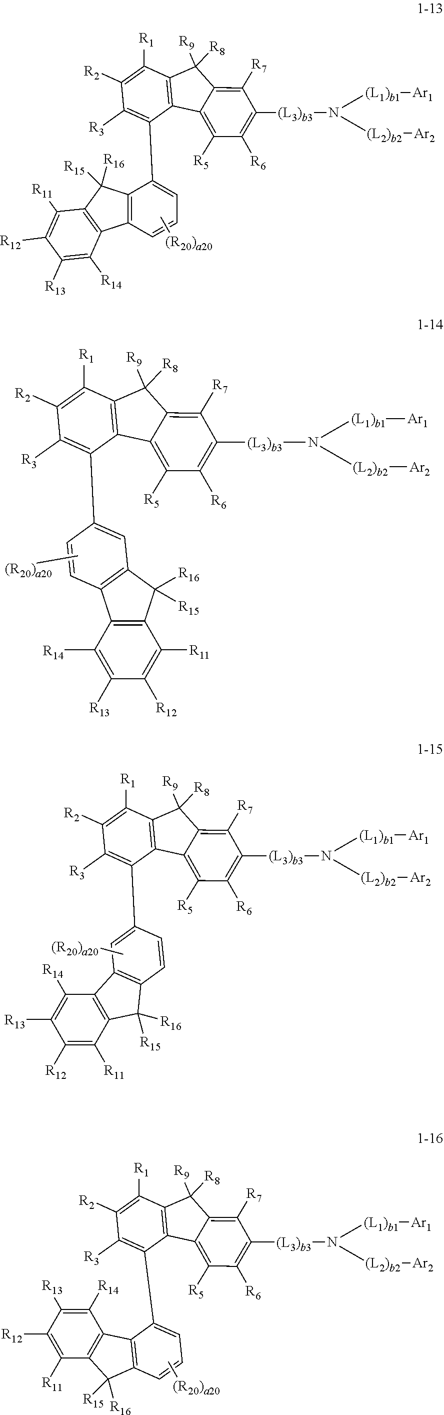
wherein, in Formulae 1-1 to 1-16,
R₁ to R₉, Ar₁, Ar₂, L₁ to L₃, b1 to b3, R₁₁ to R₁₆, R₂₀, and a20 are each as described in Formula 1.

20. The amine-containing compound of claim 11, wherein the amine-containing compound is one selected from among Compounds 1 to 240: 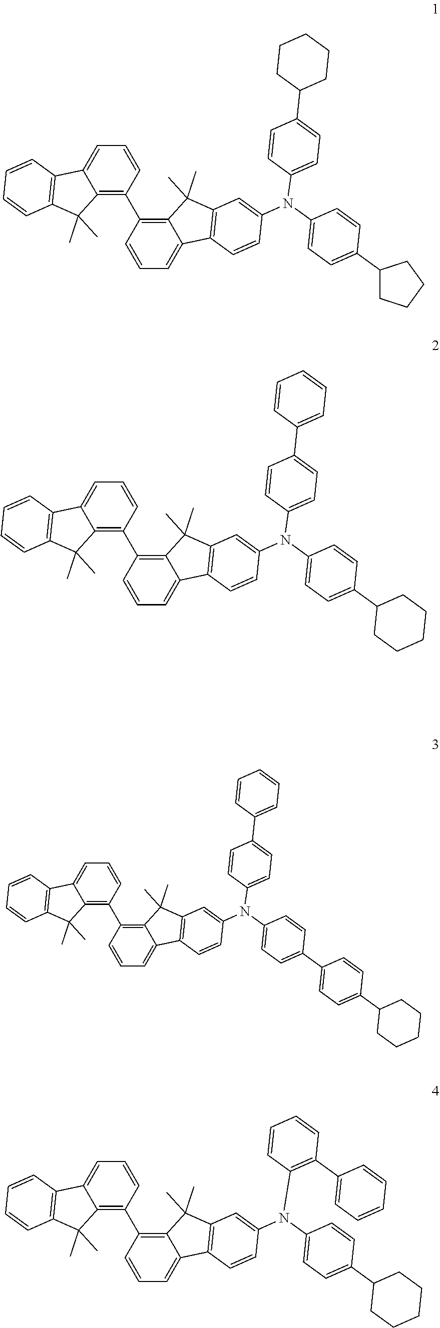 [SMILES: CC1(C)c2ccccc2-c2cccc(-c3cccc4c3C(C)(C)c3cc(N(c5ccc(C6CCCCC6)cc5)c5ccc(C6CCCC6)cc5)ccc3-4)c21 ; CC1(C)c2ccccc2-c2cccc(-c3cccc4c3C(C)(C)c3cc(N(c5ccc(-c6ccccc6)cc5)c5ccc(C6CCCCC6)cc5)ccc3-4)c21 ; CC1(C)c2ccccc2-c2cccc(-c3cccc4c3C(C)(C)c3cc(N(c5ccc(-c6ccccc6)cc5)c5ccc(-c6ccc(C7CCCCC7)cc6)cc5)ccc3-4)c21 ; CC1(C)c2ccccc2-c2cccc(-c3cccc4c3C(C)(C)c3cc(N(c5ccc(C6CCCCC6)cc5)c5ccccc5-c5ccccc5)ccc3-4)c21] 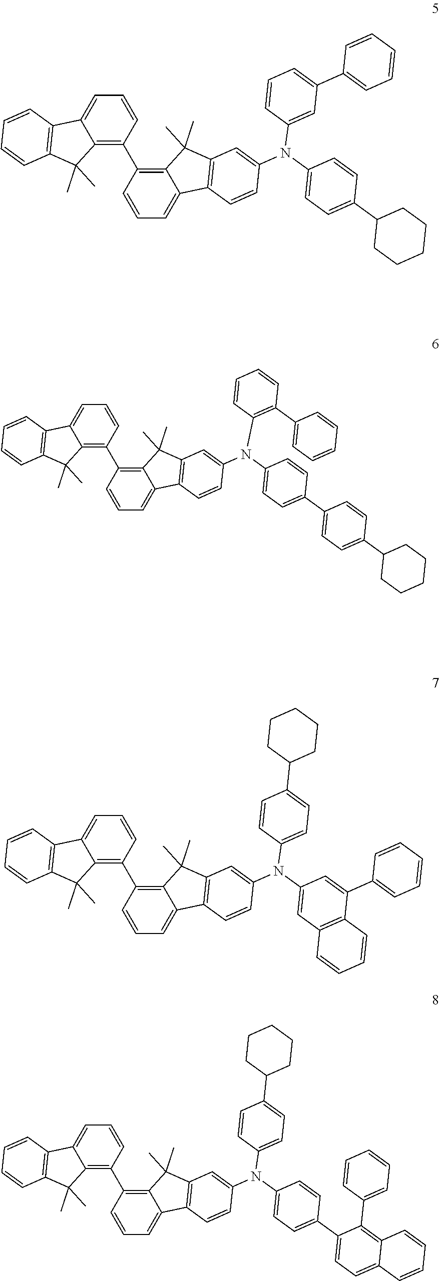 [SMILES: CC1(C)c2ccccc2-c2cccc(-c3cccc4c3C(C)(C)c3cc(N(c5ccc(C6CCCCC6)cc5)c5cccc(-c6ccccc6)c5)ccc3-4)c21 ; CC1(C)c2ccccc2-c2cccc(-c3cccc4c3C(C)(C)c3cc(N(c5ccc(-c6ccc(C7CCCCC7)cc6)cc5)c5ccccc5-c5ccccc5)ccc3-4)c21 ; CC1(C)c2ccccc2-c2cccc(-c3cccc4c3C(C)(C)c3cc(N(c5ccc(C6CCCCC6)cc5)c5cc(-c6ccccc6)c6ccccc6c5)ccc3-4)c21 ; CC1(C)c2ccccc2-c2cccc(-c3cccc4c3C(C)(C)c3cc(N(c5ccc(-c6ccc7ccccc7c6-c6ccccc6)cc5)c5ccc(C6CCCCC6)cc5)ccc3-4)c21] 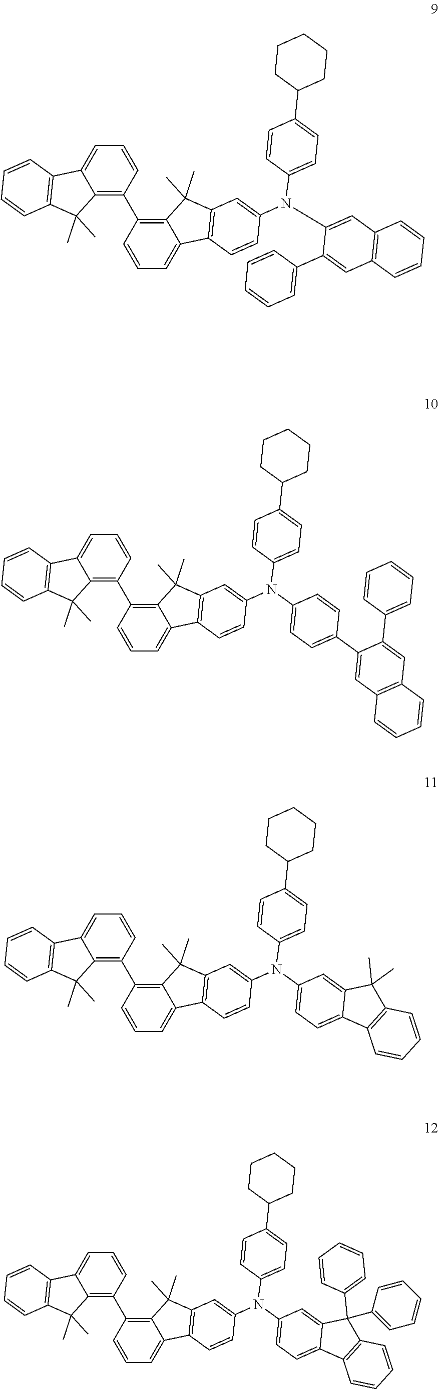 [SMILES: CC1(C)c2ccccc2-c2cccc(-c3cccc4c3C(C)(C)c3cc(N(c5ccc(C6CCCCC6)cc5)c5cc6ccccc6cc5-c5ccccc5)ccc3-4)c21 ; CC1(C)c2ccccc2-c2cccc(-c3cccc4c3C(C)(C)c3cc(N(c5ccc(-c6cc7ccccc7cc6-c6ccccc6)cc5)c5ccc(C6CCCCC6)cc5)ccc3-4)c21 ; CC1(C)c2ccccc2-c2ccc(N(c3ccc(C4CCCCC4)cc3)c3ccc4c(c3)C(C)(C)c3c-4cccc3-c3cccc4c3C(C)(C)c3ccccc3-4)cc21 ; CC1(C)c2ccccc2-c2cccc(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)c21] 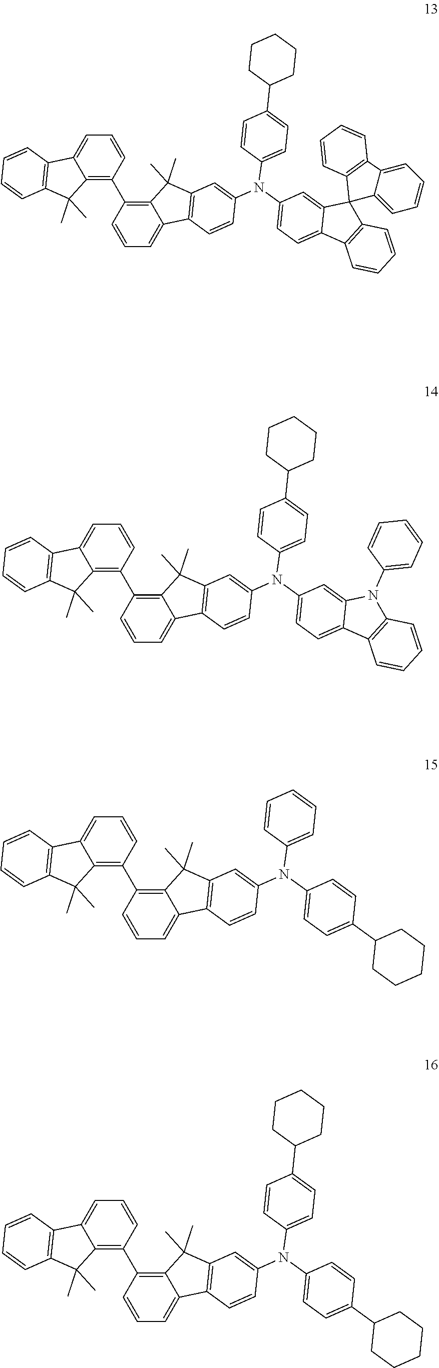 [SMILES: CC1(C)c2ccccc2-c2cccc(-c3cccc4c3C(C)(C)c3cc(N(c5ccc(C6CCCCC6)cc5)c5ccc6c(c5)C5(c7ccccc7-c7ccccc75)c5ccccc5-6)ccc3-4)c21 ; CC1(C)c2ccccc2-c2cccc(-c3cccc4c3C(C)(C)c3cc(N(c5ccc(C6CCCCC6)cc5)c5ccc6c7ccccc7n(-c7ccccc7)c6c5)ccc3-4)c21 ; CC1(C)c2ccccc2-c2cccc(-c3cccc4c3C(C)(C)c3cc(N(c5ccccc5)c5ccc(C6CCCCC6)cc5)ccc3-4)c21 ; CC1(C)c2ccccc2-c2cccc(-c3cccc4c3C(C)(C)c3cc(N(c5ccc(C6CCCCC6)cc5)c5ccc(C6CCCCC6)cc5)ccc3-4)c21] 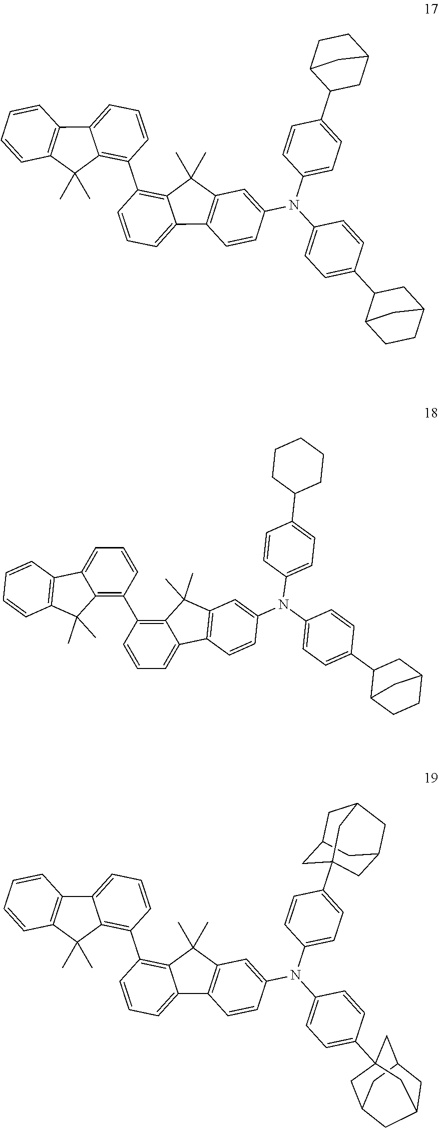 [SMILES: CC1(C)c2ccccc2-c2cccc(-c3cccc4c3C(C)(C)c3cc(N(c5ccc(C6CC7CCC6C7)cc5)c5ccc(C6CC7CCC6C7)cc5)ccc3-4)c21 ; CC1(C)c2ccccc2-c2cccc(-c3cccc4c3C(C)(C)c3cc(N(c5ccc(C6CCCCC6)cc5)c5ccc(C6CC7CCC6C7)cc5)ccc3-4)c21 ; CC1(C)c2ccccc2-c2cccc(-c3cccc4c3C(C)(C)c3cc(N(c5ccc(C67CC8CC(CC(C8)C6)C7)cc5)c5ccc(C67CC8CC(CC(C8)C6)C7)cc5)ccc3-4)c21] 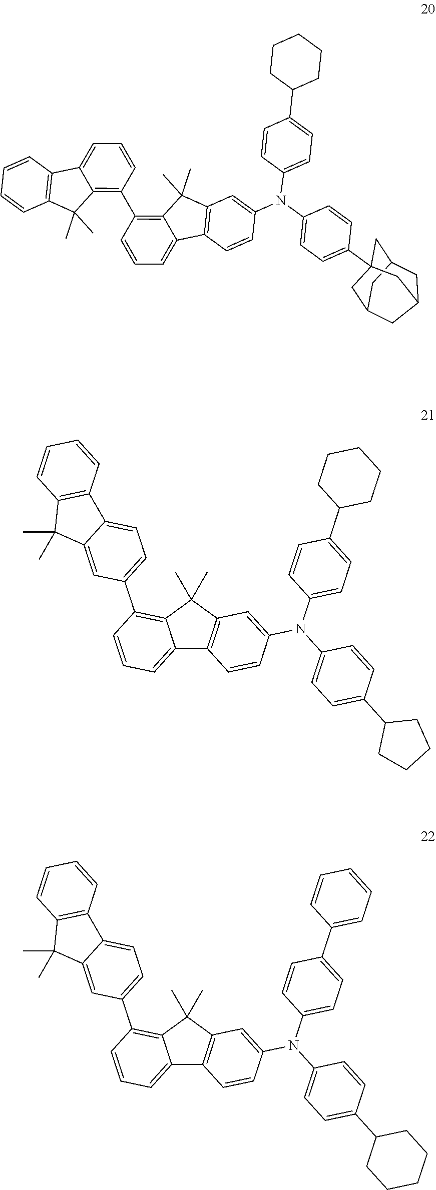 [SMILES: CC1(C)c2ccccc2-c2cccc(-c3cccc4c3C(C)(C)c3cc(N(c5ccc(C6CCCCC6)cc5)c5ccc(C67CC8CC(CC(C8)C6)C7)cc5)ccc3-4)c21 ; CC1(C)c2ccccc2-c2ccc(-c3cccc4c3C(C)(C)c3cc(N(c5ccc(C6CCCCC6)cc5)c5ccc(C6CCCC6)cc5)ccc3-4)cc21 ; CC1(C)c2ccccc2-c2ccc(-c3cccc4c3C(C)(C)c3cc(N(c5ccc(-c6ccccc6)cc5)c5ccc(C6CCCCC6)cc5)ccc3-4)cc21] 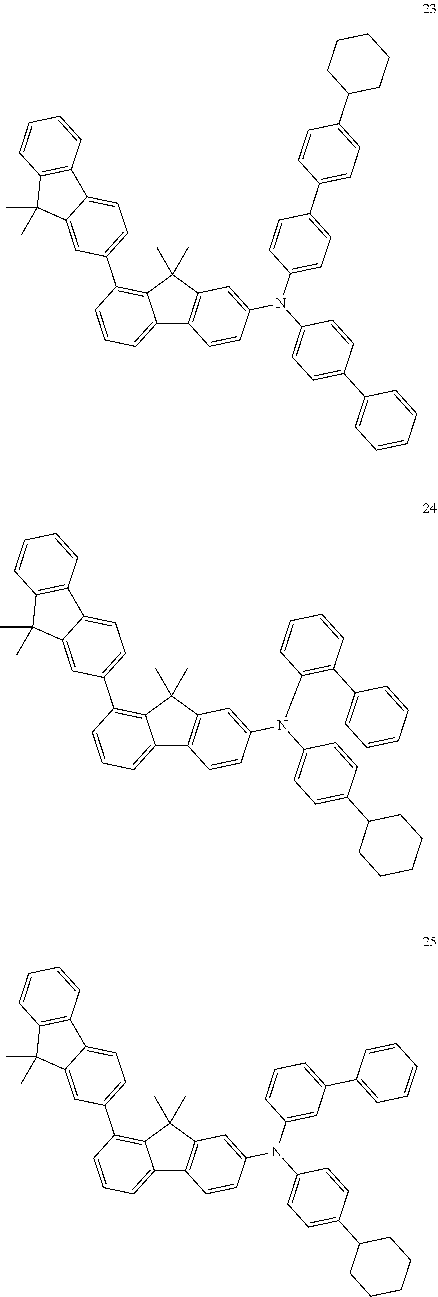 [SMILES: CC1(C)c2ccccc2-c2ccc(-c3cccc4c3C(C)(C)c3cc(N(c5ccc(-c6ccccc6)cc5)c5ccc(-c6ccc(C7CCCCC7)cc6)cc5)ccc3-4)cc21 ; CC1(C)c2ccccc2-c2ccc(-c3cccc4c3C(C)(C)c3cc(N(c5ccc(C6CCCCC6)cc5)c5ccccc5-c5ccccc5)ccc3-4)cc21 ; CC1(C)c2ccccc2-c2ccc(-c3cccc4c3C(C)(C)c3cc(N(c5ccc(C6CCCCC6)cc5)c5cccc(-c6ccccc6)c5)ccc3-4)cc21] 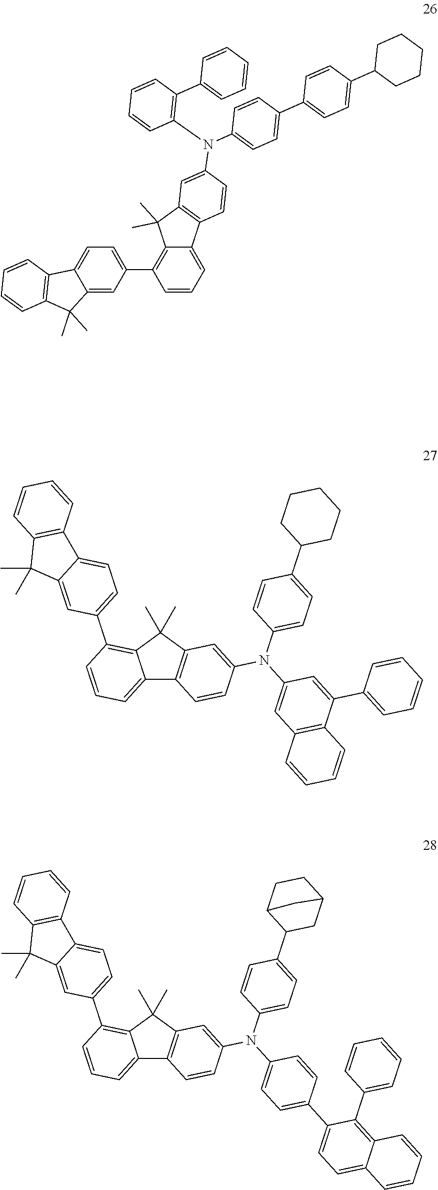 [SMILES: CC1(C)c2ccccc2-c2ccc(-c3cccc4c3C(C)(C)c3cc(N(c5ccc(-c6ccc(C7CCCCC7)cc6)cc5)c5ccccc5-c5ccccc5)ccc3-4)cc21 ; CC1(C)c2ccccc2-c2ccc(-c3cccc4c3C(C)(C)c3cc(N(c5ccc(C6CCCCC6)cc5)c5cc(-c6ccccc6)c6ccccc6c5)ccc3-4)cc21 ; CC1(C)c2ccccc2-c2ccc(-c3cccc4c3C(C)(C)c3cc(N(c5ccc(-c6ccc7ccccc7c6-c6ccccc6)cc5)c5ccc(C6CC7CCC6C7)cc5)ccc3-4)cc21] 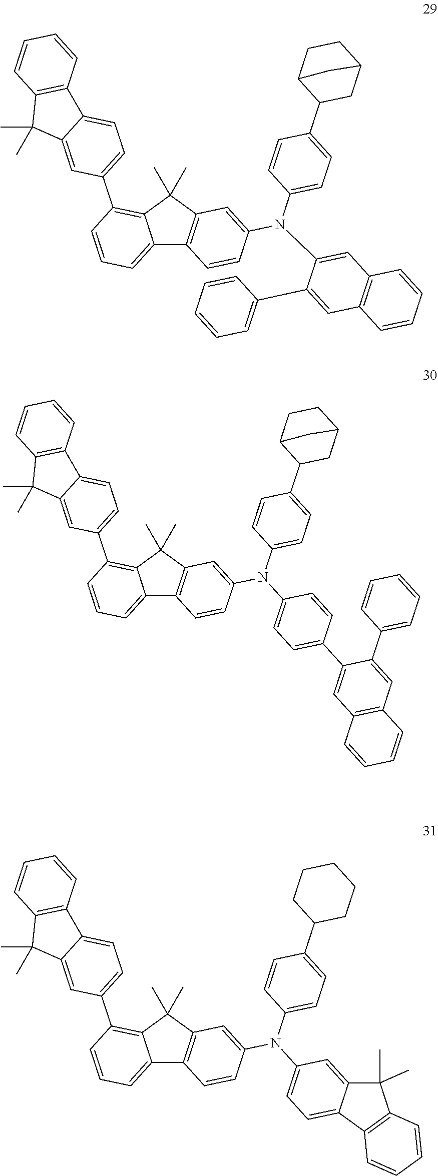 [SMILES: CC1(C)c2ccccc2-c2ccc(-c3cccc4c3C(C)(C)c3cc(N(c5ccc(C6CC7CCC6C7)cc5)c5cc6ccccc6cc5-c5ccccc5)ccc3-4)cc21 ; CC1(C)c2ccccc2-c2ccc(-c3cccc4c3C(C)(C)c3cc(N(c5ccc(-c6cc7ccccc7cc6-c6ccccc6)cc5)c5ccc(C6CC7CCC6C7)cc5)ccc3-4)cc21 ; CC1(C)c2ccccc2-c2ccc(-c3cccc4c3C(C)(C)c3cc(N(c5ccc(C6CCCCC6)cc5)c5ccc6c(c5)C(C)(C)c5ccccc5-6)ccc3-4)cc21] 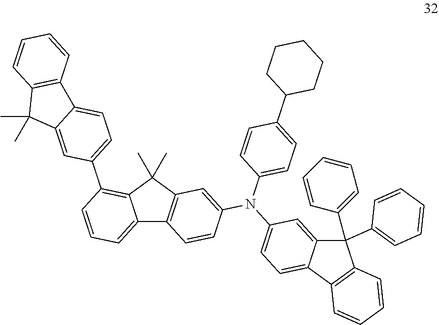 [SMILES: CC1(C)c2ccccc2-c2ccc(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)cc21] 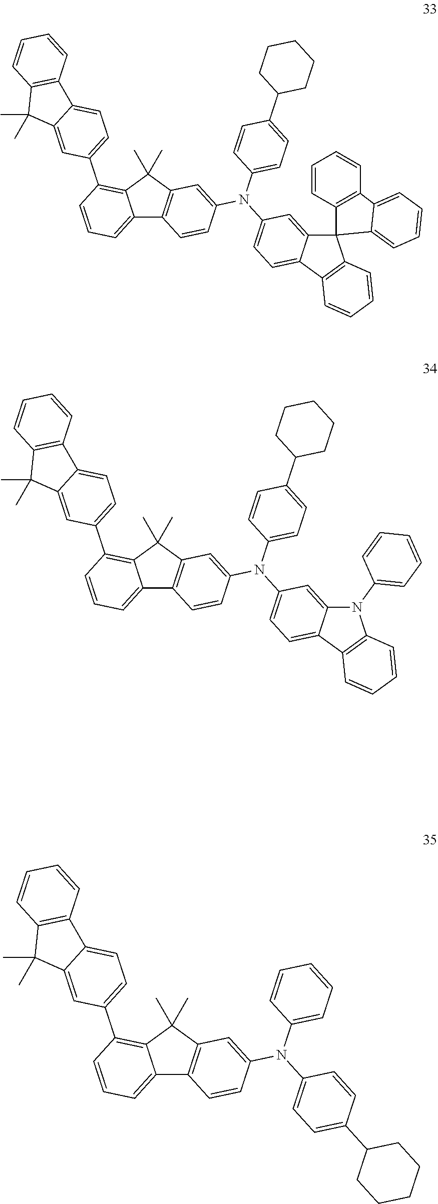 [SMILES: CC1(C)c2ccccc2-c2ccc(-c3cccc4c3C(C)(C)c3cc(N(c5ccc(C6CCCCC6)cc5)c5ccc6c(c5)C5(c7ccccc7-c7ccccc75)c5ccccc5-6)ccc3-4)cc21 ; CC1(C)c2ccccc2-c2ccc(-c3cccc4c3C(C)(C)c3cc(N(c5ccc(C6CCCCC6)cc5)c5ccc6c7ccccc7n(-c7ccccc7)c6c5)ccc3-4)cc21 ; CC1(C)c2ccccc2-c2ccc(-c3cccc4c3C(C)(C)c3cc(N(c5ccccc5)c5ccc(C6CCCCC6)cc5)ccc3-4)cc21] 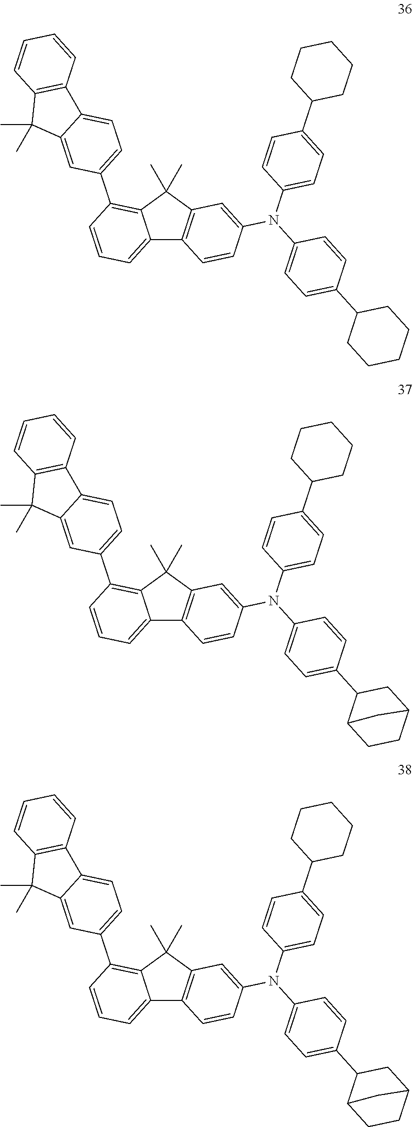 [SMILES: CC1(C)c2ccccc2-c2ccc(-c3cccc4c3C(C)(C)c3cc(N(c5ccc(C6CCCCC6)cc5)c5ccc(C6CCCCC6)cc5)ccc3-4)cc21 ; CC1(C)c2ccccc2-c2ccc(-c3cccc4c3C(C)(C)c3cc(N(c5ccc(C6CCCCC6)cc5)c5ccc(C6CC7CCC6C7)cc5)ccc3-4)cc21 ; CC1(C)c2ccccc2-c2ccc(-c3cccc4c3C(C)(C)c3cc(N(c5ccc(C6CCCCC6)cc5)c5ccc(C6CC7CCC6C7)cc5)ccc3-4)cc21] 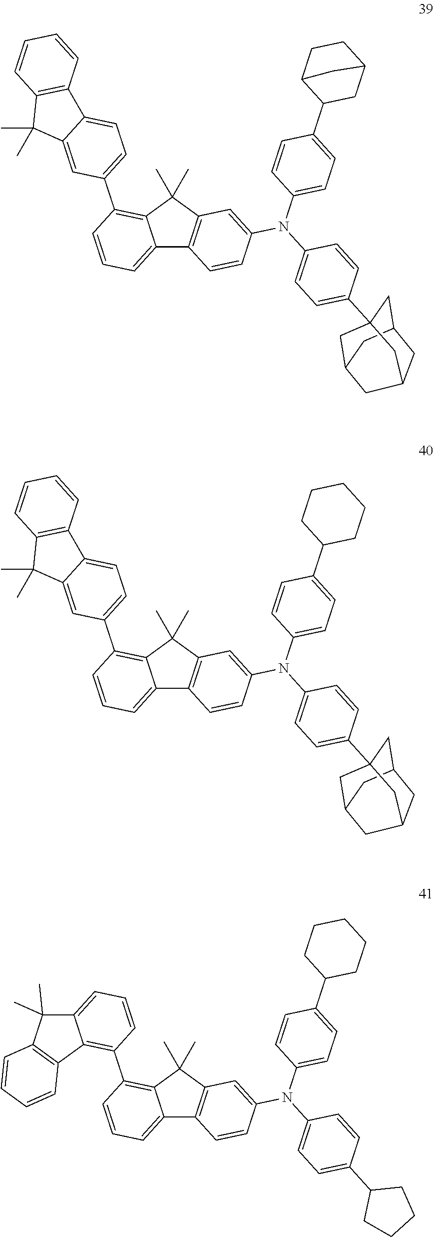 [SMILES: CC1(C)c2ccccc2-c2ccc(-c3cccc4c3C(C)(C)c3cc(N(c5ccc(C6CC7CCC6C7)cc5)c5ccc(C67CC8CC(CC(C8)C6)C7)cc5)ccc3-4)cc21 ; CC1(C)c2ccccc2-c2ccc(-c3cccc4c3C(C)(C)c3cc(N(c5ccc(C6CCCCC6)cc5)c5ccc(C67CC8CC(CC(C8)C6)C7)cc5)ccc3-4)cc21 ; CC1(C)c2ccccc2-c2c(-c3cccc4c3C(C)(C)c3cc(N(c5ccc(C6CCCCC6)cc5)c5ccc(C6CCCC6)cc5)ccc3-4)cccc21] 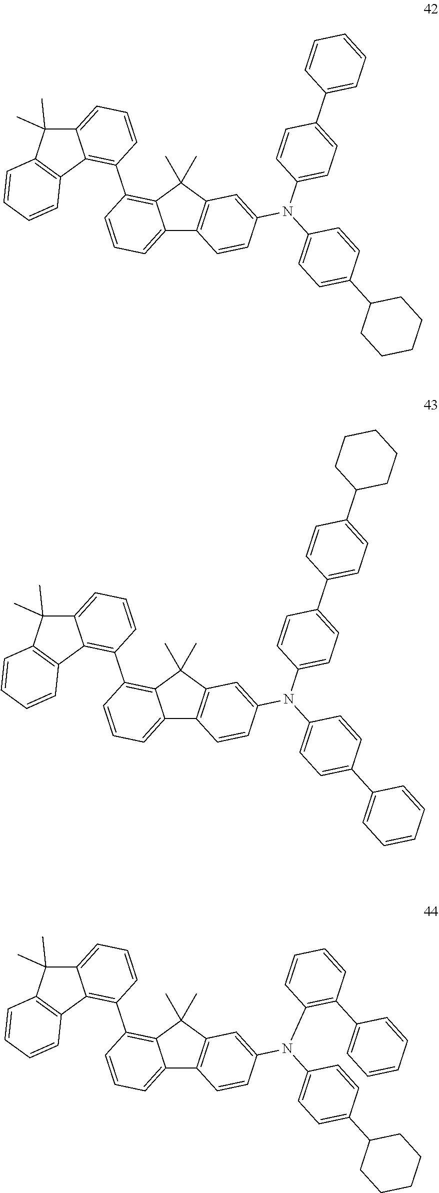 [SMILES: CC1(C)c2ccccc2-c2c(-c3cccc4c3C(C)(C)c3cc(N(c5ccc(-c6ccccc6)cc5)c5ccc(C6CCCCC6)cc5)ccc3-4)cccc21 ; CC1(C)c2ccccc2-c2c(-c3cccc4c3C(C)(C)c3cc(N(c5ccc(-c6ccccc6)cc5)c5ccc(-c6ccc(C7CCCCC7)cc6)cc5)ccc3-4)cccc21 ; CC1(C)c2ccccc2-c2c(-c3cccc4c3C(C)(C)c3cc(N(c5ccc(C6CCCCC6)cc5)c5ccccc5-c5ccccc5)ccc3-4)cccc21] 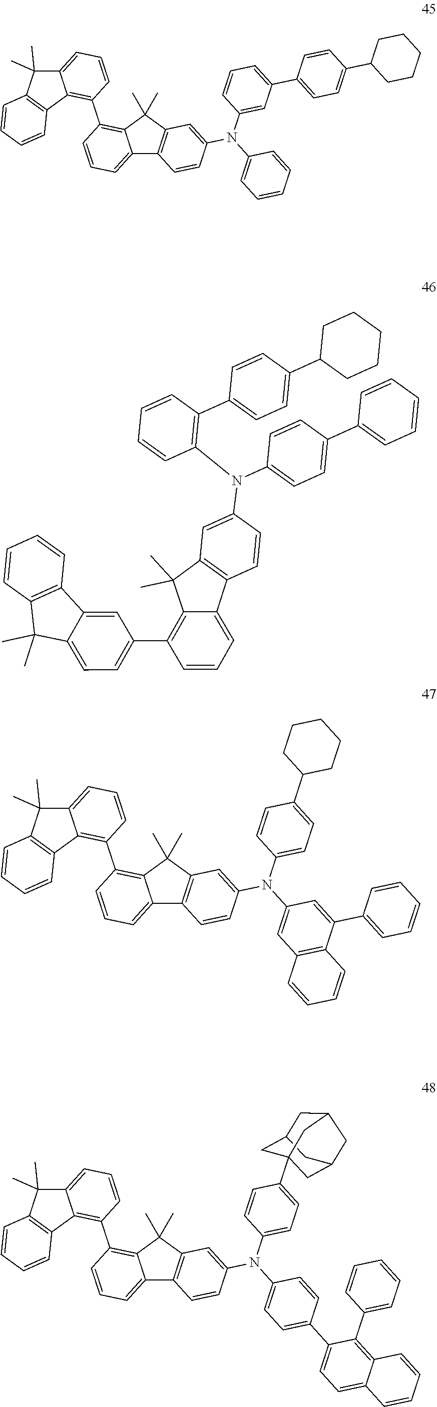 [SMILES: CC1(C)c2ccccc2-c2c(-c3cccc4c3C(C)(C)c3cc(N(c5ccccc5)c5cccc(-c6ccc(C7CCCCC7)cc6)c5)ccc3-4)cccc21 ; CC1(C)c2ccccc2-c2cc(-c3cccc4c3C(C)(C)c3cc(N(c5ccc(-c6ccccc6)cc5)c5ccccc5-c5ccc(C6CCCCC6)cc5)ccc3-4)ccc21 ; CC1(C)c2ccccc2-c2c(-c3cccc4c3C(C)(C)c3cc(N(c5ccc(C6CCCCC6)cc5)c5cc(-c6ccccc6)c6ccccc6c5)ccc3-4)cccc21 ; CC1(C)c2ccccc2-c2c(-c3cccc4c3C(C)(C)c3cc(N(c5ccc(-c6ccc7ccccc7c6-c6ccccc6)cc5)c5ccc(C67CC8CC(CC(C8)C6)C7)cc5)ccc3-4)cccc21] 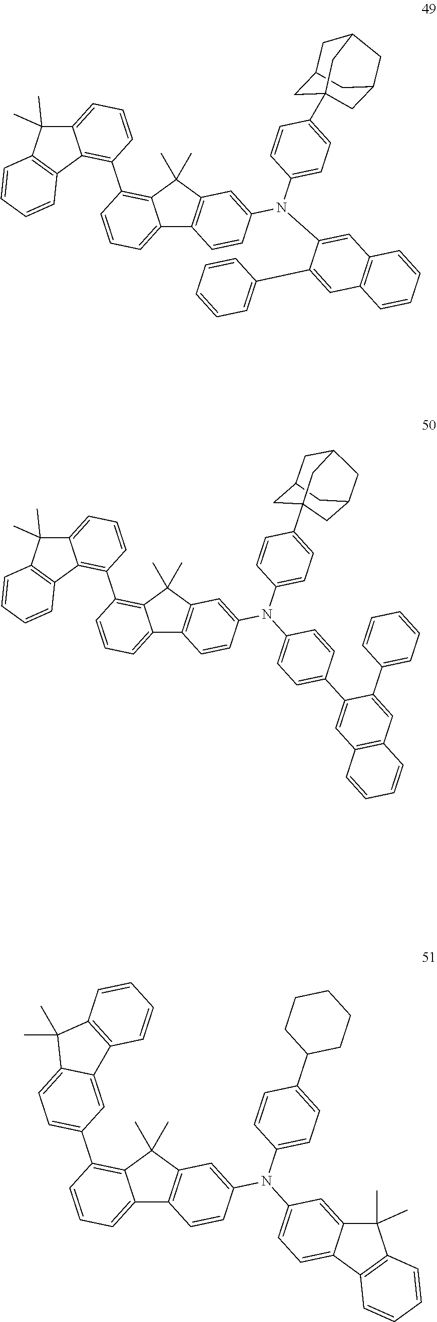 [SMILES: CC1(C)c2ccccc2-c2c(-c3cccc4c3C(C)(C)c3cc(N(c5ccc(C67CC8CC(CC(C8)C6)C7)cc5)c5cc6ccccc6cc5-c5ccccc5)ccc3-4)cccc21 ; CC1(C)c2ccccc2-c2c(-c3cccc4c3C(C)(C)c3cc(N(c5ccc(-c6cc7ccccc7cc6-c6ccccc6)cc5)c5ccc(C67CC8CC(CC(C8)C6)C7)cc5)ccc3-4)cccc21 ; CC1(C)c2ccccc2-c2cc(-c3cccc4c3C(C)(C)c3cc(N(c5ccc(C6CCCCC6)cc5)c5ccc6c(c5)C(C)(C)c5ccccc5-6)ccc3-4)ccc21] 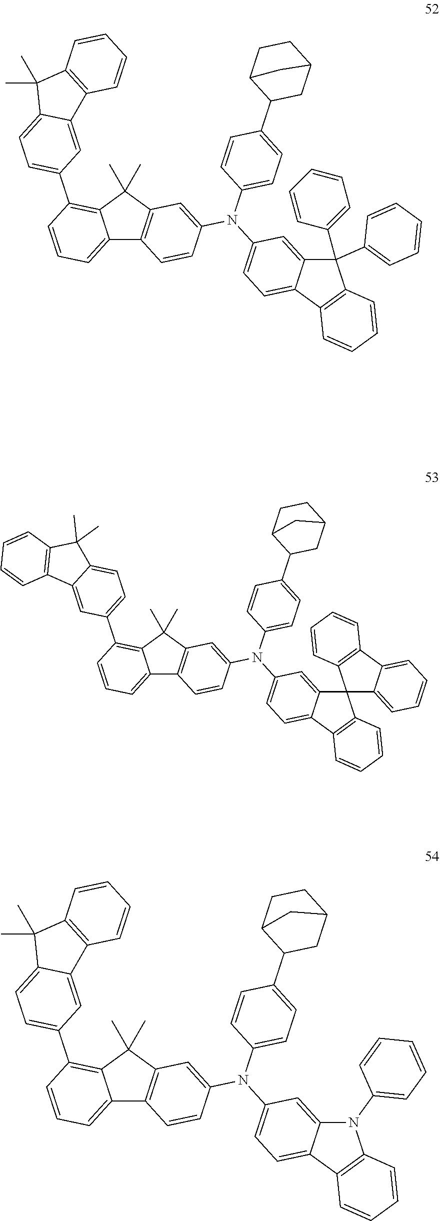 [SMILES: CC1(C)c2ccccc2-c2cc(-c3cccc4c3C(C)(C)c3cc(N(c5ccc(C6CC7CCC6C7)cc5)c5ccc6c(c5)C(c5ccccc5)(c5ccccc5)c5ccccc5-6)ccc3-4)ccc21 ; CC1(C)c2ccccc2-c2cc(-c3cccc4c3C(C)(C)c3cc(N(c5ccc(C6CC7CCC6C7)cc5)c5ccc6c(c5)C5(c7ccccc7-c7ccccc75)c5ccccc5-6)ccc3-4)ccc21 ; CC1(C)c2ccccc2-c2cc(-c3cccc4c3C(C)(C)c3cc(N(c5ccc(C6CC7CCC6C7)cc5)c5ccc6c7ccccc7n(-c7ccccc7)c6c5)ccc3-4)ccc21] 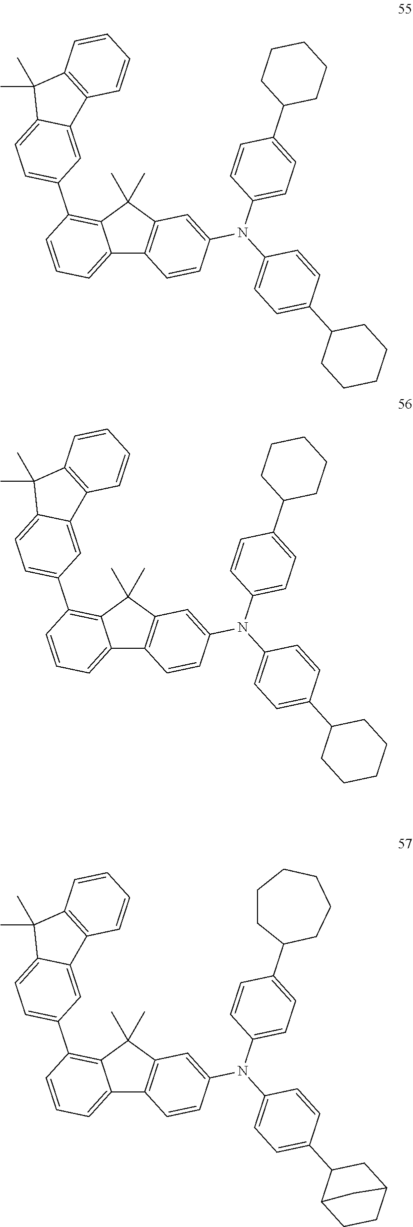 [SMILES: CC1(C)c2ccccc2-c2cc(-c3cccc4c3C(C)(C)c3cc(N(c5ccc(C6CCCCC6)cc5)c5ccc(C6CCCCC6)cc5)ccc3-4)ccc21 ; CC1(C)c2ccccc2-c2cc(-c3cccc4c3C(C)(C)c3cc(N(c5ccc(C6CCCCC6)cc5)c5ccc(C6CCCCC6)cc5)ccc3-4)ccc21 ; CC1(C)c2ccccc2-c2cc(-c3cccc4c3C(C)(C)c3cc(N(c5ccc(C6CCCCCC6)cc5)c5ccc(C6CC7CCC6C7)cc5)ccc3-4)ccc21] 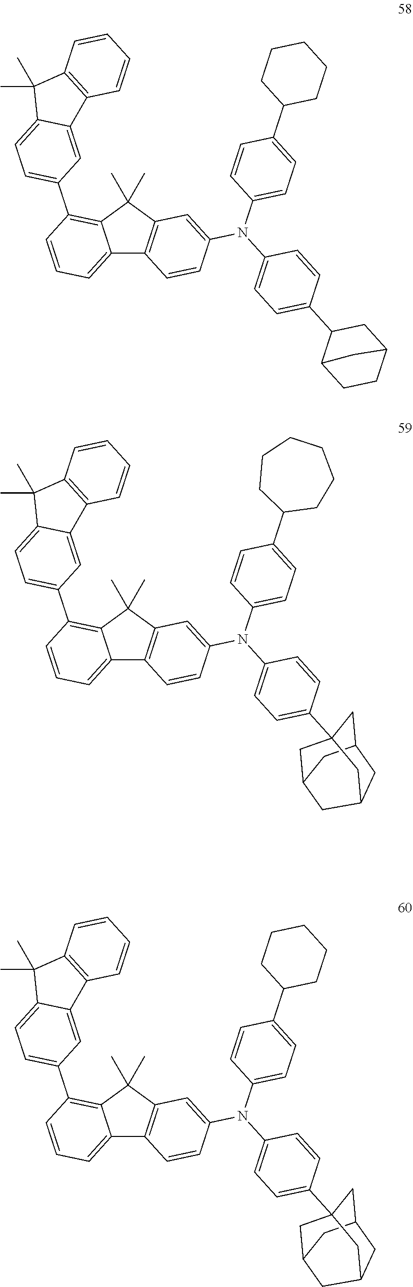 [SMILES: CC1(C)c2ccccc2-c2cc(-c3cccc4c3C(C)(C)c3cc(N(c5ccc(C6CCCCC6)cc5)c5ccc(C6CC7CCC6C7)cc5)ccc3-4)ccc21 ; CC1(C)c2ccccc2-c2cc(-c3cccc4c3C(C)(C)c3cc(N(c5ccc(C6CCCCCC6)cc5)c5ccc(C67CC8CC(CC(C8)C6)C7)cc5)ccc3-4)ccc21 ; CC1(C)c2ccccc2-c2cc(-c3cccc4c3C(C)(C)c3cc(N(c5ccc(C6CCCCC6)cc5)c5ccc(C67CC8CC(CC(C8)C6)C7)cc5)ccc3-4)ccc21] 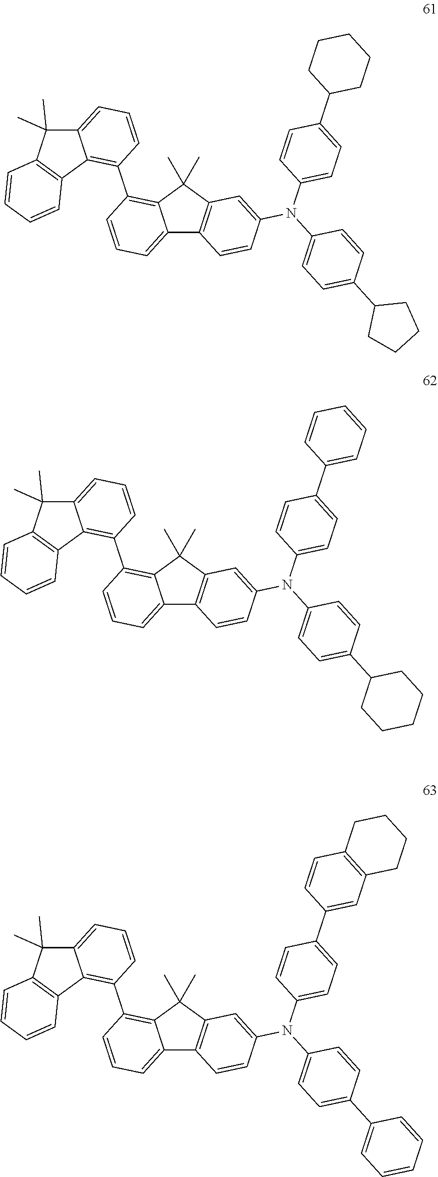 [SMILES: CC1(C)c2ccccc2-c2c(-c3cccc4c3C(C)(C)c3cc(N(c5ccc(C6CCCCC6)cc5)c5ccc(C6CCCC6)cc5)ccc3-4)cccc21 ; CC1(C)c2ccccc2-c2c(-c3cccc4c3C(C)(C)c3cc(N(c5ccc(-c6ccccc6)cc5)c5ccc(C6CCCCC6)cc5)ccc3-4)cccc21 ; CC1(C)c2ccccc2-c2c(-c3cccc4c3C(C)(C)c3cc(N(c5ccc(-c6ccccc6)cc5)c5ccc(-c6ccc7c(c6)CCCC7)cc5)ccc3-4)cccc21] 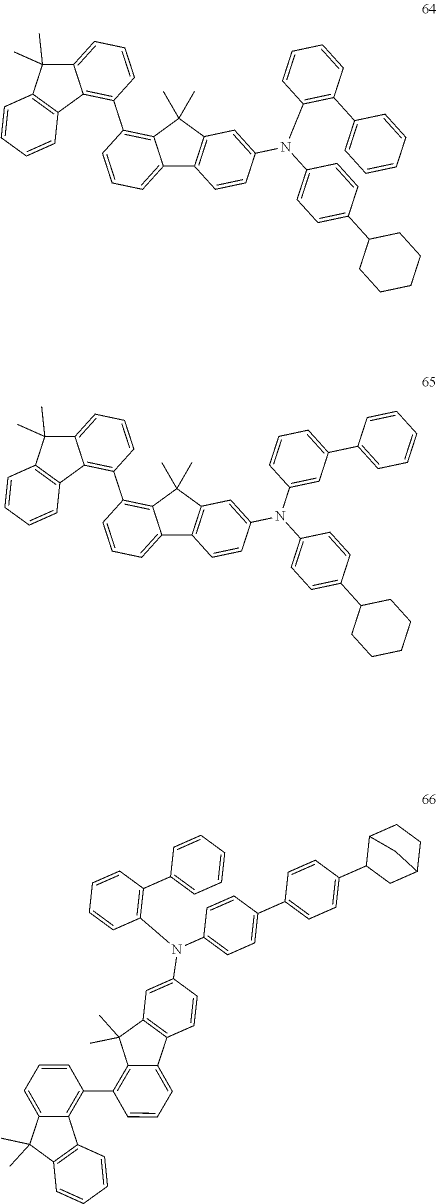 [SMILES: CC1(C)c2ccccc2-c2c(-c3cccc4c3C(C)(C)c3cc(N(c5ccc(C6CCCCC6)cc5)c5ccccc5-c5ccccc5)ccc3-4)cccc21 ; CC1(C)c2ccccc2-c2c(-c3cccc4c3C(C)(C)c3cc(N(c5ccc(C6CCCCC6)cc5)c5cccc(-c6ccccc6)c5)ccc3-4)cccc21 ; CC1(C)c2ccccc2-c2c(-c3cccc4c3C(C)(C)c3cc(N(c5ccc(-c6ccc(C7CC8CCC7C8)cc6)cc5)c5ccccc5-c5ccccc5)ccc3-4)cccc21] 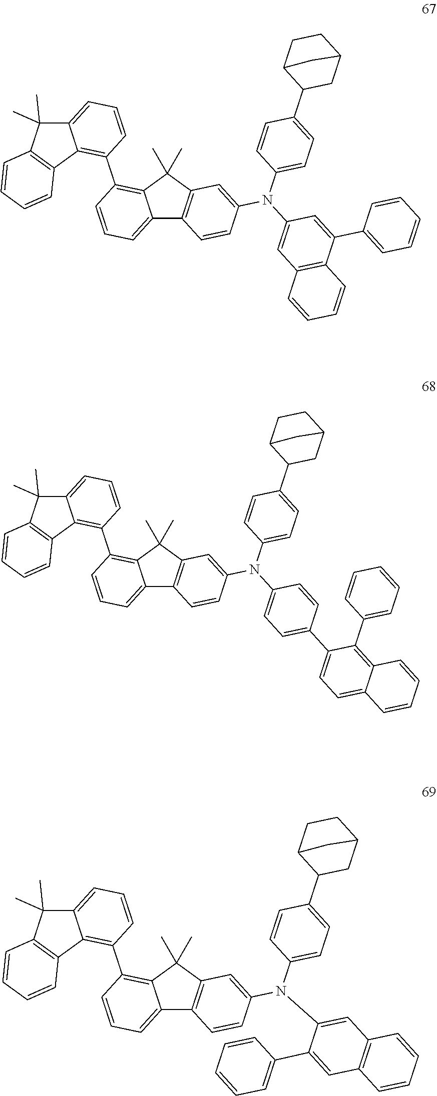 [SMILES: CC1(C)c2ccccc2-c2c(-c3cccc4c3C(C)(C)c3cc(N(c5ccc(C6CC7CCC6C7)cc5)c5cc(-c6ccccc6)c6ccccc6c5)ccc3-4)cccc21 ; CC1(C)c2ccccc2-c2c(-c3cccc4c3C(C)(C)c3cc(N(c5ccc(-c6ccc7ccccc7c6-c6ccccc6)cc5)c5ccc(C6CC7CCC6C7)cc5)ccc3-4)cccc21 ; CC1(C)c2ccccc2-c2c(-c3cccc4c3C(C)(C)c3cc(N(c5ccc(C6CC7CCC6C7)cc5)c5cc6ccccc6cc5-c5ccccc5)ccc3-4)cccc21] 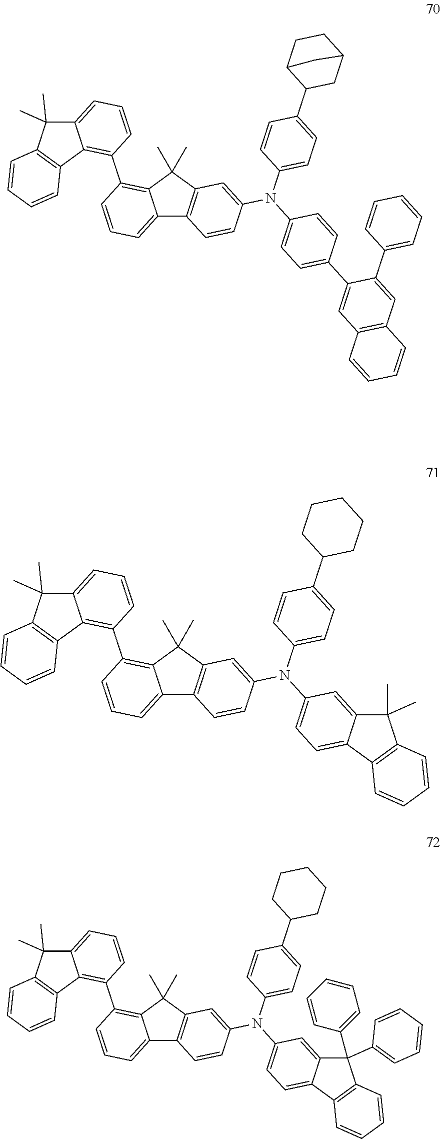 [SMILES: CC1(C)c2ccccc2-c2c(-c3cccc4c3C(C)(C)c3cc(N(c5ccc(-c6cc7ccccc7cc6-c6ccccc6)cc5)c5ccc(C6CC7CCC6C7)cc5)ccc3-4)cccc21 ; CC1(C)c2ccccc2-c2ccc(N(c3ccc(C4CCCCC4)cc3)c3ccc4c(c3)C(C)(C)c3c-4cccc3-c3cccc4c3-c3ccccc3C4(C)C)cc21 ; CC1(C)c2ccccc2-c2c(-c3cccc4c3C(C)(C)c3cc(N(c5ccc(C6CCCCC6)cc5)c5ccc6c(c5)C(c5ccccc5)(c5ccccc5)c5ccccc5-6)ccc3-4)cccc21] 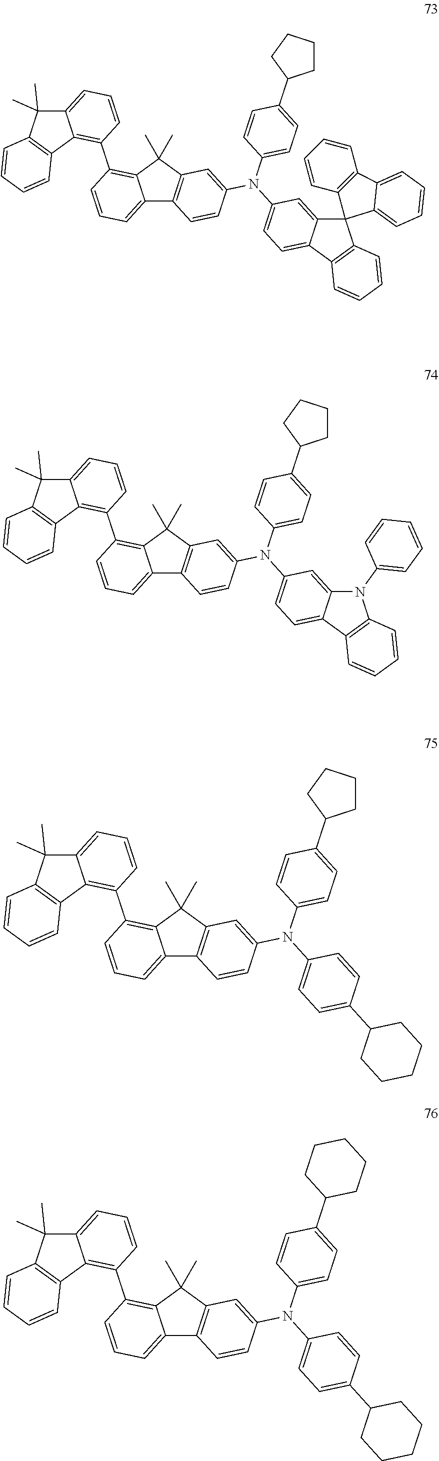 [SMILES: CC1(C)c2ccccc2-c2c(-c3cccc4c3C(C)(C)c3cc(N(c5ccc(C6CCCC6)cc5)c5ccc6c(c5)C5(c7ccccc7-c7ccccc75)c5ccccc5-6)ccc3-4)cccc21 ; CC1(C)c2ccccc2-c2c(-c3cccc4c3C(C)(C)c3cc(N(c5ccc(C6CCCC6)cc5)c5ccc6c7ccccc7n(-c7ccccc7)c6c5)ccc3-4)cccc21 ; CC1(C)c2ccccc2-c2c(-c3cccc4c3C(C)(C)c3cc(N(c5ccc(C6CCCCC6)cc5)c5ccc(C6CCCC6)cc5)ccc3-4)cccc21 ; CC1(C)c2ccccc2-c2c(-c3cccc4c3C(C)(C)c3cc(N(c5ccc(C6CCCCC6)cc5)c5ccc(C6CCCCC6)cc5)ccc3-4)cccc21] 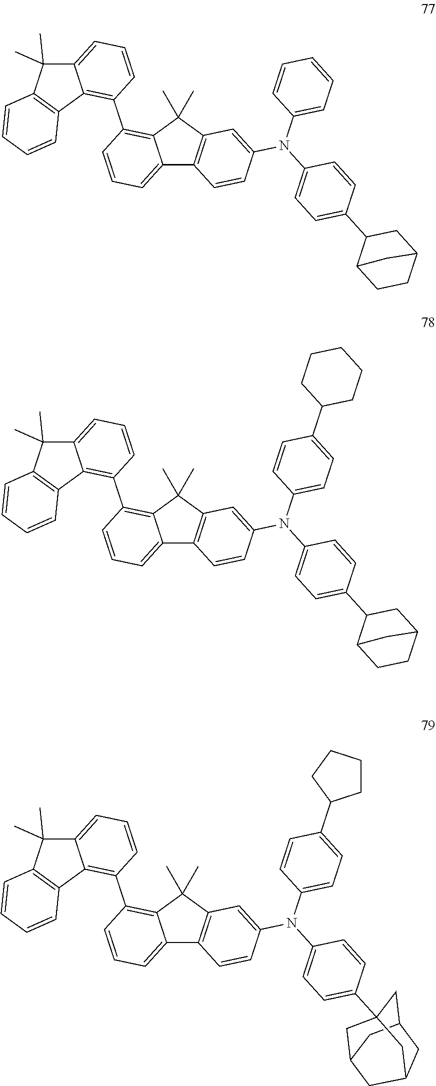 [SMILES: CC1(C)c2ccccc2-c2c(-c3cccc4c3C(C)(C)c3cc(N(c5ccccc5)c5ccc(C6CC7CCC6C7)cc5)ccc3-4)cccc21 ; CC1(C)c2ccccc2-c2c(-c3cccc4c3C(C)(C)c3cc(N(c5ccc(C6CCCCC6)cc5)c5ccc(C6CC7CCC6C7)cc5)ccc3-4)cccc21 ; CC1(C)c2ccccc2-c2c(-c3cccc4c3C(C)(C)c3cc(N(c5ccc(C6CCCC6)cc5)c5ccc(C67CC8CC(CC(C8)C6)C7)cc5)ccc3-4)cccc21] 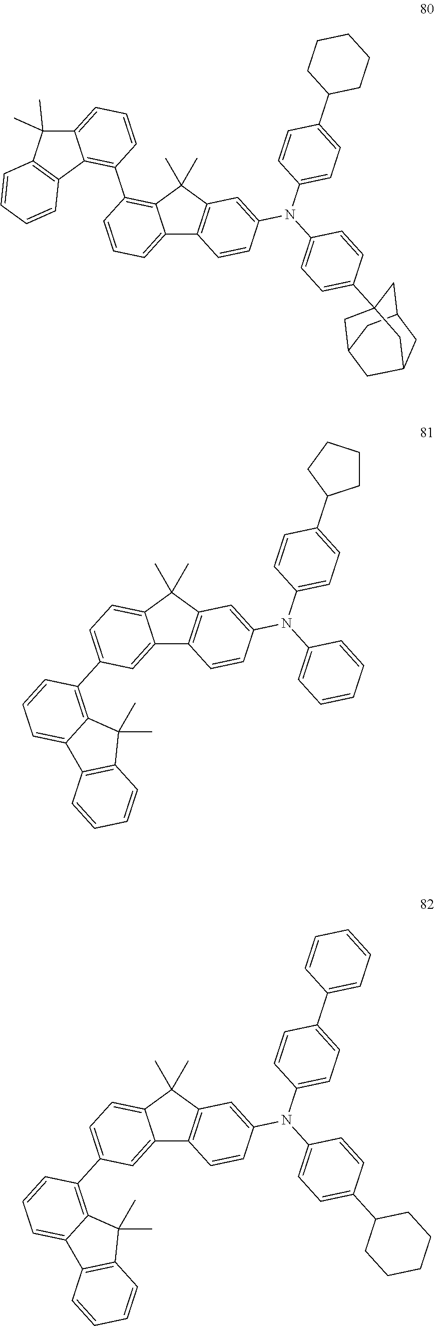 [SMILES: CC1(C)c2ccccc2-c2c(-c3cccc4c3C(C)(C)c3cc(N(c5ccc(C6CCCCC6)cc5)c5ccc(C67CC8CC(CC(C8)C6)C7)cc5)ccc3-4)cccc21 ; CC1(C)c2ccc(-c3cccc4c3C(C)(C)c3ccccc3-4)cc2-c2ccc(N(c3ccccc3)c3ccc(C4CCCC4)cc3)cc21 ; CC1(C)c2ccc(-c3cccc4c3C(C)(C)c3ccccc3-4)cc2-c2ccc(N(c3ccc(-c4ccccc4)cc3)c3ccc(C4CCCCC4)cc3)cc21] 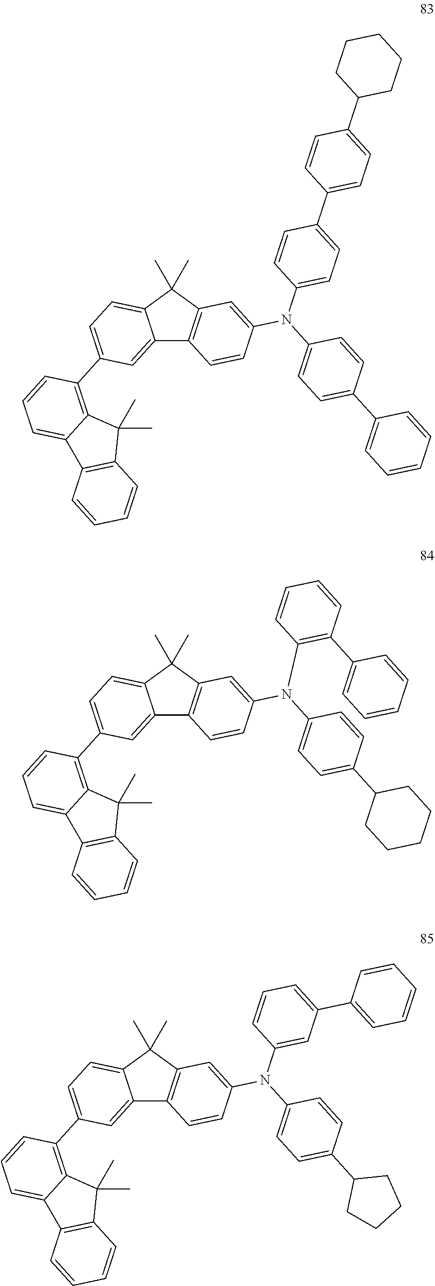 [SMILES: CC1(C)c2ccc(-c3cccc4c3C(C)(C)c3ccccc3-4)cc2-c2ccc(N(c3ccc(-c4ccccc4)cc3)c3ccc(-c4ccc(C5CCCCC5)cc4)cc3)cc21 ; CC1(C)c2ccc(-c3cccc4c3C(C)(C)c3ccccc3-4)cc2-c2ccc(N(c3ccc(C4CCCCC4)cc3)c3ccccc3-c3ccccc3)cc21 ; CC1(C)c2ccc(-c3cccc4c3C(C)(C)c3ccccc3-4)cc2-c2ccc(N(c3ccc(C4CCCC4)cc3)c3cccc(-c4ccccc4)c3)cc21] 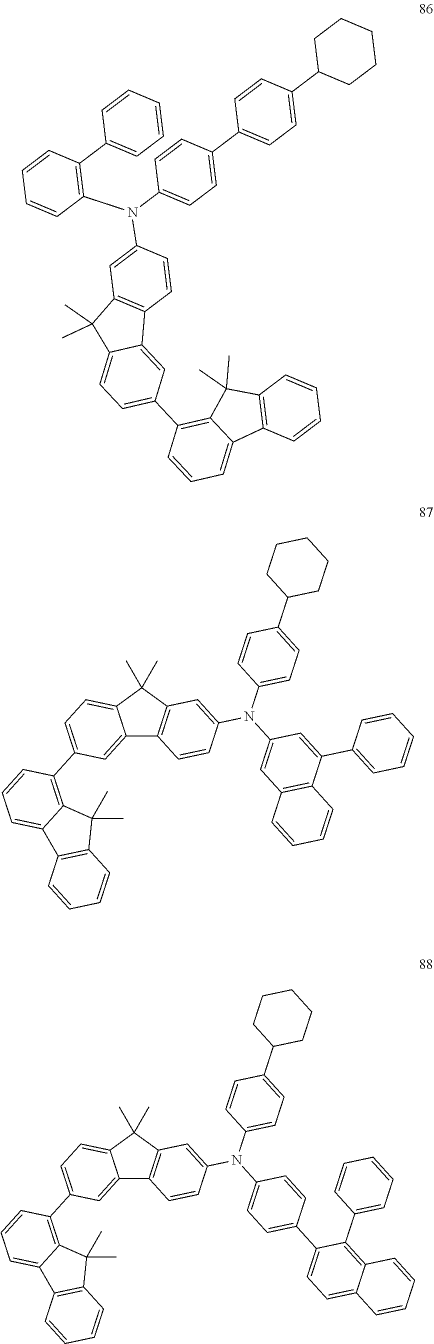 [SMILES: CC1(C)c2ccc(-c3cccc4c3C(C)(C)c3ccccc3-4)cc2-c2ccc(N(c3ccc(-c4ccc(C5CCCCC5)cc4)cc3)c3ccccc3-c3ccccc3)cc21 ; CC1(C)c2ccc(-c3cccc4c3C(C)(C)c3ccccc3-4)cc2-c2ccc(N(c3ccc(C4CCCCC4)cc3)c3cc(-c4ccccc4)c4ccccc4c3)cc21 ; CC1(C)c2ccc(-c3cccc4c3C(C)(C)c3ccccc3-4)cc2-c2ccc(N(c3ccc(-c4ccc5ccccc5c4-c4ccccc4)cc3)c3ccc(C4CCCCC4)cc3)cc21] 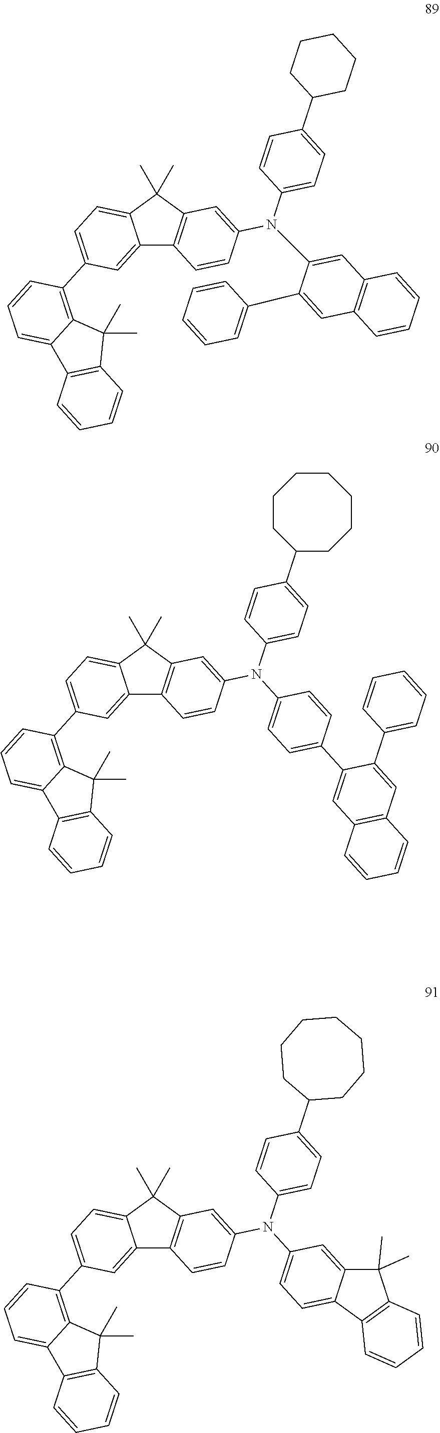 [SMILES: CC1(C)c2ccc(-c3cccc4c3C(C)(C)c3ccccc3-4)cc2-c2ccc(N(c3ccc(C4CCCCC4)cc3)c3cc4ccccc4cc3-c3ccccc3)cc21 ; CC1(C)c2ccc(-c3cccc4c3C(C)(C)c3ccccc3-4)cc2-c2ccc(N(c3ccc(-c4cc5ccccc5cc4-c4ccccc4)cc3)c3ccc(C4CCCCCCC4)cc3)cc21 ; CC1(C)c2ccccc2-c2ccc(N(c3ccc(C4CCCCCCC4)cc3)c3ccc4c(c3)C(C)(C)c3ccc(-c5cccc6c5C(C)(C)c5ccccc5-6)cc3-4)cc21] 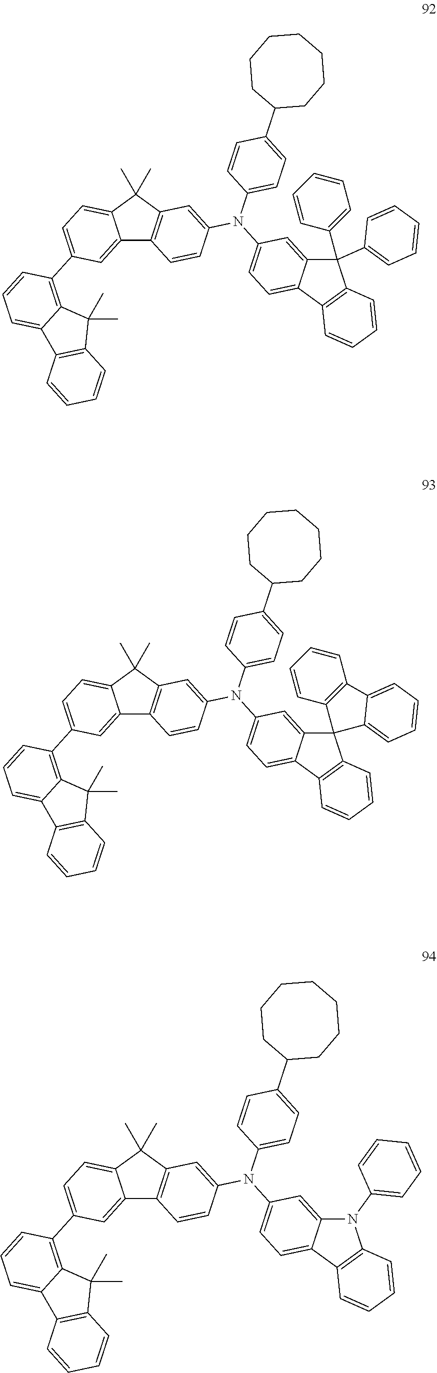 [SMILES: CC1(C)c2ccc(-c3cccc4c3C(C)(C)c3ccccc3-4)cc2-c2ccc(N(c3ccc(C4CCCCCCC4)cc3)c3ccc4c(c3)C(c3ccccc3)(c3ccccc3)c3ccccc3-4)cc21 ; CC1(C)c2ccc(-c3cccc4c3C(C)(C)c3ccccc3-4)cc2-c2ccc(N(c3ccc(C4CCCCCCC4)cc3)c3ccc4c(c3)C3(c5ccccc5-c5ccccc53)c3ccccc3-4)cc21 ; CC1(C)c2ccc(-c3cccc4c3C(C)(C)c3ccccc3-4)cc2-c2ccc(N(c3ccc(C4CCCCCCC4)cc3)c3ccc4c5ccccc5n(-c5ccccc5)c4c3)cc21] 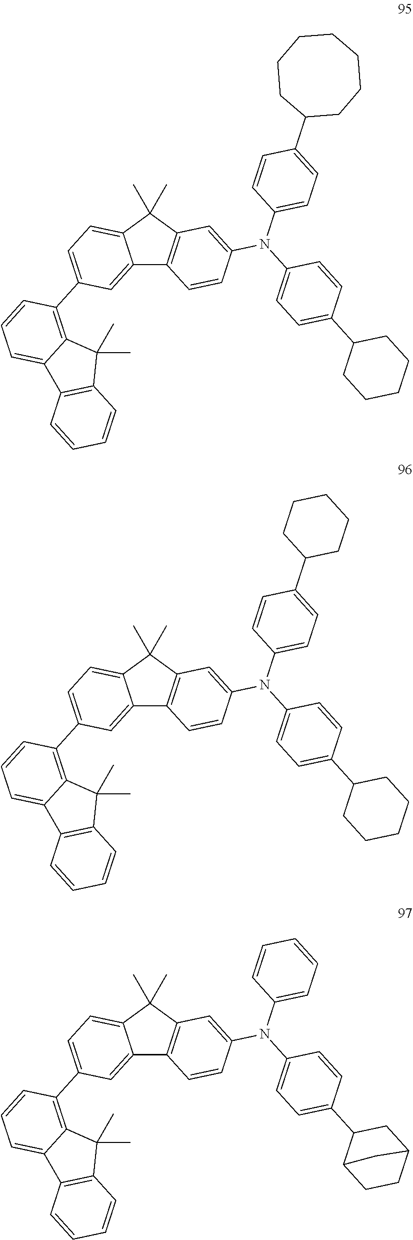 [SMILES: CC1(C)c2ccc(-c3cccc4c3C(C)(C)c3ccccc3-4)cc2-c2ccc(N(c3ccc(C4CCCCCCC4)cc3)c3ccc(C4CCCCC4)cc3)cc21 ; CC1(C)c2ccc(-c3cccc4c3C(C)(C)c3ccccc3-4)cc2-c2ccc(N(c3ccc(C4CCCCC4)cc3)c3ccc(C4CCCCC4)cc3)cc21 ; CC1(C)c2ccc(-c3cccc4c3C(C)(C)c3ccccc3-4)cc2-c2ccc(N(c3ccccc3)c3ccc(C4CC5CCC4C5)cc3)cc21] 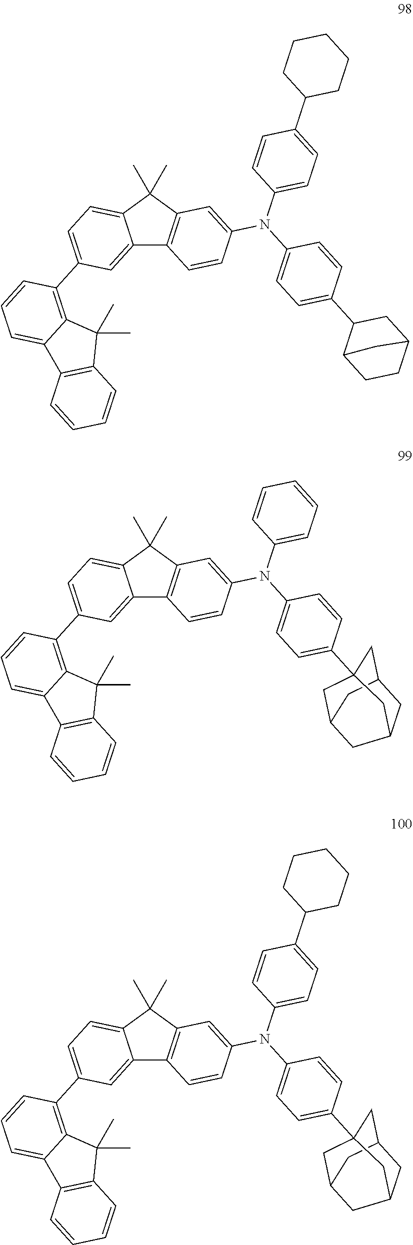 [SMILES: CC1(C)c2ccc(-c3cccc4c3C(C)(C)c3ccccc3-4)cc2-c2ccc(N(c3ccc(C4CCCCC4)cc3)c3ccc(C4CC5CCC4C5)cc3)cc21 ; CC1(C)c2ccc(-c3cccc4c3C(C)(C)c3ccccc3-4)cc2-c2ccc(N(c3ccccc3)c3ccc(C45CC6CC(CC(C6)C4)C5)cc3)cc21 ; CC1(C)c2ccc(-c3cccc4c3C(C)(C)c3ccccc3-4)cc2-c2ccc(N(c3ccc(C4CCCCC4)cc3)c3ccc(C45CC6CC(CC(C6)C4)C5)cc3)cc21] 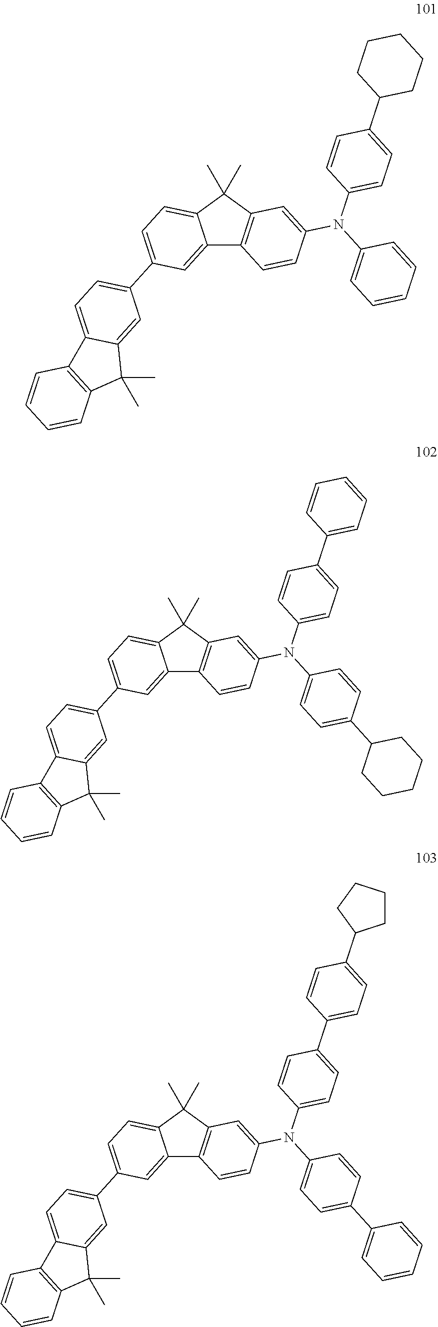 [SMILES: CC1(C)c2ccccc2-c2ccc(-c3ccc4c(c3)-c3ccc(N(c5ccccc5)c5ccc(C6CCCCC6)cc5)cc3C4(C)C)cc21 ; CC1(C)c2ccccc2-c2ccc(-c3ccc4c(c3)-c3ccc(N(c5ccc(-c6ccccc6)cc5)c5ccc(C6CCCCC6)cc5)cc3C4(C)C)cc21 ; CC1(C)c2ccccc2-c2ccc(-c3ccc4c(c3)-c3ccc(N(c5ccc(-c6ccccc6)cc5)c5ccc(-c6ccc(C7CCCC7)cc6)cc5)cc3C4(C)C)cc21] 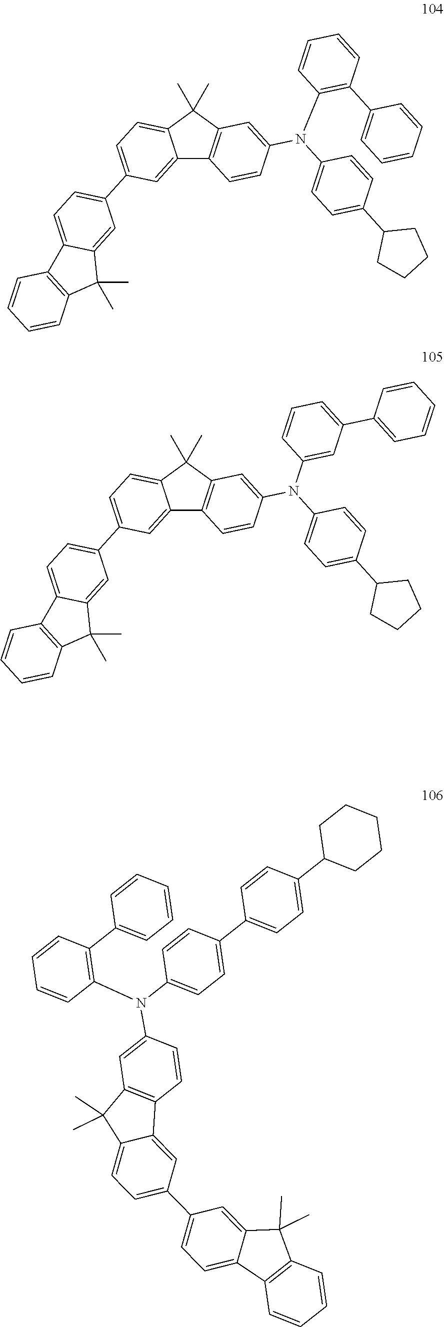 [SMILES: CC1(C)c2ccccc2-c2ccc(-c3ccc4c(c3)-c3ccc(N(c5ccc(C6CCCC6)cc5)c5ccccc5-c5ccccc5)cc3C4(C)C)cc21 ; CC1(C)c2ccccc2-c2ccc(-c3ccc4c(c3)-c3ccc(N(c5ccc(C6CCCC6)cc5)c5cccc(-c6ccccc6)c5)cc3C4(C)C)cc21 ; CC1(C)c2ccccc2-c2ccc(-c3ccc4c(c3)-c3ccc(N(c5ccc(-c6ccc(C7CCCCC7)cc6)cc5)c5ccccc5-c5ccccc5)cc3C4(C)C)cc21] 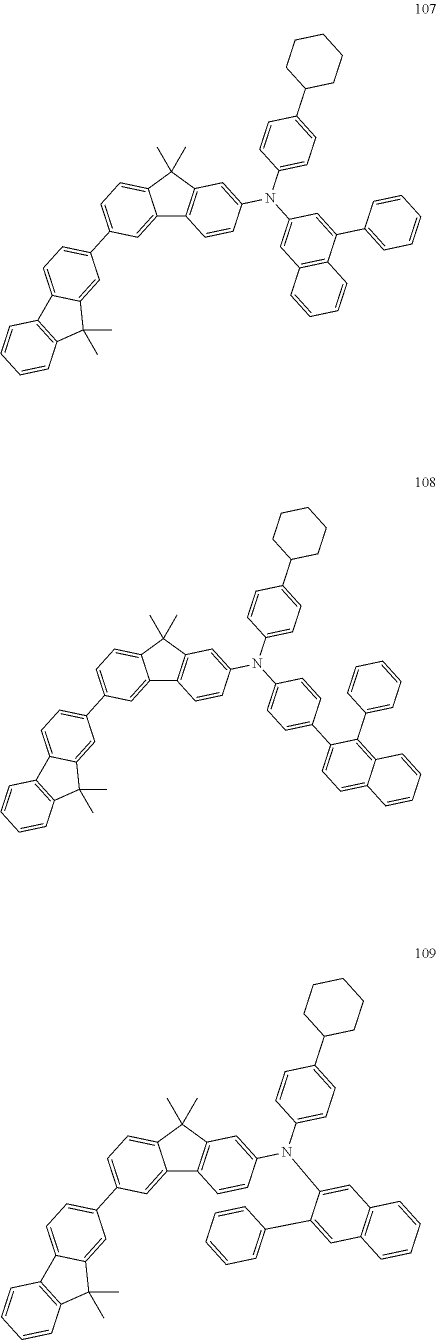 [SMILES: CC1(C)c2ccccc2-c2ccc(-c3ccc4c(c3)-c3ccc(N(c5ccc(C6CCCCC6)cc5)c5cc(-c6ccccc6)c6ccccc6c5)cc3C4(C)C)cc21 ; CC1(C)c2ccccc2-c2ccc(-c3ccc4c(c3)-c3ccc(N(c5ccc(-c6ccc7ccccc7c6-c6ccccc6)cc5)c5ccc(C6CCCCC6)cc5)cc3C4(C)C)cc21 ; CC1(C)c2ccccc2-c2ccc(-c3ccc4c(c3)-c3ccc(N(c5ccc(C6CCCCC6)cc5)c5cc6ccccc6cc5-c5ccccc5)cc3C4(C)C)cc21] 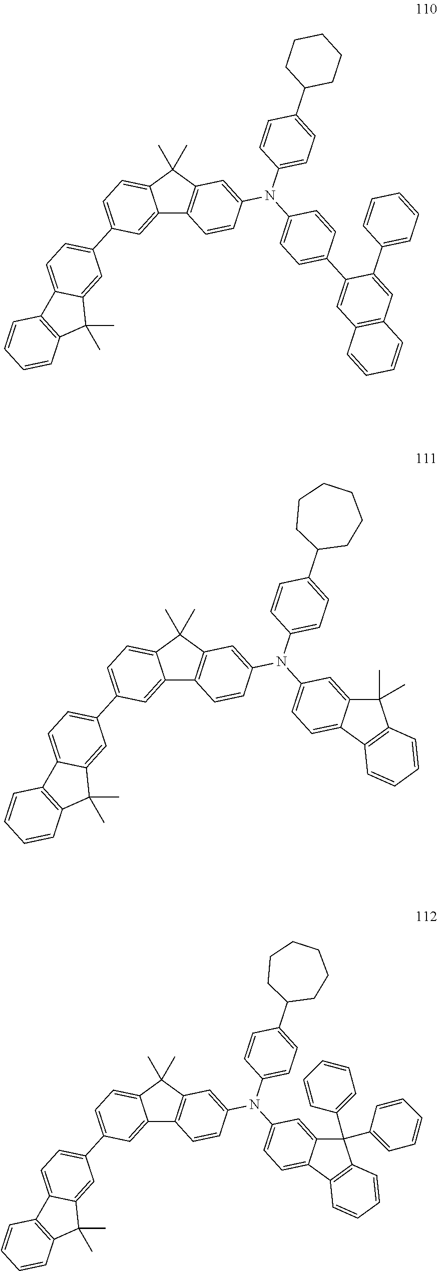 [SMILES: CC1(C)c2ccccc2-c2ccc(-c3ccc4c(c3)-c3ccc(N(c5ccc(-c6cc7ccccc7cc6-c6ccccc6)cc5)c5ccc(C6CCCCC6)cc5)cc3C4(C)C)cc21 ; CC1(C)c2ccccc2-c2ccc(-c3ccc4c(c3)-c3ccc(N(c5ccc(C6CCCCCC6)cc5)c5ccc6c(c5)C(C)(C)c5ccccc5-6)cc3C4(C)C)cc21 ; CC1(C)c2ccccc2-c2ccc(-c3ccc4c(c3)-c3ccc(N(c5ccc(C6CCCCCC6)cc5)c5ccc6c(c5)C(c5ccccc5)(c5ccccc5)c5ccccc5-6)cc3C4(C)C)cc21] 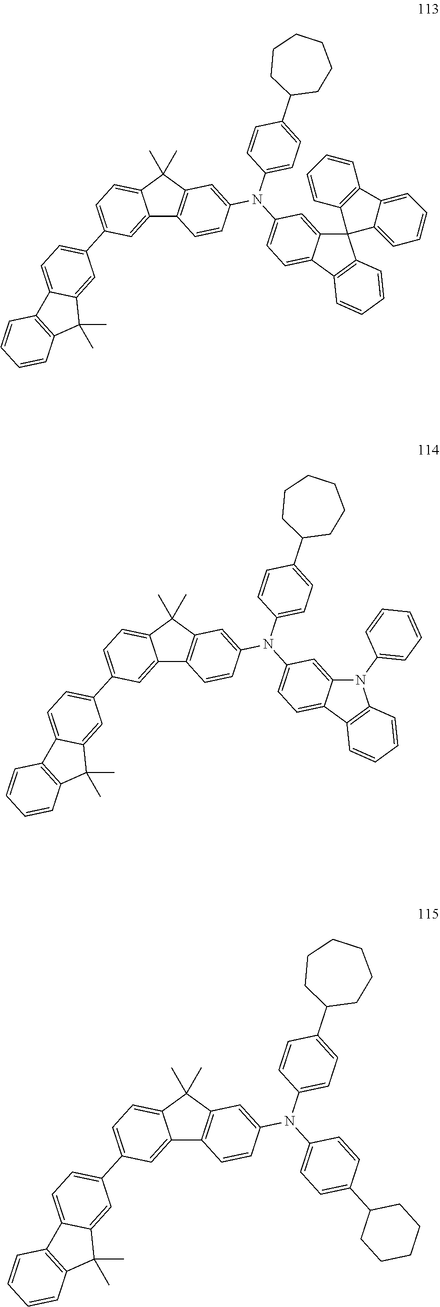 [SMILES: CC1(C)c2ccccc2-c2ccc(-c3ccc4c(c3)-c3ccc(N(c5ccc(C6CCCCCC6)cc5)c5ccc6c(c5)C5(c7ccccc7-c7ccccc75)c5ccccc5-6)cc3C4(C)C)cc21 ; CC1(C)c2ccccc2-c2ccc(-c3ccc4c(c3)-c3ccc(N(c5ccc(C6CCCCCC6)cc5)c5ccc6c7ccccc7n(-c7ccccc7)c6c5)cc3C4(C)C)cc21 ; CC1(C)c2ccccc2-c2ccc(-c3ccc4c(c3)-c3ccc(N(c5ccc(C6CCCCCC6)cc5)c5ccc(C6CCCCC6)cc5)cc3C4(C)C)cc21] 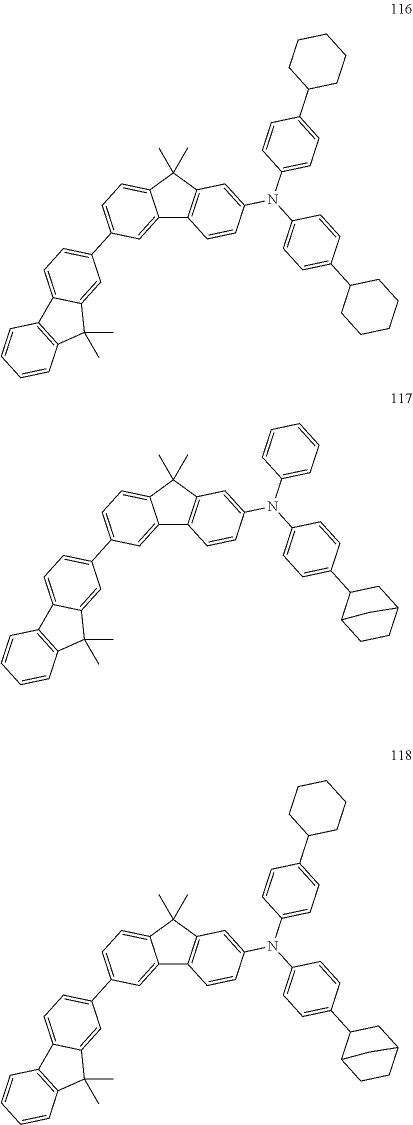 [SMILES: CC1(C)c2ccccc2-c2ccc(-c3ccc4c(c3)-c3ccc(N(c5ccc(C6CCCCC6)cc5)c5ccc(C6CCCCC6)cc5)cc3C4(C)C)cc21 ; CC1(C)c2ccccc2-c2ccc(-c3ccc4c(c3)-c3ccc(N(c5ccccc5)c5ccc(C6CC7CCC6C7)cc5)cc3C4(C)C)cc21 ; CC1(C)c2ccccc2-c2ccc(-c3ccc4c(c3)-c3ccc(N(c5ccc(C6CCCCC6)cc5)c5ccc(C6CC7CCC6C7)cc5)cc3C4(C)C)cc21] 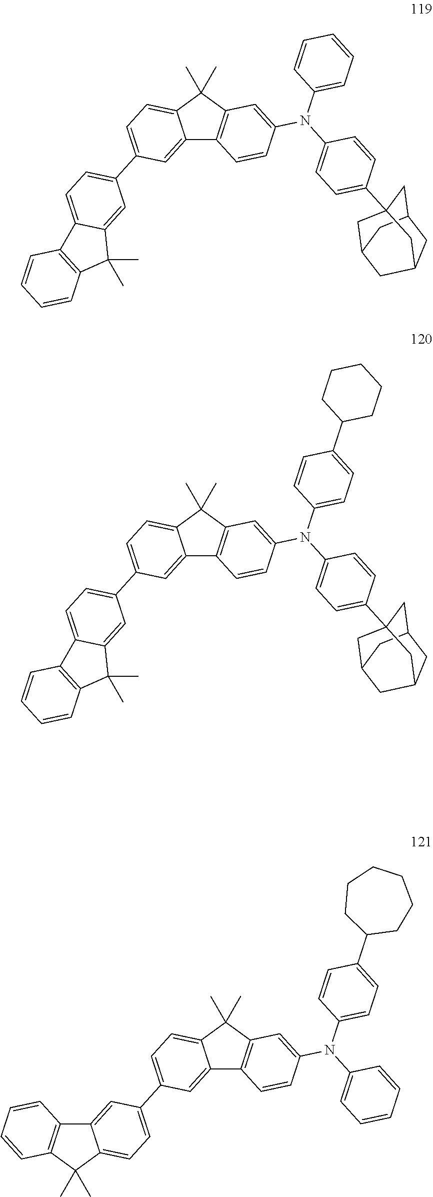 [SMILES: CC1(C)c2ccccc2-c2ccc(-c3ccc4c(c3)-c3ccc(N(c5ccccc5)c5ccc(C67CC8CC(CC(C8)C6)C7)cc5)cc3C4(C)C)cc21 ; CC1(C)c2ccccc2-c2ccc(-c3ccc4c(c3)-c3ccc(N(c5ccc(C6CCCCC6)cc5)c5ccc(C67CC8CC(CC(C8)C6)C7)cc5)cc3C4(C)C)cc21 ; CC1(C)c2ccccc2-c2cc(-c3ccc4c(c3)-c3ccc(N(c5ccccc5)c5ccc(C6CCCCCC6)cc5)cc3C4(C)C)ccc21] 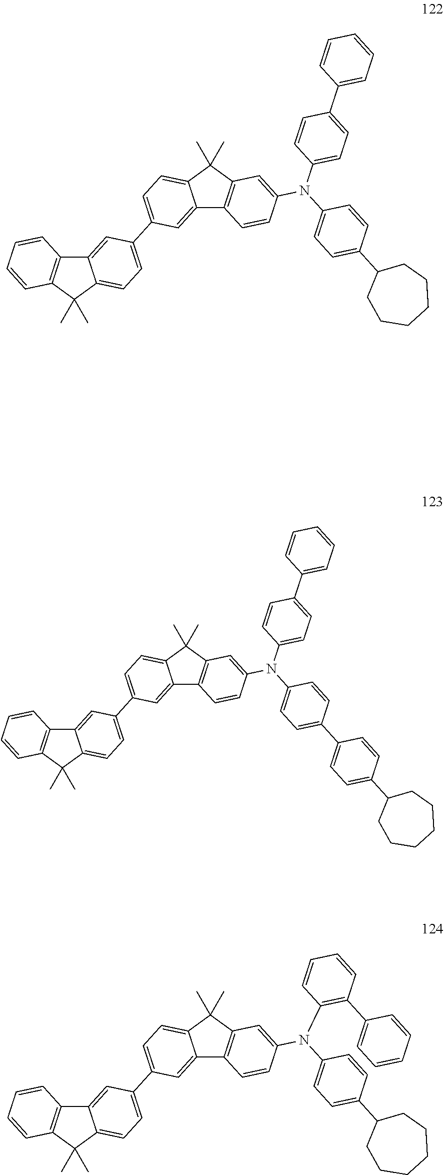 [SMILES: CC1(C)c2ccccc2-c2cc(-c3ccc4c(c3)-c3ccc(N(c5ccc(-c6ccccc6)cc5)c5ccc(C6CCCCCC6)cc5)cc3C4(C)C)ccc21 ; CC1(C)c2ccccc2-c2cc(-c3ccc4c(c3)-c3ccc(N(c5ccc(-c6ccccc6)cc5)c5ccc(-c6ccc(C7CCCCCC7)cc6)cc5)cc3C4(C)C)ccc21 ; CC1(C)c2ccccc2-c2cc(-c3ccc4c(c3)-c3ccc(N(c5ccc(C6CCCCCC6)cc5)c5ccccc5-c5ccccc5)cc3C4(C)C)ccc21] 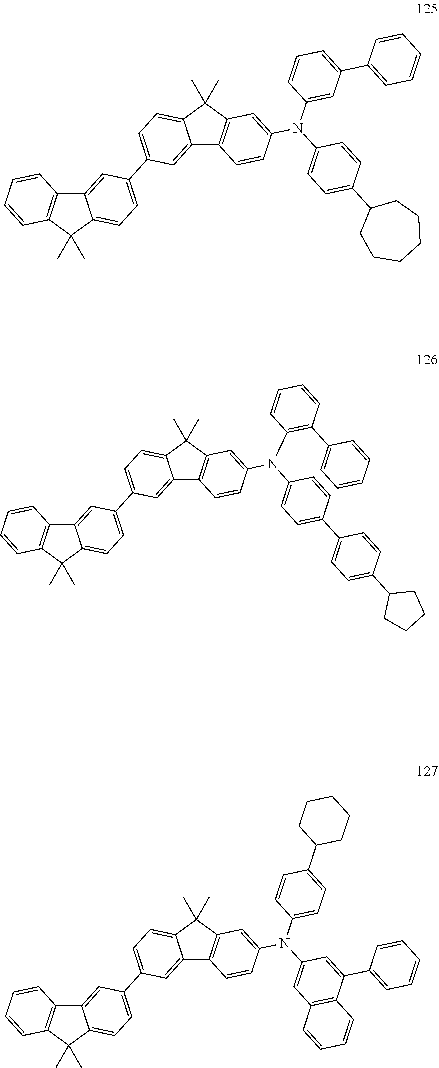 [SMILES: CC1(C)c2ccccc2-c2cc(-c3ccc4c(c3)-c3ccc(N(c5ccc(C6CCCCCC6)cc5)c5cccc(-c6ccccc6)c5)cc3C4(C)C)ccc21 ; CC1(C)c2ccccc2-c2cc(-c3ccc4c(c3)-c3ccc(N(c5ccc(-c6ccc(C7CCCC7)cc6)cc5)c5ccccc5-c5ccccc5)cc3C4(C)C)ccc21 ; CC1(C)c2ccccc2-c2cc(-c3ccc4c(c3)-c3ccc(N(c5ccc(C6CCCCC6)cc5)c5cc(-c6ccccc6)c6ccccc6c5)cc3C4(C)C)ccc21] 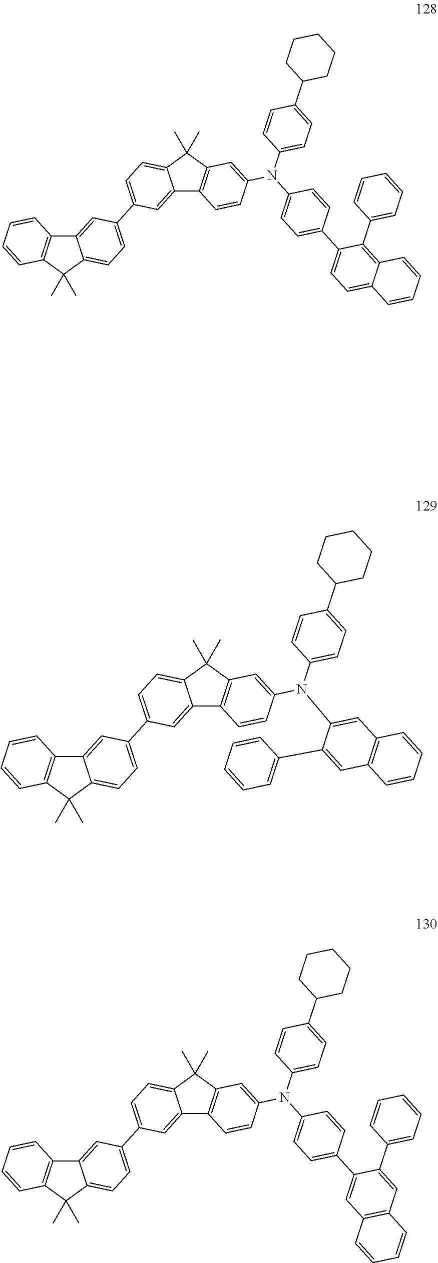 [SMILES: CC1(C)c2ccccc2-c2cc(-c3ccc4c(c3)-c3ccc(N(c5ccc(-c6ccc7ccccc7c6-c6ccccc6)cc5)c5ccc(C6CCCCC6)cc5)cc3C4(C)C)ccc21 ; CC1(C)c2ccccc2-c2cc(-c3ccc4c(c3)-c3ccc(N(c5ccc(C6CCCCC6)cc5)c5cc6ccccc6cc5-c5ccccc5)cc3C4(C)C)ccc21 ; CC1(C)c2ccccc2-c2cc(-c3ccc4c(c3)-c3ccc(N(c5ccc(-c6cc7ccccc7cc6-c6ccccc6)cc5)c5ccc(C6CCCCC6)cc5)cc3C4(C)C)ccc21] 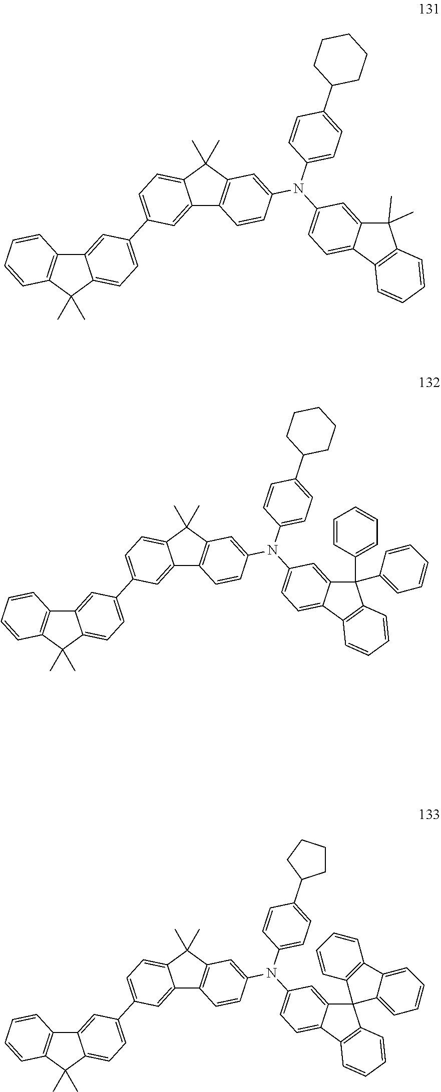 [SMILES: CC1(C)c2ccccc2-c2cc(-c3ccc4c(c3)-c3ccc(N(c5ccc(C6CCCCC6)cc5)c5ccc6c(c5)C(C)(C)c5ccccc5-6)cc3C4(C)C)ccc21 ; CC1(C)c2ccccc2-c2cc(-c3ccc4c(c3)-c3ccc(N(c5ccc(C6CCCCC6)cc5)c5ccc6c(c5)C(c5ccccc5)(c5ccccc5)c5ccccc5-6)cc3C4(C)C)ccc21 ; CC1(C)c2ccccc2-c2cc(-c3ccc4c(c3)-c3ccc(N(c5ccc(C6CCCC6)cc5)c5ccc6c(c5)C5(c7ccccc7-c7ccccc75)c5ccccc5-6)cc3C4(C)C)ccc21] 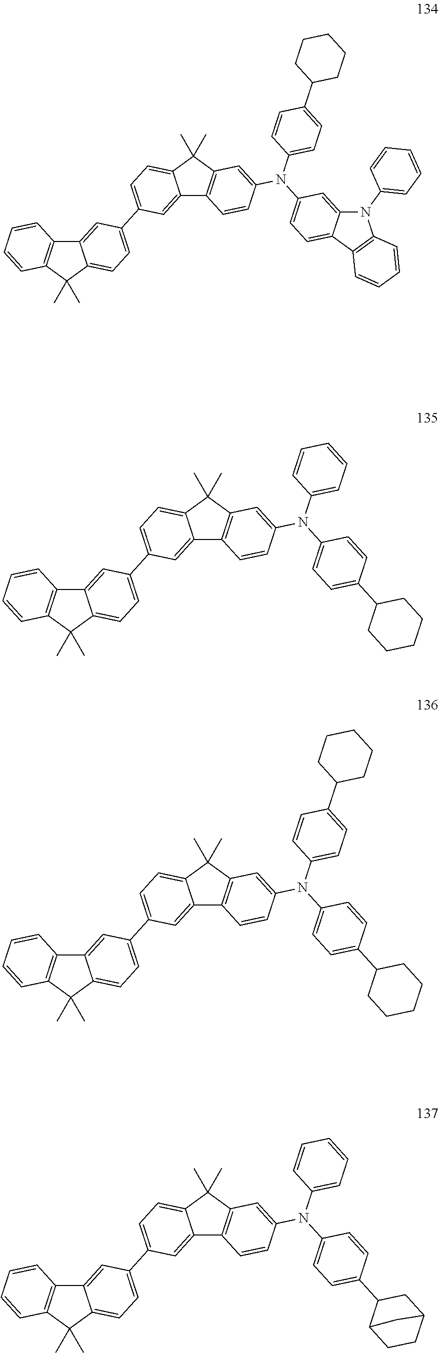 [SMILES: CC1(C)c2ccccc2-c2cc(-c3ccc4c(c3)-c3ccc(N(c5ccc(C6CCCCC6)cc5)c5ccc6c7ccccc7n(-c7ccccc7)c6c5)cc3C4(C)C)ccc21 ; CC1(C)c2ccccc2-c2cc(-c3ccc4c(c3)-c3ccc(N(c5ccccc5)c5ccc(C6CCCCC6)cc5)cc3C4(C)C)ccc21 ; CC1(C)c2ccccc2-c2cc(-c3ccc4c(c3)-c3ccc(N(c5ccc(C6CCCCC6)cc5)c5ccc(C6CCCCC6)cc5)cc3C4(C)C)ccc21 ; CC1(C)c2ccccc2-c2cc(-c3ccc4c(c3)-c3ccc(N(c5ccccc5)c5ccc(C6CC7CCC6C7)cc5)cc3C4(C)C)ccc21] 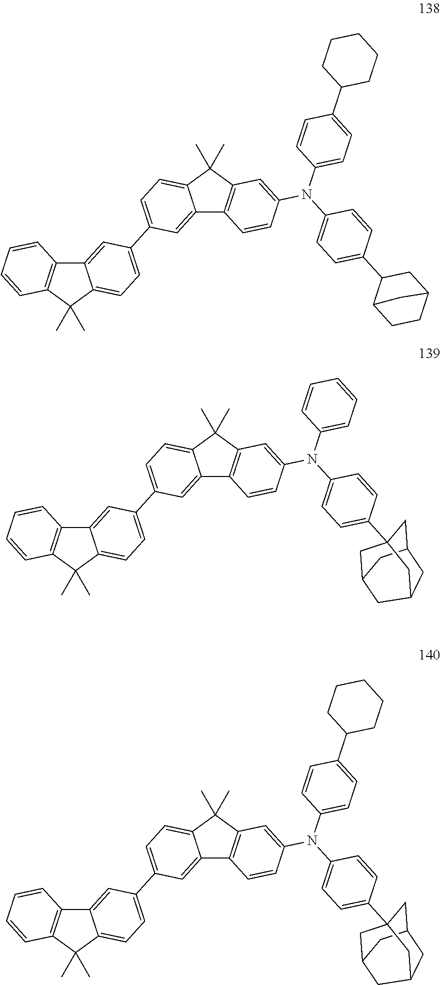 [SMILES: CC1(C)c2ccccc2-c2cc(-c3ccc4c(c3)-c3ccc(N(c5ccc(C6CCCCC6)cc5)c5ccc(C6CC7CCC6C7)cc5)cc3C4(C)C)ccc21 ; CC1(C)c2ccccc2-c2cc(-c3ccc4c(c3)-c3ccc(N(c5ccccc5)c5ccc(C67CC8CC(CC(C8)C6)C7)cc5)cc3C4(C)C)ccc21 ; CC1(C)c2ccccc2-c2cc(-c3ccc4c(c3)-c3ccc(N(c5ccc(C6CCCCC6)cc5)c5ccc(C67CC8CC(CC(C8)C6)C7)cc5)cc3C4(C)C)ccc21] 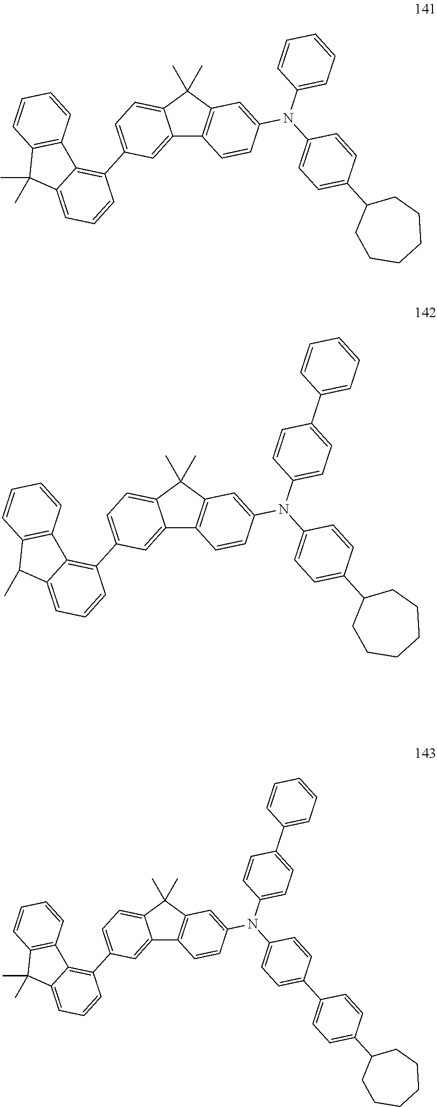 [SMILES: CC1(C)c2ccc(-c3cccc4c3-c3ccccc3C4(C)C)cc2-c2ccc(N(c3ccccc3)c3ccc(C4CCCCCC4)cc3)cc21 ; CC1c2ccccc2-c2c(-c3ccc4c(c3)-c3ccc(N(c5ccc(-c6ccccc6)cc5)c5ccc(C6CCCCCC6)cc5)cc3C4(C)C)cccc21 ; CC1(C)c2ccc(-c3cccc4c3-c3ccccc3C4(C)C)cc2-c2ccc(N(c3ccc(-c4ccccc4)cc3)c3ccc(-c4ccc(C5CCCCCC5)cc4)cc3)cc21] 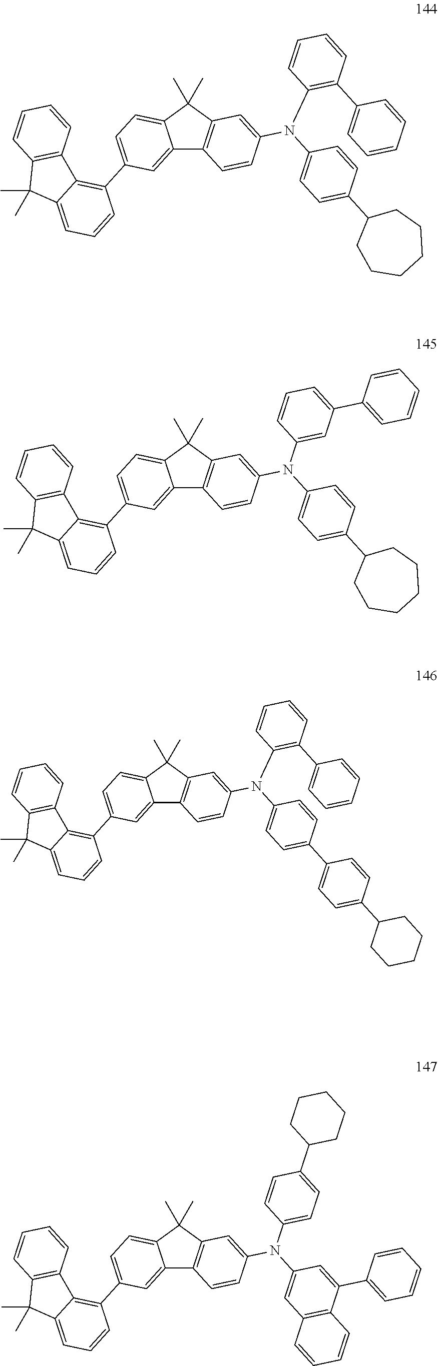 [SMILES: CC1(C)c2ccc(-c3cccc4c3-c3ccccc3C4(C)C)cc2-c2ccc(N(c3ccc(C4CCCCCC4)cc3)c3ccccc3-c3ccccc3)cc21 ; CC1(C)c2ccc(-c3cccc4c3-c3ccccc3C4(C)C)cc2-c2ccc(N(c3ccc(C4CCCCCC4)cc3)c3cccc(-c4ccccc4)c3)cc21 ; CC1(C)c2ccc(-c3cccc4c3-c3ccccc3C4(C)C)cc2-c2ccc(N(c3ccc(-c4ccc(C5CCCCC5)cc4)cc3)c3ccccc3-c3ccccc3)cc21 ; CC1(C)c2ccc(-c3cccc4c3-c3ccccc3C4(C)C)cc2-c2ccc(N(c3ccc(C4CCCCC4)cc3)c3cc(-c4ccccc4)c4ccccc4c3)cc21] 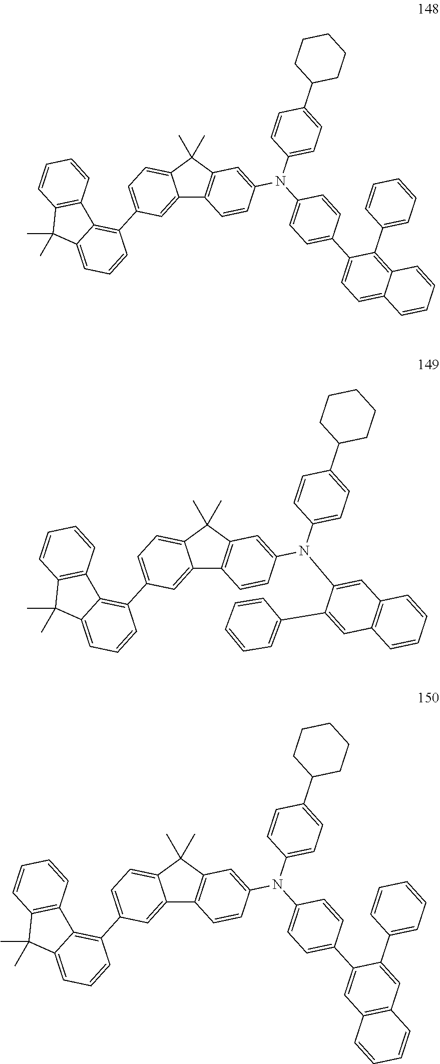 [SMILES: CC1(C)c2ccc(-c3cccc4c3-c3ccccc3C4(C)C)cc2-c2ccc(N(c3ccc(-c4ccc5ccccc5c4-c4ccccc4)cc3)c3ccc(C4CCCCC4)cc3)cc21 ; CC1(C)c2ccc(-c3cccc4c3-c3ccccc3C4(C)C)cc2-c2ccc(N(c3ccc(C4CCCCC4)cc3)c3cc4ccccc4cc3-c3ccccc3)cc21 ; CC1(C)c2ccc(-c3cccc4c3-c3ccccc3C4(C)C)cc2-c2ccc(N(c3ccc(-c4cc5ccccc5cc4-c4ccccc4)cc3)c3ccc(C4CCCCC4)cc3)cc21] 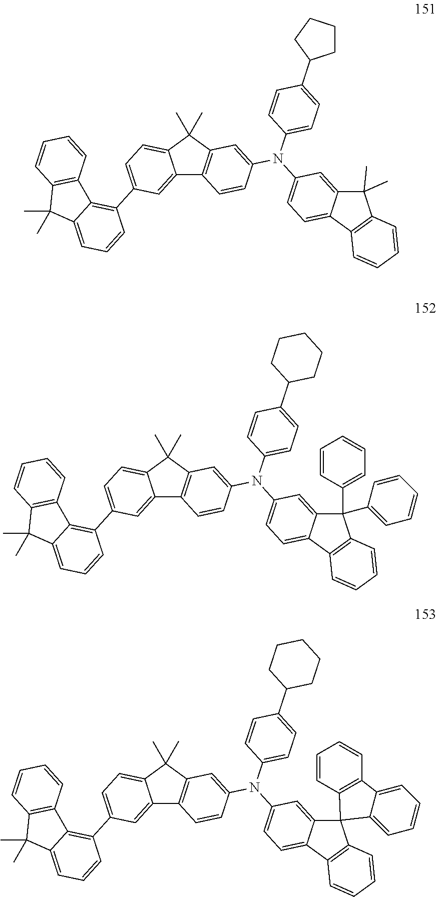 [SMILES: CC1(C)c2ccccc2-c2ccc(N(c3ccc(C4CCCC4)cc3)c3ccc4c(c3)C(C)(C)c3ccc(-c5cccc6c5-c5ccccc5C6(C)C)cc3-4)cc21 ; CC1(C)c2ccc(-c3cccc4c3-c3ccccc3C4(C)C)cc2-c2ccc(N(c3ccc(C4CCCCC4)cc3)c3ccc4c(c3)C(c3ccccc3)(c3ccccc3)c3ccccc3-4)cc21 ; CC1(C)c2ccc(-c3cccc4c3-c3ccccc3C4(C)C)cc2-c2ccc(N(c3ccc(C4CCCCC4)cc3)c3ccc4c(c3)C3(c5ccccc5-c5ccccc53)c3ccccc3-4)cc21] 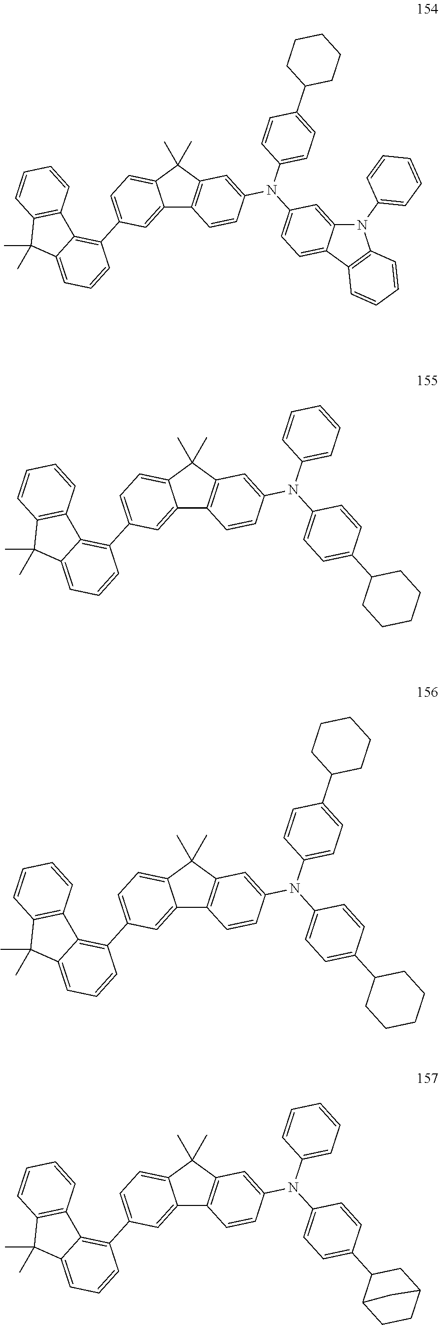 [SMILES: CC1(C)c2ccc(-c3cccc4c3-c3ccccc3C4(C)C)cc2-c2ccc(N(c3ccc(C4CCCCC4)cc3)c3ccc4c5ccccc5n(-c5ccccc5)c4c3)cc21 ; CC1(C)c2ccc(-c3cccc4c3-c3ccccc3C4(C)C)cc2-c2ccc(N(c3ccccc3)c3ccc(C4CCCCC4)cc3)cc21 ; CC1(C)c2ccc(-c3cccc4c3-c3ccccc3C4(C)C)cc2-c2ccc(N(c3ccc(C4CCCCC4)cc3)c3ccc(C4CCCCC4)cc3)cc21 ; CC1(C)c2ccc(-c3cccc4c3-c3ccccc3C4(C)C)cc2-c2ccc(N(c3ccccc3)c3ccc(C4CC5CCC4C5)cc3)cc21] 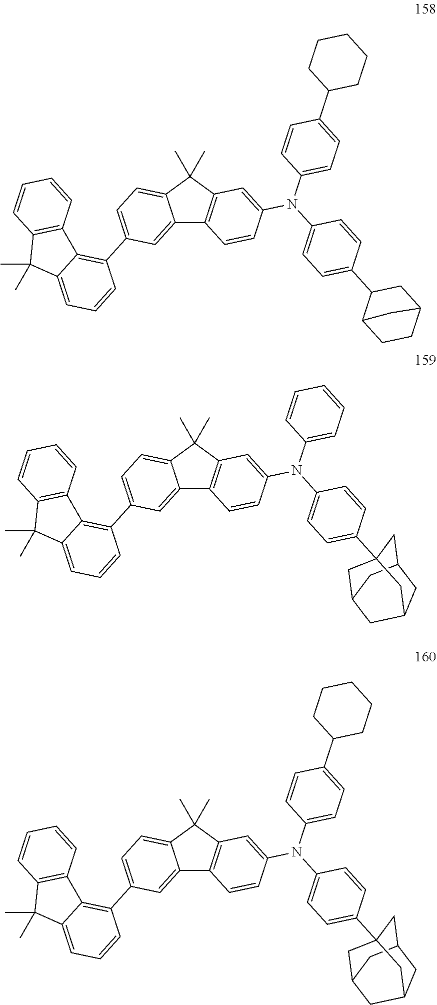 [SMILES: CC1(C)c2ccc(-c3cccc4c3-c3ccccc3C4(C)C)cc2-c2ccc(N(c3ccc(C4CCCCC4)cc3)c3ccc(C4CC5CCC4C5)cc3)cc21 ; CC1(C)c2ccc(-c3cccc4c3-c3ccccc3C4(C)C)cc2-c2ccc(N(c3ccccc3)c3ccc(C45CC6CC(CC(C6)C4)C5)cc3)cc21 ; CC1(C)c2ccc(-c3cccc4c3-c3ccccc3C4(C)C)cc2-c2ccc(N(c3ccc(C4CCCCC4)cc3)c3ccc(C45CC6CC(CC(C6)C4)C5)cc3)cc21] 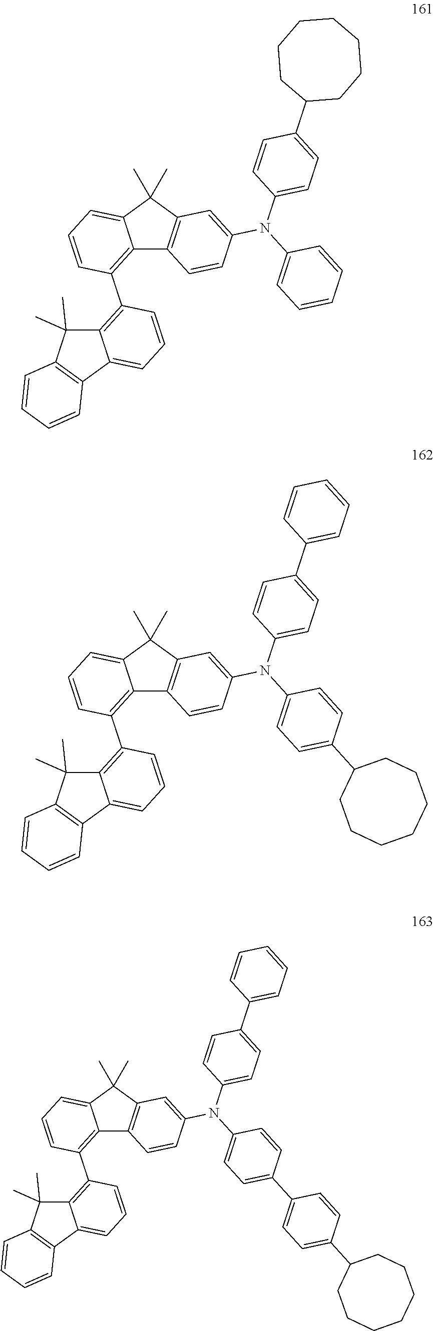 [SMILES: CC1(C)c2cc(N(c3ccccc3)c3ccc(C4CCCCCCC4)cc3)ccc2-c2c(-c3cccc4c3C(C)(C)c3ccccc3-4)cccc21 ; CC1(C)c2cc(N(c3ccc(-c4ccccc4)cc3)c3ccc(C4CCCCCCC4)cc3)ccc2-c2c(-c3cccc4c3C(C)(C)c3ccccc3-4)cccc21 ; CC1(C)c2cc(N(c3ccc(-c4ccccc4)cc3)c3ccc(-c4ccc(C5CCCCCCC5)cc4)cc3)ccc2-c2c(-c3cccc4c3C(C)(C)c3ccccc3-4)cccc21] 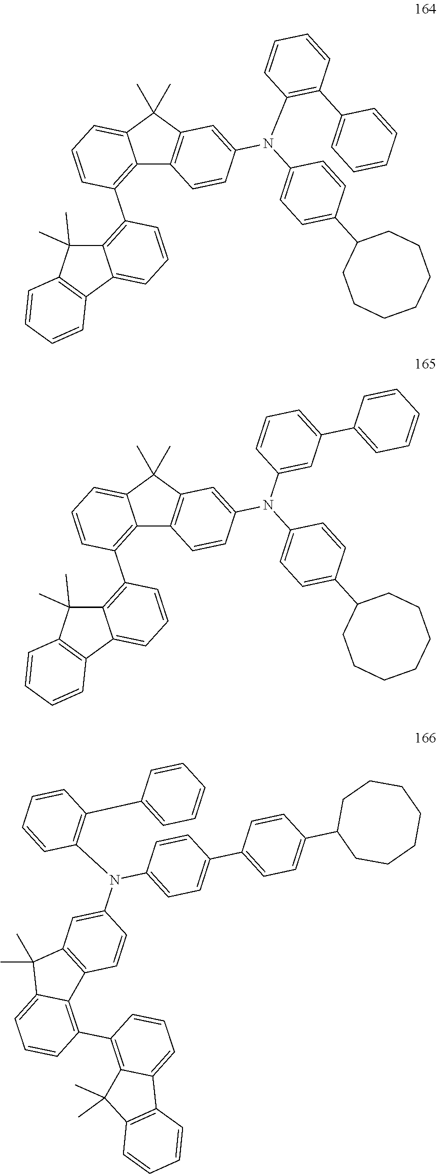 [SMILES: CC1(C)c2cc(N(c3ccc(C4CCCCCCC4)cc3)c3ccccc3-c3ccccc3)ccc2-c2c(-c3cccc4c3C(C)(C)c3ccccc3-4)cccc21 ; CC1(C)c2cc(N(c3ccc(C4CCCCCCC4)cc3)c3cccc(-c4ccccc4)c3)ccc2-c2c(-c3cccc4c3C(C)(C)c3ccccc3-4)cccc21 ; CC1(C)c2cc(N(c3ccc(-c4ccc(C5CCCCCCC5)cc4)cc3)c3ccccc3-c3ccccc3)ccc2-c2c(-c3cccc4c3C(C)(C)c3ccccc3-4)cccc21] 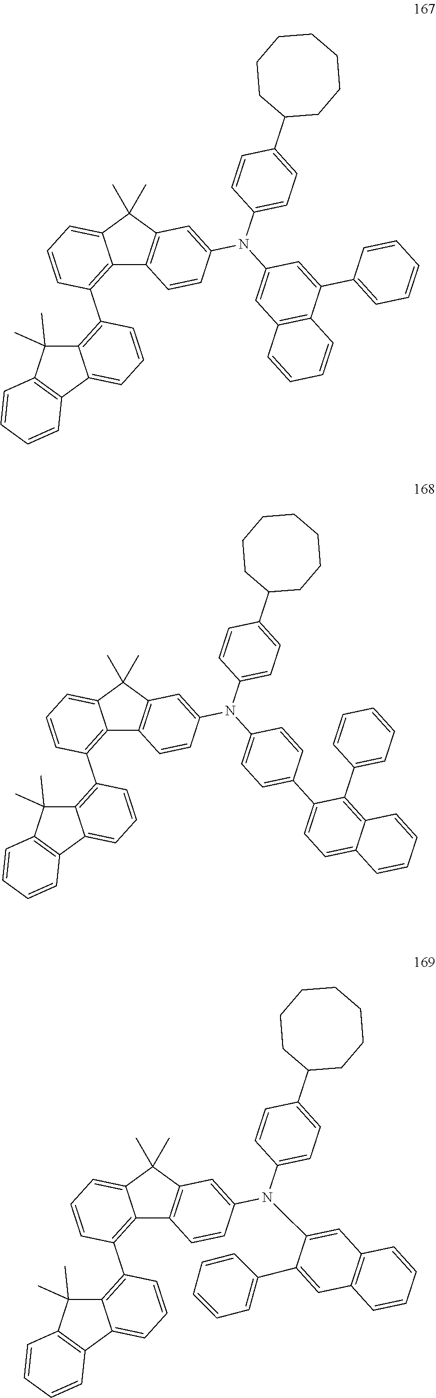 [SMILES: CC1(C)c2cc(N(c3ccc(C4CCCCCCC4)cc3)c3cc(-c4ccccc4)c4ccccc4c3)ccc2-c2c(-c3cccc4c3C(C)(C)c3ccccc3-4)cccc21 ; CC1(C)c2cc(N(c3ccc(-c4ccc5ccccc5c4-c4ccccc4)cc3)c3ccc(C4CCCCCCC4)cc3)ccc2-c2c(-c3cccc4c3C(C)(C)c3ccccc3-4)cccc21 ; CC1(C)c2cc(N(c3ccc(C4CCCCCCC4)cc3)c3cc4ccccc4cc3-c3ccccc3)ccc2-c2c(-c3cccc4c3C(C)(C)c3ccccc3-4)cccc21] 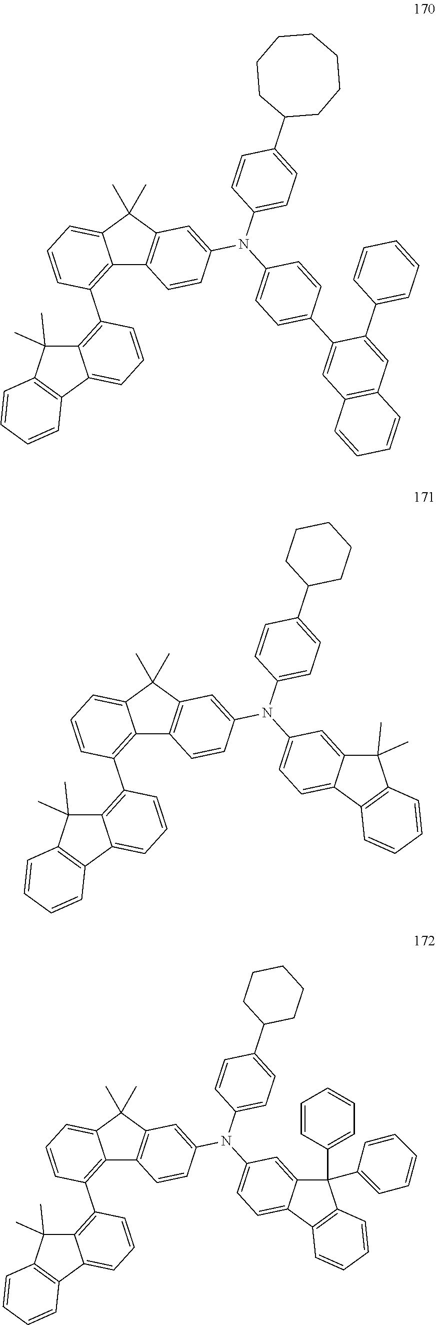 [SMILES: CC1(C)c2cc(N(c3ccc(-c4cc5ccccc5cc4-c4ccccc4)cc3)c3ccc(C4CCCCCCC4)cc3)ccc2-c2c(-c3cccc4c3C(C)(C)c3ccccc3-4)cccc21 ; CC1(C)c2ccccc2-c2ccc(N(c3ccc(C4CCCCC4)cc3)c3ccc4c(c3)C(C)(C)c3cccc(-c5cccc6c5C(C)(C)c5ccccc5-6)c3-4)cc21 ; CC1(C)c2cc(N(c3ccc(C4CCCCC4)cc3)c3ccc4c(c3)C(c3ccccc3)(c3ccccc3)c3ccccc3-4)ccc2-c2c(-c3cccc4c3C(C)(C)c3ccccc3-4)cccc21] 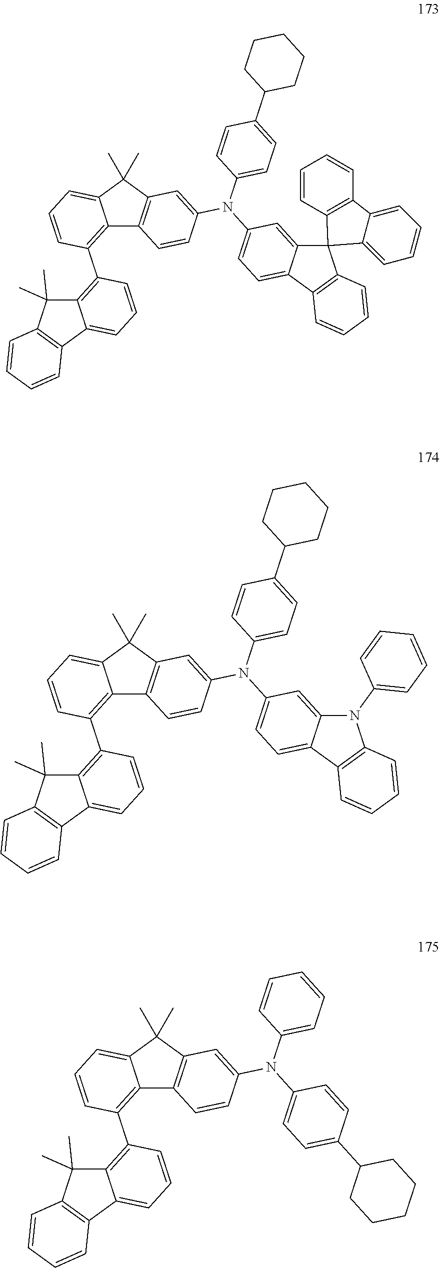 [SMILES: CC1(C)c2cc(N(c3ccc(C4CCCCC4)cc3)c3ccc4c(c3)C3(c5ccccc5-c5ccccc53)c3ccccc3-4)ccc2-c2c(-c3cccc4c3C(C)(C)c3ccccc3-4)cccc21 ; CC1(C)c2cc(N(c3ccc(C4CCCCC4)cc3)c3ccc4c5ccccc5n(-c5ccccc5)c4c3)ccc2-c2c(-c3cccc4c3C(C)(C)c3ccccc3-4)cccc21 ; CC1(C)c2cc(N(c3ccccc3)c3ccc(C4CCCCC4)cc3)ccc2-c2c(-c3cccc4c3C(C)(C)c3ccccc3-4)cccc21] 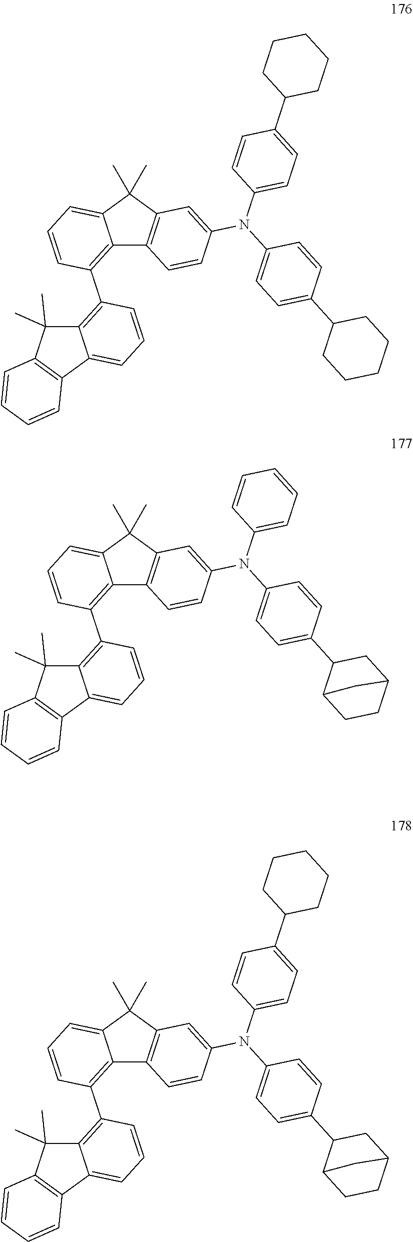 [SMILES: CC1(C)c2cc(N(c3ccc(C4CCCCC4)cc3)c3ccc(C4CCCCC4)cc3)ccc2-c2c(-c3cccc4c3C(C)(C)c3ccccc3-4)cccc21 ; CC1(C)c2cc(N(c3ccccc3)c3ccc(C4CC5CCC4C5)cc3)ccc2-c2c(-c3cccc4c3C(C)(C)c3ccccc3-4)cccc21 ; CC1(C)c2cc(N(c3ccc(C4CCCCC4)cc3)c3ccc(C4CC5CCC4C5)cc3)ccc2-c2c(-c3cccc4c3C(C)(C)c3ccccc3-4)cccc21] 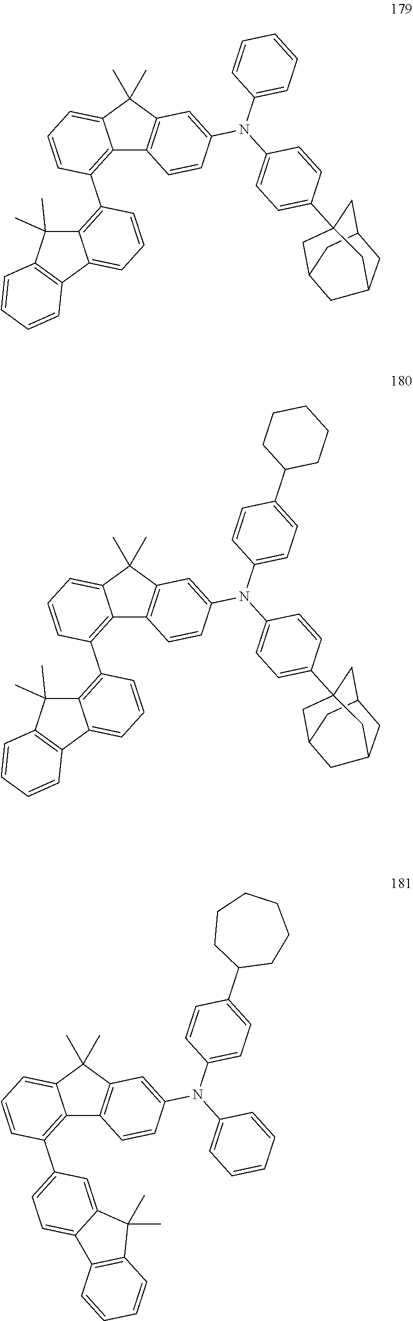 [SMILES: CC1(C)c2cc(N(c3ccccc3)c3ccc(C45CC6CC(CC(C6)C4)C5)cc3)ccc2-c2c(-c3cccc4c3C(C)(C)c3ccccc3-4)cccc21 ; CC1(C)c2cc(N(c3ccc(C4CCCCC4)cc3)c3ccc(C45CC6CC(CC(C6)C4)C5)cc3)ccc2-c2c(-c3cccc4c3C(C)(C)c3ccccc3-4)cccc21 ; CC1(C)c2ccccc2-c2ccc(-c3cccc4c3-c3ccc(N(c5ccccc5)c5ccc(C6CCCCCC6)cc5)cc3C4(C)C)cc21] 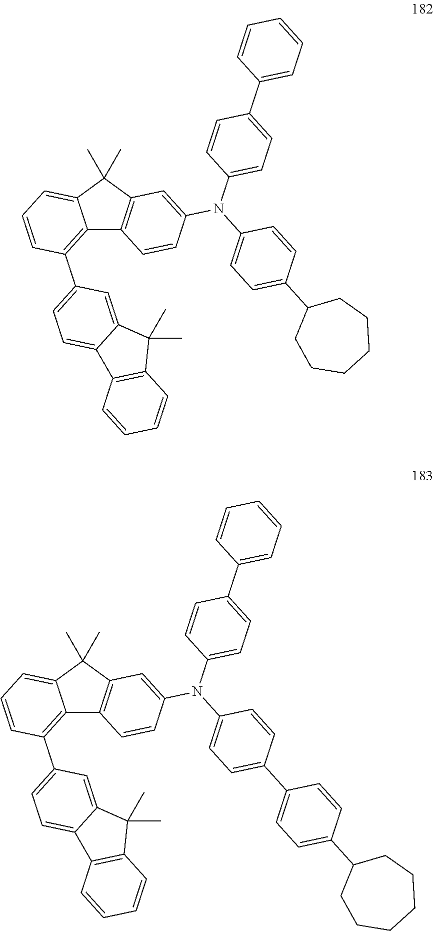 [SMILES: CC1(C)c2ccccc2-c2ccc(-c3cccc4c3-c3ccc(N(c5ccc(-c6ccccc6)cc5)c5ccc(C6CCCCCC6)cc5)cc3C4(C)C)cc21 ; CC1(C)c2ccccc2-c2ccc(-c3cccc4c3-c3ccc(N(c5ccc(-c6ccccc6)cc5)c5ccc(-c6ccc(C7CCCCCC7)cc6)cc5)cc3C4(C)C)cc21] 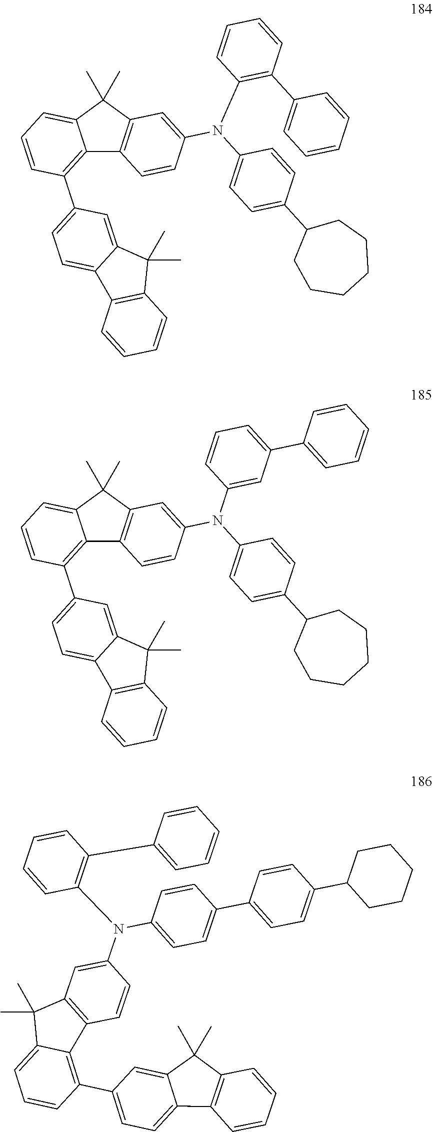 [SMILES: CC1(C)c2ccccc2-c2ccc(-c3cccc4c3-c3ccc(N(c5ccc(C6CCCCCC6)cc5)c5ccccc5-c5ccccc5)cc3C4(C)C)cc21 ; CC1(C)c2ccccc2-c2ccc(-c3cccc4c3-c3ccc(N(c5ccc(C6CCCCCC6)cc5)c5cccc(-c6ccccc6)c5)cc3C4(C)C)cc21 ; CC1(C)c2ccccc2-c2ccc(-c3cccc4c3-c3ccc(N(c5ccc(-c6ccc(C7CCCCC7)cc6)cc5)c5ccccc5-c5ccccc5)cc3C4(C)C)cc21] 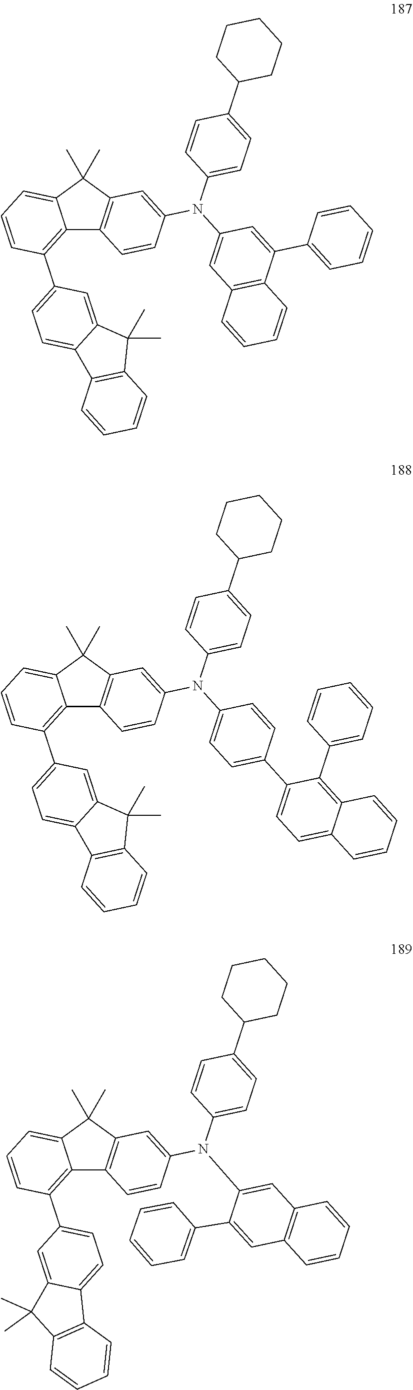 [SMILES: CC1(C)c2ccccc2-c2ccc(-c3cccc4c3-c3ccc(N(c5ccc(C6CCCCC6)cc5)c5cc(-c6ccccc6)c6ccccc6c5)cc3C4(C)C)cc21 ; CC1(C)c2ccccc2-c2ccc(-c3cccc4c3-c3ccc(N(c5ccc(-c6ccc7ccccc7c6-c6ccccc6)cc5)c5ccc(C6CCCCC6)cc5)cc3C4(C)C)cc21 ; CC1(C)c2ccccc2-c2ccc(-c3cccc4c3-c3ccc(N(c5ccc(C6CCCCC6)cc5)c5cc6ccccc6cc5-c5ccccc5)cc3C4(C)C)cc21] 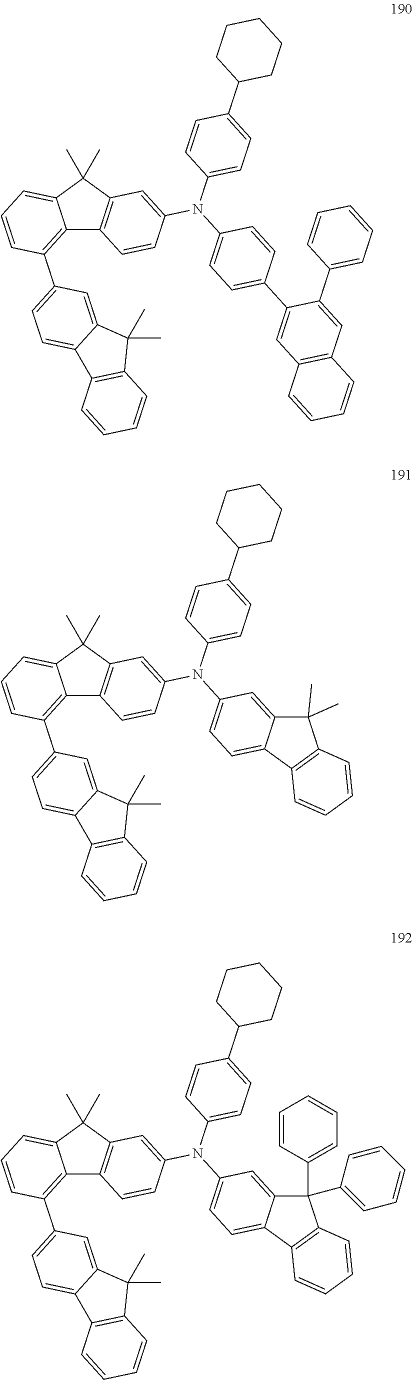 [SMILES: CC1(C)c2ccccc2-c2ccc(-c3cccc4c3-c3ccc(N(c5ccc(-c6cc7ccccc7cc6-c6ccccc6)cc5)c5ccc(C6CCCCC6)cc5)cc3C4(C)C)cc21 ; CC1(C)c2ccccc2-c2ccc(-c3cccc4c3-c3ccc(N(c5ccc(C6CCCCC6)cc5)c5ccc6c(c5)C(C)(C)c5ccccc5-6)cc3C4(C)C)cc21 ; CC1(C)c2ccccc2-c2ccc(-c3cccc4c3-c3ccc(N(c5ccc(C6CCCCC6)cc5)c5ccc6c(c5)C(c5ccccc5)(c5ccccc5)c5ccccc5-6)cc3C4(C)C)cc21] 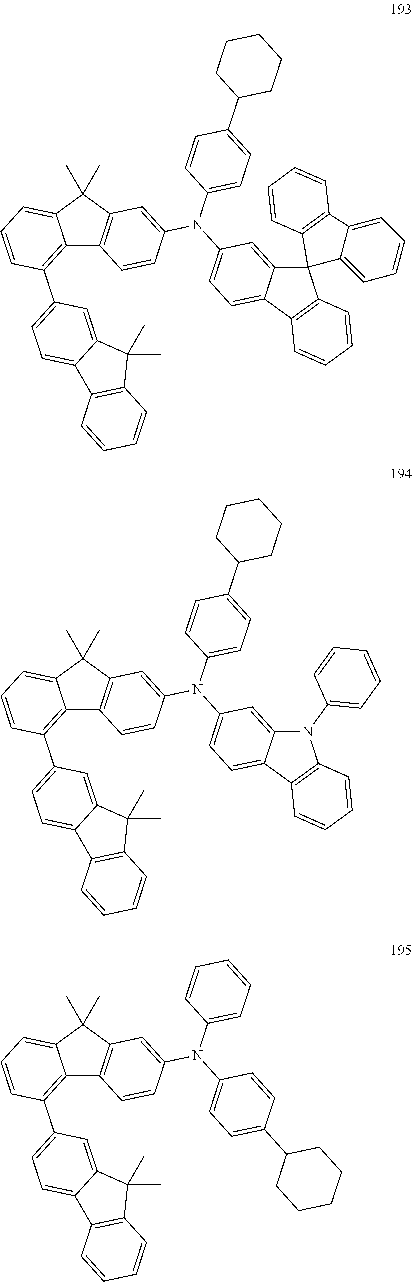 [SMILES: CC1(C)c2ccccc2-c2ccc(-c3cccc4c3-c3ccc(N(c5ccc(C6CCCCC6)cc5)c5ccc6c(c5)C5(c7ccccc7-c7ccccc75)c5ccccc5-6)cc3C4(C)C)cc21 ; CC1(C)c2ccccc2-c2ccc(-c3cccc4c3-c3ccc(N(c5ccc(C6CCCCC6)cc5)c5ccc6c7ccccc7n(-c7ccccc7)c6c5)cc3C4(C)C)cc21 ; CC1(C)c2ccccc2-c2ccc(-c3cccc4c3-c3ccc(N(c5ccccc5)c5ccc(C6CCCCC6)cc5)cc3C4(C)C)cc21] 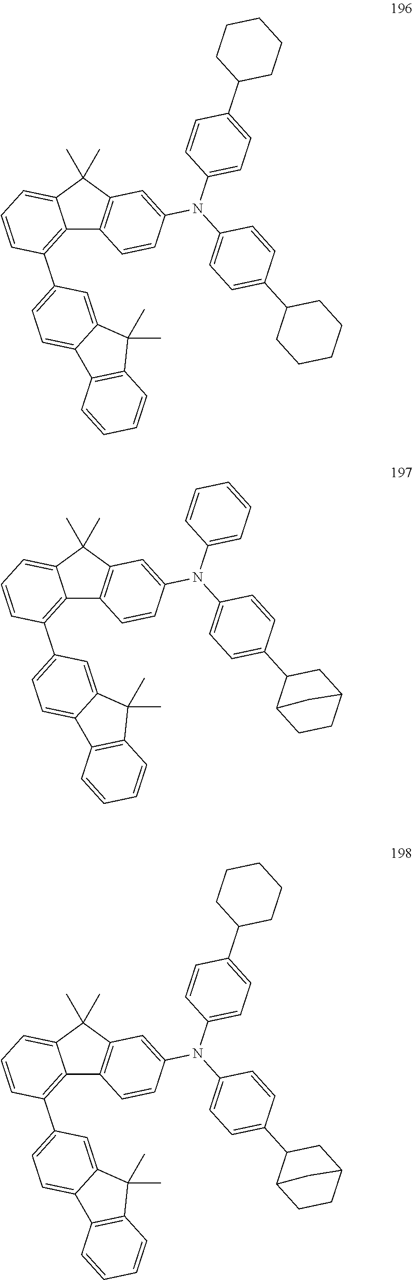 [SMILES: CC1(C)c2ccccc2-c2ccc(-c3cccc4c3-c3ccc(N(c5ccc(C6CCCCC6)cc5)c5ccc(C6CCCCC6)cc5)cc3C4(C)C)cc21 ; CC1(C)c2ccccc2-c2ccc(-c3cccc4c3-c3ccc(N(c5ccccc5)c5ccc(C6CC7CCC6C7)cc5)cc3C4(C)C)cc21 ; CC1(C)c2ccccc2-c2ccc(-c3cccc4c3-c3ccc(N(c5ccc(C6CCCCC6)cc5)c5ccc(C6CC7CCC6C7)cc5)cc3C4(C)C)cc21] 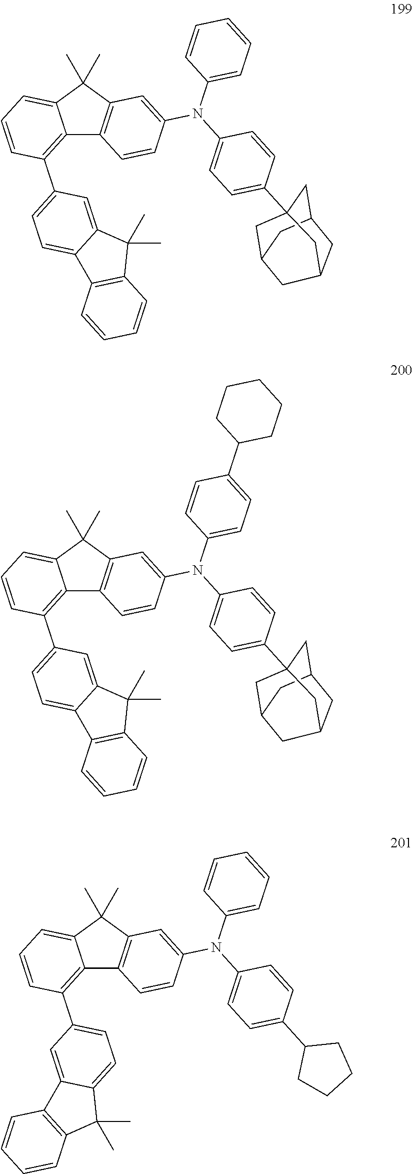 [SMILES: CC1(C)c2ccccc2-c2ccc(-c3cccc4c3-c3ccc(N(c5ccccc5)c5ccc(C67CC8CC(CC(C8)C6)C7)cc5)cc3C4(C)C)cc21 ; CC1(C)c2ccccc2-c2ccc(-c3cccc4c3-c3ccc(N(c5ccc(C6CCCCC6)cc5)c5ccc(C67CC8CC(CC(C8)C6)C7)cc5)cc3C4(C)C)cc21 ; CC1(C)c2ccccc2-c2cc(-c3cccc4c3-c3ccc(N(c5ccccc5)c5ccc(C6CCCC6)cc5)cc3C4(C)C)ccc21] 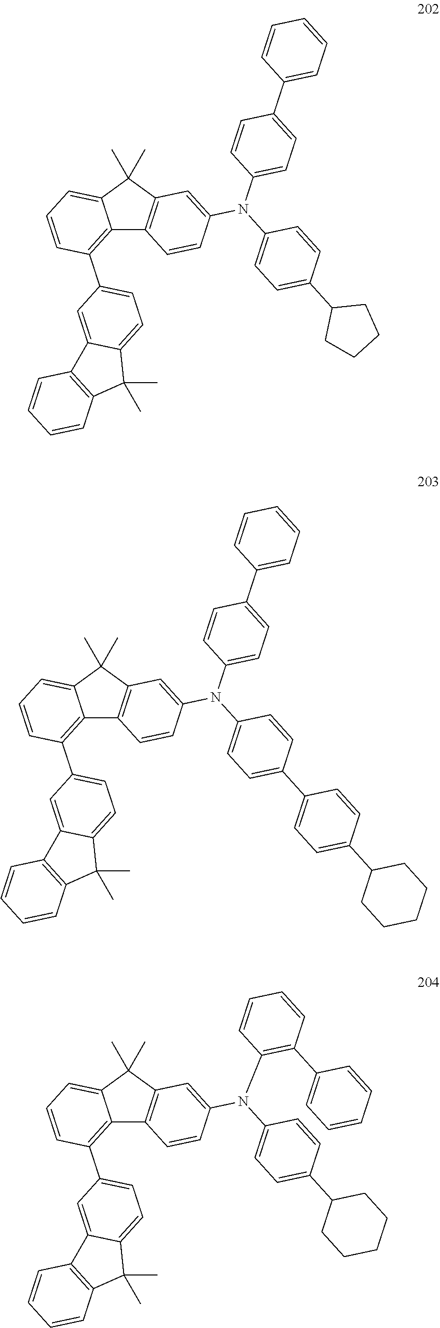 [SMILES: CC1(C)c2ccccc2-c2cc(-c3cccc4c3-c3ccc(N(c5ccc(-c6ccccc6)cc5)c5ccc(C6CCCC6)cc5)cc3C4(C)C)ccc21 ; CC1(C)c2ccccc2-c2cc(-c3cccc4c3-c3ccc(N(c5ccc(-c6ccccc6)cc5)c5ccc(-c6ccc(C7CCCCC7)cc6)cc5)cc3C4(C)C)ccc21 ; CC1(C)c2ccccc2-c2cc(-c3cccc4c3-c3ccc(N(c5ccc(C6CCCCC6)cc5)c5ccccc5-c5ccccc5)cc3C4(C)C)ccc21] 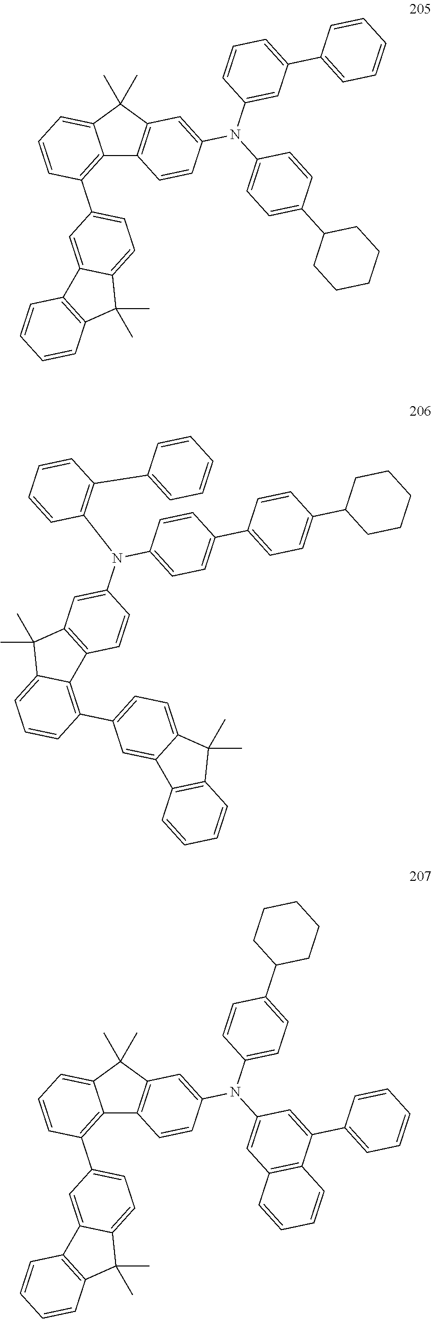 [SMILES: CC1(C)c2ccccc2-c2cc(-c3cccc4c3-c3ccc(N(c5ccc(C6CCCCC6)cc5)c5cccc(-c6ccccc6)c5)cc3C4(C)C)ccc21 ; CC1(C)c2ccccc2-c2cc(-c3cccc4c3-c3ccc(N(c5ccc(-c6ccc(C7CCCCC7)cc6)cc5)c5ccccc5-c5ccccc5)cc3C4(C)C)ccc21 ; CC1(C)c2ccccc2-c2cc(-c3cccc4c3-c3ccc(N(c5ccc(C6CCCCC6)cc5)c5cc(-c6ccccc6)c6ccccc6c5)cc3C4(C)C)ccc21] 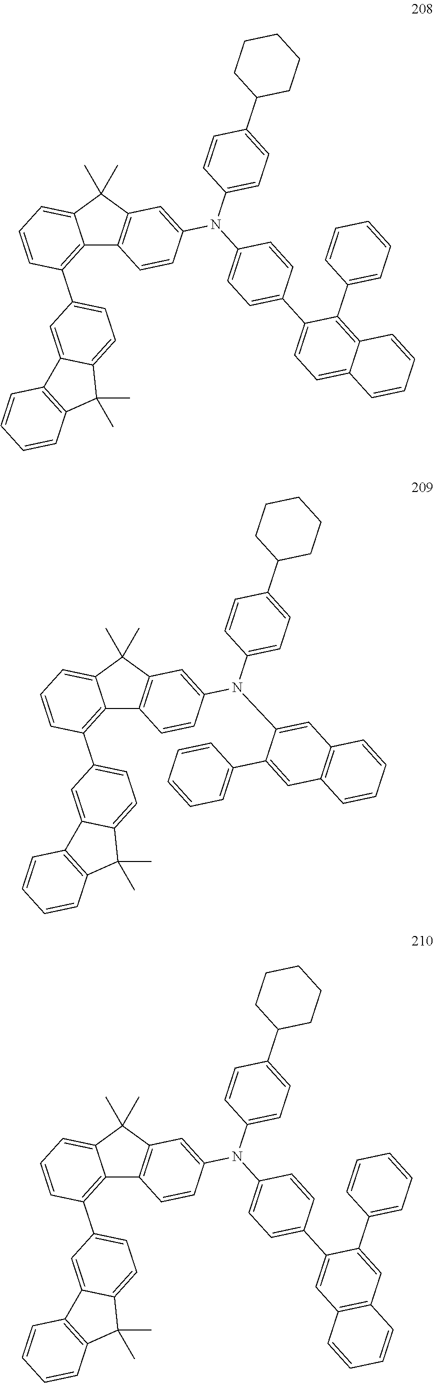 [SMILES: CC1(C)c2ccccc2-c2cc(-c3cccc4c3-c3ccc(N(c5ccc(-c6ccc7ccccc7c6-c6ccccc6)cc5)c5ccc(C6CCCCC6)cc5)cc3C4(C)C)ccc21 ; CC1(C)c2ccccc2-c2cc(-c3cccc4c3-c3ccc(N(c5ccc(C6CCCCC6)cc5)c5cc6ccccc6cc5-c5ccccc5)cc3C4(C)C)ccc21 ; CC1(C)c2ccccc2-c2cc(-c3cccc4c3-c3ccc(N(c5ccc(-c6cc7ccccc7cc6-c6ccccc6)cc5)c5ccc(C6CCCCC6)cc5)cc3C4(C)C)ccc21] 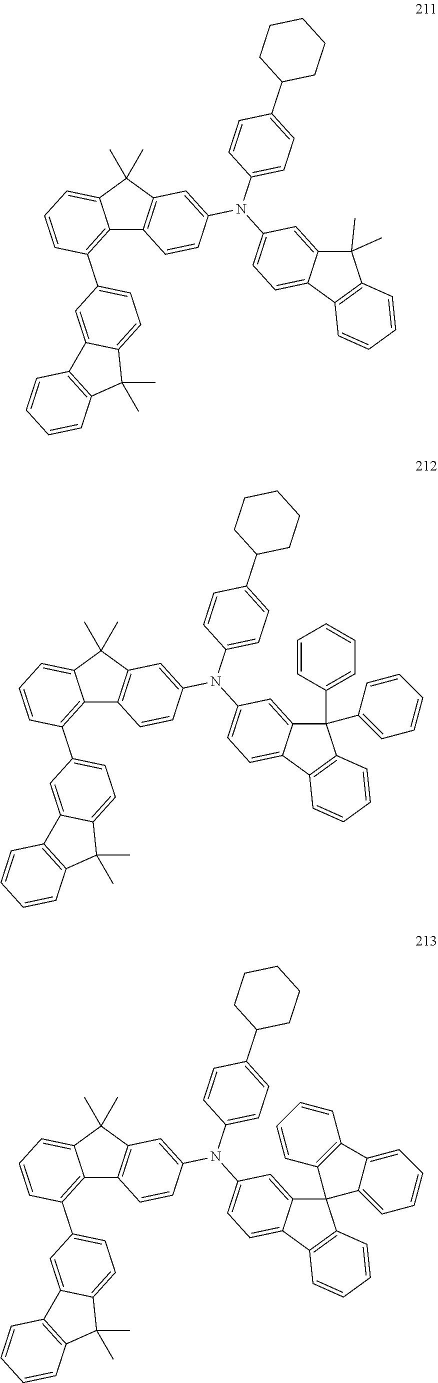 [SMILES: CC1(C)c2ccccc2-c2cc(-c3cccc4c3-c3ccc(N(c5ccc(C6CCCCC6)cc5)c5ccc6c(c5)C(C)(C)c5ccccc5-6)cc3C4(C)C)ccc21 ; CC1(C)c2ccccc2-c2cc(-c3cccc4c3-c3ccc(N(c5ccc(C6CCCCC6)cc5)c5ccc6c(c5)C(c5ccccc5)(c5ccccc5)c5ccccc5-6)cc3C4(C)C)ccc21 ; CC1(C)c2ccccc2-c2cc(-c3cccc4c3-c3ccc(N(c5ccc(C6CCCCC6)cc5)c5ccc6c(c5)C5(c7ccccc7-c7ccccc75)c5ccccc5-6)cc3C4(C)C)ccc21] 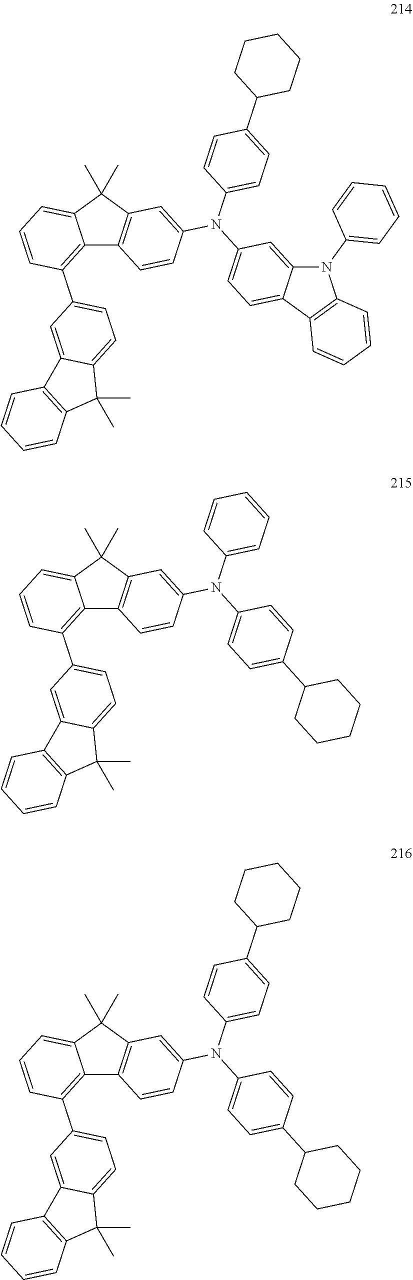 [SMILES: CC1(C)c2ccccc2-c2cc(-c3cccc4c3-c3ccc(N(c5ccc(C6CCCCC6)cc5)c5ccc6c7ccccc7n(-c7ccccc7)c6c5)cc3C4(C)C)ccc21 ; CC1(C)c2ccccc2-c2cc(-c3cccc4c3-c3ccc(N(c5ccccc5)c5ccc(C6CCCCC6)cc5)cc3C4(C)C)ccc21 ; CC1(C)c2ccccc2-c2cc(-c3cccc4c3-c3ccc(N(c5ccc(C6CCCCC6)cc5)c5ccc(C6CCCCC6)cc5)cc3C4(C)C)ccc21] 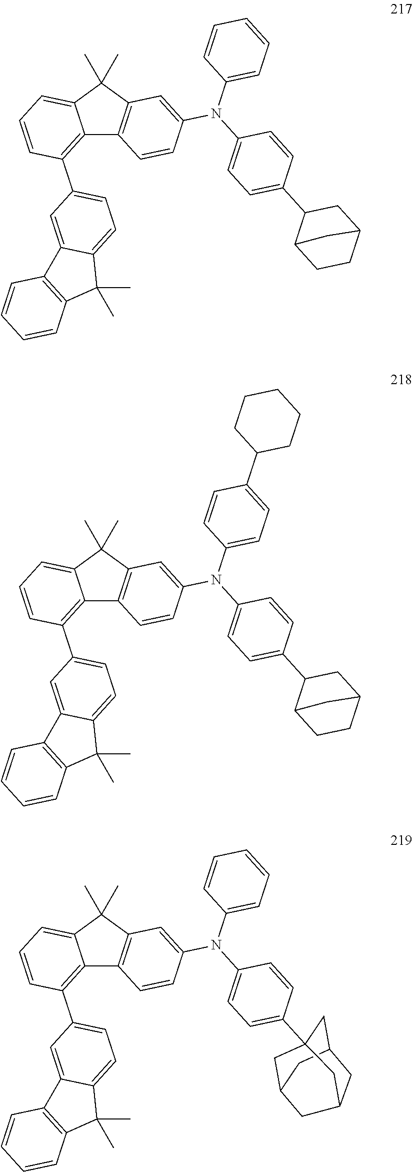 [SMILES: CC1(C)c2ccccc2-c2cc(-c3cccc4c3-c3ccc(N(c5ccccc5)c5ccc(C6CC7CCC6C7)cc5)cc3C4(C)C)ccc21 ; CC1(C)c2ccccc2-c2cc(-c3cccc4c3-c3ccc(N(c5ccc(C6CCCCC6)cc5)c5ccc(C6CC7CCC6C7)cc5)cc3C4(C)C)ccc21 ; CC1(C)c2ccccc2-c2cc(-c3cccc4c3-c3ccc(N(c5ccccc5)c5ccc(C67CC8CC(CC(C8)C6)C7)cc5)cc3C4(C)C)ccc21] 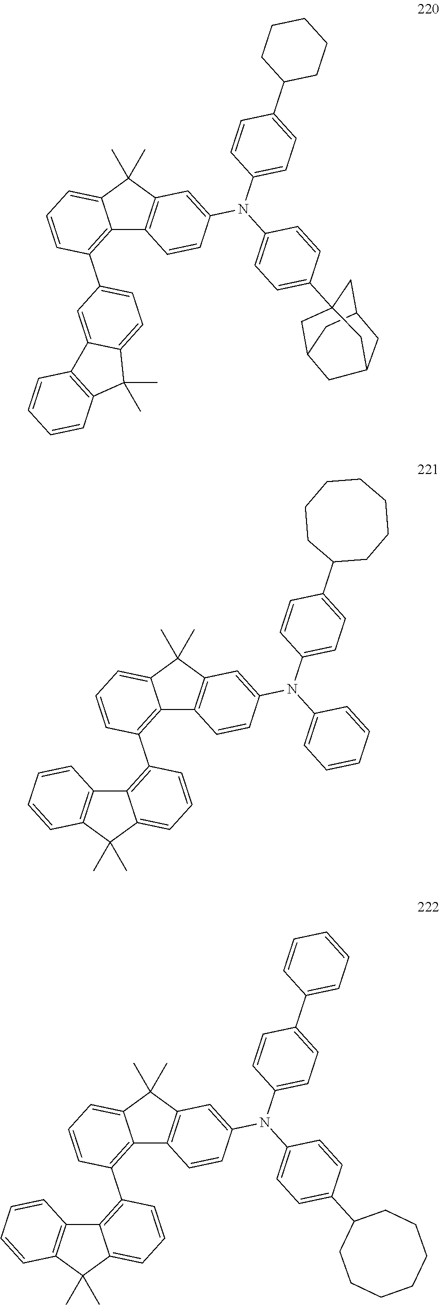 [SMILES: CC1(C)c2ccccc2-c2cc(-c3cccc4c3-c3ccc(N(c5ccc(C6CCCCC6)cc5)c5ccc(C67CC8CC(CC(C8)C6)C7)cc5)cc3C4(C)C)ccc21 ; CC1(C)c2ccccc2-c2c(-c3cccc4c3-c3ccc(N(c5ccccc5)c5ccc(C6CCCCCCC6)cc5)cc3C4(C)C)cccc21 ; CC1(C)c2ccccc2-c2c(-c3cccc4c3-c3ccc(N(c5ccc(-c6ccccc6)cc5)c5ccc(C6CCCCCCC6)cc5)cc3C4(C)C)cccc21] 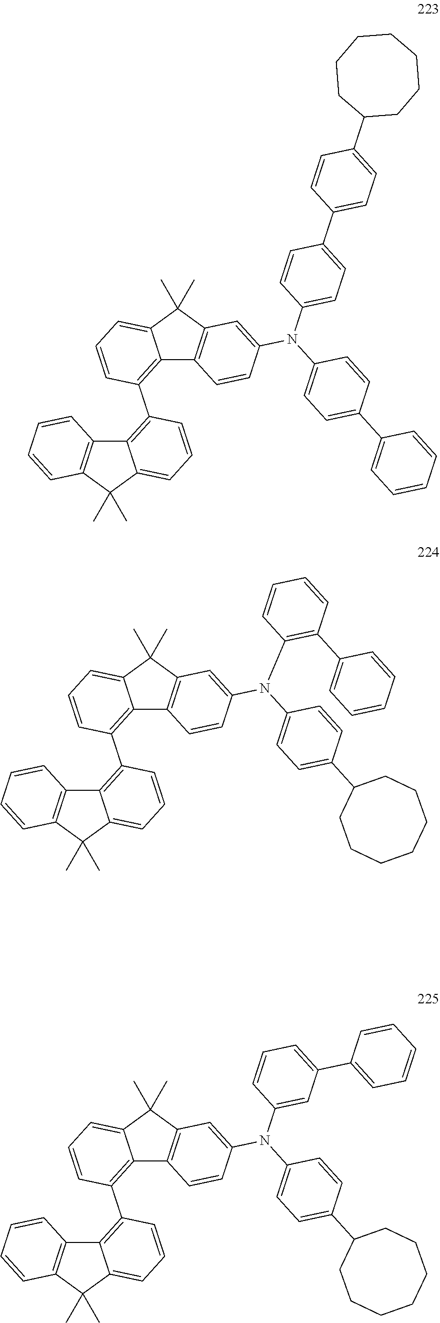 [SMILES: CC1(C)c2ccccc2-c2c(-c3cccc4c3-c3ccc(N(c5ccc(-c6ccccc6)cc5)c5ccc(-c6ccc(C7CCCCCCC7)cc6)cc5)cc3C4(C)C)cccc21 ; CC1(C)c2ccccc2-c2c(-c3cccc4c3-c3ccc(N(c5ccc(C6CCCCCCC6)cc5)c5ccccc5-c5ccccc5)cc3C4(C)C)cccc21 ; CC1(C)c2ccccc2-c2c(-c3cccc4c3-c3ccc(N(c5ccc(C6CCCCCCC6)cc5)c5cccc(-c6ccccc6)c5)cc3C4(C)C)cccc21] 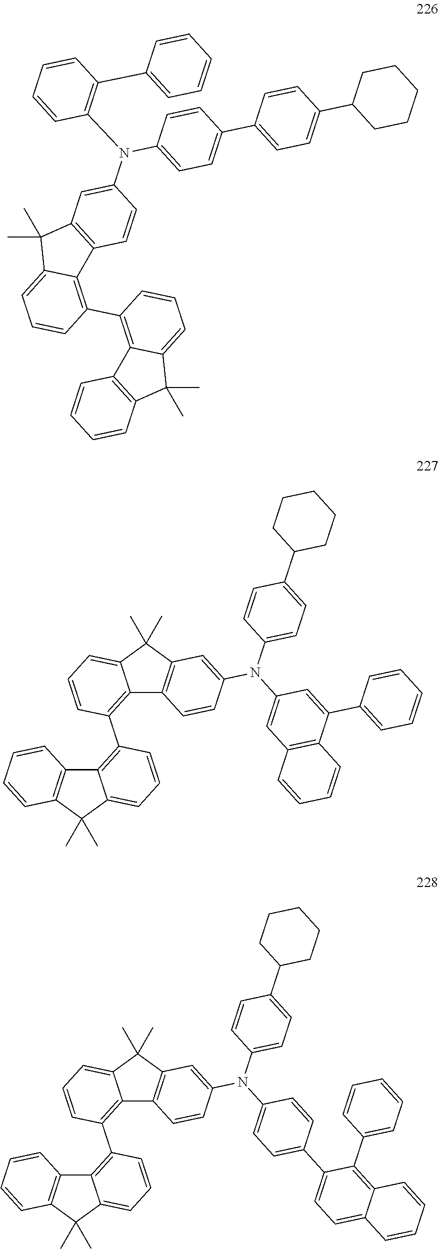 [SMILES: CC1(C)c2ccccc2-c2c(-c3cccc4c3-c3ccc(N(c5ccc(-c6ccc(C7CCCCC7)cc6)cc5)c5ccccc5-c5ccccc5)cc3C4(C)C)cccc21 ; CC1(C)c2ccccc2-c2c(-c3cccc4c3-c3ccc(N(c5ccc(C6CCCCC6)cc5)c5cc(-c6ccccc6)c6ccccc6c5)cc3C4(C)C)cccc21 ; CC1(C)c2ccccc2-c2c(-c3cccc4c3-c3ccc(N(c5ccc(-c6ccc7ccccc7c6-c6ccccc6)cc5)c5ccc(C6CCCCC6)cc5)cc3C4(C)C)cccc21] 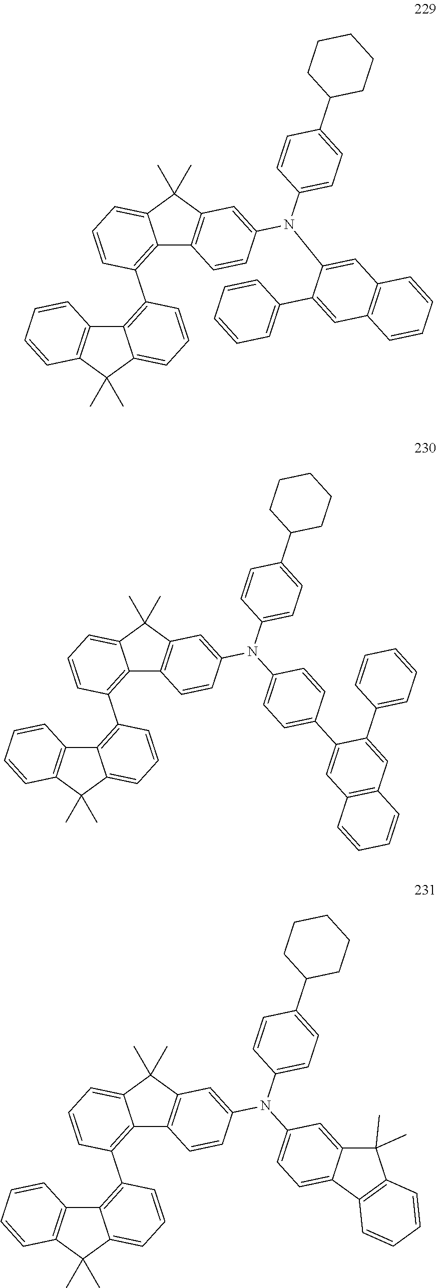 [SMILES: CC1(C)c2ccccc2-c2c(-c3cccc4c3-c3ccc(N(c5ccc(C6CCCCC6)cc5)c5cc6ccccc6cc5-c5ccccc5)cc3C4(C)C)cccc21 ; CC1(C)c2ccccc2-c2c(-c3cccc4c3-c3ccc(N(c5ccc(-c6cc7ccccc7cc6-c6ccccc6)cc5)c5ccc(C6CCCCC6)cc5)cc3C4(C)C)cccc21 ; CC1(C)c2ccccc2-c2ccc(N(c3ccc(C4CCCCC4)cc3)c3ccc4c(c3)C(C)(C)c3cccc(-c5cccc6c5-c5ccccc5C6(C)C)c3-4)cc21] 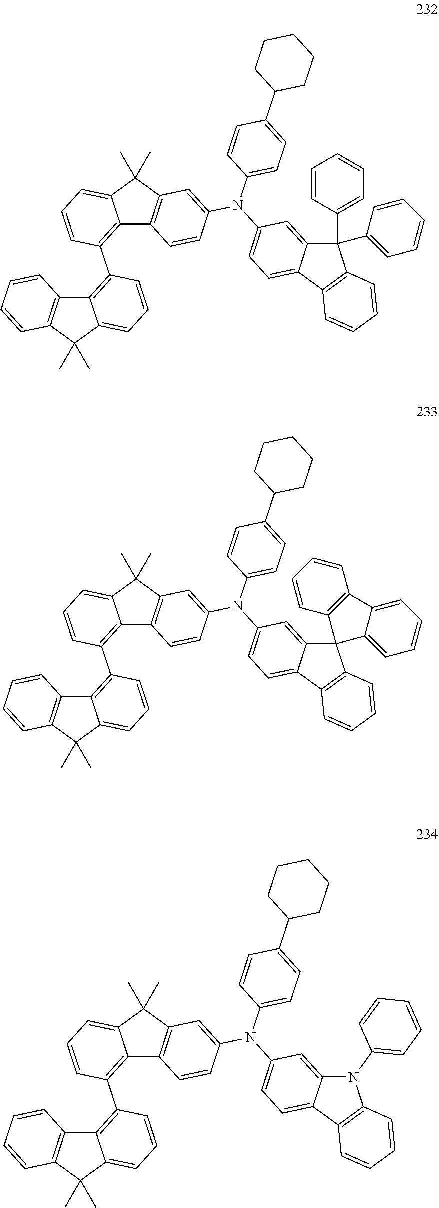 [SMILES: CC1(C)c2ccccc2-c2c(-c3cccc4c3-c3ccc(N(c5ccc(C6CCCCC6)cc5)c5ccc6c(c5)C(c5ccccc5)(c5ccccc5)c5ccccc5-6)cc3C4(C)C)cccc21 ; CC1(C)c2ccccc2-c2c(-c3cccc4c3-c3ccc(N(c5ccc(C6CCCCC6)cc5)c5ccc6c(c5)C5(c7ccccc7-c7ccccc75)c5ccccc5-6)cc3C4(C)C)cccc21 ; CC1(C)c2ccccc2-c2c(-c3cccc4c3-c3ccc(N(c5ccc(C6CCCCC6)cc5)c5ccc6c7ccccc7n(-c7ccccc7)c6c5)cc3C4(C)C)cccc21] 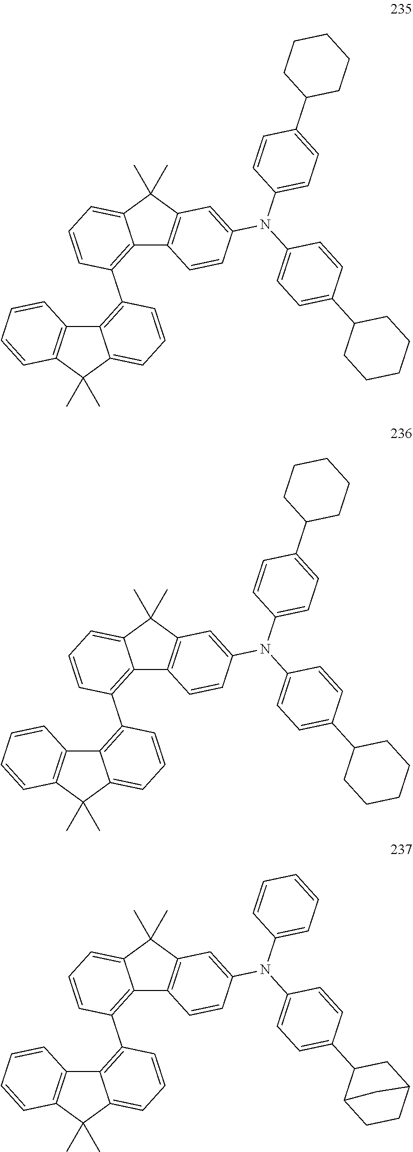 [SMILES: CC1(C)c2ccccc2-c2c(-c3cccc4c3-c3ccc(N(c5ccc(C6CCCCC6)cc5)c5ccc(C6CCCCC6)cc5)cc3C4(C)C)cccc21 ; CC1(C)c2ccccc2-c2c(-c3cccc4c3-c3ccc(N(c5ccc(C6CCCCC6)cc5)c5ccc(C6CCCCC6)cc5)cc3C4(C)C)cccc21 ; CC1(C)c2ccccc2-c2c(-c3cccc4c3-c3ccc(N(c5ccccc5)c5ccc(C6CC7CCC6C7)cc5)cc3C4(C)C)cccc21] 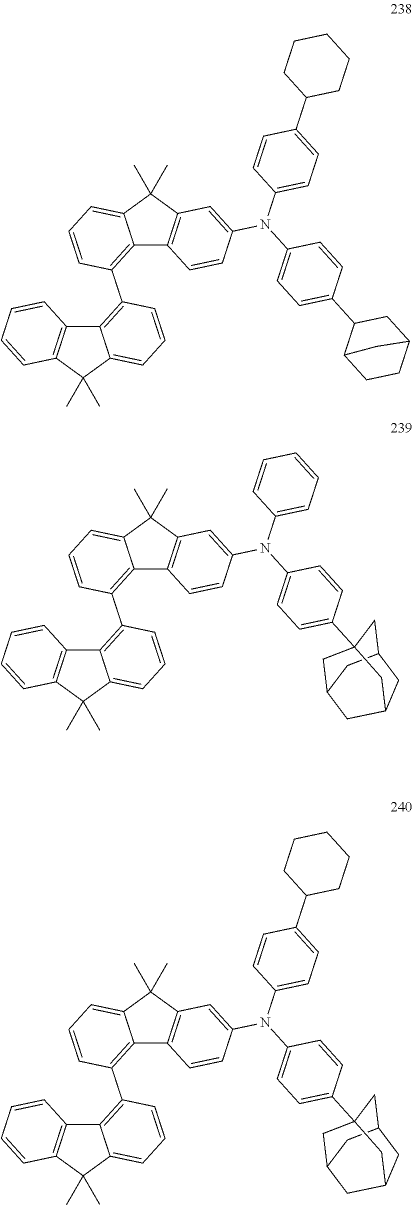 [SMILES: CC1(C)c2ccccc2-c2c(-c3cccc4c3-c3ccc(N(c5ccc(C6CCCCC6)cc5)c5ccc(C6CC7CCC6C7)cc5)cc3C4(C)C)cccc21 ; CC1(C)c2ccccc2-c2c(-c3cccc4c3-c3ccc(N(c5ccccc5)c5ccc(C67CC8CC(CC(C8)C6)C7)cc5)cc3C4(C)C)cccc21 ; CC1(C)c2ccccc2-c2c(-c3cccc4c3-c3ccc(N(c5ccc(C6CCCCC6)cc5)c5ccc(C67CC8CC(CC(C8)C6)C7)cc5)cc3C4(C)C)cccc21]
